(19) United States
(12) Patent Application Publication
(10) Pub. No.: US 2024/0254501 A1
(43) Pub. Date: Aug. 1, 2024

(54) COMPOSITIONS AND METHODS FOR ALTERING FLOWERING AND PLANT ARCHITECTURE TO IMPROVE YIELD POTENTIAL

(71) Applicant: Monsanto Technology LLC, St. Louis, MO (US)

(72) Inventors: Brent BROWER-TOLAND, St. Louis, MO (US); Shunhong DAI, Creve Coeur, MO (US); Karen GABBERT, St. Louis, MO (US); Alexander GOLDSHMIDT, Rehovot (IL); Miya HOWELL, Ballwin, MO (US); Brad MCDILL, Carlsbad, CA (US); Dan OVADYA, Davis, CA (US); Beth SAVIDGE, Davis, CA (US); Vijay SHARMA, Wildwood, MO (US)

(73) Assignee: Monsanto Technology LLC, St. Louis, MO (US)

(21) Appl. No.: 18/584,757

(22) Filed: Feb. 22, 2024

Related U.S. Application Data
(62) Division of application No. 18/059,292, filed on Nov. 28, 2022.
Application No. 18/059,292 is a division of application No. 15/787,359, filed on Oct. 18, 2017, now Pat. No. 11,555,201.
(60) Provisional application No. 62/411,408, filed on Oct. 21, 2016.
(60) Provisional application No. 62/410,355, filed on Oct. 19, 2016.

(51) Int. Cl. C12N 15/82; C07K 14/415

(52) U.S. Cl. CPC C12N 15/827; C07K 14/415; C12N 15/8218; C12N 15/8261; Y02A 40/146

(57) ABSTRACT

The present invention provides recombinant DNA constructs, vectors and molecules useful for attenuating and/or refining the expression of a florigenic FT gene or transgene using targeting sequences of small RNA molecules. Transgenic plants, plant cells and tissues, and plant parts comprising the recombinant constructs, vectors, and molecules are also provided. Transgenic plants comprising a florigenic FT transgene may produce more bolls, siliques, fruits, nuts, or pods per node on the transgenic plant via suppression, relative to a control or wild type plant. Methods are further provided for introducing the recombinant DNA constructs, vectors, and molecules into a plant, and planting transgenic plants in the field including at higher densities. Transgenic plants of the present invention may provide greater yield potential than wild type or control plants.

### CROSS-REFERENCE TO RELATED APPLICATIONS

This application is a divisional of U.S. application Ser. No. 18/059,292, filed Nov. 28, 2022, which is a divisional of U.S. application Ser. No. 15/787,359, filed Oct. 18, 2017, now U.S. Pat. No. 11,555,201, which claims the benefit of priority of U.S. Provisional Application No. 62/410,355, filed Oct. 19, 2016 and U.S. Provisional Application No. 62/411,408, filed Oct. 21, 2016, all of which are incorporated by reference in their entirety.

### FIELD OF THE INVENTION

The present invention relates to compositions and methods for modulating floral development and vegetative growth by genetic modification of crop plants to increase yield.

### REFERENCE TO AN ELECTRONIC SEQUENCE LISTING

The contents of the electronic sequence listing (777052057811SEQLIST.xml; Size: 211,658 bytes; and Date of Creation: Feb. 15, 2024) is herein incorporated by reference in its entirety.

### BACKGROUND

The transition from vegetative growth to flowering is a crucial process during plant development that is necessary for the production of grain yield in crop plants. There are several major pathways controlling flowering time in land plants that respond to environmental or developmental cues, including photoperiodism (i.e., day length), vernalization (i.e., response to winter cold), and plant hormones (e.g., gibberellins or GA), in addition to the autonomous (environmentally independent) pathways. Molecular networks controlling flowering time in plants involve the vernalization and photoperiod pathways. Under inductive photoperiodic conditions, CONSTANS (CO) activity in source leaves increases expression of FLOWERING LOCUS T (FT), which translocates to the meristem to trigger expression of downstream floral activating genes, including LEAFY (LFY), APETALA1 (AP1) and SUPPRESSOR OF OVEREXPRESSION OF CO 1 (SOC1). Other genes, such as FLOWERING LOCUS C (FLC) and TERMINAL FLOWER 1 (TFL1), act to inhibit the expression or activity of these genes.

Except for day length neutral plants, most flowering plants respond to daily photoperiodic cycles and are classified as either short day (SD) or long day (LD) plants based on the photoperiod conditions required to induce flowering. The photoperiod refers to the relative length or duration of light and dark periods within a 24-hour cycle. In general, long day plants tend to flower when the day length exceeds a photoperiod threshold (e.g., as the days are getting longer in the spring), whereas short day plants tend to flower when the day length falls below a photoperiod threshold (e.g., as the days are getting shorter after the summer solstice). In other words, SD plants flower as the days are getting shorter, while LD plants flower as the days are getting longer. Soybean is an example of a short day (SD) plant in which flowering is induced when plants are exposed to shorter daylight conditions.

Plant growers are always looking for new methods to manipulate the yield of a plant, especially to enhance the seed yield of agronomically important crops. Thus, there is a continuing need in the art for improved compositions and methods for increasing yields of various crop plants. It is presently proposed that improved crop yields may be achieved by enhancing agronomic traits related to flowering and reproductive development.

### SUMMARY

According to an aspect, the present disclosure provides a recombinant DNA construct comprising a first expression cassette and a second expression cassette, wherein the first expression cassette comprises a polynucleotide sequence encoding a florigenic FT protein operably linked to a first plant expressible promoter, and the second expression cassette comprises a transcribable DNA sequence encoding a RNA molecule comprising a targeting sequence that is at least 80% complementary to at least 15 consecutive nucleotides of the polynucleotide sequence of the first expression cassette, and wherein the transcribable DNA sequence is operably linked to a second plant expressible promoter.

According to an aspect, the present disclosure provides a recombinant DNA construct comprising a polynucleotide sequence encoding a florigenic FT protein operably linked to a plant expressible promoter, wherein the polynucleotide sequence comprises a sequence that encodes a target site or sensor in a mRNA transcript encoded by the polynucleotide sequence, and wherein the target site of the mRNA transcript is at least 80% complementary to an endogenous RNA molecule, such as an endogenous miRNA or siRNA molecule.

According to an aspect, the present disclosure provides a recombinant DNA construct comprising a transcribable DNA sequence encoding a RNA molecule comprising a targeting sequence that is at least 80% complementary to at least 15 consecutive nucleotides of a polynucleotide sequence encoding a florigenic FT protein, wherein the transcribable DNA sequence is operably linked to plant expressible promoter.

According to an aspect, the present disclosure provides transgenic plants, plant cells, plant tissues, and plant parts comprising an insertion of a recombinant DNA construct of the present disclosure into the genome of such plants, plant cells, plant tissues, and plant parts.

According to an aspect, the present disclosure provides methods for producing a transgenic plant comprising (a) transforming at least one cell of an explant with a recombinant DNA construct of the instant disclosure; and (b) regenerating or developing the transgenic plant from the transformed explant. The methods may further comprise (c) selecting a transgenic plant having one or more of the following traits or phenotypes: earlier flowering, longer reproductive or flowering duration, increased number of flowers per node, increased number of floral racemes per node, increased number of pods, bolls, siliques, fruits, or nuts per node, and increased number of seeds per node, as compared to a control plant not having the recombinant DNA construct.

According to an aspect, the instant disclosure provides methods of planting a transgenic crop plant, comprising planting the transgenic crop plant at a higher density in the field, where the transgenic crop plant comprises an insertion of a recombinant DNA construct of the instant disclosure.

According to an aspect, the present disclosure provides a transgenic plant comprising a polynucleotide sequence encoding a polynucleotide sequence encoding a florigenic FT protein operably linked to a first plant expressible promoter, and the second expression cassette comprises a transcribable DNA sequence encoding a RNA molecule comprising a targeting sequence that is at least 80% complementary to at least 15 consecutive nucleotides of the polynucleotide sequence of the first expression cassette, and wherein the transcribable DNA sequence is operably linked to a second plant expressible promoter. According to an aspect, the present disclosure provides a transgenic plant comprising a transcribable DNA sequence encoding a RNA molecule comprising a targeting sequence that is at least 80% complementary to at least 15 consecutive nucleotides of a polynucleotide sequence encoding a florigenic FT protein, wherein the transcribable DNA sequence is operably linked to plant expressible promoter.

According to an aspect, the present disclosure provides a transgenic plant may have more seeds, pods, bolls, siliques, fruits, nuts or tubers per node on average than a non-transgenic control plant, such as an average of at least 1%, at least 2%, at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more seeds, pods, bolls, siliques, fruits, nuts or tubers per node than a non-transgenic control plant. According to an aspect, a transgenic plant may have an average of at least 2, at least 3, at least 4, at least 5, at least 6, at least 7, at least 8, at least 9, or at least 10 more seeds, pods, bolls, siliques, fruits, nuts or tubers per node than a non-transgenic control plant. According to an aspect, a transgenic plant may have an average of 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, or 4 to 5 more seeds, pods, bolls, siliques, fruits, nuts or tubers per node than a non-transgenic control plant. According to an aspect, a transgenic plant may have an average of at least 1, at least 2, at least 3, at least 4, at least 5, at least 6, at least 7, at least 8, at least 9, or at least 10 more seeds, pods, bolls, siliques, fruits, nuts or tubers per node as compared to a wild type or non-transgenic control plant. According to an aspect, a transgenic plant may flower at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier than a non-transgenic control plant.

According to an aspect, the present disclosure provides a transgenic plant is provided comprising a polynucleotide sequence encoding a florigenic FT protein operably linked to a vegetative stage promoter, wherein expression of the florigenic FT protein is suppressed in a late vegetative and/or reproductive tissue.

According to an aspect, the present disclosure provides a recombinant DNA construct is provided comprising a polynucleotide sequence encoding a florigenic FT protein and operably linked to a vegetative stage promoter, and at least one sequence encoding an RNA targeting sequence that is complementary to at least a portion of the polynucleotide sequence.

According to an aspect, the present disclosure provides a transgenic plant is provided comprising a recombinant polynucleotide sequence encoding a florigenic FT protein operably linked to a vegetative stage promoter, wherein expression of the polynucleotide sequence is spatially and temporally restricted by a small RNA molecule.

According to an aspect, the present disclosure provides a recombinant DNA construct is provided comprising an expression cassette, wherein the expression cassette comprises a polynucleotide sequence encoding a florigenic FT protein operably linked to a promoter, wherein the promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a polynucleotide sequence selected from the group consisting of SEQ ID NOs: 39, 40, 41, 42, 43, 44, 45, 46, 47, 48, 49, 50, 51, 52, 53 or 54, or a functional portion thereof.

### BRIEF DESCRIPTION OF THE DRAWINGS

FIG. 1A provides a matrix table showing a comparison of nucleotide sequences for each combination of the various FT genes including their percent identity.

FIG. 1B provides a matrix table showing a comparison of protein sequences for each combination of the various FT proteins including their percent identity.

FIG. 1C provides a CLUSTAL 2.0.9 multiple sequence alignment of various FT proteins identified as Gm.FT2a with SEQ ID NO: 2, Gm.FT2b with SEQ ID NO: 6, Le.FT with SEQ ID NO: 12, Pt.FT with SEQ ID NO: 20, Os.HD3a with SEQ ID NO: 18, At.FT with SEQ ID NO: 14, At. TSF with SEQ ID NO: 16, Nt.FT with SEQ ID NO: 10, Gm.FT5a with SEQ ID NO: 4 and Zm.ZCN8 with SEQ ID NO: 8.

FIG. 2 shows the total FT transcript levels in soybean leaf and apex tissues collected at 1, 3 and 5 days after either a short day or long day light treatment.

FIGS. 3A to 3O and FIGS. 4A to 4O show the expression pattern of the pAt.Erecta promoter by monitoring GUS activity during early soybean development. FIGS. 3A to 3O are a set of black and white images of stained tissues, and the images in FIGS. 4A to 4O correspond to FIGS. 3A to 3O but are filtered for blue GUS staining. FIGS. 3A to 3C and 4A to 4C show expression in a 3-day-old germinating seedling; FIGS. 3D to 3I and 4D to 4I show expression in a 10-day-old vegetative shoot (grown in 14 hour light/10 hour dark photoperiod); FIGS. 3J to 3L and 4J to 4L show expression in a 16-day-old reproductive shoot; and FIGS. 3M to 3O and 4M to 4O show expression in the 30 d old mature and immature leaves of the reproductive shoot. Bars are 100 μm.

FIGS. 5A to 5F and FIGS. 6A to 6F show the GUS expression pattern with the pAT.Erecta promoter during R1 and floral stages of development (35-40 days after germination). FIGS. 5A to 5F are a set of black and white images of stained tissues, and the images in FIGS. 6A to 6F correspond to FIGS. 5A to 5F but are filtered for blue GUS staining. FIGS. 5A and 6A show expression in the inflorescence stems or pedicels (arrows), and FIGS. 5B and 6B show expression in the floral peduncle (arrows). Expression is also shown in the vasculature and parenchyma cells (FIGS. 5C and 6C), in stamen filaments (FIGS. 5D and 6D; arrow), and un-pollinated ovules (FIGS. 5E, 5F, 6E and 6F; arrows). Bars are 1 mm.

FIG. 7 shows section imaging of the shoot apical meristem (SAM) from wild type versus GmFT2a-expressing transgenic plants at 7 days after planting using scanning electron microscopy (eSEM) analysis.

FIG. 8 shows scanning electron microscopy (eSEM) micrographs of an axillary inflorescence primordia from a wild type plant (collected at 27 days after planting), in comparison to an axillary inflorescence primordia from a transgenic event expressing Gm.FT2a (collected at 9 days after planting).

FIGS. 9A to 9C show the effects of Gm.FT2a expression driven by the At.Erecta promoter in soybean. FIG. 9A depicts a null segregant showing normal axillary buds, whereas FIG. 9B and FIG. 9C (corresponding to plants homozygous or hemizygous for the Gm.FT2a transgene, respectively) each show early flowering and increased pods per node relative to the null segregant.

FIG. 10 shows a whole plant image of a wild type null segregant next to plants hemizygous and homozygous for the Gm.FT2a transgene as indicated.

FIG. 11 shows images of the main stem of plants that are homozygous or hemizygous for the pAt.Erecta-Gm.FT2a transgene in comparison to a null segregant as indicated.

FIG. 12 shows whole plant images of plants transformed with either pAt.Erecta::Gm.FT2a or pAt.Erecta::Gm.FT2a/pAP1::miRNA-FT2a::T-Apx.

FIG. 13 shows whole plant images of plants transformed with either pAt.Erecta::Gm.FT2a alone or pAt.Erecta::Gm.FT2a+miR172 target site.

### DETAILED DESCRIPTION

The goal of improving yield is common to all crops across agriculture. The present invention includes methods and compositions for improving yield in flowering (angiosperm) or seed-bearing plants by modification of traits associated with flowering time, reproductive development, and vegetative growth to improve one or more flowering and/or yield-related traits or phenotypes, such as the number of flowers, seeds and/or pods per plant, and/or the number of flowers, seeds and/or pods per node (and/or per main stem) of the plant. Without being bound by any theory, compositions and methods of the present invention may operate to improve yield of a plant by increasing the number of floral meristems, increasing synchronization of lateral meristem release, and/or extending the time period for pod or seed development in the plant (e.g., reproductive duration).

Previously, it was discovered that growing short day plants, such as soybean, under long day conditions (e.g., about 14-16 hours of light per day) and then briefly subjecting those plants to short day growing conditions (e.g., about 9-11 hours of light per day for about 3-21 days) before returning the plants to long day (non-inductive) growing conditions, produced plants having increased numbers of pods/seeds per plant (and pods/seeds per node and/or per branch). See, e.g., U.S. Pat. No. 8,935,880 and U.S. Patent Application Publication No. 2014/0259905, the entire contents and disclosures of which are incorporated herein by reference. The artificial early “short day” inductive light treatments during vegetative stages of development revealed not only that flowering time could be altered in a way that alters one or more yield-related traits or phenotypes (e.g., by causing an increased number of pods or seeds per node on a plant), but also that the effect of these treatments was dosage-dependent with the number of flowers, seeds and/or pods per plant (and/or per node of the plant) depending on (i) the duration of the short day exposure (i.e., floral induction signal dosage) and (ii) the length of the post-short day photoperiods under long day conditions (i.e., the dosage or length of the vegetative growth inducing signal after the short day induction signal). Soybean plants experiencing a lower or less prolonged early short day induction (eSDI) treatment (prior to returning to long day growing conditions) had more flowers, pods and seeds per plant with more normal plant height and maturity, whereas soybean plants exposed to a greater or more prolonged eSDI treatment produced shorter, earlier-terminating plants with fewer pods and seeds per plant (albeit perhaps with an increased number of pods and/or seeds per node).

This short day induction phenotype in soybean was used to identify genes having altered expression in these plants through transcriptional profiling. These studies identified several genes with altered expression in these treated soybean plants including an endogenous FT gene, Gm.FT2a, having increased expression in response to the short day induction treatment. Thus, it is proposed that transgenic FT expression may be used in place of short day induction to increase seed yield, alter reproductive traits or phenotypes in plants, or both. Ectopic expression of a Gm.FT2a transgene or other FT sequence, or a functional fragment, homolog or ortholog thereof, in a flowering or seed-bearing plant may be used to increase seed yield and/or alter one or more reproductive phenotypes or traits, which may involve an increase in the number of pods/seeds per plant (and/or the number of pods/seeds per node or main stem of the plant). As explained further below and depending on the particular plant species, these yield-related or reproductive phenotypes or traits may also apply to other botanical structures analogous to pods of leguminous plants, such as bolls, siliques, fruits, nuts, tubers, etc. Thus, a plant ectopically expressing a FT sequence may instead have an increased number of bolls, siliques, fruits, nuts, tubers, etc., per node(s), main stem, and/or branch(es) of the plant, and/or an increased number of bolls, siliques, fruits, nuts, tubers, etc., per plant.

Flowering Locus T (FT) genes play a key role in higher plants and function to integrate floral pathways. FT proteins have been shown to function as a mobile signal or florigen transported from leaves to the shoot apical apex where it triggers initiation of reproductive development in diverse species. See, e.g., Jaeger, K. E. et al., “Interlocking feedback loops govern the dynamic behavior of the floral transition in Arabidopsis,” The Plant Cell, 25:820-833 (2013); Corbesier, L et al., “FT protein movement contributes to long distance signaling in floral induction of Arabidopsis,” Science 316: 1030-1033 (2007); Jaeger, K E et al., “FT protein acts as a long range signal in Arabidopsis,” Curr Biol 17: 1050-1054 (2007); and Amasino, R. M. et al., “The Timing of Flowering,” Plant Physiology, 154(2):516-520 (2010), the entire contents and disclosures of which are incorporated herein by reference. In Arabidopsis, FT protein binds to 14-3-3 and Flowering Locus D (FD) proteins in the meristem to form a flowering complex triggering activation of key floral meristem identity genes, such as APETATAL1 (AP1) and SOC1 at the shoot apex. See, e.g., Taoka, K. et al., “14-3-3 protein act as intracellular receptors for rice Hd3a florigen.” Nature 476:332-335 (2011). The TERMINAL FLOWER 1 (TFL1) gene is a key repressor of FT targets that maintains the center of the shoot apical meristem (SAM) in a vegetative state. TFL1 acts by repressing the LEAFY (LFY) and AP1 genes. Thus, the relative concentrations of FT and TFL1 in the target tissues act competitively to control the timing of the reproductive transition of meristems from a vegetative state that may terminate further vegetative growth. See, e.g., Abe, M et al., Science 309:1052-1055 (2005); and McGarry, R C et al., Plant Science 188/189: 71-81 (2012).

FT genes have been identified from many diverse species, and ectopic FT expression has been reported to induce early flowering. See, e.g., Kong, F. et al., “Two Coordinately Regulated Homologs of Flowering Locus T Are Involved in the Control of Photoperiodic Flowering in Soybean,” Plant Physiology 154: 1220-1231 (2010); Turck, F. et al., “Regulation and identity of florigen: Flowering Locus T moves center stage,” Ann Rev Plant Biol 59: 573-594 (2008); Blackman, B K et al., “The role of recently derived FT paralogs in sunflower domestication,” Curr Biol 20: 629-635 (2010); Lifschitz, E. et al., “The tomato FT orthologs triggers systemic signals that regulate growth and flowering and substitute for diverse environmental stimuli,” PNAS 103: 6398-6403 (2006); Trankner, C. et al., “Over-expression of an FT-homologous gene of apple induces early flowering in annual and perennial plants,” Planta 232: 1309-1324 (2010); and Xiang, L. et al., “Functional analysis of Flowering Locus T orthologs from spring orchid (Cymbidium goeringii Rchb. f.) that regulates the vegetative to reproductive transition,” Plant Cell & Biochem 58: 98-105 (2012), the entire contents and disclosures of which are incorporated herein by reference. However, prior studies with expression of FT transgenes used constitutive or tissue specific promoters that produced either very severe phenotypes, non-cell autonomous (systemic) phenotypes, or autonomous leaf specific phenotypes with plants or seedlings flowering earlier than controls and terminating at early stages of development. Given these findings, ectopic FT expression was generally not seen as a viable approach to increasing yield in plants by inducing flowers or altering flowering time.

Without being bound by theory, an early florigenic signal (e.g., short days for soybean and other SD plants) may trigger an early vegetative to reproductive transition in plants but may also cause termination of a subset of its primary meristems. However, by returning those plants to non-inductive growth conditions (e.g., long days for SD plants) after the initial SD signal, the remaining meristematic reserves of the plant may be preserved to allow for continued vegetative growth of the plant. Thus, a greater number of productive flowers, pods and/or seeds per node (and/or per plant) may develop during the extended reproductive phase. With early floral induction, a greater overlap may also be created between reproductive development and vegetative growth of the plant, which may further promote or coincide with an extended reproductive and/or flowering duration. As used herein, “reproductive duration” refers to the length of time from the initiation of flowering until the end of seed/pod development and/or filling, whereas “flowering duration” or “duration of flowering” refers the length of time from the appearance of the first open flower until the last open flower closes. By returning to non-inductive growth conditions after early floral induction, more abundant resources may be available and directed toward the production of an increased number of earlier synchronized and successful (i.e., non-aborting) flowers, pods and/or seeds per plant, unlike normal floral development in short day plants, which may later coincide with declining plant resources due to termination of meristematic growth and maturation of the plant.

As mentioned above, however, a floral induction signal (e.g., early short day conditions) may also cause early termination of the plant in addition to early flowering. Therefore, it is proposed that an optimal dosage and timing of the floral induction signal may be needed to maximize yield by balancing (i) the early vegetative to reproductive transition and/or synchronization of flowering with the early floral induction signal (leading to potential yield gains at each node of the plant) against (ii) earlier growth termination (leading to smaller plants with fewer internodes, less branching, and fewer nodes and/or flowers per plant). Lower dosages of a floral induction signal may be sufficient to induce flowering while lessening or minimizing earlier termination of the plant, such that larger plants are produced with increased numbers of flowers, pods and/or seeds per node (and/or per plant). On the other hand, higher dosages of a floral induction signal may cause early termination of the plant (in addition to early flowering) to produce smaller plants with relatively fewer numbers of flowers, pods and/or seeds per plant due to the smaller plant size with fewer internodes and/or branches per plant, despite having perhaps a greater number of flowers, pods and/or seeds per node (and/or per plant) relative to wild-type or control plants under normal growth conditions. As stated above, these effects of ectopic FT expression may also include an increased number of bolls, siliques, fruits, nuts, tubers, etc., per node (and/or per plant), depending on the particular plant species.

The “short day” light induction phenotype mentioned above in soybean was used to screen for genes having altered expression in those plants through transcriptional profiling, which led to the identification of an endogenous FT gene, Gm.FT2a, having increased expression in response to the short day induction treatment. Accordingly, it is proposed that expression of a florigenic FT transgene, such as Gm.FT2a, may be used as a floral induction signal to cause early flowering and increased flowers, pods and/or seeds per node (and/or per plant) relative to a wild type or control plant not having the FT transgene. According to embodiments of the present invention, appropriate control of the timing, location and dosage of florigenic FT expression during vegetative stages of development can be used to induce flowering and produce plants having increased flowers, pods and/or seeds per node relative to a wild type or control plant not having the FT transgene. Instead of the eSDI light treatment, FT may be expressed at a low level in the vegetative meristem to provide the early floral induction signal. Accordingly, a promoter from the Erecta gene (pErecta or pEr) having lower meristematic expression during vegetative stages of development was selected for initial testing with a Gm.FT2a transgene. However, given that prior studies showed that constitutive FT expression produced plants having a severe, early termination phenotype, and further that the site of action for FT produced peripherally and translocated from the leaves is in the meristem, it was possible that direct meristematic expression of FT could produce even more potent and severe phenotypes (and/or non-viable plants) relative to constitutive FT expression.

The effects of Gm.FT2a overexpression with the pErecta promoter were immediately seen in R₀ transformed soybean plants, which had early flowering, reduced seed yield (e.g., only about 8 seeds/plant), and very early termination, suggesting that the balance between floral induction and floral repression/vegetative growth was strongly in favor of flowering and early termination. However, enough R1 seed was salvaged from these plants to allow for additional experiments to be performed. It was proposed that growing the R₁ soybean seed under long day (floral repressive) photoperiod conditions in the greenhouse might delay the early flowering and termination phenotypes observed in the R₀ plants. Given the theorized dosage response, it was further proposed that segregating FT2a homozygous, hemizygous and null soybean plants could be tested together in the greenhouse to evaluate the dosage response resulting from FT overexpression. In these experiments (as described further below), it was observed that segregating plants did have different phenotypes: null plants were similar to wild-type plants in terms of plant architecture and pods per node (and per plant), while homozygous plants terminated early with a severe dwarf phenotype (although possibly with an increased number of pods per node). However, hemizygous plants were larger and more similar to null or wild-type plants but exhibited the increased flowering phenotype with an increased number of pods per node (and/or per plant). These findings show that vegetative stage and/or meristematic expression of a florigenic FT transgene may be used to produce a high yielding plant (similar to the eSDI treatment), and that the effect of FT expression may be dosage-dependent since soybean plants hemizygous for the FT2a transgene under the control of a weak meristematic promoter displayed the high yield phenotype of increased pods per node without the more severe early termination and short plant height phenotypes observed with homozygous FT2a plants when grown under long day (vegetative) conditions.

Accordingly, vegetative stage expression of an FT transgene at the appropriate dosage level may be used to induce early flowering and produce plants having increased flowers, pods bolls, siliques, fruits, nuts, tubers, and/or seeds per node relative to a wild type or control plant not having the FT transgene. The appropriate dosage level of FT may be achieved based on the promoter selected to drive expression of the FT transgene. A weaker or lower expression level of the FT transgene in the vegetative meristem may be used to provide the early floral induction signal while maintaining or prolonging duration of reproductive and/or floral development and not causing termination of plant development to occur too early. Again, the promoter from the Erecta gene (pErecta or pEr) having low meristematic expression during vegetative stages of development was selected for initial testing with a Gm.FT2a transgene in soybean plants. As described further below, other promoters with similar patterns and levels of expression in the vegetative meristem had similar effects in soybean plants, such as early flowering and/or increased pods per node. Separately or in addition to promoter selection to affect the expression level of the transgene, different FT transgenes from soybean or other species may be used, which may reduce the “dosage” of the early FT signal being delivered to the vegetative meristem depending on the level of activity of the transgenic FT protein in a plant cell. Indeed, several FT transgenes from soybean and other plant species were tested and shown to have variable effects on flowering and other reproductive traits when transgenically expressed in soybean plants.

Without being bound by theory, it is further proposed that increased numbers of pods per node in transgenic FT plants may result at least in part from an increase in the number of inflorescence and floral meristems induced from vegetative shoot apical and axillary meristems at each of the affected node(s), which may give rise to a greater number of flowers and/or released floral racemes at those node(s). Such an increase in the number of floral meristems induced at each node of the plant in response to FT overexpression may operate through one or more mechanisms or pathways, which may be independent of flowering time and/or reproductive duration. However, meristematic changes may be microscopic at first, and thus not observed to cause “early flowering” at such stage by simple visual inspection even though reproductive changes to the meristem may have already begun to occur. Early vegetative FT expression may cause more reproductive meristems to form and develop earlier than normal at one or more node(s) of the transgenic plant. These reproductive meristems may then allow or cause a greater number of floral racemes to form and elongate with flowers at each node. Without being bound by theory, it is further theorized that later expression of FT during reproductive stages may function to repress further floral development at each node. Thus, later developing flowers within the respective raceme may become terminated, and thus more of the plant's resources may be directed to the earlier developing flowers and reproductive structures within the raceme to more effectively produce full-sized pods. The early floral induction signal may also cause a greater proportion of the existing meristem potential to become reproductive and undergo floral development. Accordingly, increased synchronization of floral development may occur with a greater number of mature pods being formed per node of the plant.

As mentioned above, however, the floral induction signal also causes earlier termination of plant development in addition to early flowering. Although soybean plants expressing various FT transgene(s) with a vegetative stage meristem promoter, such as the pErecta promoter, have increased pods per node on the main stem of the plant, many of these transgenic FT plants still exhibit reduced plant height and/or branching, leading to fewer nodes per plant, main stem and/or branch(es). Thus, while the level of FT expression in the plant may be controlled by selection of a particular vegetative stage promoter to mitigate developmental off types and enhance yield by delaying early termination, transgenic FT-expressing soybean plants may still have a reduced number of flowering nodes per plant, which may decrease the overall yield of the plant, despite the increased number of pods per existing node on the main stem.

It is therefore presently proposed that despite the increased pods per node observed in soybean plants with vegetative stage FT expression, the level of FT transgene expression may need to be further attenuated, controlled or limited to mitigate these early termination phenotypes, and thus achieve a more optimal yield. If the increased number of pods (or other botanical structures, such as bolls, siliques, fruits, nuts, tubers, etc.) per node of the plant can be maintained while increasing the number of nodes per plant, such as by avoiding or delaying early termination, then the overall yield of the plant may be further optimized or improved.

According to embodiments of the present invention, it is presently proposed that plant yield may be increased or enhanced by attenuating or modifying the level and/or timing of FT transgene expression through suppression. As described further below, the amount and/or spatiotemporal pattern of transgenic FT may be reduced and/or refined via naturally occurring and/or artificially created RNA molecules that target the transgenic FT for suppression. It is theorized that while transgenic FT expression in axillary and apical meristems may initiate their transition into floral meristems, continued FT expression, such as during reproductive stages of development, may cause early termination of meristems and a stunting of overall plant height and branching. Although a vegetative stage promoter to express the FT transgene may be selected and used to lessen the early termination phenotype and maintain or prolong vegetative growth and reproductive duration of the plant, additional suppression of the FT transgene may further mitigate the early termination phenotype and improve or enhance the growth, development and reproductive duration of the plant. Indeed, the present inventors have observed that suppression of the FT transgene in soybean can result in a more normal plant height and branching with a greater number of nodes per plant, relative to transgenic FT expression alone (i.e., without suppression). These soybean plants with the combined expression/suppression of the FT transgene still maintain a higher number of pods per node while further mitigating the earlier termination phenotype with transgenic FT expression alone.

Suppression of an FT transgene may be achieved in different ways. According to a first approach, the FT transgene of a first expression cassette may be suppressed by a second expression cassette encoding a RNA molecule that targets the FT transgene for suppression. The RNA molecule may be encoded by a transcribable DNA sequence operably linked to a plant expressible promoter, wherein the transcribable DNA sequence comprises a targeting sequence that corresponds to at least a portion of the FT transgene, and/or to a sequence complementary thereto. According to a second approach, an FT transgene may encode a target site for an endogenous RNA molecule, wherein the target site is complementary to the endogenous RNA molecule, such that the endogenous RNA molecule targets the FT transgene for suppression. The endogenous RNA molecule may be naturally occurring in the plant cell in which the FT transgene is expressed. According to this approach, a second expression cassette may not be needed for suppression. Both of these suppression approaches may also be used together. For example, an FT transgene may be present in a first expression cassette and used in combination with a second expression cassette comprising a transcribable DNA sequence encoding a RNA molecule comprising a targeting sequence that corresponds to at least a portion of the FT transgene (and/or a sequence complementary thereto) (i.e., a first target site), such that the RNA molecule targets the FT transgene for suppression, and wherein the FT transgene further comprises a second target site for an endogenous RNA molecule that also targets the FT transgene for suppression. The first and second target sites may be the same or different in sequence and may be present at the same or different location(s) within the FT transcript. As used herein, a first polynucleotide sequence or molecule “corresponds” to a second polynucleotide sequence or molecule if the first sequence or molecule is similar, identical and/or complementary to the second sequence or molecule, such as greater than 30%, greater than 40%, greater than 50%, greater than 60%, greater than 70%, greater than 80%, greater than 90%, greater than 95% or 100% identical and/or complementary.

According to an aspect of the present invention, a recombinant DNA molecule, vector or construct is provided comprising at least two expression cassettes including a first expression cassette and a second expression cassette, wherein the first expression cassette comprises a polynucleotide sequence encoding a FT protein (i.e., an FT transgene) operably linked to a first plant expressible promoter, and wherein the second expression cassette comprises a transcribable DNA sequence operably linked to a second plant expressible promoter, wherein the transcribable DNA sequence comprises a sequence that corresponds to at least a portion of the FT transgene. The transcribable DNA sequence may encode a RNA molecule comprising a targeting sequence that is complementary to at least a portion of the pre-mRNA or mature mRNA encoded by the polynucleotide sequence encoding the FT protein (i.e., the FT transgene) of the first expression cassette, such that the RNA molecule functions to suppress the FT transgene. The “targeting sequence” of the RNA molecule encoded by the transcribable DNA sequence may comprise all or a portion of the RNA molecule and is encoded by the sequence of the transcribable DNA sequence that corresponds to at least a portion of the FT transgene and/or to a sequence complementary thereto. Thus, a transcribable DNA sequence may encode a RNA molecule that comprises a targeting sequence that is complementary to at least a portion of an mRNA transcript of a FT transgene. Depending on the particular FT transgene and/or targeting sequence of the RNA molecule encoded by the transcribable DNA sequence, an endogenous FT gene may also be suppressed by the second expression cassette in addition to the FT transgene, or an endogenous FT gene may be suppressed by the second expression cassette instead of the FT transgene. Many of the FT gene and protein sequences may be identical or similar to one or more native or endogenous FT gene(s) in a plant being transformed, and thus can serve as a basis for designing RNA molecules and targeting sequences for suppression of those native and endogenous gene(s). Whether a transcribable DNA sequences encodes a RNA molecule that targets and suppresses an FT transgene, an endogenous FT gene, or both, the total expression level and activity of FT genes and transgenes may be controlled, limited or reduced in one or more tissues of a transgenic plant. In one aspect, a nucleic acid molecule comprising a targeting sequence is capable of hybridizing to a complementary nucleic acid sequence (e.g., a target site) to form a double-stranded nucleic acid (e.g., dsRNA). In one aspect, hybridization of a targeting sequence of a first nucleic acid molecule (e.g., a suppression RNA molecule) to a target site sequence of a second nucleic acid molecule (e.g., an mRNA transcript of an FT transgene) can lead to the suppression of the second nucleic acid molecule. For example, if the polynucleotide sequence of the FT transgene is native to the plant in which it is expressed (or closely related), then the RNA molecule may further target the corresponding native FT gene for suppression, especially if the RNA molecule targets a coding (exon) sequence of the FT transgene. In such cases, the combined suppression of the native and transgenic FT genes via the RNA molecule encoded by the transcribable DNA sequence may further reduce the dosage of FT protein in relevant tissues of the plant. However, even with transgenic expression of a native FT gene, the non-protein coding sequences, such as the 5′ UTR, 3′ UTR, leader, and/or intron sequence(s), can be varied without affecting the sequence of the encoded FT protein. As used herein, a “polynucleotide coding sequence” or “polynucleotide sequence” of a transgene may comprise not only the protein coding (or exon) sequence(s) but also any other transcribable sequences associated with the coding sequence of the transgene that might form part of the encoded pre-mRNA or mature mRNA sequence, such as a 5′ UTR, 3′ UTR, leader, and/or intron sequence(s). Thus, as used herein, a “polynucleotide coding sequence” of an FT transgene and a “polynucleotide sequence” encoding an FT protein may be used interchangeably.

According to embodiments of the present invention, the first expression cassette and the second expression cassette may be present in the same recombinant DNA molecule, vector or construct, or the first expression cassette and the second expression cassette may be present in separate recombinant DNA molecules, vectors and/or constructs. Thus, according to some embodiments, two recombinant DNA molecules, vectors or constructs may be provided comprising a first recombinant DNA molecule, vector or construct and a second recombinant DNA molecule, vector or construct, wherein the first recombinant DNA molecule, vector or construct comprises a first expression cassette comprising a polynucleotide sequence encoding a FT protein (i.e., a FT transgene) operably linked to a first plant expressible promoter, and wherein the second recombinant DNA molecule, vector or construct comprises a second expression cassette comprising a transcribable DNA sequence operably linked to a second plant expressible promoter, wherein the transcribable DNA sequence corresponds to at least a portion of the FT transgene, and/or to a sequence complementary thereto. The transcribable DNA sequence may encode a RNA molecule comprising a targeting sequence that is complementary to at least a portion of the pre-mRNA or mature mRNA encoded by the FT transgene of the first expression cassette, such that the RNA molecule functions to suppress the FT transgene.

A recombinant DNA molecule, vector or construct comprising an FT transgene as provided herein may be used in plant transformation to generate a transgenic plant comprising the FT transgene. According to some embodiments, the FT transgene may be present in a first expression cassette and used in combination with a second expression cassette comprising a transcribable DNA sequence encoding a RNA molecule comprising a targeting sequence that corresponds to at least a portion of the FT transgene and/or a sequence complementary thereto, wherein both the first and second expression cassettes are transformed into a plant as the same or separate transformation event(s). According to some embodiments, an FT transgene transformed into a plant may comprise a target site for an endogenous RNA molecule that may target and trigger suppression of the FT transgene.

The polynucleotide coding sequence of an FT transgene may include Gm.FT2a (SEQ ID NO: 1), or any polynucleotide sequence encoding the Gm.FT2a protein (SEQ ID NO: 2). The polynucleotide coding sequence of an FT transgene may also correspond to other FT genes in soybean or other plants. For example, other polynucleotide coding sequences from soybean that may be used as an FT transgene according to present embodiments include: Gm.FT5a (SEQ ID NO: 3) or a polynucleotide sequence encoding a Gm.FT5a protein (SEQ ID NO: 4), or Gm.FT2b (SEQ ID NO: 5) or a polynucleotide sequence encoding a Gm.FT2b protein (SEQ ID NO: 6). In addition, examples of polynucleotide coding sequences from other plant species that may be used include: Zm.ZCN8 (SEQ ID NO: 7) from maize or a polynucleotide sequence encoding Zm.ZCN8 protein (SEQ ID NO: 8), Nt.FT-like or Nt.FT4 (SEQ ID NO: 9) from tobacco or a polynucleotide sequence encoding Nt.FT-like or Nt.FT4 protein (SEQ ID NO: 10), Le.FT or SFT (SEQ ID NO: 11) from tomato or a polynucleotide sequence encoding Le.FT or SFT protein (SEQ ID NO: 12), At.FT (SEQ ID NO: 13) from Arabidopsis or a polynucleotide sequence encoding At.FT protein (SEQ ID NO: 14), At.TSF (SEQ ID NO: 15) from Arabidopsis or a polynucleotide sequence encoding At.TSF protein (SEQ ID NO: 16), Os.HD3a (SEQ ID NO: 17) from rice or a polynucleotide sequence encoding Os.HD3a protein (SEQ ID NO: 18), or Pt.FT (SEQ ID NO: 19) from Populus trichocarpa or a polynucleotide sequence encoding Pt.FT protein (SEQ ID NO: 20). Additional examples of polynucleotide coding sequences for FT transgenes and proteins that may also be used include the following: Gm.FT5b (SEQ ID NO: 21) from soybean, or any polynucleotide sequence encoding the Gm.FT5b protein (SEQ ID NO: 22); Gh.FT1 (SEQ ID NO: 23) from cotton, or any polynucleotide sequence encoding the Gh.FT1 protein (SEQ ID NO: 24); Bn.FTA2a (SEQ ID NO: 25) from canola, or any polynucleotide sequence encoding the Bn.FTA2a protein (SEQ ID NO: 26); Ta.FT3B1 (SEQ ID NO: 27) from wheat, or any polynucleotide sequence encoding the Ta.FT3B1 protein (SEQ ID NO: 28); or Ps.FTa1 (SEQ ID NO: 29) from pea, or any polynucleotide sequence encoding the Ps.FTa1 protein (SEQ ID NO: 30). Polynucleotide coding sequences for FT transgenes encoding additional FT proteins from other species having known amino acid sequences may also be used according to embodiments of the present invention, which may, for example, include the following: Md.FT1 and Md.FT2 from apple (Malus domestica); Hv.FT2 and Hv.FT3 from barley (Hordeum vulgare); Cs.FTL3 from Chrysanthemum; Ls.FT from lettuce (Lactuca sativa); Pn.FT1 and Pn.FT2 from Lombardy poplar (Populus nigra); Pa.FT from London plane tree (Platanus acerifolia); D1.FT1 from Longan (Dimocarpus longan); Ps.FTa1, Ps.FTa2, Ps.FTb1, Ps.FTb2, and Ps.FTc from pea (Pisum sativum); Ac.FT from pineapple (Ananas comosus); Cm-FTL1 and Cm-FTL2 from pumpkin (Cucurbita maxima); Ro.FT from rose; Cg.FT from spring orchid (Cymbidium); Fv.FT1 from strawberry (Fragaria vesca); Bv.FT2 from sugar beet (Beta Vulgaris); Ha.FT4 from sunflower (Helianthus annuus); and Ta.FT or TaFT1 from wheat (Triticum aestivum), and sequences that are at least 60% identical, at least 65% identical, at least 70% identical, at least 75% identical, at least 80% identical, at least 85% identical, or at least 90%, at least 91%, at least 92%, at least 93%, at least 94%, at least 95%, at least 96%, at least 97%, at least 98%, or at least 99% identical to one or more of such known polynucleotide and/or protein sequences. See, e.g., Wickland, D P et al., “The Flowering Locus T/Terminal Flower 1 Gene Family: Functional Evolution and Molecular Mechanisms”, Molecular Plant 8: 983-997 (2015), the content and disclosure of which is incorporated herein by reference.

Unless otherwise stated, nucleic acid or polynucleotide sequences described herein are provided (left-to-right) in the 5′ to 3′ direction, and amino acid or protein sequences are provided (left-to-right) in the N-terminus to C-terminus direction. Additional known or later discovered FT genes and proteins from these or other species may also be used according to embodiments of the present invention. These FT genes may be known or inferred from their nucleotide and/or protein sequences, which may be determined by visual inspection or by use of a computer-based searching and identification tool or software (and database) based on a comparison algorithm with known FT sequences, structural domains, etc., and according to any known sequence alignment technique, such as BLAST, FASTA, etc.

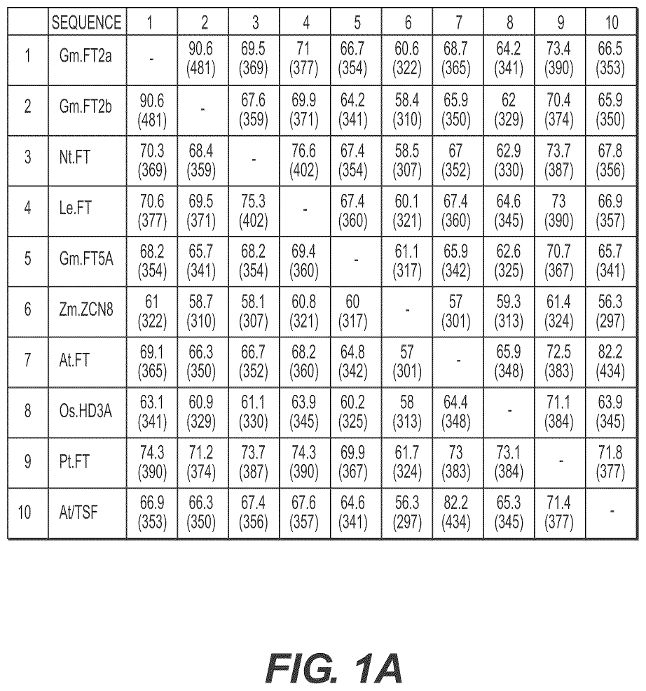

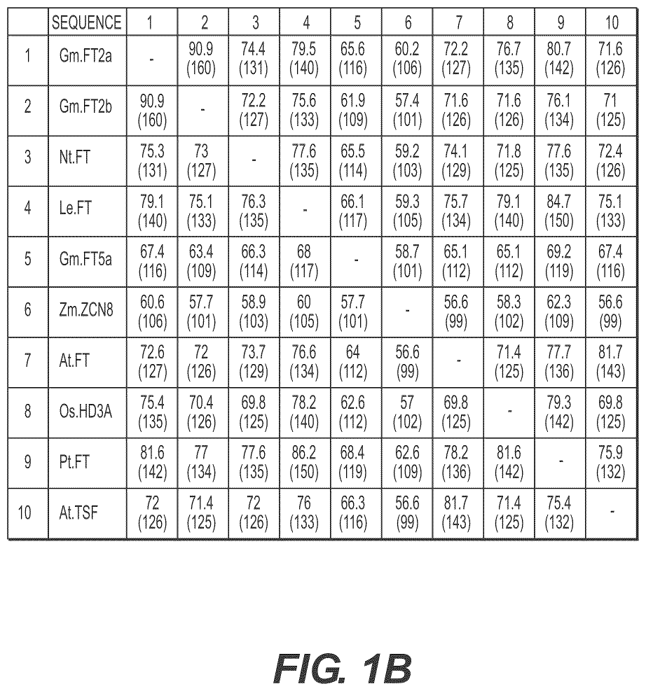

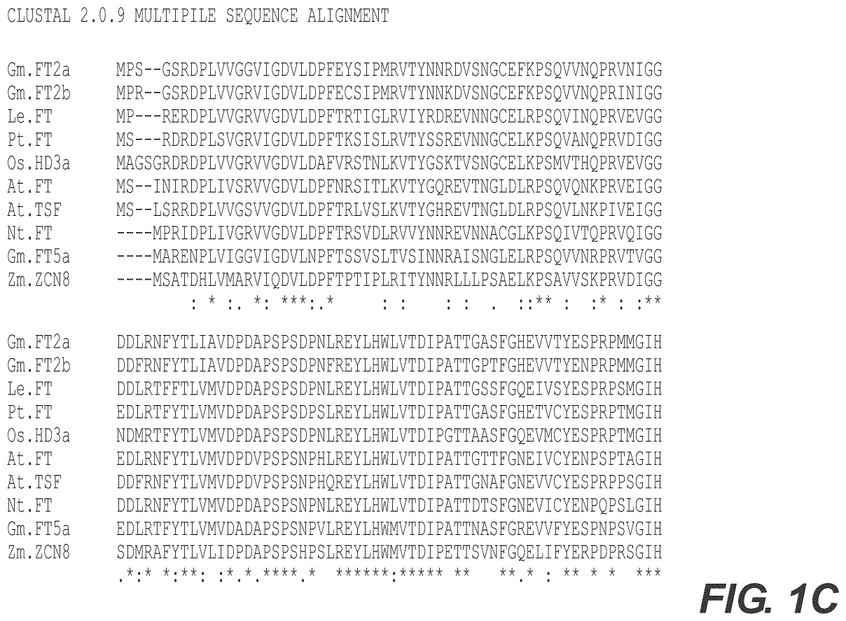

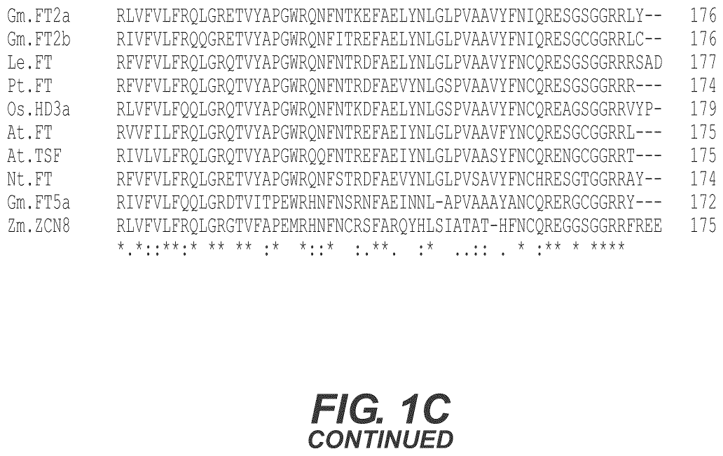

According to embodiments of the present invention, an FT transgene of a recombinant DNA molecule, vector or construct may comprise a polynucleotide sequence that (when optimally aligned) is at least 60% identical, at least 65% identical, at least 70% identical, at least 75% identical, at least 80% identical, at least 85% identical, or at least 90%, at least 91%, at least 92%, at least 93%, at least 94%, at least 95%, at least 96%, at least 97%, at least 98%, at least 99%, or 100% identical to one or more of the polynucleotide FT coding sequences listed above (e.g., SEQ ID NOS: 1, 3, 5, 7, 9, 11, 13, 15, 17, 19, 21, 23, 25, 27, or 29), or to any other known florigenic FT coding sequence. Sequence identity percentages among polynucleotide sequences of the above listed full length coding sequences of FT genes are presented in FIG. 1A. Each cell in the table in FIG. 1A shows the percentage identity for the FT gene in the corresponding row (query sequence) as compared to the FT gene in the corresponding column (subject sequence) divided by the total length of the query sequence, and the number in parenthesis is the total number of identical bases between the query and subject sequences. As shown in this figure, the percentage identities among polynucleotide sequences for these sampled FT genes range from about 60% to about 90% identity. Thus, a polynucleotide sequence that is within one or more of these sequence identity ranges or has a higher sequence identity may be used according to embodiments of the present invention to induce flowering, increase yield, and/or alter one or more reproductive traits of a plant. Similar polynucleotide coding sequences for FT may be designed or chosen based on known FT protein sequences, conserved amino acid residues and domains, the degeneracy of the genetic code, and any known codon optimizations for the particular plant species to be transformed.

According to embodiments of the present invention, an FT transgene may comprise a polynucleotide sequence encoding an amino acid or protein sequence that (when optimally aligned) is at least 60% identical, at least 65% identical, at least 70% identical, at least 75% identical, at least 80% identical, at least 85% identical, or at least 90%, at least 91%, at least 92%, at least 93%, at least 94%, at least 95%, at least 96%, at least 97%, at least 98%, at least 99%, or 100% identical to any one or more of the FT protein or amino acid sequences listed above (e.g., SEQ ID NOS: 2, 4, 6, 8, 10, 12, 14, 16, 18, 20, 22, 24, 26, 28, or 30) or any other known florigenic FT protein sequence, or a functional fragment thereof. Such a “functional fragment” is defined as a protein having a polypeptide sequence that is identical or highly similar to a full-length FT protein but lacking one or more amino acid residues, portions, protein domains, etc., of the full-length FT protein, as long as the fragment remains active in causing one or more of the phenotypic effects or changes similar to the full-length protein when transgenically expressed in a plant. Sequence identity percentages among the above listed full length FT proteins are presented in FIG. 1B. The percentages are calculated as described above in reference to FIG. 1A based on the number of identical amino acid residues (in parenthesis) between the query and subject FT protein sequences. Multiple sequence alignment of these FT proteins is also shown in FIG. 1C. As can be seen from these figures, the percentage identity among protein sequences for these FT genes ranges from about 60% to about 90% identity. Thus, a polynucleotide sequence encoding an amino acid or protein sequence that is within one or more of these sequence identity ranges or has a higher sequence identity may be used according to embodiments of the present invention to induce flowering, increase seed yield, and/or alter one or more reproductive traits of a plant. These FT protein sequences encoded by a polynucleotide sequence of the present invention may be designed or chosen based on known FT protein sequences and their conserved amino acid residues and domains.

As described below, an FT transgene comprising any one of the above coding sequences may further include one or more expression and/or regulatory element(s), such as enhancer(s), promoter(s), leader(s), intron(s), etc., and an FT transgene may comprise a genomic sequence encoding an FT protein or amino acid sequence, or a fragment or portion thereof.

As used herein, the term “sequence identity” or “percent identity” refers to the extent to which two optimally aligned DNA or protein sequences are identical. Various pair-wise or multiple sequence alignment algorithms and programs are known in the art, such as ClustalW, etc., that may be used to compare the sequence identity or similarity between two or more sequences, such as between two or more FT genes or protein sequences, or an FT gene (nucleotide) or protein sequence and another nucleotide or protein sequence. For example, the percentage identity of one sequence (query) to another sequence (subject) may be calculated as described above in reference to FIGS. 1A and 1B (i.e., with the sequences optimally aligned, divide the number of identical bases or residues by the total number of bases or residues for the query sequence, and multiply by 100%). Although other alignment and comparison methods are known in the art, the alignment and percent identity between two sequences (including the percent identity ranges described above) may be as determined by the ClustalW algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007), the entire contents and disclosures of which are incorporated herein by reference. For purposes of the present invention, when two sequences are optimally aligned (with allowance for gaps in their alignment), the “percent identity” for the query sequence is calculated as described above in reference to FIGS. 1A and 1B—i.e., Percent Identity=(Number of Identical Positions between query and subject sequences/Total Number of Positions in the Query Sequence)×100%, with each sequence consisting of a series of positions (nucleotide bases or amino acid residues). The two optimally aligned sequences may also be described as being a certain percent identical. A percent identity may optionally be described instead in reference to a defined window of comparison (e.g., an alignment window) between the two sequences, in which case the number of identical positions within the window of comparison is divided by the nucleotide length of the window of comparison, and multiplied by 100%. An alignment window may be defined as the region of identity, similarity or overlap between the two sequences.

A recombinant polynucleotide or protein molecule, construct or vector may be isolated. As used herein, the term “isolated” refers to at least partially separating a molecule from other molecules typically associated with it in its natural state. In one embodiment, the term “isolated” refers to a DNA molecule that is separated from the nucleic acids that normally flank the DNA molecule in its natural state. For example, a DNA molecule encoding a protein that is naturally present in a bacterium would be an isolated DNA molecule if it was not within the DNA of the bacterium from which the DNA molecule encoding the protein is naturally found. Thus, a DNA molecule fused to or operably linked to one or more other DNA molecule(s) with which it would not be associated in nature, for example as the result of recombinant DNA or plant transformation techniques, is considered isolated herein. Such molecules are considered isolated even when integrated into the chromosome of a host cell or present in a nucleic acid solution with other DNA molecules.

An FT protein sequence encoded by a polynucleotide sequence or transgene of the present invention may also be designed or chosen to have one or more amino acid substitution(s) known to be chemically and/or structurally conservative (e.g., replacing one amino acid with another having similar chemical or physical properties, such as hydrophobicity, polarity, charge, steric effect, acid/base chemistry, similar side chain group, such as hydroxyl, sulfhydryl, amino, etc.) to avoid or minimize structural changes to the protein that might affect its function. For example, valine is often a conservative substitute for alanine, and threonine may be a conservative substitute for serine. Additional examples of conservative amino acid substitutions in proteins include: valine/leucine, valine/isoleucine, phenylalanine/tyrosine, lysine/arginine, aspartic acid/glutamic acid, and asparagine/glutamine. An FT protein sequence encoded by a polynucleotide sequence or transgene of the present invention may also include proteins that differ in one or more amino acids from those of a known FT protein or similar sequence as a result of deletion(s) and/or insertion(s) involving one or more amino acids.

Various FT genes and proteins from different plant species may be identified and considered FT homologs or orthologs for use in the present invention if they have a similar nucleic acid and/or protein sequence and share conserved amino acids and/or structural domain(s) with at least one known FT gene or protein. As used herein, the term “homolog” in reference to a FT gene or protein is intended to collectively include any homologs, analogs, orthologs, paralogs, etc., of the FT gene or protein, and the term “homologous” in reference to polynucleotide or protein sequences is intended to mean similar or identical sequences including synthetic, artificial or engineered polynucleotide or protein sequences. Such a FT homolog may also be defined as having the same or similar biological function as known FT genes (e.g., acting to similarly influence flowering and/or other reproductive or yield-related traits or phenotypes when ectopically expressed in a plant).

Sequence analysis and alignment of FT protein sequences from different plant species further reveals a number of conserved amino acid residues and at least one conserved structural domain. By subjecting the various aligned FT protein sequences (see, e.g., FIGS. 1B and 1C) to a protein domain identification tool using a Pfam database (e.g., Pfam version 26.0, released November 2011, or later version), these FT proteins have been found to contain and share at least a portion of a putative phosphatidyl ethanolamine-binding protein (PEBP) domain (Pfam domain name: PBP_N; Accession number: PF01161). See, e.g., Banfield, M J et al., “The structure of Antirrhinum centroradialis protein (CEN) suggests a role as a kinase inhibitor,” Journal of Mol Biol., 297(5): 1159-1170 (2000), the entire contents and disclosure of which are incorporated herein by reference. This PEBP domain was found to correspond, for example, to amino acids 28 through 162 of the full length Gm.FT2a protein (See Table 5 below). Thus, FT proteins encompassed by embodiments of the present invention may include those identified or characterized as having or containing at least a PEBP domain (Accession number: PF01161) according to Pfam analysis. Accordingly, the present invention may further include a polynucleotide sequence(s) encoding an FT protein having at least a PEBP domain. As known in the art, the “Pfam” database is a large collection of multiple sequence alignments and hidden Markov models covering many common protein families and containing information about various protein families and their domain structure(s). By identifying a putative Pfam structural domain(s) for a given protein sequence, the classification and function of the protein may be inferred or determined. See, e.g., Finn, R D et al., “The Pfam protein families database,” Nucleic Acids Research (Database Issue), 42:D222-D230 (2014), the entire contents and disclosure of which are incorporated herein by reference.

Embodiments of the present invention may further include polynucleotide sequence(s) encoding inductive or florigenic FT proteins. An FT protein encoded by a polynucleotide sequence may be “inductive” or “florigenic” if the FT protein, when ectopically expressed in a plant, is able to cause earlier flowering and/or an increased prolificacy in the number of flowers, pods, bolls, siliques, fruits, nuts, tubers, and/or seeds per one or more node(s) of the plant. Without being bound by theory, such increased prolificacy in the number of flowers, pods, bolls, siliques, fruits, nuts, tubers, and/or seeds per node(s) of the plant may result from an increase in the number of meristems at those node(s) that undergo a vegetative to reproductive transition and produce flowers. Such an increased prolificacy at each node due to ectopic expression of a “florigenic” FT may be due to increased synchronization of the release and floral development of early racemes and lateral meristems at each node. Although a “florigenic” FT protein may function to induce earlier flowering when ectopically expressed in a plant, a transgenically expressed “florigenic” FT protein may increase the number of flowers, pods, bolls, siliques, fruits, nuts, tubers, and/or seeds per node(s) of a plant through one or more pathways or mechanisms that are independent of, or in addition to, any florigenic effects related to flowering time and/or reproductive duration.

Florigenic FT-like genes from various plant species are generally well conserved. However, many proteins in the PEBP family have amino acid sequences that are substantially similar to florigenic FT proteins but do not behave as florigens. For example, Terminal Flower (TFL) genes from various plant species have similar protein sequences to florigenic FT genes but actually delay flowering. Recent work has identified specific amino acid residues that are generally not shared between florigenic FT proteins and other PEBP proteins, such as TFLs, and substitutions at many of these positions have been shown to convert florigenic FT proteins into floral repressor proteins. See, e.g., Ho and Weigel, Plant Cell 26: 552-564 (2014); Danilevskaya et al., Plant Physiology 146(1): 250-264 (2008); Harig et al., Plant Journal 72: 908-921 (2012); Hsu et al., Plant Cell 18: 1846-1861 (2006); Kojima et al., Plant Cell Physiology 43(10): 1096-1105 (2002); Kong et al., Plant Physiology 154: 1220-1231(2010); Molinero-Rosales et al., Planta 218: 427-434 (2004); Zhai et al., PLoS ONE, 9(2): e89030 (2014), and Wickland D P et al. (2015), supra, the entire contents and disclosures of which are incorporated herein by reference. Thus, these amino acid residues can serve as signatures to further define and distinguish florigenic FT proteins of the present invention.

According to embodiments of the present invention, an “inductive” or “florigenic” FT protein may be further defined or characterized as comprising one or more of the following amino acid residue(s) (amino acid positions refer to corresponding or optimally aligned positions of the full-length Arabidopsis FT protein, SEQ ID NO: 14): a proline at amino acid position 21 (P21); an arginine or lysine at amino acid position 44 (R44 or K44); a glycine at amino acid position 57 (G57); a glutamic acid or an aspartic acid at amino acid position 59 (E59 or D59); a tyrosine at amino acid position 85 (Y85); a leucine at amino acid position 128 (L128); a glycine at amino acid position 129 (G129); a threonine at amino acid position 132 (T132); an alanine at amino acid position 135 (A135); a tryptophan at amino acid position 138 (W138); a glutamic acid or an aspartic acid at amino acid position 146 (E146 or D146); and/or a cysteine at amino acid position 164 (C164). Corresponding amino acid positions of other FT proteins can be determined by alignment with the Arabidopsis FT sequence (see, e.g., FIG. 1C). One skilled in the art would be able to identify corresponding amino acid positions of other FT proteins based on their sequence alignment. Several of these key residues fall within an external loop domain of FT-like proteins, defined as amino acids 128 through 145 of the Arabidopsis full-length FT sequence (SEQ ID NO: 14) and corresponding sequences of other FT proteins (see, e.g., FIG. 1C). Thus, polynucleotides of the present invention may encode florigenic FT proteins having one or more of these conserved amino acid residues.

Florigenic FT proteins of the present invention may also have one or more other amino acids at one or more of the above identified residue positions. For example, in reference to the above amino acid positions of the Arabidopsis FT (At.FT) protein sequence (SEQ ID NO: 14), a florigenic FT protein may alternatively have one or more of the following amino acids: an alanine (in place of proline) at the position corresponding to position 21 of the At.FT protein sequence (P21A), or possibly other small, nonpolar residues, such as glycine or valine, at this position; a histidine (in place of lysine or arginine) at the amino acid position corresponding to position 44 of the At.FT protein sequence, or possibly other polar amino acids at this position; an alanine or cysteine (in place of glycine) at the amino acid position corresponding to position 57 of the At.FT protein sequence, or possibly other small, nonpolar residues, such proline or valine, at this position; an asparagine or serine (in place of glutamic acid or aspartic acid) at the amino acid position corresponding to position 59 of the At.FT protein sequence, or possibly other small, polar residues, such as glutamine, cysteine, or threonine, at this position; a variety of polar and nonpolar uncharged residues (other than tyrosine) at the amino acid position corresponding to position 85 of the At.FT protein sequence; a nonpolar or hydrophobic uncharged residue (other than leucine), such as isoleucine, valine, or methionine, at the amino acid position corresponding to position 128 of the At.FT protein sequence; a variety of smaller nonpolar and uncharged residues (other than glycine), such as alanine, valine, leucine, isoleucine, methionine, etc., at the amino acid position corresponding to position 129 of the At.FT protein sequence, although some polar and charged residues may be tolerated at this position; a polar uncharged residue (other than threonine) at the amino acid position corresponding to position 132 of the At.FT protein sequence; a variety of amino acids other than proline, such as threonine, at the amino acid position corresponding to position 135 of the At.FT protein sequence; a variety of other bulky nonpolar or hydrophobic amino acids (in place of tryptophan), such as methionine or phenylalanine, at the amino acid position corresponding to position 138 of the At.FT protein sequence; a variety of other polar or non-positively charged amino acids, such as asparagine or serine, at the amino acid position corresponding to position 146 of the At.FT protein sequence; and/or a variety of other polar or nonpolar amino acids (in place of cysteine, such as isoleucine, at the amino acid position corresponding to position 164 of the At.FT protein sequence. One skilled in the art would be able to identify corresponding amino acid positions and substitutions of FT proteins based on their sequence alignment to the Arabidopsis FT protein sequence. In addition, other chemically conservative amino acid substitutions are also contemplated within the scope of florigenic FT proteins based on the knowledge of one skilled in the art of protein biochemistry. Accordingly, polynucleotides of the present invention may further encode florigenic FT proteins having one or more conservative amino acid substitutions. Indeed, florigenic FT proteins encoded by polynucleotides of the present invention include native sequences and artificial sequences containing one or more conservative amino acid substitutions, as well as functional fragments thereof.

Florigenic FT proteins of the present invention may also be defined as excluding (i.e., not having) one or more amino acid substitutions that may be characteristic of, or associated with, TFL or other non-florigenic or anti-florigenic proteins. For example, in reference to the amino acid positions of the Arabidopsis FT protein sequence (SEQ ID NO: 14), a florigenic FT protein may exclude one or more of the following amino acids (i.e., at corresponding or optimally aligned positions of the florigenic FT protein): a phenylalanine or serine at the position corresponding to position 21 of the At.FT protein sequence (e.g., in place of proline or alanine); a phenylalanine at the position corresponding to position 44 of the At.FT protein sequence (e.g., in place of arginine or lysine); a histidine, glutamic acid, or aspartic acid at the position corresponding to position 57 of the At.FT protein sequence (e.g., in place of glycine); a glycine or alanine at the position corresponding to position 59 of the At.FT protein sequence (e.g., in place of glutamic acid or aspartic acid); a histidine at the position corresponding to position 85 of the At.FT protein sequence (e.g., in place of tyrosine); a lysine, arginine, alanine, or methionine at the position corresponding to position 109 of the At.FT protein sequence; a lysine or arginine at the position corresponding to position 128 of the At.FT protein sequence (e.g., in place of leucine); a glutamine or asparagine at the position corresponding to position 129 of the At.FT protein sequence (e.g., in place of glycine); a valine or cysteine at the position corresponding to position 132 of the At.FT protein sequence (e.g., in place of threonine); a lysine, arginine, or alanine at the position corresponding to position 134 of the At.FT protein sequence (e.g., in place of tyrosine); a proline at the position corresponding to position 135 of the At.FT protein sequence (e.g., in place of alanine or threonine); a serine, aspartic acid, glutamic acid, alanine, lysine, or arginine at the position corresponding to position 138 of the At.FT protein sequence (e.g., in place of tryptophan or methionine); a lysine or arginine at the position corresponding to position 140 of the At.FT protein sequence; a lysine or arginine at the position corresponding to position 146 of the At.FT protein sequence (e.g., in place of acidic or uncharged polar residues); a lysine or arginine at the position corresponding to position 152 of the At.FT protein sequence; and/or an alanine at the position corresponding to position 164 of the At.FT protein sequence (e.g., in place of cysteine or isoleucine). One skilled in the art would be able to identify corresponding amino acid positions and substitutions of other FT proteins based on their sequence alignment. Accordingly, embodiments of the present invention may exclude polynucleotides that encode FT-like proteins having one or more of the above amino acid substitutions associated with TFL or other anti-florigens. However, an FT protein may tolerate one or some of these amino acid substitutions while still maintaining florigenic activity.

A florigenic FT protein of the present invention may also be defined as being similar to a known FT protein in addition to having one or more of the above signature or conserved amino acid residues. For example, a florigenic protein may be defined as having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28, and 30, or a functional fragment thereof, in addition to one or more of the following signature residues: a tyrosine or other uncharged polar or nonpolar residue (e.g., alanine, tryptophan, methionine, leucine, threonine, cysteine, serine, or asparagine) at the amino acid position corresponding to position 85 of the At.FT protein sequence; a leucine or other nonpolar or hydrophobic residue (e.g., isoleucine, valine, or methionine) at the amino acid position corresponding to position 128 of the At.FT protein sequence; and/or a tryptophan or other large nonpolar or hydrophobic residue (e.g., methionine or phenylalanine) at the amino acid position corresponding to position 138 of the At.FT protein sequence. Such a florigenic FT protein may be further defined as having additional signature amino acid residue(s), such as one or more of the following: a glycine or other small nonpolar and uncharged residue (e.g., alanine, valine, leucine, isoleucine, or methionine) at the amino acid position corresponding to position 129 of the At.FT protein sequence; and/or a threonine at the amino acid position corresponding to position 132 of the At.FT protein sequence.

A florigenic FT protein of the present invention may also be defined as having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28, and 30, or a functional fragment thereof, but not having (i.e., excluding) one or more non-florigenic or anti-florigenic residues, such as one or more of the following: a histidine at the amino acid position corresponding to position 85 of the At.FT protein sequence; a lysine or arginine at the amino acid position corresponding to position 128 of the At.FT protein sequence; and/or a serine, aspartic acid, glutamic acid, lysine or arginine at the amino acid position corresponding to position 138 of the At.FT protein sequence. Such a florigenic FT protein may be further defined as not having (i.e., excluding) one or more additional residues, such as one or more of the following: a glutamine or asparagine at the amino acid position corresponding to position 129 of the At.FT protein sequence; and/or a valine or cysteine at the amino acid position corresponding to position 132 of the At.FT protein sequence.

According to embodiments of the present invention, a recombinant DNA molecule, vector or construct is provided comprising a polynucleotide sequence encoding a FT protein that is operably linked to one or more promoter(s) and/or other regulatory element(s) that are operable in a plant cell to control or bias the timing and/or location of FT expression when transformed into a plant. According to some embodiments, the FT transgene may be present in a first expression cassette and used with a second expression cassette comprising a transcribable DNA sequence that corresponds to at least a portion of the FT transgene, and/or to a sequence complementary thereto, and encodes a RNA molecule that targets the FT transgene for suppression. According to some embodiments, the FT transgene may comprise a target site for an endogenous RNA molecule that may target and trigger suppression of the FT transgene.

As commonly understood in the art, the term “promoter” may generally refer to a DNA sequence that contains an RNA polymerase binding site, transcription start site, and/or TATA box and causes, initiates, directs, assists and/or promotes the transcription and expression of an associated transcribable polynucleotide sequence and/or gene (or transgene). A promoter may be synthetically produced, engineered, varied and/or derived from a known or naturally occurring promoter sequence or other promoter sequence (e.g., as provided herein). A promoter may also include a chimeric promoter comprising a combination of two or more heterologous sequences. A promoter of the present invention may thus include variants of promoter sequences that are similar in composition, but not identical to, other promoter sequence(s) known or provided herein. As used herein, the term “operably linked” refers to a functional linkage between a promoter or other regulatory element and an associated transcribable polynucleotide sequence or coding sequence of a gene (or transgene), such that the promoter, etc., operates to initiate, assist, affect, cause, direct, and/or promote the transcription and expression of the associated coding or transcribable polynucleotide sequence, at least in particular tissue(s), developmental stage(s), and/or under certain condition(s). A “plant expressible promoter” refers to a promoter that may be used to express in a plant, plant cell and/or plant tissue an associated coding sequence, transgene or transcribable polynucleotide sequence that is operably linked to the promoter.

A promoter may be classified according to a variety of criteria relating to the pattern of expression of a coding sequence or gene (including a transgene) operably linked to the promoter, such as constitutive, developmental, tissue-specific, inducible, etc. Promoters that initiate transcription in all or most tissues of the plant are referred to as “constitutive” promoters. Promoters that initiate transcription during certain periods or stages of development are referred to as “developmental” promoters. Promoters whose expression is enhanced in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A promoter that expresses in a certain cell type of the plant is referred to as a “cell type specific” promoter. An “inducible” promoter is a promoter that initiates transcription in response to an environmental stimulus such as cold, drought, heat or light, or other stimuli, such as wounding or chemical application. A promoter may also be classified in terms of its origin, such as being heterologous, homologous, chimeric, synthetic, etc. The term “heterologous” in reference to a promoter or other regulatory sequence in relation to an associated polynucleotide sequence (e.g., a transcribable DNA sequence, coding sequence, gene or transgene) is a promoter or regulatory sequence that is not operably linked to such associated polynucleotide sequence in nature—e.g., the promoter or regulatory sequence has a different origin relative to its associated polynucleotide sequence, and/or not naturally occurring in a plant species to be transformed with the promoter or regulatory sequence. The term “heterologous” more broadly includes a combination of two or more DNA molecules or sequences when such a combination is not normally found in nature. As used herein, the phrase “not normally found in nature” means not found in nature without human introduction. For example, two or more DNA molecules or sequences would be heterologous with respect to each other if they are normally found in different genomes or at different loci in the same genome, or if they are not identically combined in nature. According to many embodiments, a plant expressible promoter operably linked to a polynucleotide sequence encoding an FT protein is heterologous with respect to the polynucleotide sequence encoding the FT protein.

According to embodiments of the present invention, a recombinant DNA molecule, vector or construct is provided comprising a florigenic FT transgene or coding sequence operably linked to a promoter that functions in a plant, which may be introduced or transformed into a plant to cause the plant to have an altered flowering, reproductive and/or yield-related trait or phenotype. Embodiments of the present invention provide a recombinant DNA molecule comprising an FT transgene or coding sequence operably linked to a “vegetative stage” promoter to cause, when introduced or transformed into a plant, expression of the FT transgene earlier in the development of the plant (i.e., during the vegetative growth phase of the plant) to produce an increased level of FT in target tissues than would otherwise occur in a wild type plant at the same stage of development. Timing FT transgene expression during the vegetative stage(s) of development may be important for affecting one or more reproductive, flowering and/or yield-related traits or phenotypes by providing a timely inductive signal for the production of an increased number of floral meristems and successful flowers at one or more node(s) of the plant. Vegetative stage expression may be necessary to trigger early flowering and allow for improved reproductive, flowering and/or yield-related traits or phenotypes, such as increased flowers, pods, etc., per node of the plant. Without being bound by any theory, vegetative stage expression of an FT transgene in a plant may operate to synchronize and/or increase early flowering at one or more node(s) to produce more flowers per node of the plant. The promoters described below as a part of the present invention provide options for timing FT expression.

As used herein, a “vegetative stage” promoter includes any promoter that initiates, causes, drives, etc., transcription or expression of its associated gene, transgene or transcribable DNA sequence during one or more vegetative stage(s) of plant development, such as during one or more of Ve, Vc, V1, V2, V3, V4, etc., and/or any or all later vegetative stages of development (e.g., up to Vₙ stage). In other words, the term “vegetative stage” is in reference to the vegetative developmental stage(s) of the plant as a whole. Such a “vegetative stage” promoter may be further defined as initiating, causing, driving, etc., transcription or expression of its associated gene/transgene or transcribable DNA sequence in one or more vegetative tissue(s) of a plant, such as one or more vegetative meristem tissue(s). Such a “vegetative stage” promoter may be further defined as a “vegetative stage preferred” promoter that initiates, causes, drives, etc., transcription or expression of its associated gene, transgene or transcribable DNA sequence at least preferentially or mostly, if not exclusively, during one or more vegetative stage(s) of plant development (as opposed to reproductive stages). However, a “vegetative stage” and a “vegetative stage preferred” promoter may each also permit, allow, cause, drive, etc., transcription or expression of its associated gene, transgene or transcribable DNA sequence during reproductive phase(s) or stage(s) of development in one or more cells or tissues of the plant, such as in one or more floral or reproductive tissue(s). In fact, a “vegetative stage” promoter may even initiate, cause, drive, etc., transcription or expression of its associated gene, transgene or transcribable DNA sequence in one or more reproductive or floral tissues at a greater level or extent than in vegetative tissue(s), provided that the “vegetative stage” promoter also initiates, causes, drives, etc., transcription or expression of its associated gene, transgene or transcribable DNA sequence during one or more vegetative stage(s) of plant development.

The features and characteristics associated with vegetative stages of development for a given plant species are known in the art. For dicot plants, vegetative morphological features and characteristics of the plant during vegetative stages of development may include cotyledon form, vegetative meristems (apical, lateral/axillary, and root), leaf arrangement, leaf shape, leaf margin, leaf venation, petioles, stipules, ochrea, hypocotyl, and roots. According to embodiments of the present invention, a “vegetative stage” promoter may also be further defined by the particular vegetative stage during which observable or pronounced transcription or expression of its associated gene (or transgene) is first caused, initiated, etc. For example, a vegetative stage promoter may be a Vc stage promoter, a V1 stage promoter, a V2 stage promoter, a V3 stage promoter, etc. As such, a “Vc stage” promoter is defined as a vegetative stage promoter that first initiates or causes transcription of its associated gene, transgene or transcribable DNA sequence during the Vc stage of plant development, a “V1 stage” promoter is defined as a vegetative stage promoter that first initiates or causes transcription of its associated gene, transgene or transcribable DNA sequence during the V1 stage of plant development, a “V2 stage” promoter is defined as a vegetative stage promoter that first initiates or causes transcription of its associated gene, transgene or transcribable DNA sequence during the V2 stage of plant development, and so on, although expression of the associated gene, transgene or transcribable DNA sequence may be present continuously or discontinuously in one or more tissues during later vegetative (and or reproductive) stage(s) of development. One skilled in the art would be able to determine the timing of expression of a given gene, transgene or transcribable DNA sequence during plant development using various molecular assays and techniques known in the art.

According to embodiments of the present invention, a “vegetative stage” promoter may include a constitutive, tissue-preferred, or tissue-specific promoter. For example, a vegetative stage promoter may drive expression of its associated FT gene/transgene or transcribable DNA sequence in one or more plant tissue(s), such as in one or more of the root(s), stem(s), leaf/leaves, meristem(s), etc., during a vegetative stage(s) of plant development. However, such a vegetative stage promoter may preferably drive expression of its associated FT transgene or coding sequence or transcribable DNA sequence in one or more meristem(s) of the plant. According to many embodiments, a “vegetative stage” promoter may be a “meristem-specific” or “meristem-preferred” promoter to cause expression of the FT transgene or coding sequence or transcribable DNA sequence in meristematic tissue. FT proteins are known to operate in the meristems of a plant to help trigger the transition from vegetative to reproductive growth after translocation of the FT protein from the leaves. In contrast, embodiments of the present invention provide for expression of an FT transgene directly in the meristem of a plant to induce flowering and cause the plant to adopt an altered reproductive and/or yield-related trait or phenotype. Thus, according to embodiments of the present invention, a recombinant DNA molecule, construct or vector is provided comprising an FT transgene or coding sequence operably linked to a “meristem-specific” or “meristem-preferred” promoter that drives expression of the FT transgene at least preferentially in one or more meristematic tissues of a plant when transformed into the plant. As used herein, “meristem-preferred promoter” refers to promoters that preferentially cause expression of an associated gene, transgene or transcribable DNA sequence in at least one meristematic tissue of a plant relative to other plant tissues, such as in one or more apical and/or axillary meristems, whereas a “meristem-specific promoter” refers to promoters that cause expression of an associated gene, transgene or transcribable DNA sequence exclusively (or almost exclusively) in at least one meristematic tissue of a plant.

According to embodiments of the present invention, a recombinant DNA molecule is provided comprising an FT coding sequence operably linked to a vegetative stage promoter, which may also be a meristem-preferred and/or meristem-specific promoter. For example, the promoter may include the pAt.Erecta promoter from Arabidopsis (SEQ ID NO: 31), or a functional fragment or portion thereof. Two examples of a truncated portion of the pAt.Erecta promoter according to embodiments of the present invention are provided as SEQ ID NO: 32 and SEQ ID NO: 48. See, e.g., Yokoyama, R. et al., “The Arabidopsis ERECTA gene is expressed in the shoot apical meristem and organ primordia,” The Plant Journal 15(3): 301-310 (1998). pAt.Erecta is an example of a vegetative stage promoter that is also meristem-preferred. Other vegetative stage, meristem-preferred or meristem-specific promoters have been identified based on their characterized expression profile (see, e.g., Examples 4 and 7 below) that may also be used to drive FT expression according to embodiments of the present invention. For example, promoters from the following soybean receptor like kinase (RLK) genes were identified that could be used as vegetative stage, meristem-preferred promoters: Glyma10g38730 (SEQ ID NO: 33), Glyma09g27950 (SEQ ID NO: 34), Glyma06g05900 (SEQ ID NO: 35), and Glyma17g34380 (SEQ ID NO: 36), and any functional portion thereof. Vegetative stage, meristem-preferred promoters according to embodiments of the present invention may also include receptor like kinase (RLK) gene promoters from potato: PGSC0003DMP400032802 (SEQ ID NO: 37) and PGSC0003DMP400054040 (SEQ ID NO: 38), and any functional portion thereof. Given the characterization provided herein of the pAt.Erecta promoter driving FT expression and the similar expression profiles identified for other RLK, Erecta or Erecta-like (Erl) genes, vegetative-stage, meristem-preferred or meristem-specific promoters of the present invention may further comprise any known or later identified promoter sequences of RLK, Erecta and Erecta-like genes from other dicotyledonous species having vegetative-stage pattern of expression in the meristems of plants.

Additional examples of vegetative stage, meristem-preferred or meristem-specific promoters may include those from the following Arabidopsis genes: Pinhead (At.PNH) (SEQ ID NO: 39), Angustifolia 3 or At.AN3 (SEQ ID NO: 40), At.MYB17 (At.L/MI2 or Late Meristem Identity 2; At3g61250) (SEQ ID NO: 41), Kinesin-like gene (At5g55520) (SEQ ID NO: 42), AP2/B3-like genes, including At.REM17 (SEQ ID NO: 43) or At.REM19, and Erecta-like 1 and 2 genes, At.Erl1 (SEQ ID NO: 44) and At.Erl2 (SEQ ID NO: 45), and any functional portion thereof. Another example is an At.AP1 promoter (pAt.AP1 or pAP1) from Arabidopsis (SEQ ID NO: 49), or a functional portion thereof. However, the pAt.AP1 promoter may be considered more of a late vegetative and reproductive stage promoter. Given the later pattern of vegetative and reproductive stage expression, the pAt.AP1 and related promoters may be useful for driving expression of an FT transgene and/or FT suppression element. Further examples identified from similar genes and/or genes having a similar expression pattern as the pAt.AP1 promoter in their native plant species may include a promoter from one of the following genes: AT1G26310.1 (SEQ ID NO: 50), AT3G30260.1 (SEQ ID NO: 51), or AT5G60910.1 (SEQ ID NO: 52) from Arabidopsis; Glyma01g08150 (SEQ ID NO: 53), Glyma02g13420 (SEQ ID NO: 54), Glyma08g36380 (SEQ ID NO: 55), or Glyma16g13070 (SEQ ID NO: 56) from soybean; Solyc02g065730 (SEQ ID NO: 57), Solyc02g089210 (SEQ ID NO: 58), Solyc03g114830 (SEQ ID NO: 59), or Solyc06g069430 (SEQ ID NO: 60) from tomato; or GRMZM2G148693 (SEQ ID NO: 61), GRMZM2G553379 (SEQ ID NO: 62), GRMZM2G072582 (SEQ ID NO: 63), or GRMZM2G147716 (SEQ ID NO: 64) from corn, or any functional portion thereof.

A vegetative stage promoter, which may also be a meristem-preferred or meristem-specific promoter, may include both early and late vegetative stage promoters depending on their pattern of expression during vegetative stages of development. An “early vegetative stage” promoter first initiates or causes observable or detectable transcription or expression of its associated gene/transgene or transcribable DNA sequence during one or more earlier vegetative stages (i.e., Ve through V5 stages), whereas a “late vegetative stage” first initiates or causes observable or detectable transcription or expression of its associated gene/transgene or transcribable DNA sequence during one or more later vegetative stages (i.e., V6 stage and later). An early or late vegetative stage promoter may also be an early or late vegetative stage preferred promoter. An “early vegetative stage preferred” promoter initiates, causes, drives, etc., transcription or expression of its associated gene/transgene or transcribable DNA sequence more predominantly or to a greater extent during one or more earlier vegetative stages (i.e., Ve through V5 stages) as compared to later vegetative stages. Likewise, a “late vegetative stage preferred” promoter initiates, causes, drives, etc., transcription or expression of its associated gene/transgene or transcribable DNA sequence more predominantly or to a greater extent during one or more later vegetative stages (i.e., V6 stage and later) as compared to earlier vegetative stages. Accordingly, an early vegetative stage promoter may also be a late vegetative stage preferred promoter if the promoter first initiates or causes observable or detectable transcription or expression of its associated gene/transgene or transcribable DNA sequence during earlier vegetative stages, but also initiates, causes, drives, etc., transcription or expression of its associated gene/transgene or transcribable DNA sequence more predominantly or to a greater extent during later vegetative stages. The vegetative stage promoter examples listed above may include early and late vegetative stage promoters, which may also be early vegetative stage preferred or late vegetative stage preferred.

The polynucleotide sequence of a vegetative stage promoter (or a functional fragment or portion thereof) may also have a relaxed sequence identity relative to any of the foregoing vegetative stage promoters while still maintaining a similar or identical pattern of expression of an associated transcribable DNA sequence, gene or transgene operably linked to the promoter. For example, a vegetative stage promoter may comprise a polynucleotide sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to a polynucleotide sequence selected from the above SEQ ID NOs: 31, 32, 33, 34, 35, 36, 37, 38, 39, 40, 41, 42, 43, 44, 45, 48, 49, 50, 51, 52, 53, 54, 55, 56, 57, 58, 59, 60, 61, 62, 63, or 64, or a functional portion thereof. A “functional portion” of a known or provided promoter sequence is defined as one or more continuous or discontinuous portion(s) of the known or provided promoter sequence that may functionally drive, cause, promote, etc., expression of its associated gene, transgene or transcribable DNA sequence in a manner that is identical or similar to the known or provided promoter sequence. Based on the present disclosure, one skilled in the art would be able to determine if a promoter comprising one or more portion(s) of a known or provided promoter sequence, and/or having a shorter sequence and/or a sequence with a more relaxed sequence identity relative to a known or provided promoter sequence, causes a similar pattern of expression and/or similar phenotypes or effects when its associated reporter gene or FT transgene is expressed in a plant as compared to the known or provided promoter sequence.

According to some embodiments, a “reproductive stage” promoter (defined below) may be operably linked and used to express an FT transgene or coding sequence, as long as the reproductive stage promoter provides (i.e., initiates, causes, drives, etc.) at least some level of FT transgene expression during a vegetative stage(s) of plant development to provide an early floral induction signal. Examples of reproductive stage promoters are provided below. Whether a given promoter should be categorized as an early or late vegetative stage promoter and/or a reproductive stage promoter depends on the particular plant species in which the promoter is used. A promoter having a defined pattern of expression in one plant species, such as its native plant species, may have a different, altered or shifted pattern of expression when expressed in a different plant species (e.g., heterologously in a different plant species), although it is anticipated that the pattern of expression with a given promoter would most often be similar (if not identical or nearly identical) between different plant species. For example, a reproductive stage promoter in one plant species may function as an earlier vegetative stage promoter when used to express a transgene or transcribable DNA sequence in another plant species. Thus, a reproductive stage promoter may be used heterologously in some cases to express an FT transgene and induce early flowering. For example, the pAt.AP1 promoter (SEQ ID NO: 49) has a more reproductive stage preferred pattern of expression in its native Arabidopsis plant species, but may drive an earlier pattern of vegetative stage expression in the meristem when used heterologously in soybean plants, in addition to driving reproductive stage expression.

As stated above, a recombinant DNA molecule, construct or vector of the present invention may comprise an expression cassette comprising a polynucleotide sequence encoding an FT protein (i.e., a FT transgene) that is operably linked to a vegetative stage promoter, which may also be a meristem-preferred or meristem-specific promoter. The polynucleotide coding sequence of the FT transgene or expression cassette may also be operably linked to one or more additional regulatory element(s), such as an enhancer(s), leader, transcription start site (TSS), linker, 5′ and 3′ untranslated region(s), intron(s), polyadenylation signal, termination region or sequence, etc., that are suitable or necessary for regulating or allowing expression of the FT transgene or cassette to effectively produce an FT protein in a plant cell. Such additional regulatory element(s) may be optional and used to enhance or optimize expression of the transgene. For purposes of the present invention, an “enhancer” may be distinguished from a “promoter” in that an enhancer typically lacks a transcription start site, TATA box, or equivalent sequence and is thus insufficient alone to drive transcription. As used herein, a “leader” may be defined generally as the DNA sequence of the untranslated 5′ region (5′ UTR) of a gene (or transgene) between the transcription start site (TSS) and the protein coding sequence start site.

As used herein in reference to a polynucleotide, a “construct” is a polynucleotide segment or sequence comprising one or more sequence elements, such as a coding sequence or a transcribable DNA sequence and one or more expression or regulatory elements, such as a promoter, enhancer, etc. An “expression cassette” is a type of construct comprising a coding sequence or a transcribable DNA sequence that can express the coding sequence or transcribable DNA sequence in a suitable host cell, such as a plant or bacterial cell, and one or more promoter and/or regulatory elements operably linked to the coding sequence or transcribable DNA sequence. A “vector” is a polynucleotide or DNA molecule that may comprise one or more constructs and/or expression cassettes and that is suitable for stability, storage or another use or purpose, such as delivery to, transformation of, and/or maintenance in, a plant or host cell. A “vector” may include a plasmid or circular DNA molecule, a linear DNA molecule, a transformation vector suitable for plant transformation, etc. A DNA molecule or vector may comprise one or more construct(s), expression cassette(s), selectable marker(s), replication and/or maintenance element(s), etc.

According to embodiments of the present invention, the term “recombinant” in reference to a polynucleotide (DNA or RNA) molecule, protein, construct, vector, etc., refers to a polynucleotide or protein molecule or sequence that is not normally found in nature and/or is present in a context in which it is not normally found in nature, including a polynucleotide (DNA or RNA) molecule, protein, construct, etc., comprising a combination of two or more polynucleotide or protein sequences that would not naturally occur contiguously or in close proximity together without human intervention, and/or a DNA molecule, construct, etc., comprising at least two DNA sequences that are heterologous with respect to each other. A recombinant DNA molecule, construct, etc., may comprise DNA sequence(s) that is/are separated from other polynucleotide sequence(s) that exist in proximity to such DNA sequence(s) in nature, and/or a DNA sequence that is adjacent to (or contiguous with) other polynucleotide sequence(s) that are not naturally in proximity with each other. A recombinant DNA molecule, construct, etc., may also refer to a DNA molecule or sequence that has been genetically engineered and constructed outside of a cell. For example, a recombinant DNA molecule may comprise any suitable plasmid, vector, etc., and may include a linear or circular DNA molecule. Such plasmids, vectors, etc., may contain various maintenance elements including a prokaryotic origin of replication and selectable marker, as well as a FT expressing transgene or expression cassette perhaps in addition to a plant selectable marker gene, etc.

According to embodiments of the present invention, a second expression cassette is provided comprising a transcribable polynucleotide or DNA sequence operably linked to a plant expressible promoter, wherein the transcribable DNA sequence comprises a sequence that corresponds to at least a portion of an FT transgene and/or a sequence complementary thereto, and targets the FT transgene for suppression. The transcribable DNA sequence may encode a RNA molecule comprising a targeting sequence that is complementary to at least a portion of the pre-mRNA or mature mRNA encoded by a polynucleotide sequence encoding an FT protein (i.e., an FT transgene), such that the RNA molecule suppresses the FT transgene. Accordingly, a recombinant DNA molecule, construct or vector is provided for transformation into a plant comprising the second expression cassette. Such a recombinant DNA molecule, construct or vector may further comprise a first expression cassette comprising a polynucleotide coding sequence encoding a FT protein (i.e., a FT transgene) operably linked to a first plant expressible promoter, and wherein the second expression cassette comprises the transcribable DNA sequence operably linked to a second plant expressible promoter. Alternatively, two recombinant DNA molecules, constructs or vectors may be provided for plant transformation comprising a first recombinant DNA molecule, construct or vector and a second recombinant DNA molecule, construct or vector, wherein the first recombinant DNA molecule, construct or vector comprises the first expression cassette comprising an FT transgene, and the second recombinant DNA molecule, construct or vector comprises the second expression cassette comprising a transcribable DNA sequence that includes a sequence that corresponds to at least a portion of the FT transgene and/or a sequence complementary thereto. According to some embodiments, a plant expressible promoter operably linked to a transcribable DNA sequence that encodes a RNA molecule for suppression of an FT gene or transgene is heterologous with respect to the transcribable DNA sequence.

Any method known in the art for suppression of a target gene may be used to suppress the FT transgene according to embodiments of the present invention including expression of antisense RNAs, double stranded RNAs (dsRNAs) or inverted repeat RNA sequences, or via co-suppression or RNA intereference (RNAi) through expression of small interfering RNAs (siRNAs), short hairpin RNAs (shRNAs), trans-acting siRNAs (ta-siRNAs), or micro RNAs (miRNAs). See, e.g., U.S. Patent Application Publication Nos. 2009/0070898, 2011/0296555, and 2011/0035839, the contents and disclosures of which are incorporated herein by reference. Accordingly, the RNA molecule encoded by a transcribable DNA sequence may be an antisense RNA, double stranded RNA (dsRNA) or inverted repeat RNA, a small interfering RNA (siRNA), a short hairpin RNA (shRNA), a trans-acting siRNA (ta-siRNA), or a micro RNA (miRNA), and including precursor RNAs, such as a precursor siRNA or miRNA, that may be processed or cleaved into a mature RNA molecule, such as a mature siRNA or miRNA. The term “suppression” as used herein, refers to a lowering, reduction or elimination of the expression level of the mRNA and/or protein encoded by the targeted gene and/or transgene in a plant, plant cell or plant tissue, which may be limited to a particular tissue and/or stage of plant development depending on the promoter used to express the RNA molecule.

According to embodiments of the present invention, a recombinant DNA molecule, construct or vector is provided comprising a transcribable DNA sequence and/or suppression element(s) encoding a RNA molecule or sequence that targets an FT transgene for suppression, wherein the transcribable DNA sequence is operably linked to a plant-expressible promoter. Since the RNA molecule is for suppression, the RNA molecule encoded by a transcribable DNA sequence may be a non-coding RNA molecule. For purposes of the present invention, a “non-coding RNA molecule” is a RNA molecule that does not encode a protein. According to some embodiments, a recombinant DNA molecule, construct or vector may comprise a first expression cassette comprising an FT transgene and a second expression cassette comprising a transcribable DNA sequence encoding a RNA molecule that targets the FT transgene for suppression. Alternatively, a first expression cassette comprising an FT transgene and a second expression cassette comprising a transcribable DNA sequence for suppression of the FT transgene may be present in two different recombinant DNA molecules, constructs or vectors.

The transcribable DNA sequence may comprise a suppression element that is at least 15 nucleotides in length, such as from about 15 nucleotides in length to about 27 nucleotides in length, or 15, 16, 17, 18, 19, 20, 21, 22, 23, 24, 25, 26, or 27 nucleotides in length, wherein the suppression element corresponds to at least a portion of the target FT transgene to be suppressed, and/or to a DNA sequence complementary thereto. In many embodiments, the transcribable DNA sequence or suppression element may be at least 17, at least 19, at least 20, at least 21, at least 22, or at least 23 nucleotides (or more) in length (e.g., at least 25, at least 30, at least 50, at least 100, at least 200, at least 300, at least 500, at least 1000, at least 1500, at least 2000, at least 3000, at least 4000, or at least 5000 nucleotides in length). Depending on the length and sequence of a transcribable DNA sequence or suppression element, one or more sequence mismatches or non-complementary bases may be tolerated without a loss of suppression. Indeed, even shorter RNAi suppression elements ranging from about 15 nucleotides to about 27 nucleotides in length may have one or more mismatches or non-complementary bases, yet still be effective at suppressing a target FT transgene. Accordingly, a sense or anti-sense suppression element may comprise a sequence that is at least 80%, at least 85%, at least 90%, at least 95%, at least 96%, at least 97%, at least 98%, at least 99%, at least 99.5%, or 100% identical or complementary to a corresponding sequence of at least a segment or portion of the targeted FT transgene, or its complementary sequence, respectively.

A transcribable DNA sequence of the present invention for targeted suppression of a FT transgene may include one or more of the following suppression element(s) and/or targeting sequence(s): (a) a DNA sequence that includes at least one anti-sense DNA sequence that is anti-sense or complementary to at least one segment or portion of the targeted FT transgene; (b) a DNA sequence that includes multiple copies of at least one anti-sense DNA sequence that is anti-sense or complementary to at least one segment or portion of the targeted FT transgene; (c) a DNA sequence that includes at least one sense DNA sequence that comprises at least one segment or portion of the targeted FT transgene; (d) a DNA sequence that includes multiple copies of at least one sense DNA sequence that each comprise at least one segment or portion of the targeted FT transgene; (e) a DNA sequence that includes an inverted repeat of a segment or portion of a targeted FT transgene and/or transcribes into RNA for suppressing the targeted FT transgene by forming double-stranded RNA, wherein the transcribed RNA includes at least one anti-sense DNA sequence that is anti-sense or complementary to at least one segment or portion of the targeted FT transgene and at least one sense DNA sequence that comprises at least one segment or portion of the targeted FT transgene; (f) a DNA sequence that is transcribed into RNA for suppressing the targeted FT transgene by forming a single double-stranded RNA and includes multiple serial anti-sense DNA sequences that are each anti-sense or complementary to at least one segment or portion of the targeted FT transgene and multiple serial sense DNA sequences that each comprise at least one segment or portion of the targeted FT transgene; (g) a DNA sequence that is transcribed into RNA for suppressing the targeted FT transgene by forming multiple double strands of RNA and includes multiple anti-sense DNA sequences that are each anti-sense or complementary to at least one segment or portion of the targeted FT transgene and multiple sense DNA sequences that each comprise at least one segment or portion of the targeted FT transgene, wherein the multiple anti-sense DNA segments and multiple sense DNA segments are arranged in a series of inverted repeats; (h) a DNA sequence that includes nucleotides derived from a miRNA, preferably a plant miRNA; (i) a DNA sequence that includes nucleotides of a siRNA; (j) a DNA sequence that is transcribed into an RNA aptamer capable of binding to a ligand; and (k) a DNA sequence that is transcribed into an RNA aptamer capable of binding to a ligand and DNA that transcribes into a regulatory RNA capable of regulating expression of the targeted FT transgene, wherein the regulation of the targeted FT transgene is dependent on the conformation of the regulatory RNA, and the conformation of the regulatory RNA is allosterically affected by the binding state of the RNA aptamer by the ligand. A transcribable DNA sequence may comprise one or more of the above suppression elements and/or targeting sequence(s), which may correspond to one or more sequences of the FT transgene, and/or its complementary sequence.

Multiple sense and/or anti-sense suppression sequences of a transcribable DNA sequence for more than one FT transgene target sequence may be arranged serially in tandem or arranged in tandem segments or repeats, such as tandem inverted repeats, which may also be interrupted by one or more spacer sequence(s). Furthermore, a sense or anti-sense sequence of a transcribable DNA sequence or suppression element may not be perfectly matched or complementary to the targeted FT transgene sequence, depending on the sequence and length of the transcribable DNA sequence or suppression element. Indeed, even shorter RNAi suppression elements from about 15 nucleotides to about 27 nucleotides in length may have one or more mismatches or non-complementary bases depending on the length of the suppression element or targeting sequence, such as 1, 2, 3, 4, 5, 6, 7, 8, 9 or 10 (or more) mismatches, yet still be effective at suppressing the target FT transgene. Accordingly, a sense or anti-sense suppression element may comprise a sequence that is at least 80%, at least 85%, at least 90%, at least 95%, at least 96%, at least 97%, at least 98%, at least 99%, at least 99.5% or 100% identical or complementary to a corresponding sequence of at least a segment or portion of the targeted FT transgene, or its complementary sequence, respectively.

For anti-sense suppression, a transcribable DNA sequence may comprise a sequence that is anti-sense or complementary to at least a portion or segment of the targeted FT transgene. The transcribable DNA sequence and/or suppression element(s) may comprise multiple anti-sense sequences that are complementary to one or more portions or segments of the targeted FT transgene, or multiple copies of an anti-sense sequence that is complementary to a targeted FT transgene. An anti-sense sequence may be at least 80%, at least 85%, at least 90%, at least 95%, at least 96%, at least 97%, at least 98%, at least 99%, at least 99.5%, or 100% identical to a DNA sequence that is complementary to at least a segment or portion of a targeted FT transgene mRNA. In other words, an anti-sense sequence may be at least 80%, at least 85%, at least 90%, at least 95%, at least 96%, at least 97%, at least 98%, at least 99%, at least 99.5%, or 100% complementary to a targeted FT transgene.

The terms “percent complementarity” or “percent complementary” as used herein in reference to two nucleotide sequences is similar to the concept of percent identity but refers to the percentage of nucleotides of a sequence that optimally base-pair or hybridize to nucleotides a reference sequence when the two sequences are linearly arranged and optimally base paired without secondary folding structures, such as loops, stems or hairpins, but with tolerance for mismatches and gaps in base-pairing between the two sequences. Such a percent complementarity may be between two DNA strands, two RNA strands, or a DNA strand and a RNA strand. The “percent complementarity” may be calculated by (i) optimally base-pairing or hybridizing the two nucleotide sequences in a linear and fully extended arrangement (i.e., without folding or secondary structures) over a window of comparison (e.g., alignment window), (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. For these purposes, an alignment window is defined as the region of complementarity between the two sequences. Optimal base pairing of two sequences may be determined based on the known pairings of nucleotide bases, such as G-C, A-T, and A-U, through hydrogen bonding. If the “percent complementarity” is being calculated in relation to a reference or query sequence without specifying a particular comparison window, then the percent identity is determined by dividing the number of complementary positions between the two linear sequences by the total length of the reference sequence. For purposes of the present invention, when two sequences (query and subject) are optimally base-paired (with allowance for gaps and mismatches or non-base-paired nucleotides), the “percent complementarity” for a query sequence (when a comparison window is not defined) is equal to the number of base-paired positions between the two sequences divided by the total number of positions in the query sequence over its length, which is then multiplied by 100%.

For suppression of an FT transgene using an inverted repeat or a transcribed dsRNA, a transcribable DNA sequence may comprise a sense sequence that comprises a segment or portion of a targeted FT transgene and an anti-sense sequence that is complementary to a segment or portion of the targeted FT transgene, wherein the sense and anti-sense DNA sequences are arranged in tandem. The sense and/or anti-sense sequences, respectively, may each be less than 100% identical or complementary to a segment or portion of the targeted FT transgene as described above. The sense and anti-sense sequences may be separated by a spacer sequence, such that the RNA molecule transcribed from the transcribable DNA sequence forms a stem, loop or stem-loop structure between the sense and anti-sense sequences. The transcribable DNA sequence may instead comprise multiple sense and anti-sense sequences that are arranged in tandem, which may also be separated by one or more spacer sequences. A transcribable DNA sequence comprising multiple sense and anti-sense sequences may be arranged as a series of sense sequences followed by a series of anti-sense sequences, or as a series of tandemly arranged sense and anti-sense sequences.

For suppression of an FT transgene using a microRNA (miRNA), a transcribable DNA sequence may comprise a DNA sequence derived from a miRNA sequence native to a virus or eukaryote, such as an animal or plant, or modified or derived from such a native miRNA sequence. Such native or native-derived miRNA sequences may form a fold back structure and serve as a scaffold for the precursor miRNA, and may correspond to the stem region of a native miRNA precursor sequence, such as from a native (or native-derived) pri-miRNA or pre-miRNA sequence. However, in addition to these native or native-derived miRNA scaffold or preprocessed sequences, engineered miRNAs of the present invention further comprise a sequence corresponding to a segment or portion of the targeted FT transgene. Thus, in addition to the pre-processed or scaffold sequences, the suppression element may be further engineered to comprise a sense and/or anti-sense sequence that corresponds to a segment or portion of a targeted FT transgene, and/or a sequence that is complementary thereto, although one or more sequence mismatches may be tolerated.

Engineered miRNAs are useful for targeted gene suppression with increased specificity. See, e.g., Parizotto et al., Genes Dev. 18:2237-2242 (2004), and U.S. Patent Application Publication Nos. 2004/0053411, 2004/0268441, 2005/0144669, and 2005/0037988, the contents and disclosures of which are incorporated herein by reference. miRNAs are non-coding RNAs. When a miRNA precursor molecule is cleaved, a mature miRNA is formed that is typically from about 19 to about 25 nucleotides in length (commonly from about 20 to about 24 nucleotides in length in plants), such as 19, 20, 21, 22, 23, 24, or 25 nucleotides in length, and has a sequence corresponding to the gene targeted for suppression and/or its complement. The mature miRNA hybridizes to target mRNA transcripts and guides the binding of a complex of proteins to the target transcripts, which may function to inhibit translation and/or result in degradation of the transcript, thus negatively regulating or suppressing expression of the targeted gene. miRNA precursors are also useful in plants for directing in-phase production of siRNAs, trans-acting siRNAs (ta-siRNAs), in a process that requires a RNA-dependent RNA polymerase to cause suppression of a target gene. See, e.g., Allen et al., Cell 121:207-221 (2005), Vaucheret Science STKE, 2005:pe43 (2005), and Yoshikawa et al. Genes Dev., 19:2164-2175 (2005), the contents and disclosures of which are incorporated herein by reference.

According to embodiments of the present invention, a recombinant DNA molecule, construct or vector is provided comprising a transcribable DNA sequence encoding a miRNA or precursor miRNA molecule for targeted suppression of a FT transgene. Such a transcribable DNA sequence may comprise a sequence of at least 19 nucleotides in length that corresponds to a FT transgene and/or a sequence complementary to the FT transgene, although one or more sequence mismatches and/or non-base-paired nucleotides may be tolerated.

An FT transgene may also be suppressed using one or more small interfering RNAs (siRNAs). The siRNA pathway involves the non-phased cleavage of a longer double-stranded RNA intermediate (“RNA duplex”) into small interfering RNAs (siRNAs). The size or length of siRNAs typically ranges from about 19 to about 25 nucleotides or base pairs, but common classes of siRNAs include those containing 21 base pairs or 24 base pairs. Thus, a transcribable DNA sequence of the present invention may encode a RNA molecule that is at least about 19 to about 25 nucleotides in length, such as 19, 20, 21, 22, 23, 24, or 25 nucleotides in length. For siRNA suppression, a recombinant DNA molecule, construct or vector is thus provided comprising a transcribable DNA sequence and/or suppression element encoding a siRNA molecule for targeted suppression of a FT transgene.

According to embodiments of the present invention, a recombinant DNA molecule, vector or construct is provided comprising a transcribable DNA sequence encoding a non-coding RNA molecule that binds or hybridizes to a sequence of a target mRNA in a plant cell, such as a coding (exon) and/or untranslated (UTR) sequence of the target mRNA, wherein the target mRNA molecule encodes an FT protein, and wherein the transcribable DNA sequence is operably linked to a plant expressible promoter. In addition to targeting a mature mRNA sequence, a non-coding RNA molecule encoded by a transcribable DNA sequence may target an intron sequence of a FT transgene or transcript. According to other embodiments, a recombinant DNA molecule, vector or construct is provided comprising a transcribable DNA sequence encoding a non-coding RNA (precursor) molecule that is cleaved or processed into a mature non-coding RNA molecule that binds or hybridizes to a target mRNA in a plant cell, wherein the target mRNA molecule encodes an FT protein, and wherein the transcribable DNA sequence is operably linked to a plant expressible promoter, which may be a tissue-specific, tissue-preferred, developmental, and/or other type of promoter.

According to embodiments of the present invention, a recombinant DNA molecule, vector or construct is provided comprising a transcribable DNA sequence encoding a non-coding RNA molecule, wherein the non-coding RNA molecule is at least 80%, at least 85%, at least 90%, at least 95%, at least 96%, at least 97%, at least 98%, at least 99%, at least 99.5%, or 100% complementary to at least a segment or portion of a mRNA molecule (i) expressed from an FT transgene and/or (ii) encoding an FT protein in a plant or plant cell, wherein the transcribable DNA sequence is operably linked to a plant-expressible promoter. A non-coding RNA molecule may target a mature mRNA or pre-mRNA sequence, a 5′ or 3′ untranslated region (UTR), a coding (exon) sequence and/or an intron or intronic sequence of a FT transgene or transcript. According to some embodiments, the non-coding RNA molecule targets a FT transgene for suppression and is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a polynucleotide (coding) sequence encoding an FT protein (e.g., SEQ ID NO: 1, 3, 5, 7, 9, 11, 13, 15, 17, 19, 21, 23, 25, 27, or 29), or to any other known florigenic FT coding sequence. According to other embodiments, the non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an FT protein that is at least 80%, at least 85%, at least 90%, at least 95%, at least 96%, at least 97%, at least 98%, at least 99%, at least 99.5%, or 100% identical to SEQ ID NO: 2, 4, 6, 8, 10, 12, 14, 16, 18, 20, 22, 24, 26, 28, or 30, or to any other known florigenic FT protein, or a functional fragment thereof. As used herein, the term “consecutive” in reference to a polynucleotide or protein sequence means without deletions or gaps in the sequence. According to embodiments of the present invention, a non-coding RNA molecule encoded by a transcribable DNA sequence of a recombinant DNA molecule, vector or construct provided herein may be a mature miRNA or siRNA, or a precursor miRNA or siRNA that may be processed or cleaved in a plant cell to form a mature miRNA or siRNA.

According to some embodiments of the present invention, the transcribable DNA sequence may comprise a sequence encoding a targeting sequence of a RNA molecule that is complementary and/or hybridizes to a particular Gm.FT2a gene or transgene mRNA to target the Gm.FT2a gene or transgene for suppression. The transcribable DNA sequence may comprise a sequence (e.g., SEQ ID NO: 65) encoding a targeting sequence (e.g., SEQ ID NO: 66) of an RNA molecule encoded by, and transcribed from, the transcribable DNA sequence. The targeting sequence of the RNA molecule may be any sequence of sufficient length that is complementary to a segment or portion of the mRNA encoded by the FT transgene, and the transcribable DNA sequence may comprise a sequence that encodes, or is transcribed into, the targeting sequence of the RNA molecule. For example, a transcribable DNA sequence encoding a precursor miRNA may comprise SEQ ID NO: 67, which may be processed into a mature miRNA comprising SEQ ID NO: 67 that targets a Gm.FT2a gene or transgene for suppression. The mRNA encoded by the targeted FT gene or transgene may comprise a target site for a RNA molecule encoded by the transcribable DNA sequence. Such a target site in the mRNA of the FT transgene may comprise, for example, SEQ ID NO: 68, which may be encoded by a sequence (e.g., SEQ ID NO: 69) of the FT gene or transgene. Thus, the polynucleotide coding sequence of the FT transgene may comprise a sequence encoding the target site for the RNA molecule.

In addition to a plant expressible promoter, a recombinant DNA molecule, construct, vector or expression cassette comprising a transcribable DNA sequence encoding a non-coding RNA molecule for suppression of an FT transgene may also be operably linked to one or more additional regulatory element(s), such as an enhancer(s), transcription start site (TSS), linker, polyadenylation signal, 5′ and/or 3′ scaffold or backbone sequences, termination region or sequence, etc., that are suitable, necessary or preferred for regulating or allowing expression of the transcribable DNA sequence in a plant cell or tissue. Such additional regulatory element(s) may be optional and used to enhance or optimize expression of the transcribable DNA sequence.

According to some embodiments, a transcribable DNA sequence may comprise a sequence that corresponds to at least a portion of a non-coding sequence of an FT transgene and/or a sequence complementary thereto, such as a 5′ or 3′ untranslated region (UTR) or intronic sequence of the FT transgene, which may allow for selective suppression of the FT transgene over an endogenous FT gene. The “non-coding” sequences of an FT transgene (not to be confused with a “non-coding RNA molecule” encoded by a transcribable DNA sequence for suppression of an FT gene or transgene) are the sequences of the FT transgene that are transcribed and form part of the pre-mRNA and/or mature mRNA, but do not encode the transgenic FT protein. Accordingly, the transcribable DNA sequence may encode a RNA molecule comprising a targeting sequence that corresponds to at least a portion of a non-coding sequence of the FT transgene and/or a sequence complementary thereto. Thus, the transcribable DNA sequence may comprise a sequence that corresponds to at least a portion of a pre-mRNA or mature mRNA encoded by the FT transgene. The sequence of the transcribable DNA sequence and encoded targeting sequence of the RNA molecule depend on the particular non-coding sequences of the FT transgene, which may be the same or different or unique relative to endogenous FT gene(s). According to some embodiments, a recombinant DNA molecule, vector or construct is provided comprising an expression cassette that comprises a transcribable DNA sequence having a sequence that corresponds to at least a portion of a non-coding sequence of an FT transgene and/or a sequence complementary thereto. Similarly as described above, two or more expression cassettes may be provided comprising a first expression cassette and a second expression cassette, wherein the first expression cassette comprises a polynucleotide sequence encoding a FT protein (i.e., a FT transgene) operably linked to a first plant expressible promoter, and the second expression cassette comprising a transcribable DNA sequence operably linked to a second plant expressible promoter, wherein the transcribable DNA sequence comprises a sequence that corresponds to at least a portion of a non-coding sequence of the FT transgene and/or a sequence complementary thereto. The first and second expression cassettes may be present in the same DNA molecule, vector or construct, or in separate DNA molecules, vectors or constructs.

A transcribable DNA sequence encoding a RNA molecule that targets an FT transgene for suppression may be operably linked to a plant expressible promoter. The pattern of expression of the RNA molecule may depend on the particular plant expressible promoter. As described above, expression of an FT transgene under the control of a vegetative stage promoter may be used to trigger early flowering and increase the number of flowers, pods, etc., per node of a plant, but may also cause early termination of the plant. It is presently proposed that additional expression of a RNA molecule that targets the FT transgene for suppression may be used to refine and/or attenuate the pattern and level of expression of the FT transgene to further mitigate the early termination phenotypes. This may occur through decreasing the quantity of transcript and protein from the FT transgene (i.e., reducing its level of expression) and/or modifying its pattern of expression (i.e., refinement or restriction of the pattern of transgenic FT expression). Without being bound by theory, a reduced expression of the FT transgene may be sufficient to induce early flowering while mitigating the early termination phenotypes. Likewise, a restricted spatiotemporal pattern of FT expression may reduce FT expression in particular tissues and/or stages of development where transgenic FT may cause earlier termination. Thus, according to embodiments of the present invention, the timings and patterns of expression of the FT transgene and the RNA molecule (targeting the FT transgene for suppression) may be the same, overlapping or more distinct.

According to many embodiments as introduced above, at least two expression cassettes may be provided comprising a first expression cassette and a second expression cassette, wherein the first expression cassette comprises an FT transgene operably linked to a first plant expressible promoter and the second expression cassette comprises a transcribable DNA sequence operably linked to a second plant expressible promoter, wherein the transcribable DNA sequence encodes a RNA molecule that targets the FT transgene for suppression. The two expression cassettes may be present in the same recombinant DNA molecule, construct or vector, or present in separate recombinant DNA molecules, constructs or vectors. As described above, the first plant expressible promoter operably linked to the FT transgene may be a vegetative stage promoter, which may also be a meristem-specific or meristem-preferred promoter. The second plant expressible promoter operably linked to the transcribable DNA sequence may comprise a variety of different promoter types including constitutive, inducible, developmental, tissue-specific, tissue-preferred, vegetative stage, reproductive stage, etc., but the timing and pattern of expression of the RNA molecule should at least partially overlap with the timing and pattern of expression of the FT transgene. According to some embodiments, the second plant expressible promoter may be a constitutive or vegetative stage promoter to reduce the level of expression of the FT transgene. Such a constitutive or vegetative stage promoter may also be a tissue-specific or tissue-preferred promoter and/or may broadly overlap with the timing and pattern of expression of the FT transgene. Indeed, a constitutive or overlapping expression pattern of the transcribable DNA sequence (and RNA suppression molecule) with respect to the FT transgene may be effective at reducing the quantity or dosage of FT expression, especially if suppression of the FT transgene is imperfect or incomplete. In some cases, the FT transgene and suppression construct may even be operably linked to a same or similar promoter. For example, the first plant expressible promoter driving expression of the FT transgene may be an early or late vegetative stage and/or reproductive stage promoter, and the second plant expressible promoter driving expression of the transcribable DNA sequence encoding the RNA molecule for suppression may also be an early or late vegetative stage and/or reproductive stage promoter.

Many examples of constitutive promoters are known in the art, such as a cauliflower mosaic virus (CaMV) 35S and 19S promoter (see, e.g., U.S. Pat. No. 5,352,605), an enhanced CaMV 35S promoter, such as a CaMV 35S promoter with Omega region (see, e.g., Holtorf, S. et al., Plant Molecular Biology, 29: 637-646 (1995) or a dual enhanced CaMV promoter (see, e.g., U.S. Pat. No. 5,322,938), a Figwort Mosaic Virus (FMV) 35S promoter (see, e.g., U.S. Pat. No. 6,372,211), a Mirabilis Mosaic Virus (MMV) promoter (see, e.g., U.S. Pat. No. 6,420,547), a Peanut Chlorotic Streak Caulimovirus promoter (see, e.g., U.S. Pat. No. 5,850,019), a nopaline or octopine promoter, a ubiquitin promoter, such as a soybean polyubiquitin promoter (see, e.g., U.S. Pat. No. 7,393,948), an Arabidopsis S-Adenosylmethionine synthetase promoter (see, e.g., 8,809,628), etc., or any functional portion of the foregoing promoters, the contents and disclosures of each of the above references are incorporated herein by reference. Alternatively, the second plant expressible promoter may be a vegetative and/or reproductive stage promoter, examples of which are provided herein.

According to other embodiments, the second plant expressible promoter may have a more distinct timing and/or pattern of expression, such as in different plant tissues and/or developmental stages, relative to the FT transgene. Thus, the effective spatiotemporal pattern of expression of the FT transgene may be modified, altered and/or refined depending on the relative expression timings and patterns of the FT transgene and the transcribable DNA sequence encoding the RNA molecule for suppression of the FT transgene (as well as the specific targeting sequence of the RNA molecule). According to many embodiments, however, the transcribable DNA sequence (and RNA molecule) may be expressed at a later developmental stage or tissue relative to the onset of expression of the FT transgene, such that the FT transgene is still able to provide the early floral induction signal before being suppressed by the later expression of the RNA suppression molecule, or stated differently the FT transgene may be suppressed after the early floral induction signal to reduce or mitigate early termination. For example, the first plant expressible promoter driving expression of the FT transgene may be an early vegetative stage promoter, and the second plant expressible promoter driving expression of the transcribable DNA sequence encoding the RNA molecule for suppression may be a late vegetative stage and/or reproductive stage promoter, or the first plant expressible promoter may be a late vegetative stage promoter, and the second plant expressible promoter may be a reproductive stage promoter. More broadly, the second plant expressible promoter may initiate, cause and/or drive expression of its associated transgene or transcribable DNA sequence at a later developmental stage than the first plant expressible promoter, such that the suppression construct is generally expressed after the initial FT floral induction signal during earlier vegetative stage(s) of development. Thus, the second plant expressible promoter may be a later developmental stage promoter than the first plant expressible promoter. For example, the second plant expressible promoter may drive expression at a later developmental stage than the first plant expressible promoter, but in the same tissue type or developmental lineage (e.g., in the meristem) as the first plant expressible promoter. Such a late vegetative stage and/or reproductive stage promoter may also be a tissue-specific or tissue-preferred promoter, such as a meristem-specific or meristem-preferred promoter. Examples of late vegetative stage promoters are provided above.

By expressing the transcribable DNA sequence and RNA molecule for suppression of the FT transgene during one or more later stage(s) of plant development relative to the FT transgene, the effective expression profile of the FT transgene may be modified, altered and/or refined to earlier developmental stages and/or tissues, relative to expression of the FT transgene alone. In some cases, vegetative stage expression of the FT transgene may linger or continue during later vegetative and/or reproductive stages or tissues in the plant. Thus, later expression of the RNA molecule that targets the FT transgene for suppression may reduce the level of FT transgene in those later stage(s) and/or tissue(s) to effectively limit or confine the expression level of the FT transgene to earlier developmental stage(s) and/or tissue(s). As a result, the early floral induction signal may be maintained or preserved, while later FT expression may be attenuated or reduced to avoid or delay early termination of the remaining meristematic reserves of the plant and allow for vegetative growth and development of the plant to continue after flowering.

As used herein, a “reproductive stage” promoter is defined as any promoter that initiates, causes, drives, etc., transcription or expression of its associated gene, transgene, or transcribable DNA sequence during one or more reproductive stage(s) of plant development, such as during one or more of R1, R2, R3, R4, R5, R6, R7, and/or R8 stages of development. Such a “reproductive stage” promoter may be further defined as a “reproductive stage preferred” promoter that initiates, causes, drives, etc., transcription or expression of its associated gene/transgene or transcribable DNA sequence at least preferably or mostly, if not exclusively, during one or more reproductive stage(s) of plant development (as opposed to vegetative stages). However, a “reproductive stage” and a “reproductive stage preferred” promoter may each also initiate, permit, allow, cause, drive, etc., transcription or expression of its associated gene, transgene, or transcribable DNA sequence during vegetative phase(s) or stage(s) of development in one or more cells or tissues of the plant. Thus, a reproductive stage promoter may also be a vegetative stage promoter if expressed during both developmental phases (i.e., during both vegetative and reproductive stages of development). Such a reproductive stage promoter may also be a tissue-specific or tissue-preferred promoter, such as a meristem-specific or meristem-preferred promoter. A “reproductive stage” promoter may be further defined as initiating, causing, driving, etc., transcription or expression of its associated gene/transgene or transcribable DNA sequence in one or more reproductive tissue(s) of a plant. Such a “reproductive stage” promoter may also be defined as a “floral preferred” promoter that initiates, causes, drives, etc., transcription or expression of its associated gene/transgene or transcribable DNA sequence at least preferably or mostly, if not exclusively, in at least one floral or reproductive tissue, such as a floral meristem, or a “floral specific” promoter that initiates, causes, drives, etc., transcription or expression of its associated gene/transgene or transcribable DNA sequence exclusively (or almost exclusively) in at least one floral or reproductive tissue. The features and characteristics of these reproductive stages for a given plant species are known in the art.

Examples of reproductive stage promoters, which may also be early or late vegetative stage promoters depending on their pattern of expression in a given plant species, may include the following promoters from tomato genes: a Sl.Nod promoter (pS1.Nod, pLe.Nod or pNod) (SEQ ID NO: 70), a SL.MADS5 promoter (pSl.MADS5, pLe.MADS5 or pMADS5) (SEQ ID NO: 71), or a Sl.MADS-RIN promoter (pSl.MADS-RIN, pLe.MADS-RIN or pMADS-RIN) (SEQ ID NO: 72), or any functional portion thereof. Further examples identified as having homology and/or a similar expression pattern as the pSl.MADS5 and/or pSl.MADS-RIN promoter in their native plant species may include a promoter from one of the following genes: AT1G24260.1 (SEQ ID NO: 73), AT2G45650.1 (SEQ ID NO: 74), AT3G02310.1 (SEQ ID NO: 75), or AT5G15800.1 (SEQ ID NO: 76), or AT2G03710.1 (SEQ ID NO: 77) from Arabidopsis; Glyma05g28140 (SEQ ID NO: 78), Glyma08g11120 (SEQ ID NO: 79), Glyma11g36890 (SEQ ID NO: 80), Glyma08g27670 (SEQ ID NO: 81), Glyma13g06730 (SEQ ID NO: 82), or Glyma19g04320 (SEQ ID NO: 83) from soybean; Solyc02g089200 (SEQ ID NO: 84), Solyc03g114840 (SEQ ID NO: 85), Solyc12g038510 (SEQ ID NO: 86), Solyc04g005320 (SEQ ID NO: 87) or Solyc05g056620 (SEQ ID NO: 88) from tomato; or GRMZM2G159397 (SEQ ID NO: 89), GRMZM2G003514 (SEQ ID NO: 90), GRMZM2G160565 (SEQ ID NO: 91), GRMZM2G097059 (SEQ ID NO: 92), GRMZM2G099522 (SEQ ID NO: 93) or GRMZM2G071620 (SEQ ID NO: 94) from corn, or any functional portion of any of the foregoing promoters.

According to embodiments of the present invention, a “reproductive stage” promoter may also be further defined by the particular reproductive stage during which observable or pronounced transcription or expression of its associated gene, transgene, or transcribable DNA sequence is first caused, initiated, etc. For example, a reproductive stage promoter may be a R1 stage promoter, a R2 stage promoter, a R3 stage promoter, etc. As such, a “R1 stage” promoter is defined as a reproductive stage promoter that first initiates or causes transcription of its associated gene, transgene, or transcribable DNA sequence during the R1 stage of plant development, a “R2 stage” promoter is defined as a reproductive stage promoter that first initiates or causes transcription of its associated gene, transgene, or transcribable DNA sequence during the R2 stage of plant development, and so on, although expression of the associated gene, transgene, or transcribable DNA sequence may be present continuously or discontinuously in one or more tissues during later reproductive stage(s) of development. The transition from vegetative to reproductive stages (and onset of the R1 stage) is defined according to standard conventions in the art for a given crop plant (i.e., typically as with soybeans the visible appearance of the first open flower on the plant). One skilled in the art would be able to determine the timing of expression of a given gene, transgene, or transcribable DNA sequence during plant development using various molecular assays and techniques known in the art, if such timing of expression is not already known.

According to embodiments of the present invention, a “reproductive stage” promoter may include a constitutive, tissue-preferred, or tissue-specific promoter. For example, a reproductive stage promoter may drive expression of its associated gene, transgene or transcribable DNA sequence in one or more plant tissue(s), such as in one or more of the root(s), stem(s), leaf/leaves, meristem(s), etc., during a reproductive stage(s) of plant development. However, such a reproductive stage promoter may preferably drive expression of its associated gene, transgene or transcribable DNA sequence in one or more meristem(s) of the plant. According to many embodiments, a “reproductive stage” promoter may be a “meristem-specific” or “meristem-preferred” promoter to cause expression of its associated gene, transgene or transcribable DNA sequence in meristematic tissue to at least partially correspond to the pattern of expression of the FT transgene and attenuate and/or refine expression of the FT transgene.

The polynucleotide sequence of these promoters (or a functional portion thereof) may also have a relaxed sequence identity while still maintaining a similar or identical pattern of expression of an associated gene, transgene or transcribable DNA sequence operably linked to the promoter. For example, the late vegetative and/or reproductive stage promoter may comprise a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to a polynucleotide sequence selected from the above SEQ ID NOs: 70, 71, 72, 73, 74, 75, 76, 77, 78, 79, 80, 81, 82, 83, 84, 85, 86, 87, 88, 89, 90, 91, 92, 93, or 94, or any functional portion thereof. A “functional portion” of a promoter sequence known or provided herein is defined above.

According to embodiments of the present invention, the RNA molecule encoded by a second expression cassette comprising a transcribable DNA sequence operably linked to a second plant expressible promoter may cause a reduction or elimination in the level of expression of an mRNA transcript and/or protein encoded by an FT transgene in one or more plant tissues via suppression of the FT transgene. With the second expression cassette, the expression level of the transgenic FT transcript and/or protein may be reduced by at least 1%, at least 2%, at least 3%, at least 4%, at least 5%, at least 10%, at least 20%, at least 25%, at least 30%, at least 40%, at least 50%, at least 60%, at least 70%, at least 75%, at least 80%, at least 90%, at least 95%, at least 96%, at least 97%, at least 98%, or at least 99% in one or more plant tissues, such as one or more meristematic tissues, as compared to the mRNA transcript and/or protein level(s) of the FT transgene that would exist in the same plant tissue(s) without the second expression cassette. The mRNA transcript and/or protein level(s) of a FT transgene may be reduced by 1%-100%, 1%-75%, 1%-50%, 1%-25%, 5%-100%, 5%-95%, 5%-90%, 5%-85%, 5%-80%, 5%-75%, 5%-70%, 5%-65%, 5%-60%, 5%-55%, 5%-50%, 5%-45%, 5%-40%, 5%-35%, 5%-30%, 5%-25%, 5%-20%, 5%-15%, 5%-10%, 10%-100%, 10%-90%, 10%-80%, 10%-70%, 10%-60%, 10%-50%, 10%-40%, 10%-30%, 10%-20%, 25%-100%, 25%-75%, 25%-50%, 50%-100%, 50%-75%, or 75%-100% in one or more plant tissues as compared to the mRNA transcript and/or protein level(s) of the FT transgene that would exist in the same plant tissue(s) without the second expression cassette.

According to yet further embodiments, the second expression cassette may instead be designed to encode a RNA molecule that targets an endogenous FT gene for suppression. Selective suppression of the endogenous FT gene may be achieved by targeting its coding sequence if the FT transgene has a different coding sequence than the endogenous gene (i.e., the RNA molecule may comprise a targeting sequence that is complementary to at least a portion of a coding sequence of the endogenous FT gene). Alternatively, even if the coding sequences of the FT transgene and endogenous FT gene are the same or similar, the second expression cassette may be designed to encode a RNA molecule that targets a non-translated or non-coding sequence of a mRNA encoded by the endogenous FT gene, such as within the 5′ UTR, 3′UTR, intron, and/or leader sequence(s) of the endogenous FT mRNA transcript, if those sequences are different or lacking in the FT transgene (i.e., the RNA molecule may comprise a targeting sequence that is complementary to at least a portion of a non-translated or non-coding sequence of the endogenous FT gene). According to these embodiments, the transcribable DNA sequence of the second expression cassette may be designed according to the principles provided herein to target a particular coding or non-translated (non-coding) sequence of the mRNA encoded by the endogenous FT gene for suppression, instead of the mRNA sequence encoded by an FT transgene.

According to another broad aspect of the present invention, a recombinant DNA molecule, construct or vector is provided comprising a polynucleotide sequence encoding a FT protein (i.e., a FT transgene) operably linked to a plant expressible promoter, wherein the polynucleotide sequence further comprises a sequence encoding a target site or sensor for an endogenous RNA molecule, such as an endogenous miRNA or siRNA, the target site or sensor being present in the pre-mRNA and/or mature mRNA transcript encoded by the FT transgene, such as within the 5′ UTR, 3′UTR, intron, and/or leader sequence(s). As used herein, a “sensor” is a small noncoding RNA target site in a mRNA transcript of an FT transgene that is complementary to an endogamous RNA molecule, such as an endogenous miRNA or siRNA. The endogenous RNA molecule may be naturally occurring in a plant cell or tissue and function to suppress one or more target genes having the target site for the endogenous RNA molecule. It is presently proposed that a FT transgene may be further engineered to have a sequence encoding a mRNA target site or sensor for an endogenous RNA molecule, such that the FT transgene is suppressed by the endogenous RNA molecule. Suppression of the FT transgene may thus be used to mitigate the early termination phenotypes observed with transgenic FT expression alone (i.e., without suppression) similar to suppression via a second expression cassette encoding a RNA suppression molecule. The endogenous RNA molecule may be any known naturally occurring small RNA molecule, such as a miRNA, siRNA, etc., that functions to trigger suppression of one or more target genes in a plant cell. According to many embodiments, the endogenous RNA molecule may be naturally expressed during late vegetative and/or reproductive stages of development (e.g., in one or more late vegetative, reproductive, and/or floral tissue(s)), such that the endogenous RNA molecule causes suppression of the FT transgene after providing the initial floral induction signal. According to many embodiments, the plant expressible promoter, the target site, or both of the FT transgene are heterologous with respect to the polynucleotide coding sequence of the FT transgene.

According to some embodiments, the endogenous RNA molecule may be one or more endogenous miRNA molecules, such as one or more miR156 and/or miR172 RNA molecule(s). The sequence of the endogenous miR156 and miR172 molecule(s) will depend on the particular plant species in which the FT transgene will be expressed. An FT transgene may be designed to encode a mRNA target site or sensor for a miR156 or miR172 molecule present in the plant species of interest, which may be selected based on the expression level and timing of the one or more miR156 and miR172 molecule(s). In soybean, there are three miR172 molecules that are expressed at higher levels with timing near the vegetative-to-reproductive transition, miRNA172a (SEQ ID NO: 95), miRNA172c (SEQ ID NO: 96), or miRNA172k (SEQ ID NO: 97), and there are three miR156 molecules that are abundant at the juvenile-to-adult transition, miR156a (SEQ ID NO: 103), miR156c (SEQ ID NO: 104), or miR156q (SEQ ID NO: 105). Thus, a polynucleotide sequence encoding a FT protein may further comprise a sequence encoding one or more target site(s) or sensor(s) for one or more of such endogenous miR156 or miR172 RNA molecule(s). Each of the miR156 or miR172 target site(s) or sensor(s) may be present in the pre-mRNA and/or mature mRNA transcript encoded by the FT transgene, such as within a coding, 5′ UTR, 3′ UTR, and/or intronic mRNA sequence encoded by the polynucleotide sequence of the FT transgene, although miR156 or miR172 target site(s) or sensor(s) will more commonly be present in a non-coding and/or untranslated sequence. Examples of sequences encoding a target site or sensor for a soybean miR156 molecule include SEQ ID NOs: 106, 108, 109, and 110, which are complementary to one or more miR156 molecules. For example, the sequence provided as SEQ ID NO: 106 encodes SEQ ID NO: 107 as a mRNA target site or sensor for miR156. Examples of sequences encoding a target site or sensor for a soybean miR172 include SEQ ID NOs: 98, 100, and 101, which are complementary to one or more miR172 molecules. For example, the sequence provided as SEQ ID NO: 98 encodes SEQ ID NO: 99 as a mRNA target site or sensor for miR172. However, the sequence of a target site or sensor of a FT transgene may be determined based on the complementary sequence of a known miR156 or miR172 molecule. Indeed, depending on the sequence of the one or more endogenous miR156, miR172 and/or other small RNA molecule(s) naturally present in a given plant species, the transgenic FT expression cassette transformed into such a plant species may be engineered to have a sequence that encodes a target site or sensor for such an endogenous miR156, miR172, or other small RNA molecule. According to some embodiments, a first expression cassette comprising an FT transgene may be engineered to have a sequence encoding a target site or sensor for an endogenous miR156, miR172, or other small RNA molecule, even if a second expression cassette is present in the same recombinant DNA molecule construct or vector, or in the same transgenic plant, that comprises a transcribable DNA sequence encoding a RNA molecule that further targets the same FT transgene for suppression.

According to embodiments of the present invention, the target site for an endogenous RNA molecule will depend on the plant in which the FT transgene will be expressed. miR156 and miR172 sequences (and their target sites or sensors) are known for a variety of dicot species. The target site or sensor of the transgenic FT mRNA that is complementary to an endpgenpos RNA molecule may be 17, 18, 19, 20, 21, 22, 23, 24, 25, 26 or 27 (or more) nucleotides in length. Typically, a target site or sensor of an FT transgene will be designed to be 100% complementary to an endogenous miR156 or miR172. However, a target site or sensor for an endogenous RNA suppression molecule may not need to be 100% complementary to an endogenous miR156 or miR172 to be effective (i.e., to become hybridized by a miR156 or miR172 and targeted for suppression). For example, less than perfect complementarity may allow for more than one miR156, miR172 and/or other endogenous RNA molecule to hybridize to the target site or sensor. For any given plant species, the target site or sensor encoded by the polynucleotide coding sequence of a FT transgene may vary somewhat and still become bound by, or hybridized with, an endogenous RNA molecule, such as an endogenous miR156 or miR172 RNA molecule, when expressed in a plant cell. Accordingly, the target site of the mRNA transcript encoded by the FT transgene may contain one or more mismatches, such as 1, 2, 3, 4, 5, 6, 7, 8 or more mismatches depending on the alignment length between the endogenous RNA molecule (e.g., miR156, miR172) and the mRNA transcript. Indeed, the target site encoded by a polynucleotide coding sequence of an FT transgene may be at least 80%, at least 85%, at least 90%, at least 91%, at least 92%, at least 93%, at least 94%, at least 95%, at least 96%, at least 97% at least 98%, at least 99%, at least 99.5%, at least 99.9% or 100% complementary to the targeting sequence of an endogenous RNA molecule (e.g., miR156 or miR172), such as the target site or sensor for a miR172 molecule in soybean (SEQ ID NOs: 98, 100 or 101) or the target site or sensor for a miR156 molecule in soybean (SEQ ID NO: 106, 108, 109 or 110).

According to another broad aspect of the present invention, methods are provided for transforming a plant cell, tissue or explant with a recombinant DNA molecule, construct or vector provided herein to produce a transgenic plant. The recombinant DNA molecule, construct or vector may comprise an FT transgene or expression cassette. Depending on the manner of suppression, the FT transgene may further comprise a target site for an endogenous RNA molecule. The recombinant DNA molecule, construct or vector may comprise a second expression cassette comprising a transcribable DNA sequence encoding a RNA molecule that targets the FT transgene for suppression. Alternatively, the FT transgene and the transcribable DNA sequence encoding the RNA molecule may instead be present in two separate DNA molecules, constructs or vectors that may be co-transformed or transformed separately into plants. A recombinant DNA molecule, construct or vector comprising an FT transgene and the transcribable DNA sequence encoding an RNA molecule that targets the FT transgene for suppression may be transformed into a plant. According to other embodiments, a first recombinant DNA molecule, construct or vector comprising an FT transgene and a second recombinant DNA molecule, construct or vector comprising a transcribable DNA sequence encoding an RNA molecule that targets the FT transgene for suppression may each be co-transformed into a plant. According to other embodiments, a plant transformed with a first expression cassette comprising an FT transgene may be transformed with a second expression cassette comprising a transcribable DNA sequence encoding an RNA molecule that targets the FT transgene for suppression, or a plant transformed with a first expression cassette comprising a transcribable DNA sequence encoding an RNA molecule that targets the FT transgene for suppression may be transformed with a second expression cassette comprising an FT transgene. According to yet further embodiments, a first transgenic plant having a first expression cassette comprising (i) an FT transgene or (ii) a transcribable DNA sequence encoding an RNA molecule that targets an FT transgene for suppression may be crossed with a second plant having a second expression cassette comprising (i) a transcribable DNA sequence encoding an RNA molecule that targets the FT transgene for suppression or (ii) an FT transgene, such that one or more progeny plants may be produced comprising both the first and second expression cassettes (i.e., both the FT transgene and transcribable DNA sequence).

Numerous methods are known in the art for transforming chromosomes in a plant cell with a recombinant DNA molecule, construct or vector, which may be used according to methods of the present invention to produce a transgenic plant cell, plant part and plant. Any suitable method or technique for transformation of a plant cell known in the art may be used according to present methods. Effective methods for transformation of plants include bacterially mediated transformation, such as Agrobacterium-mediated or Rhizhobium-mediated transformation, and microprojectile bombardment-mediated transformation. A variety of methods are known in the art for transforming explants with a transformation vector via bacterially mediated transformation or microprojectile bombardment and then subsequently culturing, etc, those explants to regenerate or develop transgenic plants. Other methods for plant transformation, such as microinjection, electroporation, vacuum infiltration, pressure, sonication, silicon carbide fiber agitation, PEG-mediated transformation, etc., are also known in the art. Transgenic plants produced by these transformation methods may be chimeric or non-chimeric for the transformation event depending on the methods and explants used. Suitable methods for plastid transformation with a recombinant DNA molecule or construct are also known in the art.

Methods are further provided for expressing an FT transgene in one or more plant cells or tissues under the control of a vegetative-stage promoter, which may also be a meristem-preferred or meristem-specific promoter. Expression of the FT transgene may be modified, attenuated, and/or refined by the presence of a target site or sensor for an endogenous RNA molecule in the mRNA encoded by the FT transgene, such that the endogenous RNA molecule targets the FT transgene for suppression. In addition to an FT transgene, a RNA molecule that targets the FT transgene for suppression may also be expressed from a transcribable DNA sequence transformed into the plant. Such methods may be used to alter flowering time of a plant and/or the number of productive or successful flowers, fruits, pods, and/or seeds per node of the plant relative to a wild type or control plant not having the FT transgene. Indeed, methods of the present invention may be used to alter reproductive or yield-related phenotype(s) or trait(s) of the transgenic plant.

Transformation of a target plant material or explant may be practiced in tissue culture on nutrient media, for example a mixture of nutrients that allow cells to grow in vitro. Recipient cell targets or explants may include, but are not limited to, meristems, shoot tips, protoplasts, hypocotyls, calli, immature or mature embryos, shoots, buds, nodal sections, leaves, gametic cells such as microspores, pollen, sperm and egg cells, etc., or any suitable portions thereof. It is contemplated that any transformable cell or tissue from which a fertile plant can be regenerated or grown/developed may be used as a target for transformation. Transformed explants, cells or tissues may be subjected to additional culturing steps, such as callus induction, selection, regeneration, etc., as known in the art. Transformed cells, tissues or explants containing a recombinant DNA insertion may be grown, developed or regenerated into transgenic plants in culture, plugs or soil according to methods known in the art. Transgenic plants may be further crossed to themselves or other plants to produce transgenic seeds and progeny. A transgenic plant may also be prepared by crossing a first plant comprising the recombinant DNA sequence or transformation event with a second plant lacking the insertion. For example, a recombinant DNA sequence may be introduced into a first plant line that is amenable to transformation, which may then be crossed with a second plant line to introgress the recombinant DNA sequence into the second plant line. Progeny of these crosses can be further back crossed into the more desirable line multiple times, such as through 6 to 8 generations or back crosses, to produce a progeny plant with substantially the same genotype as the original parental line but for the introduction of the recombinant DNA sequence.

A recombinant DNA construct or expression cassette of the present invention may be included within a DNA transformation vector for use in transformation of a target plant cell, tissue or explant. Such a transformation vector of the present invention may generally comprise sequences or elements necessary or beneficial for effective transformation in addition to the transcribable DNA sequence and/or FT transgene or expression cassette. For Agrobacterium-mediated transformation, the transformation vector may comprise an engineered transfer DNA (or T-DNA) segment or region having two border sequences, a left border (LB) and a right border (RB), flanking at least the transcribable DNA sequence and/or FT transgene, such that insertion of the T-DNA into the plant genome will create a transformation event for the transcribable DNA sequence and/or FT transgene. In other words, the transcribable DNA sequence and/or FT transgene would be located between the left and right borders of the T-DNA, perhaps along with an additional transgene(s) or expression cassette(s), such as a plant selectable marker transgene and/or other gene(s) of agronomic interest that may confer a trait or phenotype of agronomic interest to a plant. In addition to protein encoding sequences, a gene of agronomic interest may further comprise a polynucleotide sequence encoding a RNA suppression element. According to some embodiments, the transcribable DNA sequence and/or FT transgene and the plant selectable marker transgene (or other gene of agronomic interest) may be present in separate T-DNA segments on the same or different recombinant DNA molecule(s), such as for co-transformation. A transformation vector or construct may further comprise prokaryotic maintenance elements, which for Agrobacterium-mediated transformation may be located in the vector backbone outside of the T-DNA region(s).

According to some embodiments, a first expression cassette comprising an FT transgene and a second expression cassette comprising a transcribable DNA sequence encoding a RNA molecule that targets the FT transgene for suppression may be present in the same T-DNA of a transformation vector (i.e., between the same right and left T-DNA borders); or a first expression cassette comprising an FT transgene may be present in a first T-DNA (comprising a first right border and a first left border), and a second expression cassette comprising a transcribable DNA sequence encoding a RNA molecule that targets the FT transgene for suppression may be present in a second T-DNA (comprising a second right border and a second left border), wherein the first and second T-DNAs are in the same transformation vector; or a first expression cassette comprising an FT transgene may be present in a first T-DNA of a first transformation vector, and a second expression cassette comprising a transcribable DNA sequence encoding a RNA molecule that targets the FT transgene for suppression may be present in a second T-DNA of a second transformation vector. The first and second expression cassettes present in one or two transformation vectors may be co-transformed into a plant cell, or the first and second expression cassettes may be present in two separate transformation vectors and transformed into plant cells separately. A first or second expression cassette may be transformed into one or more plant cells already having a transformation event for the other expression cassette, or the first and second expression cassettes may be transformed into different plant cells that may be developed or regenerated into a first and second transgenic plant. The first or second transgenic plants and/or their progeny may be crossed to each other, such that the first and second expression cassettes are brought together and present in the same plant.

A plant selectable marker transgene in a transformation vector or construct of the present invention may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known.

According to embodiments of the present invention, methods for transforming a plant cell, tissue or explant with a recombinant DNA molecule or construct may further include site-directed or targeted integration. According to these methods, a portion of a recombinant DNA donor template molecule (i.e., an insertion sequence) may be inserted or integrated at a desired site or locus within a plant genome. The insertion sequence of the donor template may comprise a transgene or construct, such as (i) an FT transgene or construct comprising a polynucleotide sequence encoding a florigenic FT protein operably linked to a vegetative-stage promoter, which may also be a meristem-preferred or meristem-specific promoter, and/or (ii) a transcribable DNA sequence encoding a RNA molecule that targets an FT transgene for suppression and operably linked to a vegetative stage promoter and/or reproductive stage promoter, which may also be a meristem-preferred or meristem-specific promoter. The donor template may also have one or two homology arms flanking the insertion sequence to promote the targeted insertion event through homologous recombination and/or homology-directed repair. Thus, a recombinant DNA molecule of the present invention may further include a donor template for site-directed or targeted integration of a transgene or construct, such as an FT transgene or construct, into the genome of a plant.

Any site or locus within the genome of a plant may potentially be chosen for site-directed integration of a transgene or construct of the present invention. For site-directed integration, a double-strand break or nick may first be made at a selected genomic locus with a site-specific nuclease, such as, for example, a zinc-finger nuclease (ZFN), a meganuclease, a transcription activator-like nuclease (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof); a recombinase (without being limiting, for example, a tyrosine recombinase attached to a DNA recognition motif (e.g., Cre recombinase, Flp recombinase, Tnp1 recombinase), a serine recombinase attached to a DNA recognition motif (e.g., PhiC31 integrase, R4 integrase, TP-901 integrase); a transposase (without being limiting, for example, a DNA transposase attached to a DNA binding domain); or any combination thereof. Also provided are guide RNAs (e.g., CRISPR RNAs (crRNAs), trans-activating CRISPR RNAs (tracrRNAs), guide RNAs (gRNAs), single-guide RNAs (sgRNAs)) useful for methods of using RNA-guided nucleases. Any method known in the art for site-directed integration may be used. In the presence of a donor template molecule, the double strand break or nick may then be repaired by homologous recombination between the homology arm(s) of the donor template and the plant genome, or by non-homologous end joining (NHEJ), resulting in site-directed integration of the insertion sequence into a plant genome to create the targeted insertion event at or near the site of the double strand break or nick. Thus, site-specific insertion or integration of a transgene or construct may be achieved.

As used herein, the term “insertion” in reference to plant transformation or site-directed integration refers to an insertion or integration of an exogenous polynucleotide or DNA construct, molecule or sequence, such as a transformation vector or T-DNA sequence or an insertion sequence of a donor template, into the genome of a plant. In this context, the term “exogenous” refers to a polynucleotide or DNA construct, molecule or sequence that is introduced into a plant cell or tissue using any suitable plant transformation or genome editing method or technique known in the art.

According to embodiments of the present invention, a plant that may be transformed with a recombinant DNA molecule or transformation vector comprising an FT transgene and/or a transcribable DNA sequence encoding a RNA molecule that targets an FT transgene for suppression may include a variety of flowering plants or angiosperms, which may be further defined as including various dicotyledonous (dicot) plant species, such as soybean, cotton, alfalfa, canola, sugar beets, alfalfa and other leguminous plants. A dicot plant could be a member of the Brassica sp. (e.g., B. napus, B. rapa, B. juncea), particularly those Brassica species useful as sources of seed oil, alfalfa (Medicago sativa), sunflower (Helianthus annuus), safflower (Carthamus tinctorius), oil palm (Elaeis spp.), sesame (Sesamum spp.), coconut (Cocos spp.), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus casica), banana (Musa spp.), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), sugar beet (Beta vulgaris), grape (Vitis, Muscadinia), tomato (Lycopersicon esculentum, Solanum lycopersicum), and cucumber (Cucumis sativis). Leguminous plants include beans and peas. Beans include, for example, guar, locust bean, fenugreek, soybean, garden beans, cowpea, mungbean, lima bean, fava bean, lentils, and chickpea. Given that the present invention may apply to a broad range of plant species, the present invention further applies to other botanical structures analogous to pods of leguminous plants, such as bolls, siliques, fruits, nuts, tubers, etc.

According to embodiments of the present invention and depending on the particular plant species transformed, a plant ectopically expressing a florigenic FT sequence, which may be modified via suppression as provided herein, may have an altered or greater number of bolls, siliques, fruits, nuts, tubers, etc., per node(s), main stem, and/or branch(es) of the plant, and/or an altered or greater number of bolls, siliques, fruits, nuts, tubers, etc., per plant, relative to a wild type or control plant not having the FT transgene.

According to another broad aspect of the present invention, a transgenic plant(s), plant cell(s), seed(s), and plant part(s) are provided comprising one or more transformation events or insertions into the genome of at least one plant cell thereof, the transformation event or insertion comprising (i) a recombinant DNA sequence, construct or polynucleotide including a Flowering Locus T (FT) transgene, wherein the FT transgene comprises a polynucleotide sequence encoding an FT protein operably linked to a vegetative stage promoter, which may also be a meristem-preferred or meristem-specific promoter, and/or (ii) a transcribable DNA sequence encoding a RNA molecule that targets an FT transgene for suppression operably linked to a vegetative stage and/or reproductive stage promoter, which may also be a meristem-preferred or meristem-specific promoter. As provided herein, the FT transgene may be targeted for suppression by an transgenically expressed and/or endogenous RNA molecule. The RNA molecule may be encoded by a transcribable DNA sequence that is also transformed into the plant, plant part, plant seed or plant cell. The FT protein encoded by the polynucleotide sequence may correspond to a native FT gene in the transgenic plant transformed with the polynucleotide coding sequence, or homologous or otherwise similar to a FT protein native to the transgenic plant (i.e., not native to the transgenic plant but similar to a native or endogenous FT protein), or heterologous to the transgenic plant. Such a transgenic plant may be produced by any suitable transformation method, which may be followed by selection, culturing, regeneration, development, etc., as desired or needed to produce a transgenic R₀ plant, which may then be selfed or crossed to other plants to generate R1 seed and subsequent progeny generations and seed through additional crosses, etc. Similarly, embodiments of the present invention further include a plant cell, tissue, explant, etc., comprising one or more transgenic cells having a transformation event or genomic insertion of a recombinant DNA or polynucleotide sequence comprising an FT transgene and/or a transcribable DNA sequence encoding a RNA molecule that targets an FT transgene for suppression.

Transgenic plants, plant cells, seeds, and plant parts of the present invention may be homozygous or hemizygous for a transgenic event or insertion of an FT transgene and/or transcribable DNA sequence into the genome of at least one plant cell thereof, or may contain any number of copies of a transgenic event(s) or insertion(s) comprising an FT transgene and/or transcribable DNA sequence. The dosage or amount of expression of an FT transgene may be altered by its zygosity and/or number of copies, which may affect the degree or extent of phenotypic changes in the transgenic plant, etc. According to some embodiments, a transgenic plant comprising an FT transgene and/or FT suppression element, which may be modified, attenuated and/or refined as provided herein, may be further characterized as having one or more altered flowering or reproductive phenotypes or traits, which may include altered yield-related phenotypes or traits, such as an increase in the number of flowers, pods, etc., and/or seeds per plant (and/or per node of the plant) relative to a wild type or control plant not having the FT transgene (and/or not having a FT suppression element). Such a transgenic plant may be further characterized as having an altered structure, morphology, and/or architecture due to altered plant height, branching patterns, number of floral nodes, etc., relative to a wild type or control plant. Indeed, yield-related phenotypes or traits altered by FT overexpression in a transgenic plant may include: flowering time, reproductive duration, flowering duration, amount or timing of abscission of flowers, pods, siliques, bolls, fruits, nuts, etc., number of flowers per node, number of racemes per node, number of branches per plant, number of nodes per plant, number of nodes on the main stem, number of nodes on branches, number of pods, bolls, siliques, fruits, nuts, etc., per plant, number of pods, bolls, siliques, fruits, nuts, etc., per node, number of pods, bolls, siliques, fruits, nuts, etc., on the main stem, number of pods, seeds, bolls, siliques, fruits, nuts, etc., on branches, seed weight (such as 1000 seed weight), number of seeds per plant, number of seeds on the main stem, number of seeds per node, and/or altered plant architecture, as compared to a wild type or control plant not having the FT transgene (and/or not having a FT suppression element). As used herein, the term “overexpression” in reference to a FT transgene includes ectopic expression of the transgene.

For purposes of the present invention, a “plant” may include an explant, seedling, plantlet or whole plant at any stage of regeneration or development. As used herein, a “transgenic plant” refers to a plant whose genome has been altered by the integration or insertion of a recombinant DNA molecule, construct or sequence. A transgenic plant includes an R₀ plant developed or regenerated from an originally transformed plant cell(s) as well as progeny transgenic plants in later generations or crosses from the R₀ transgenic plant. As used herein, a “plant part” may refer to any organ or intact tissue of a plant, such as a meristem, shoot organ/structure (e.g., leaf, stem and tuber), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, ground tissue, and the like), or any portion thereof. Plant parts of the present invention may be viable, nonviable, regenerable, and/or non-regenerable. A “propagule” may include any plant part that is capable of growing into an entire plant. For purposes of the present invention, a plant cell transformed with an FT transgene and/or FT suppression element according to embodiments of the present invention may include any plant cell that is competent for transformation as understood in the art based on the method of transformation, such as a meristem cell, an embryonic cell, a callus cell, etc. As used herein, a “transgenic plant cell” simply refers to any plant cell that is transformed with a stably-integrated recombinant DNA molecule or sequence. A transgenic plant cell may include an originally-transformed plant cell, a transgenic plant cell of a regenerated or developed R₀ plant, or a transgenic plant cell from any progeny plant or offspring of the transformed R₀ plant, including cell(s) of a plant seed or embryo, or a cultured plant or callus cell, etc.

According to many embodiments, a transgenic plant may comprise a polynucleotide sequence encoding a florigenic FT protein operably linked to a vegetative stage promoter. According to some embodiments, expression of a florigenic FT protein in a transgenic plant may be suppressed in a vegetative and/or reproductive stage and/or tissue of a transgenic plant, such as via an endogenous and/or transgenically or ectopically expressed RNA molecule. According to some embodiments, expression of a florigenic FT protein in a transgenic plant may be spatially and/or temporally restricted by a small RNA molecule. According to some embodiments, a transgenic plant may comprise a transcribable DNA sequence encoding a RNA molecule that targets a florigenic FT gene or transgene for suppression.

Embodiments of the present invention may further include methods for making or producing transgenic plants having altered reproductive and/or yield-related traits or phenotypes, such as by transformation, crossing, etc., wherein the method comprises introducing a recombinant DNA molecule, construct or sequence comprising an FT transgene and/or a transcribable DNA sequence encoding a RNA molecule that targets an FT transgene for suppression into a plant cell, and then regenerating or developing the transgenic plant from the transformed plant cell, which may be performed under selection pressure favoring the transgenic event. Such methods may comprise transforming a plant cell with a recombinant DNA molecule or sequence comprising an FT transgene and/or a transcribable DNA sequence, and selecting for a plant having one or more altered phenotypes or traits, such as one or more of the following: flowering time, reproductive duration, flowering duration, amount or timing of abscission of flowers, pods, bolls, siliques, fruits, nuts, etc., number of flowers per node, number of racemes per node, number of branches per plant, number of nodes per plant, number of nodes on the main stem, number of nodes on branches, number of pods, bolls, siliques, fruits, nuts, etc., per plant, number of pods, bolls, siliques, fruits, nuts, etc., per node, number of pods, bolls, siliques, fruits, nuts, etc., on the main stem, number of pods, seeds, bolls, siliques, fruits, nuts, etc., on branches, seed weight (such as 1000 seed weight), number of seeds per plant, number of seeds on the main stem, number of seeds per node, and altered plant architecture, as compared to a wild type or control plant not having the FT transgene (and/or not having an FT suppression element). For example, embodiments of the present invention may comprise methods for producing a transgenic plant having an increased number of flowers, pods, and/or seeds per plant (and/or an increased number of flowers, pods, and/or seeds per node of the plant), wherein the method comprises introducing a recombinant DNA molecule comprising an FT transgene and/or a transcribable DNA sequence into a plant cell, and then regenerating or developing the transgenic plant from the plant cell. The transgenic plant may then be selected based on one or more of the above reproductive and/or yield-related traits or phenotypes. A transgenic plant, plant cell or plant tissue may also be selected based on the presence of an FT transgene and/or FT suppression element using one or more methods or kits known in the art, such as DNA sequencing, hybridization, antibody binding, and/or other molecular techniques.

According to embodiments of the present invention, a transgenic plant may have at least 1%, at least 2%, at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more flowers, pods, seeds, bolls, siliques, fruits, nuts or tubers than a non-transgenic control plant. According to some embodiments, a transgenic plant may have an average of at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more flowers, pods, seeds, bolls, siliques, fruits, nuts or tubers per node than a non-transgenic control plant. A transgenic plant may have an average of at least 2, at least 3, at least 4, at least 5, at least 6, at least 7, at least 8, at least 9, or at least 10 pods, bolls, siliques, fruits, nuts or tubers per node. A transgenic plant may have an average of 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, or 4 to 5 pods, bolls, siliques, fruits, nuts or tubers per node. A transgenic plant may have an average of at least 1, at least 2, at least 3, at least 4, at least 5, at least 6, at least 7, at least 8, at least 9, or at least 10 more flowers, pods, seeds, bolls, siliques, fruits, nuts or tubers per node as compared to a non-transgenic control plant. A transgenic plant may be a soybean plant, and the transgenic plant may have more pods and/or seeds per node on average than a non-transgenic control plant. A transgenic plant may flower at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier than a non-transgenic control plant.

According to embodiments of the present invention, a transgenic plant is provided comprising a polynucleotide sequence encoding a florigenic FT protein operably linked to a vegetative stage promoter, wherein expression of the polynucleotide coding sequence is spatially and/or temporally attenuated, restricted, modified, and/or refined by a RNA molecule, which may be a small non-coding RNA molecule. The level of expression or translation of the florigenic FT mRNA and/or protein in the transgenic plant may be suppressed or lowered in one or more meristematic, reproductive and/or floral tissues and/or during one or more reproductive stages. According to some embodiments, the reproductive duration and/or flowering duration of a transgenic plant comprising a FT transgene and/or FT suppression element as provided herein may be at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days longer than the reproductive duration and/or flowering duration of a wild-type or control plant, but may also be no more than (i.e., not greater or more than) 3, 4, 5, 6, 7, 8, 9, 10, 11, 12, 13, 14, 15, 20, 25, 30, 35, 40, 45, 50, 55, or 60 days longer than the reproductive duration and/or flowering duration of a wild-type or control plant. According to some embodiments, the onset of flowering (i.e., the appearance of the first open flower) of a transgenic plant comprising a FT transgene and/or FT suppression element as provided herein may be or occur at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier than a wild-type or control plant, but may also be no more than (i.e., not greater or more than) 3, 4, 5, 6, 7, 8, 9, 10, 11, 12, 13, 14, 15, 20, 25, 30, 35, 40, 45, 50, 55, or 60 days earlier than the onset of flowering of a wild-type or control plant.

According to some embodiments, the number of flowers, seeds, bolls, siliques, fruits, nuts, pods or tubers on a transgenic plant comprising a FT transgene and/or FT suppression element as provided herein may be at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% greater than a wild-type or control plant. According to some embodiments, the number of flowers, seeds, bolls, siliques, fruits, nuts or pods on the main stem of a transgenic plant comprising a FT transgene and/or FT suppression element as provided herein may be at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, or at least 200%, at least 250%, at least 300%, at least 350%, or at least 400% greater than a wild-type or control plant. According to some embodiments, the number of flowers, seeds, bolls, siliques, fruits, nuts or pods per node on the main stem of a transgenic plant comprising a FT transgene and/or FT suppression element as provided herein may be at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, or at least 200%, at least 250%, at least 300%, at least 350%, or at least 400% greater than a wild-type or control plant.

Each of these trait amounts or numbers per plant, main stem or branch, such as the number of flowers, bolls, seeds, siliques, fruits, nuts or pods per plant, main stem or branch may be calculated as an average of two or more plants or determined for one plant, and the amounts or numbers of flowers, bolls, seeds, siliques, fruits, nuts or pods per node may be calculated as an average for one or more plants. Thus, the percentage changes may be calculated between two plants, between a plant and an average of two or more plants, or between two averages (with each average being of two or more plants).

According to some embodiments, the number of flowers, bolls, seeds, siliques, fruits, nuts, pods or tubers per node on a transgenic plant comprising a FT transgene and/or FT suppression element as provided herein may on average be at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, or at least 200%, at least 250%, at least 300%, at least 350%, or at least 400% greater than a wild-type or control plant. According to some embodiments, a transgenic plant comprising a FT transgene and/or FT suppression element as provided herein may have on average at least 2, at least 3, at least 4, at least 5, at least 6, at least 7, at least 8, at least 9, or at least 10 flowers, pods, bolls, seeds, siliques, fruits, nuts, pods or tubers per node. According to some embodiments, a transgenic plant comprising a FT transgene and/or FT suppression element as provided herein may have on average 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 4 to 10, 4 to 9, 4 to 8, 4 to 7, 4 to 6, or 4 to 5 pods, or about 2, about 3, about 4, about 5, about 6, about 7, about 8, about 9, or about 10 flowers, bolls, siliques, fruits, nuts, pods or tubers per node. According to some embodiments, a transgenic plant comprising a FT transgene and/or FT suppression element as provided herein may have on average at least 1, at least 2, at least 3, at least 4, at least 5, at least 6, at least 7, at least 8, at least 9, or at least 10 more flowers, bolls, seeds, siliques, fruits, nuts, pods or tubers per node as compared to a non-transgenic wild-type or control plant.

According to some embodiments, the average number of bolls, siliques, seeds, fruits, nuts, pods or tubers, and/or the average number of flowers, bolls, siliques, seeds, fruits, nuts, pods or tubers per node, on a transgenic plant comprising a FT transgene and/or FT suppression element as provided herein (or on the main stem of such a transgenic plant) may be 1%-400%, 1%-350%, 1%-300%, 1%-250%, 1%-200%, 1%-150%, 1%-100%, 1%-75%, 1%-50%, 1%-25%, 5%-400%, 5%-350%, 5%-300%, 5%-250%, 5%-200%, 5%-150%, 5%-100%, 5%-95%, 5%-90%, 5%-85%, 5%-80%, 5%-75%, 5%-70%, 5%-65%, 5%-60%, 5%-55%, 5%-50%, 5%-45%, 5%-40%, 5%-35%, 5%-30%, 5%-25%, 5%-20%, 5%-15%, 5%-10%, 10%-400%, 10%-350%, 10%-300%, 10%-250%, 10%-200%, 10%-150%, 10%-100%, 10%-90%, 10%-80%, 10%-70%, 10%-60%, 10%-50%, 10%-40%, 10%-30%, 10%-20%, 25%-400%, 25%-350%, 25%-300%, 25%-250%, 25%-200%, 25%-150%, 25%-100%, 25%-75%, 25%-50%, 50%-400%, 50%-350%, 50%-300%, 50%-250%, 50%-200%, 50%-150%, 50%-100%, 50%-75%, 75%-400%, 75%-350%, 75%-300%, 75%-250%, 75%-200%, 75%-150%, 75%-100%, 100%-400%, 100%-350%, 100%-300%, 100%-250%, 100%-200%, or 100%-150% greater than a wild-type or control plant. According to some embodiments, the reproductive duration of a transgenic plant comprising a FT transgene and/or FT suppression element as provided herein may be 1%-400%, 1%-350%, 1%-300%, 1%-250%, 1%-200%, 1%-150%, 1%-100%, 1%-75%, 1%-50%, 1%-25%, 5%-400%, 5%-350%, 5%-300%, 5%-250%, 5%-200%, 5%-150%, 5%-100%, 5%-95%, 5%-90%, 5%-85%, 5%-80%, 5%-75%, 5%-70%, 5%-65%, 5%-60%, 5%-55%, 5%-50%, 5%-45%, 5%-40%, 5%-35%, 5%-30%, 5%-25%, 5%-20%, 5%-15%, 5%-10%, 10%-400%, 10%-350%, 10%-300%, 10%-250%, 10%-200%, 10%-150%, 10%-100%, 10%-90%, 10%-80%, 10%-70%, 10%-60%, 10%-50%, 10%-40%, 10%-30%, 10%-20%, 25%-400%, 25%-350%, 25%-300%, 25%-250%, 25%-200%, 25%-150%, 25%-100%, 25%-75%, 25%-50%, 50%-400%, 50%-350%, 50%-300%, 50%-250%, 50%-200%, 50%-150%, 50%-100%, 50%-75%, 75%-400%, 75%-350%, 75%-300%, 75%-250%, 75%-200%, 75%-150%, 75%-100%, 100%-400%, 100%-350%, 100%-300%, 100%-250%, 100%-200%, or 100%-150% greater in terms of number of days than a wild-type or control plant. According to some embodiments, the onset of flowering of a transgenic plant comprising a FT transgene and/or FT suppression element as provided herein may be 1%-400%, 1%-350%, 1%-300%, 1%-250%, 1%-200%, 1%-150%, 1%-100%, 1%-75%, 1%-50%, 1%-25%, 5%-400%, 5%-350%, 5%-300%, 5%-250%, 5%-200%, 5%-150%, 5%-100%, 5%-95%, 5%-90%, 5%-85%, 5%-80%, 5%-75%, 5%-70%, 5%-65%, 5%-60%, 5%-55%, 5%-50%, 5%-45%, 5%-40%, 5%-35%, 5%-30%, 5%-25%, 5%-20%, 5%-15%, 5%-10%, 10%-400%, 10%-350%, 10%-300%, 10%-250%, 10%-200%, 10%-150%, 10%-100%, 10%-90%, 10%-80%, 10%-70%, 10%-60%, 10%-50%, 10%-40%, 10%-30%, 10%-20%, 25%-400%, 25%-350%, 25%-300%, 25%-250%, 25%-200%, 25%-150%, 25%-100%, 25%-75%, 25%-50%, 50%-400%, 50%-350%, 50%-300%, 50%-250%, 50%-200%, 50%-150%, 50%-100%, 50%-75%, 75%-400%, 75%-350%, 75%-300%, 75%-250%, 75%-200%, 75%-150%, 75%-100%, 100%-400%, 100%-350%, 100%-300%, 100%-250%, 100%-200%, or 100%-150% earlier in terms of number of days than a wild-type or control plant.

According to embodiments of the present invention, transgenic plants provided herein may have a combination of two or more traits or phenotypes described herein, such as two or more of increased pods, bolls, siliques, seeds, fruits, nuts or tubers per node, increased pods, bolls, siliques, seeds, fruits, nuts or tubers on the main stem, increased reproductive duration, earlier onset of flowering, minimal plant height, such as at least 900 or more millimeters (i.e., greater than or equal to 0.9 meters) in the case of soybean, and/or reduced branching, relative to a wild-type or control plant. According to some embodiments, transgenic plants may have an increased number of pods, bolls, siliques, seeds, fruits, or nuts per node on average and an earlier onset of flowering, relative to a wild-type or control plant. Such transgenic plants may further have an increase in the number of pods, bolls, siliques, seeds, fruits, nuts or tubers on the mainstem and/or an increased reproductive duration, relative to a wild-type or control plant.

For example, a transgenic plant provided herein may have at least 1%, at least 2%, at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more flowers, pods, seeds, bolls, siliques, fruits, nuts or tubers per node, and an onset of flowering that is at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier, relative to a wild-type or control plant. A transgenic plant provided herein may have an average of at least 1, at least 2, at least 3, at least 4, at least 5, at least 6, at least 7, at least 8, at least 9, or at least 10 more flowers, pods, seeds, bolls, siliques, fruits, nuts or tubers per node, and an onset of flowering that is at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier, relative to a wild-type or control plant. Such transgenic plants may further have a reproductive and/or flowering duration that is at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days longer than the reproductive duration and/or flowering duration of a wild-type or control plant, and/or an increased number of flowers, pods, seeds, bolls, siliques, fruits or nuts on the main stem of the transgenic plant that is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, or at least 200%, at least 250%, at least 300%, at least 350%, or at least 400% greater than a wild-type or control plant.

According to embodiments of the present invention, transgenic plants provided herein may have an altered plant architecture with a minimal plant height and reduced branching, which may be accompanied by a mitigated reduction in the number of nodes per plant. Transgenic plants expressing the FT transgene alone may have a severe dwarf phenotype due to earlier termination that includes short plant height along with reduced branching and nodes per plant. By expressing the FT transgene with a miRNA sensor or second suppression element targeting the FT transgene, these severe early termination phenotypes may be mitigated to produce a more normal plant height while maintaining increased pods per node. According to these embodiments, transgenic plants may have (i) a minimal plant height that is reduced by no more than 5%, 10%, 15%, 20%, 25%, 30%, 35%, or 40% relative to a wild-type or control plant (i.e., the difference in plant heights between the transgenic plant and the wild-type or control plant is no greater than one or more of these percentages), (ii) a total number of branches that is reduced by at least 50%, at least 55%, at least 60%, at least 65%, at least 70%, at least 75%, at least 80%, at least 85%, at least 90%, or at least 95% relative to a wild-type or control plant, and/or (iii) a minimal number of nodes per plant (and/or per main stem) that is reduced by no more than 5%, 10%, 15%, 20%, 25%, 30%, 35%, or 40% relative to a wild-type or control plant (i.e., the difference in number of nodes per plant (and/or per main stem) between the transgenic plant and the wild-type or control plant is no greater than one or more of these percentages). In the case of soybean, a transgenic plant provided herein may have a minimal plant height that is at least 700 millimeters (mm), at least 750 mm, at least 800 mm, at least 850 mm, at least 900 mm, at least 950 mm, at least 1000 mm, at least 1050 mm, at least 1100 mm, at least 1150 mm, at least 1200 mm, at least 1250 mm, at least 1300 mm, at least 1350 mm, or at least 1400 mm; at least 100, at least 125, at least 150, at least 175, at least 200, at least 225, at least 250, at least 275, at least 300, at least 325, at least 350, at least 375, or at least 400 total number of nodes per plant; and/or a total number of branches that is reduced by at least 70%, at least 75%, at least 80%, at least 85%, at least 90%, or at least 95% relative to a wild-type or control plant. All of the foregoing numbers, percentages and differences for a plant further include those values calculated from an average of a plurality of plants of the same type.

According to another broad aspect of the present invention, methods are provided for planting transgenic plants of the present invention at a normal or high density in the field. According to some embodiments, the yield of a crop plant per acre (or per land area) may be increased by planting transgenic plants of the present invention at a higher density in the field. As described herein, transgenic plants of the present invention expressing a florigenic FT protein during vegetative stage(s) of development and/or with suppression of the FT transgene may exhibit increased pods and/or seeds per node (particularly on the main stem), but may also have an altered plant architecture with reduced branching and fewer nodes per branch. Thus, it is proposed that transgenic plants of the present invention may be planted at a higher density to increase yield per acre in the field. For row crops, higher density may be achieved by planting a greater number of seeds/plants per row length and/or by decreasing the spacing between rows. According to some embodiments, a transgenic crop plant of the present invention may be planted at a density in the field (plants per land/field area) that is at least 5%, 10%, 15%, 20%, 25%, 50%, 75%, 100%, 125%, 150%, 175%, 200%, 225%, or 250% higher or greater than a normal planting density for that crop plant according to standard agronomic practices.

For soybean, the typical planting density is in a range from about 100,000 to 150,000 seeds per acre, and the typical row spacing is in a range from about 26 to about 40 inches, such as 30 inch or 36 inch row spacing. Within a given row, about 6-8 soybean seeds may typically be planted per foot. In contrast, high density planting for soybean may include a range of approximately 150,000 to 250,000 seeds per acre, and the row spacing may be within a range from about 10 inches or less to about 25 inches, such as 10 inch, 15 inch or 20 inch row spacing. For high density planting, approximately 9-12 soybean seeds per foot may be planted within each row, perhaps in combination with narrower row spacing. However, high crop density may be achieved by narrow row spacing without an increase in planting density within each row.

For cotton, the typical planting density is in a range from about 28,000 to 45,000 seeds per acre, and the typical row spacing is in a range from about 38 to about 40 inches, such as 38 inch or 40 inch row spacing. Within a given row, about 2-3 cotton seeds may typically be planted per foot. In contrast, high density planting for soybean may include a range of approximately 48,000 to 60,000 seeds per acre, and the row spacing may be within a range from about 30 inches or less to about 36 inches. For high density planting, approximately 3-5 cotton seeds per foot may be planted within each row, perhaps in combination with narrower row spacing. However, high crop density for cotton may be achieved by narrow row spacing without an increase in planting density within each row.

For canola, the typical planting density is in a range from about 360,000 to 550,000 seeds per acre, and the typical row spacing between openers is in a range from about 6 inches to about 16 inches. Within a given row, about 8-12 canola seeds may typically be planted per foot. In contrast, high density planting for soybean may include a range of approximately 450,000 to 680,000 seeds per acre, and the row spacing may be within a range from about 5 inches or less to about 10 inches. For high density planting, approximately 10-16 canola seeds per foot may be planted within each row, perhaps in combination with the narrower row spacing. However, high crop density for canola may be achieved by narrow row spacing without an increase in planting density within each row.

The following are non-limiting exemplary embodiments of the present disclosure:
1. A recombinant DNA construct comprising a first expression cassette and a second expression cassette, wherein the first expression cassette comprises a polynucleotide sequence encoding a florigenic FT protein operably linked to a first plant expressible promoter, and the second expression cassette comprises a transcribable DNA sequence encoding a RNA molecule comprising a targeting sequence that is at least 80% complementary to at least 15 consecutive nucleotides of the polynucleotide sequence of the first expression cassette, and wherein the transcribable DNA sequence is operably linked to a second plant expressible promoter.
2. The recombinant DNA construct of embodiment 1, wherein the targeting sequence of the RNA molecule is from about 15 to about 27 nucleotides in length.
3. The recombinant DNA construct of embodiments 1 or 2, wherein the targeting sequence of the RNA molecule is 15, 16, 17, 18, 19, 20, 21, 22, 23, 24, 25, 26 or 27 nucleotides in length.
4. The recombinant DNA construct of any one of embodiments 1-3, wherein the targeting sequence of the RNA molecule is at least 85%, at least 90%, at least 95%, at least 96%, at least 97%, at least 98%, at least 99%, at least 99.5% or 100% complementary to at least 15 consecutive nucleotides of the polynucleotide sequence of the first expression cassette.
5. The recombinant DNA construct of any one of embodiments 1-4, wherein the targeting sequence of the RNA molecule is at least 80% complementary to at least 15 consecutive nucleotides of a mRNA transcript encoded by the polynucleotide sequence of the first expression cassette.
6. The recombinant DNA construct of any one of embodiments 1-5, wherein the targeting sequence of the RNA molecule is at least 80% complementary to at least 15 consecutive nucleotides of an exonic or coding sequence of the mRNA transcript.
7. The recombinant DNA construct of any one of embodiments 1-5, wherein the targeting sequence of the RNA molecule is at least 80% complementary to at least 15 consecutive nucleotides of a non-coding sequence of the mRNA transcript.
8. The recombinant DNA construct of any one of embodiments 1-7, wherein the RNA molecule encoded by the transcribable DNA sequence is a precursor miRNA or siRNA.
9. The recombinant DNA construct of any one of embodiments 1-8, wherein the transcribable DNA sequence comprises a sequence that is at least 80%, at least 85%, at least 90%, at least 95%, at least 96%, at least 97%, at least 98%, at least 99% or 100% complementary to SEQ ID NO: 68 or 69.
10. The recombinant DNA construct of any one of embodiments 1-9, wherein the transcribable DNA sequence comprises a sequence that is at least at least 80%, at least 85%, at least 90%, at least 95%, at least 96%, at least 97%, at least 98%, at least 99% or 100% identical to SEQ ID NO: 65.
11. The recombinant DNA construct of any one of embodiments 1-10, wherein the polynucleotide sequence of the first expression cassette comprises a sequence that is at least at least 80%, at least 85%, at least 90%, at least 95%, at least 96%, at least 97%, at least 98%, at least 99% or 100% identical to SEQ ID NO: 69.
12. The recombinant DNA construct of any one of embodiments 1-11, wherein the florigenic FT protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28, and 30, or a functional fragment thereof.
13. The recombinant DNA construct of embodiment 12, wherein the florigenic FT protein further comprises one or more of the following amino acids: a tyrosine or other uncharged polar or nonpolar residue at the amino acid position of the florigenic FT protein corresponding to amino acid position 85 of SEQ ID NO: 14; a leucine or other nonpolar residue at the amino acid position of the florigenic FT protein corresponding to amino acid position 128 of SEQ ID NO: 14; and a tryptophan or other large nonpolar residue at the amino acid position of the florigenic FT protein corresponding to amino acid position 138 of SEQ ID NO: 14.
14. The recombinant DNA construct of embodiment 12, wherein the florigenic FT protein does not have one or more of the following amino acids: a histidine at the amino acid position corresponding to a lysine or arginine at the amino acid position corresponding to position 85 of SEQ ID NO: 14; a lysine or arginine at the amino acid position corresponding to position 128 of SEQ ID NO: 14; and a serine, aspartic acid, glutamic acid, lysine or arginine at the amino acid position corresponding to position 138 of SEQ ID NO: 14.
15. The recombinant DNA construct of embodiment 12, wherein the florigenic FT protein comprises an amino acid sequence selected from the group consisting of SEQ ID NOs: 2, 4, 6, 8, 10, 12, 14, 16, 18, 20, 22, 24, 26, 28, and 30, or a functional fragment thereof.
16. The recombinant DNA construct of any one of embodiments 1-15, wherein the polynucleotide sequence is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to a sequence selected from the group consisting of SEQ ID NOs: 1, 3, 5, 7, 9, 11, 13, 15, 17, 19, 21, 23, 25, 27, and 29.
17. The recombinant DNA construct of any one of embodiments 1-16, wherein the first plant expressible promoter is a vegetative stage promoter.
18. The recombinant DNA construct of any one of embodiments 1-17, wherein the first plant expressible promoter is a meristem-preferred or meristem-specific promoter.
19. The recombinant DNA construct of any one of embodiments 1-18, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to a polynucleotide sequence selected from the group consisting of SEQ ID NOs: 31, 32, 33, 34, 35, 36, 37, 38, 39, 40, 41, 42, 43, 44, 45 or 48, or a functional portion thereof.
20. The recombinant DNA construct of embodiment 19, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 31, or a functional portion thereof.
21. The recombinant DNA construct of embodiment 19, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 32 or SEQ ID NO: 48.
22. The recombinant DNA construct of embodiment 19, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 44, or a functional portion thereof.
23. The recombinant DNA construct of any one of embodiments 1-22, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to a polynucleotide sequence selected from the group consisting of SEQ ID NOs: 49, 50, 51, 52, 53, 54, 55, 56, 57, 58, 59, 60, 61, 62, 63 or 64, or a functional portion thereof.
24. The recombinant DNA construct of embodiment 23, wherein the first plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to SEQ ID NO: 49, or a functional portion thereof.
25. The recombinant DNA construct of any one of embodiments 1-24, wherein the second plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to a polynucleotide sequence selected from the group consisting of SEQ ID NOs: 49, 50, 51, 52, 53, 54, 55, 56, 57, 58, 59, 60, 61, 62, 63 or 64, or a functional portion thereof.
26. The recombinant DNA construct of any one of embodiments 1-25, wherein the second plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to SEQ ID NO: 49, or a functional portion thereof.
27. The recombinant DNA construct of any one of embodiments 1-26, wherein the second plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to a polynucleotide sequence selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, or 94, or a functional portion thereof.
28. The recombinant DNA construct of any one of embodiments 1-27, wherein the first plant expressible promoter is a vegetative stage promoter and the second plant expressible promoter is a late vegetative stage promoter and/or reproductive stage promoter.
29. The recombinant DNA construct of embodiment 28, wherein the first plant expressible promoter is an early vegetative stage promoter.
30. The recombinant DNA construct of embodiment 28, wherein the second plant expressible promoter is a reproductive stage preferred promoter.
31. The recombinant DNA construct of any one of embodiments 1-30, wherein the first plant expressible promoter initiates detectable expression of the polynucleotide sequence encoding the florigenic FT protein at an earlier developmental stage than the second plant expressible promoter initiates detectable expression of the transcribable DNA sequence.
32. A DNA molecule or vector comprising the recombinant DNA construct of any one of embodiments 1-31.
33. A plasmid vector for Agrobacterium-mediated transformation comprising the recombinant DNA construct of any one of embodiments 1-31.
34. A donor template molecule for site-directed integration comprising the recombinant DNA construct of any one of embodiments 1-31.
35. A transgenic plant comprising an insertion of the recombinant DNA construct of any one of embodiments 1-31 into the genome of at least one cell of the transgenic plant.
36. The transgenic plant of embodiment 35, wherein the transgenic plant is homozygous for the insertion of the recombinant DNA construct.
37. The transgenic plant of embodiment 35, wherein the transgenic plant is hemizygous for the insertion of the recombinant DNA construct.
38. The transgenic plant of any one of embodiments 35-37, wherein the transgenic plant is a short day plant.
39. The transgenic plant of any one of embodiments 35-38, wherein the transgenic plant is a dicotyledonous plant.
40. The transgenic plant of any one of embodiments 35-39, wherein the transgenic plant is a leguminous plant.
41. The transgenic plant of any one of embodiments 35-40, wherein the transgenic plant is soybean.
42. The transgenic plant of embodiment 41, wherein the transgenic soybean plant produces more pods per node than a control plant not having the recombinant DNA construct.
43. The transgenic plant of any one of embodiments 35-42, wherein the transgenic plant produces more flowers per node than a control plant not having the recombinant DNA construct.
44. The transgenic plant or part thereof of any one of embodiments 35-43, wherein the transgenic plant produces more seeds, bolls, siliques, fruits, nuts or pods per node of the transgenic plant than a control plant not having the recombinant DNA construct.
45. The transgenic plant or part thereof of any one of embodiments 35-44, wherein the transgenic plant flowers earlier than a control plant not having the recombinant DNA construct.
46. The transgenic plant or part thereof of any one of embodiments 35-45, wherein the transgenic plant has more floral racemes per node than a control plant not having the recombinant DNA construct.
47. A transgenic plant part comprising an insertion of the recombinant DNA construct of any one of embodiments 1-31 into the genome of at least one cell of the transgenic plant part.
48. The transgenic plant part of any one of embodiments 44-47, wherein the transgenic plant part is one of the following: a seed, fruit, leaf, cotyledon, hypocotyl, meristem, embryo, endosperm, root, shoot, stem, pod, flower, infloresence, stalk, pedicel, style, stigma, receptacle, petal, sepal, pollen, anther, filament, ovary, ovule, pericarp, phloem, or vascular tissue.
49. A recombinant DNA construct comprising a polynucleotide sequence encoding a florigenic FT protein operably linked to a plant expressible promoter, wherein the polynucleotide sequence comprises a sequence that encodes a target site in a mRNA transcript encoded by the polynucleotide sequence, and wherein the target site of the mRNA transcript is at least 80% complementary to an endogenous RNA molecule.
50. The recombinant DNA construct of embodiment 49, wherein the target site of the mRNA transcript is at least 17 nucleotides in length.
51. The recombinant DNA construct of embodiment 49 or 50, wherein the target site of the mRNA transcript is 17, 18, 19, 20, 21, 22, 23, 24, 25, 26 or 27 nucleotides in length.
52. The recombinant DNA construct of any one of embodiments 49-51, wherein the target site of the mRNA transcript is at least 80%, at least 85%, at least 90%, at least 95%, at least 96%, at least 97%, at least 98%, at least 99%, at least 99.5% or 100% complementary to the endogenous RNA molecule.
53. The recombinant DNA construct of any one of embodiments 49-52, wherein the target site is present in a non-coding sequence of the mRNA transcript.
54. The recombinant DNA construct of any one of embodiments 49-53, wherein the target site of the mRNA transcript is at least 80%, at least 85%, at least 90%, at least 95%, at least 96%, at least 97%, at least 98%, at least 99% or 100% complementary to SEQ ID NO: 95, 96, 97, 103, 104, or 105.
55. The recombinant DNA construct of any one of embodiments 49-54, wherein the target site of the mRNA transcript is at least 80%, at least 85%, at least 90%, at least 95%, at least 96%, at least 97%, at least 98%, at least 99% or 100% complementary to SEQ ID NO: 95 or 103.
56. The recombinant DNA construct of any one of embodiments 49-55, wherein the polynucleotide sequence encoding the florigenic FT protein comprises a sequence that is at least at least 80%, at least 85%, at least 90%, at least 95%, at least 96%, at least 97%, at least 98%, at least 99% or 100% identical to SEQ ID NO: 98, 99, 100, 101, 106, 107, 108, 109 or 110.
57. The recombinant DNA construct of any one of embodiments 49-54 or 56, wherein the target site of the mRNA transcript is at least 80%, at least 85%, at least 90%, at least 95%, at least 96%, at least 97%, at least 98%, at least 99% or 100% identical to SEQ ID NO: 99 or 107.
58. The recombinant DNA construct of any one of embodiments 49-57, wherein the florigenic FT protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28, and 30, or a functional fragment thereof.
59. The recombinant DNA construct of any one of embodiments 49-58, wherein the florigenic FT protein further comprises one or more of the following amino acids: a tyrosine or other uncharged polar or nonpolar residue at the amino acid position of the florigenic FT protein corresponding to amino acid position 85 of SEQ ID NO: 14; a leucine or other nonpolar residue at the amino acid position of the florigenic FT protein corresponding to amino acid position 128 of SEQ ID NO: 14; and a tryptophan or other large nonpolar residue at the amino acid position of the florigenic FT protein corresponding to amino acid position 138 of SEQ ID NO: 14.
60. The recombinant DNA construct of any one of embodiments 49-58, wherein the florigenic FT protein does not have one or more of the following amino acids: a histidine at the amino acid position corresponding to a lysine or arginine at the amino acid position corresponding to position 85 of SEQ ID NO: 14; a lysine or arginine at the amino acid position corresponding to position 128 of SEQ ID NO: 14; and a serine, aspartic acid, glutamic acid, lysine or arginine at the amino acid position corresponding to position 138 of SEQ ID NO: 14.
61. The recombinant DNA construct of any one of embodiments 49-61, wherein the florigenic FT protein comprises an amino acid sequence selected from the group consisting of SEQ ID NOs: 2, 4, 6, 8, 10, 12, 14, 16, 18, 20, 22, 24, 26, 28, and 30, or a functional fragment thereof.
62. The recombinant DNA construct of any one of embodiments 49-62, wherein the polynucleotide sequence is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to a sequence selected from the group consisting of SEQ ID NOs: 1, 3, 5, 7, 9, 11, 13, 15, 17, 19, 21, 23, 25, 27, and 29.
63. The recombinant DNA construct of any one of embodiments 49-62, wherein the plant expressible promoter is a vegetative stage promoter.
64. The recombinant DNA construct of any one of embodiments 49-63, wherein the plant expressible promoter is a meristem-preferred or meristem-specific promoter.
65. The recombinant DNA construct of any one of embodiments 49-64, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to a polynucleotide sequence selected from the group consisting of SEQ ID NOs: 31, 32, 33, 34, 35, 36, 37, 38, 39, 40, 41, 42, 43, 44, 45 or 48, or a functional portion thereof.
66. The recombinant DNA construct of any one of embodiments 49-65, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 31, or a functional portion thereof.
67. The recombinant DNA construct of any one of embodiments 49-65, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 32 or SEQ ID NO: 48.
68. The recombinant DNA construct of any one of embodiments 49-65, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to SEQ ID NO: 44, or a functional portion thereof.
69. The recombinant DNA construct of any one of embodiments 49-68, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to a polynucleotide sequence selected from the group consisting of SEQ ID NOs: 49, 50, 51, 52, 53, 54, 55, 56, 57, 58, 59, 60, 61, 62, 63 or 64, or a functional portion thereof.
70. A DNA molecule or vector comprising the recombinant DNA construct of any one of embodiments 49-69.
71. A plasmid vector for Agrobacterium-mediated transformation comprising the recombinant DNA construct of any one of embodiments 49-69.
72. A donor template molecule for site-directed integration comprising the recombinant DNA construct of any one of embodiments 49-69.
73. A transgenic plant comprising an insertion of the recombinant DNA construct of any one of embodiments 49-69 into the genome of at least one cell of the transgenic plant.
74. The transgenic plant of embodiment 73, wherein the transgenic plant is homozygous for the insertion of the recombinant DNA construct.
75. The transgenic plant of embodiment 73, wherein the transgenic plant is hemizygous for the insertion of the recombinant DNA construct.
76. The transgenic plant of any one of embodiments 73-75, wherein the transgenic plant is a short day plant.
77. The transgenic plant of any one of embodiments 73-76, wherein the transgenic plant is a dicotyledonous plant.
78. The transgenic plant of any one of embodiments 73-77, wherein the transgenic plant is a leguminous plant.
79. The transgenic plant of any one of embodiments 73-78, wherein the transgenic plant is soybean.
80. The transgenic plant of any one of embodiments 73-79, wherein the transgenic soybean plant produces more pods per node than a control plant not having the recombinant DNA construct.
81. The transgenic plant of any one of embodiments 73-80, wherein the transgenic plant produces more flowers per node than a control plant not having the recombinant DNA construct.
82. The transgenic plant or part thereof of any one of embodiments 73-81, wherein the transgenic plant produces more bolls, siliques, fruits, nuts or pods per node of the transgenic plant than a control plant not having the recombinant DNA construct.
83. The transgenic plant or part thereof of any one of embodiments 73-82, wherein the transgenic plant flowers earlier than a control plant not having the recombinant DNA construct.
84. The transgenic plant or part thereof of any one of embodiments 73-83, wherein the transgenic plant has more floral racemes per node than a control plant not having the recombinant DNA construct.
85. A transgenic plant part comprising an insertion of the recombinant DNA construct of any one of embodiments 49-69 into the genome of at least one cell of the transgenic plant part.
86. The transgenic plant part of any one of embodiments 82-85, wherein the transgenic plant part is one of the following: a seed, fruit, leaf, cotyledon, hypocotyl, meristem, embryo, endosperm, root, shoot, stem, pod, flower, infloresence, stalk, pedicel, style, stigma, receptacle, petal, sepal, pollen, anther, filament, ovary, ovule, pericarp, phloem, or vascular tissue.
87. A recombinant DNA construct comprising a transcribable DNA sequence encoding a RNA molecule comprising a targeting sequence that is at least 80% complementary to at least 15 consecutive nucleotides of a polynucleotide sequence encoding a florigenic FT protein, wherein the transcribable DNA sequence is operably linked to a plant expressible promoter.
88. The recombinant DNA construct of embodiment 87, wherein the targeting sequence of the RNA molecule is from about 15 to about 27 nucleotides in length.
89. The recombinant DNA construct of embodiment 87 or 88, wherein the targeting sequence of the RNA molecule is at least 85%, at least 90%, at least 95%, at least 96%, at least 97%, at least 98%, at least 99%, at least 99.5% or 100% complementary to at least 15 consecutive nucleotides of the polynucleotide sequence encoding the florigenic FT protein.
90. The recombinant DNA construct of any one of embodiments 87-89, wherein the targeting sequence of the RNA molecule is at least 80% complementary to at least 15 consecutive nucleotides of a mRNA transcript encoded by the polynucleotide sequence encoding the florigenic FT protein.
91. The recombinant DNA construct of any one of embodiments 87-90, wherein targeting sequence of the RNA molecule is at least 80% complementary to at least 15 consecutive nucleotides of an exonic or coding sequence of the mRNA transcript.
92. The recombinant DNA construct of any one of embodiments 87-91, wherein targeting sequence of the RNA molecule is at least 80% complementary to at least 15 consecutive nucleotides of a non-coding sequence of the mRNA transcript.
93. The recombinant DNA construct of any one of embodiments 87-92, wherein the RNA molecule encoded by the transcribable DNA sequence is a precursor miRNA or siRNA.
94. The recombinant DNA construct of any one of embodiments 87-93, wherein the florigenic FT protein encoded by the polynucleotide sequence comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28, and 30, or a functional fragment thereof.
95. The recombinant DNA construct of any one of embodiments 87-94, wherein the florigenic FT protein further comprises one or more of the following amino acids: a tyrosine or other uncharged polar or nonpolar residue at the amino acid position of the florigenic FT protein corresponding to amino acid position 85 of SEQ ID NO: 14; a leucine or other nonpolar residue at the amino acid position of the florigenic FT protein corresponding to amino acid position 128 of SEQ ID NO: 14; and a tryptophan or other large nonpolar residue at the amino acid position of the florigenic FT protein corresponding to amino acid position 138 of SEQ ID NO: 14.
96. The recombinant DNA construct of any one of embodiments 87-94, wherein the florigenic FT protein does not have one or more of the following amino acids: a histidine at the amino acid position corresponding to a lysine or arginine at the amino acid position corresponding to position 85 of SEQ ID NO: 14; a lysine or arginine at the amino acid position corresponding to position 128 of SEQ ID NO: 14; and a serine, aspartic acid, glutamic acid, lysine or arginine at the amino acid position corresponding to position 138 of SEQ ID NO: 14.
97. The recombinant DNA construct of any one of embodiments 87-96, wherein the florigenic FT protein comprises an amino acid sequence selected from the group consisting of SEQ ID NOs: 2, 4, 6, 8, 10, 12, 14, 16, 18, 20, 22, 24, 26, 28, and 30, or a functional fragment thereof.
98. The recombinant DNA construct of any one of embodiments 87-97, wherein the polynucleotide sequence encoding the florigenic FT protein is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to a sequence selected from the group consisting of SEQ ID NOs: 1, 3, 5, 7, 9, 11, 13, 15, 17, 19, 21, 23, 25, 27, and 29.
99. The recombinant DNA construct of any one of embodiments 87-98, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% identical to a polynucleotide sequence selected from the group consisting of SEQ ID NOs: 49, 50, 51, 52, 53, 54, 55, 56, 57, 58, 59, 60, 61, 62, 63 or 64, or a functional portion thereof.
100. The recombinant DNA construct of any one of embodiments 87-99, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to SEQ ID NO: 49, or a functional portion thereof.
101. The recombinant DNA construct of any one of embodiments 87-100, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, or least 99%, at least 99.5% or 100% identical to a polynucleotide sequence selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, or 94, or a functional portion thereof.
102. The recombinant DNA construct of any one of embodiments 87-101, wherein the plant expressible promoter is a vegetative stage promoter.
103. The recombinant DNA construct of any one of embodiments 87-102, wherein the plant expressible promoter is a late vegetative stage promoter and/or reproductive stage promoter.
104. The recombinant DNA construct of any one of embodiments 87-103, wherein the plant expressible promoter is heterologous with respect to the transcribable DNA sequence.
105. A DNA molecule or vector comprising the recombinant DNA construct of any one of embodiments 87-104.
106. A transgenic plant comprising an insertion of the recombinant DNA construct of any one of embodiments 87-104 into the genome of at least one cell of the transgenic plant.
107. The transgenic plant of embodiment 106, wherein the transgenic plant is a short day plant.
108. The transgenic plant of embodiment 106 or 107, wherein the transgenic plant is a dicotyledonous plant.
109. The transgenic plant of any one of embodiments 106-108, wherein the transgenic plant is a leguminous plant.
110. The transgenic plant of any one of embodiments 106-109, wherein the transgenic plant is soybean.
111. A transgenic plant part comprising an insertion of the recombinant DNA construct of any one of embodiments 87-103 into the genome of at least one cell of the transgenic plant part.
112. The transgenic plant part of embodiment 111, wherein the transgenic plant part is one of the following: a seed, fruit, leaf, cotyledon, hypocotyl, meristem, embryo, endosperm, root, shoot, stem, pod, flower, infloresence, stalk, pedicel, style, stigma, receptacle, petal, sepal, pollen, anther, filament, ovary, ovule, pericarp, phloem, or vascular tissue.
113. A method for producing a transgenic plant, comprising
   (a) transforming at least one cell of an explant with the recombinant DNA construct of any one of embodiments 1-31, 49-69 or 87-104; and
   (b) regenerating or developing the transgenic plant from the transformed explant.
114. The method of embodiment 113, further comprising:
   (c) selecting a transgenic plant having one or more of the following traits or phenotypes: earlier flowering, longer reproductive or flowering duration, increased number of flowers per node, increased number of floral racemes per node, increased number of pods, bolls, siliques, fruits, or nuts per node, and increased number of seeds per node, as compared to a control plant not having the recombinant DNA construct.
115. The method of embodiment 113 or 114, wherein the transforming step (a) is carried out via Agrobacterium-mediated transformation or microprojectile bombardment of the explant.
116. The method of any one of embodiments 113-115, wherein the transforming step (a) comprises site-directed integration of the recombinant DNA construct.
117. A method of planting a transgenic crop plant, comprising:
   planting the transgenic crop plant at a higher density in the field, wherein the transgenic crop plant comprises an insertion of the recombinant DNA construct of any one of embodiments 1-31, 49-69 or 87-104.
118. The method of embodiment 117, wherein the transgenic crop plant is soybean, and wherein about 150,000 to 250,000 seeds of the transgenic soybean plant are planted per acre.
119. The method of embodiment 117, wherein the transgenic crop plant is cotton, and wherein about 48,000 to 60,000 seeds of the transgenic cotton plant are planted per acre.
120. The method of embodiment 117, wherein the transgenic crop plant is canola, and wherein about 450,000 to 680,000 seeds of the transgenic canola plant are planted per acre.
121. The transgenic plant of any one of embodiments 35-46, 73-84, or 106-110, wherein the transgenic plant has at least 1%, at least 2%, at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more seeds, pods, bolls, siliques, fruits, nuts or tubers than a non-transgenic control plant.
122. The transgenic plant of any one of embodiments 35-46, 73-84, or 106-110, wherein the transgenic plant has an average of at least 5%, at least 10%, at least 15%, at least 20%, at least 25%, at least 30%, at least 35%, at least 40%, at least 45%, at least 50%, at least 60%, at least 70%, at least 75%, at least 80%, at least 90%, at least 100%, at least 125%, at least 150%, at least 175%, at least 200%, at least 250%, at least 300%, at least 350%, or at least 400% more seeds, pods, bolls, siliques, fruits, nuts or tubers per node than a non-transgenic control plant.
123. The transgenic plant of any one of embodiments 35-46, 73-84, or 106-110, wherein the transgenic plant has an average of at least 2, at least 3, at least 4, at least 5, at least 6, at least 7, at least 8, at least 9, or at least 10 pods, bolls, siliques, fruits, nuts or tubers per node.
124. The transgenic plant of any one of embodiments 35-46, 73-84, or 106-110, wherein the transgenic plant has an average of 2 to 10, 2 to 9, 2 to 8, 2 to 7, 2 to 6, 2 to 5, 2 to 4, 3 to 10, 3 to 9, 3 to 8, 3 to 7, 3 to 6, 3 to 5, 4 to 10, 4 to 9, 4 to 8, 4 to 7, 4 to 6, or 4 to 5 pods, bolls, siliques, fruits, nuts or tubers per node.
125. The transgenic plant of any one of embodiments 35-46, 73-84, or 106-110, wherein the transgenic plant has an average of at least 1, at least 2, at least 3, at least 4, at least 5, at least 6, at least 7, at least 8, at least 9, or at least 10 more bolls, siliques, fruits, nuts or pods per node as compared to a non-transgenic control plant.
126. The transgenic plant of any one of embodiments 35-46, 73-84, or 106-110, wherein the transgenic plant flowers at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, or at least 45 days earlier than a non-transgenic control plant.
127. The transgenic plant of any one of embodiments 121, 122, 123, 124, 125 or 126, wherein the transgenic plant is a soybean plant, and the transgenic plant has more pods per node on average than a non-transgenic control plant.
128. A transgenic plant comprising a polynucleotide sequence encoding a florigenic FT protein operably linked to a vegetative stage promoter, wherein expression of the florigenic FT protein is suppressed in a late vegetative and/or reproductive tissue.
129. The transgenic plant of embodiment 128, wherein expression of the florigenic FT protein is suppressed by a small RNA molecule.
130. A recombinant DNA construct comprising a polynucleotide sequence encoding a florigenic FT protein and operably linked to a vegetative stage promoter, and at least one transcribable DNA sequence encoding an RNA molecule comprising a targeting sequence that is complementary to at least a portion of the polynucleotide sequence.
131. A transgenic plant comprising an insertion of the recombinant DNA construct of embodiment 130 into the genome of at least one cell of the transgenic plant.
132. A transgenic plant comprising a recombinant polynucleotide sequence encoding a florigenic FT protein operably linked to a vegetative stage promoter, wherein expression of the polynucleotide sequence is spatially and temporally restricted by a small RNA molecule.
133. A recombinant DNA construct comprising an expression cassette, wherein the expression cassette comprises a polynucleotide sequence encoding a florigenic FT protein operably linked to a plant expressible promoter, wherein the plant expressible promoter comprises a polynucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a polynucleotide sequence selected from the group consisting of SEQ ID NOs: 49, 50, 51, 52, 53, 54, 55, 56, 57, 58, 59, 60, 61, 62, 63 or 64, or a functional portion thereof.
134. The recombinant DNA construct of embodiment 130 or 133, wherein the florigenic FT protein comprises an amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or least 99% identity to a sequence selected from the group consisting of SEQ ID NOs: 2, 4, 6, 8, 10, 12, 14, 16, 18, 20, 22, 24, 26, 28 and 30, or a functional fragment thereof.
135. A DNA molecule or vector comprising the recombinant DNA construct of embodiment 130 or 133.
136. A plasmid vector for Agrobacterium-mediated transformation comprising the recombinant DNA construct of embodiment 130 or 133.
137. A donor template molecule for site-directed integration comprising the recombinant DNA construct of embodiment 130 or 133.
138. A transgenic plant comprising an insertion of the recombinant DNA construct of embodiment 130 or 133 into the genome of at least one cell of the transgenic plant.
139. The transgenic plant of any one of embodiments 131, 132, or 138, wherein the transgenic plant is homozygous for the insertion of the recombinant DNA construct.
140. The transgenic plant of any one of embodiments 131, 132, 138, or 139, wherein the transgenic plant is hemizygous for the insertion of the recombinant DNA construct.
141. The transgenic plant of any one of embodiments 131, 132, or 138-140, wherein the transgenic plant is a short day plant.
142. The transgenic plant of any one of embodiments 131, 132, or 138-141, wherein the transgenic plant is a dicotyledonous plant.
143. The transgenic plant of any one of embodiments 131, 132, or 138-142, wherein the transgenic plant is a leguminous plant.
144. The transgenic plant of any one of embodiments 131, 132, or 138-143, wherein the transgenic plant is soybean.
145. The transgenic plant of any one of embodiments 35-46, 73-84, 106-110, 131, 132, or 138-144, wherein the transgenic plant produces more seeds, bolls, siliques, fruits, nuts or pods per node of the transgenic plant than a control plant not having the recombinant DNA construct.
146. The transgenic plant of any one of embodiments 35-46, 73-84, 106-110, 131, 132, or 138-145, wherein the transgenic plant flowers earlier than a control plant not having the recombinant DNA construct.
147. The transgenic plant of any one of embodiments 35-46, 73-84, 106-110, 131, 132, or 138-146, wherein the transgenic plant is a soybean plant that has a plant height of at least 700 millimeters at R8 stage.
148. The transgenic plant of any one of embodiments 35-46, 73-84, 106-110, 131, 132, or 138-147, wherein the transgenic plant is a soybean plant that has at least 100 nodes per plant at R8 stage.
149. A transgenic plant part comprising an insertion of the recombinant DNA construct of embodiment 130 or 133 into the genome of at least one cell of the transgenic plant part.

### Examples

### Example 1. Soybean Short Day Induction Treatment and Identification of Flowering Locus T (FT) Genes by Transcriptional Profiling

This example was previously described in co-pending U.S. patent application Ser. No. 15/131,987, and International Application No. PCT/US2016/028130, which are incorporated herein by reference in their entirety. Methods for the photoperiodic light treatment (i.e., short day induction of flowering in plants) are described in U.S. Pat. No. 8,935,880 and U.S. Patent Application Publication No. 2014/0259905, which are incorporated herein by reference in their entirety. As described further therein, the early short day induction treatment produced soybean plants having altered reproductive traits including an increased number of pods/seeds per plant. Transcriptional profiling experiments were performed using gene expression microarrays to determine if particular transcripts were up-regulated in these light-induced plants to identify genes that may be responsible for mediating the short day induction phenotypes. In these experiments, an analysis of transcripts was conducted on soybean leaf and floral apex tissues collected after 1, 3 and 5 days from plants that received a short day inductive light treatment (Short day) in comparison to tissues from plants that did not receive the inductive treatment (Long day).

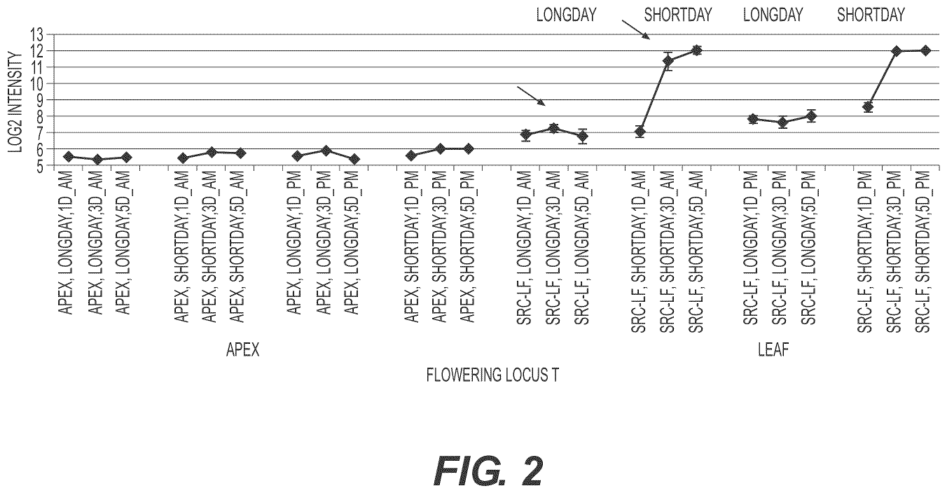

As shown in FIG. 2, an increased accumulation of transcripts was observed for a particular Flowering Locus T gene, Gm.FT2a (SEQ ID NO: 1), in leaf tissue harvested at 3 and 5 days after the early short day induction (eSDI) treatment in comparison to samples taken from either (i) floral apex tissues of the same short day induction plants, or (ii) leaf tissues and floral apex tissues of soybean plants that instead received the long day treatment. These data support the conclusion that Gm.FT2a expression is induced in leaf tissue of plants experiencing the eSDI treatment, which was not seen in plants grown under long day conditions. Gm.FT2a expression was also not observed in the floral apex of eSDI treated plants, which is consistent with the model of FT protein expression being induced in peripheral leafy tissues in response to inductive photoperiod conditions and then migrating to its site of action in the meristems to induce flowering. However, additional experiments using a more sensitive RNA sequencing analysis of transcripts did show some Gm.FT2a induction in the shoot apex and axillary buds in response to the eSDI treatment (data not shown).

### Example 2. Characterization of the pAt.Erecta Promoter Expression Patterns in Soybean

Achieving desirable traits or phenotypes by transgenic approaches may require control of the temporal and spatial patterns of ectopic FT gene expression. Soy physiological experiments identifying Gm.FT2a expression in vegetative tissues following the short day induction treatment (see FIG. 2) indicated that achieving yield positive traits may rely on earlier FT expression during the vegetative stage. On the other hand, even though FT transcripts are not detected in the vegetative apex, FT protein has been shown to move long distance from the leaves to the apical tissue where it triggers a vegetative to reproductive transition. See, e.g., Lifschitz, E. et al., (2006), supra; and Corbeiser, L. et al., “FT Protein Movement Contributes to Long-Distance Signaling in Floral Induction of Arabidopsis”, Science 316: 1030-1033 (2007). Thus, in light of our own observations, we proposed using a vegetative stage promoter that is active in the meristem to control ectopic FT expression in a plant. By expressing the morphogenetic FT signal directly in the meristem at the desired developmental stage, multiple endogenous pathways and regulatory feedbacks (e.g., control of FT transduction in the leaf and long distance translocation of the FT signal) may be bypassed or avoided. Previous experiments with the short day induction treatment (described above in Example 1) revealed up-regulation of the Gm.Erecta gene in the meristems of soybean plants. The pErecta promoter (SEQ ID NO: 31) from Arabidopsis had been shown to have weak expression in the meristem(s) of plants during vegetative stages of development. Accordingly, the pAt.Erecta promoter was selected for initial FT expression experiments.

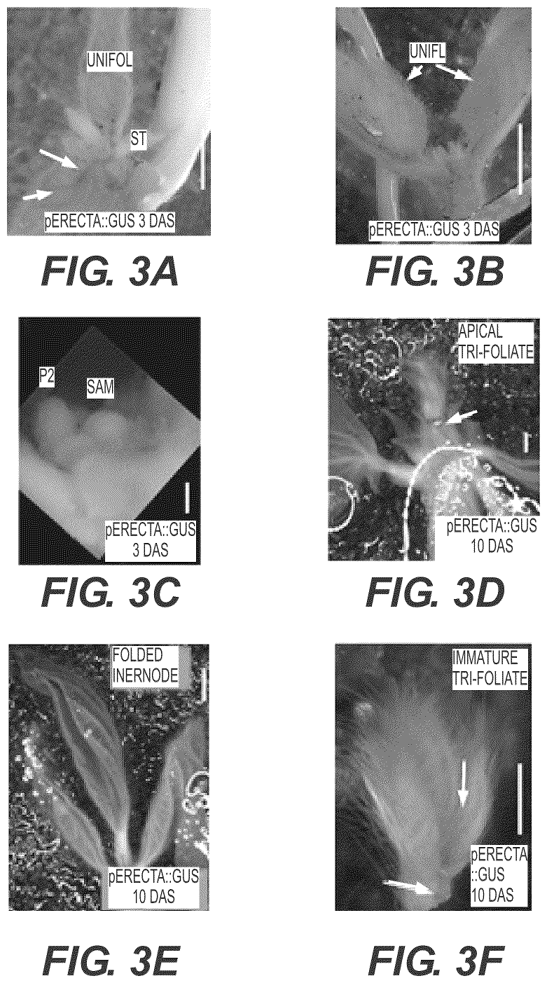

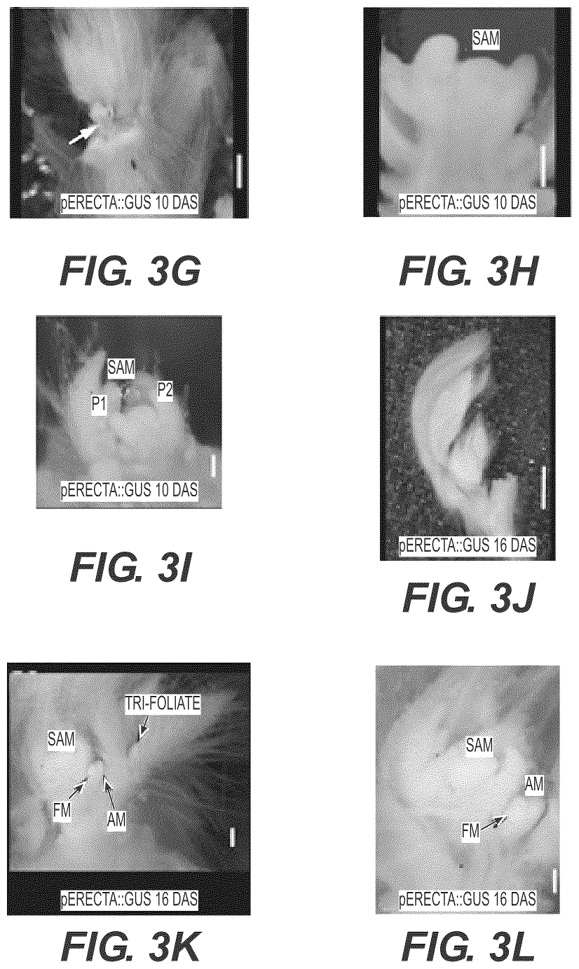

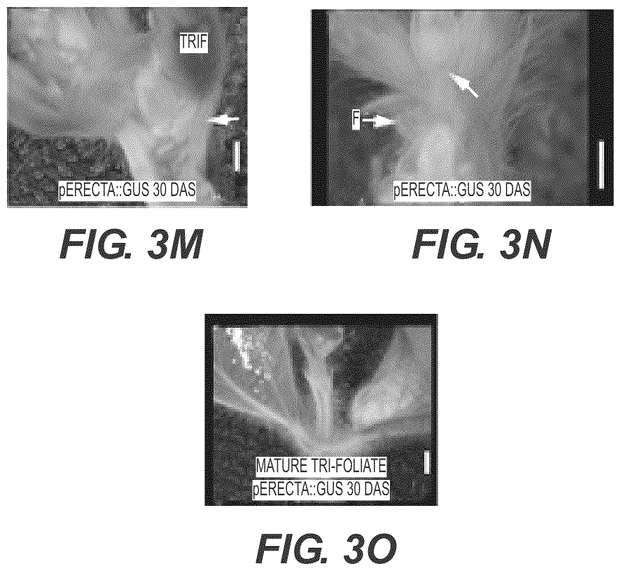

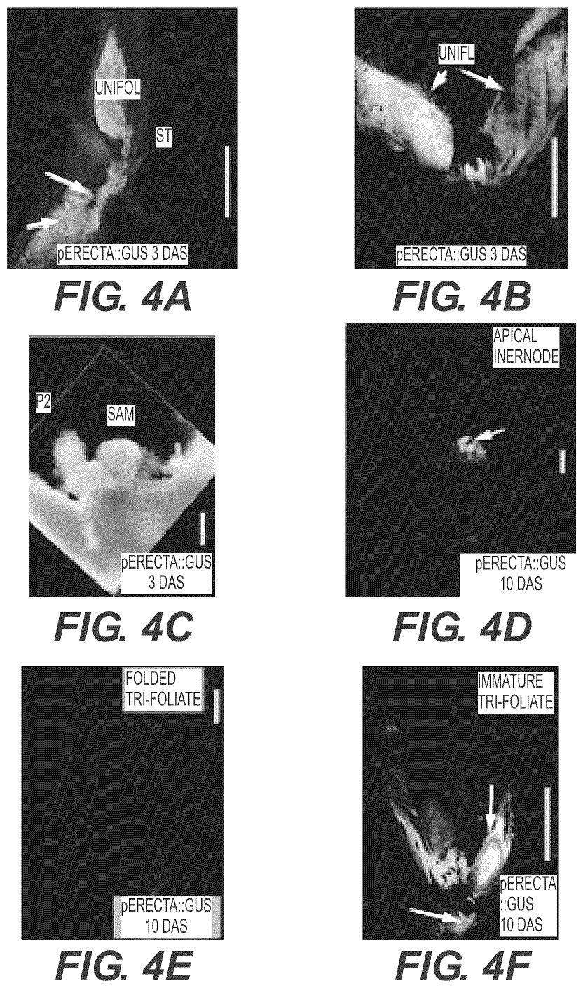

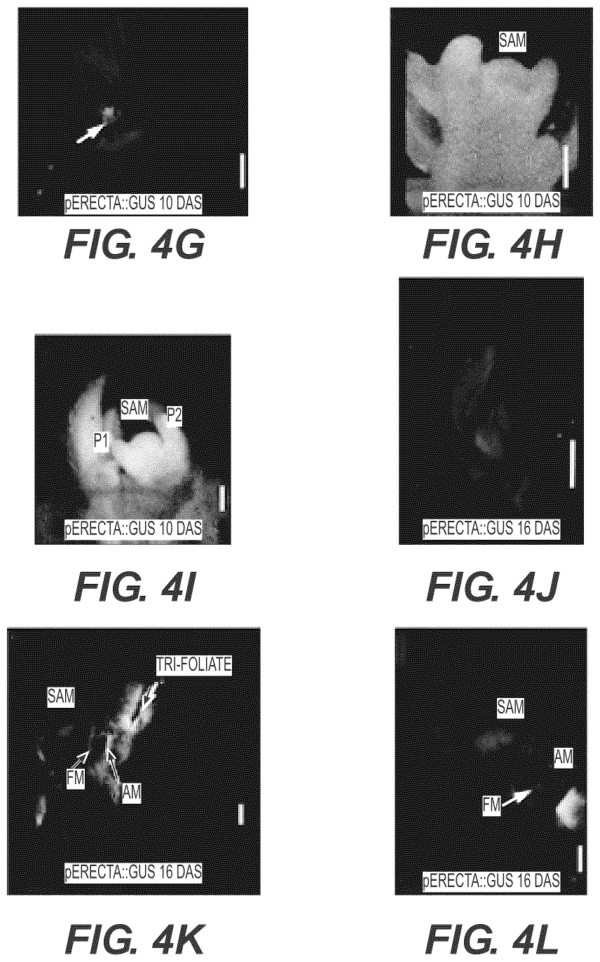

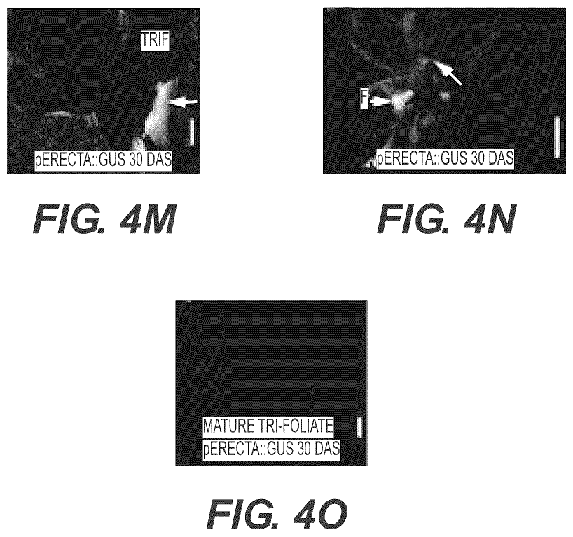

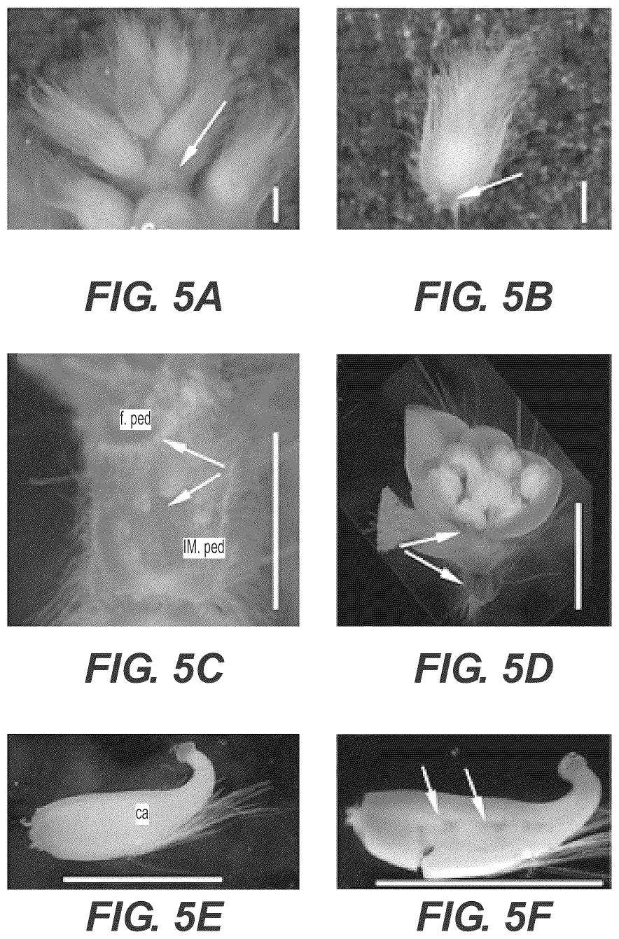

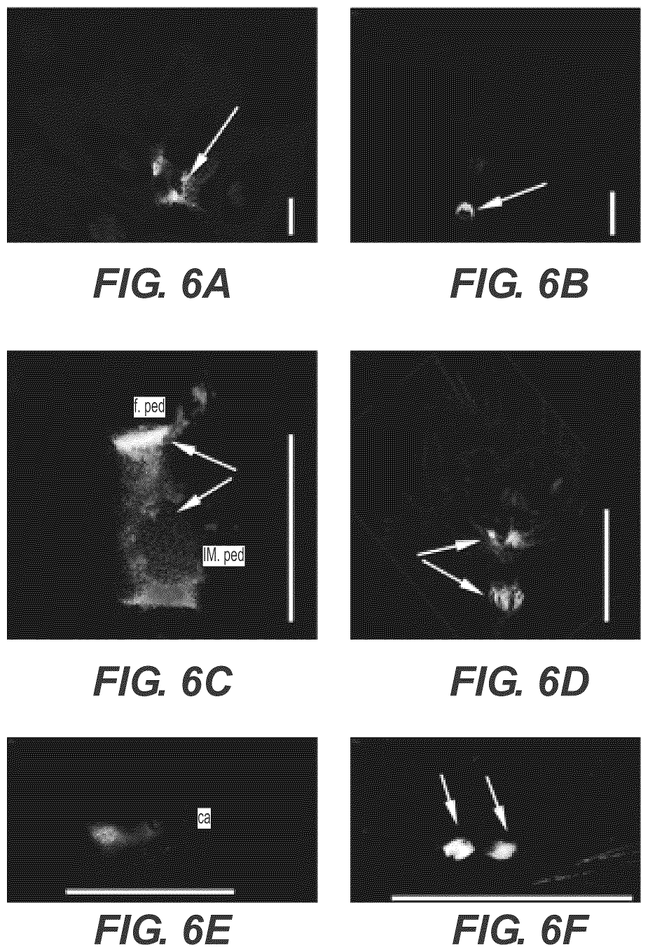

Additional experiments were performed to further characterize the expression patterns of pAt.Erecta fused to a GUS reporter gene in vegetative and floral meristematic tissues. Analysis of GUS expression patterns during the development of soy seedlings indicated that the pAt.Erecta promoter exhibits a temporal and spatial pattern of expression, preferably in the meristematic tissues during the vegetative stage of development. FIGS. 3A to 3O and 4A to 4O and FIGS. 5A to 5F and 6A to 6F include two sets of images to show the pattern of GUS staining. FIGS. 3A to 3O and 5A to 5F provide black and white images of the stained tissues, and FIGS. 4A to 4O and 6A to 6F provide black and white images corresponding to FIGS. 3A to 3O and 5A to 5F, respectively, but color filtered to show the pattern and intensity of blue GUS staining. Thus, the GUS staining pattern of expression can be viewed with these black and white images by comparing the corresponding images of FIGS. 3A to 3O and 4A to 4O or FIGS. 5A to 5F and 6A to 6F. As shown in FIGS. 3A to 3O and 4A to 4O, GUS staining was detected in the soy immature uni-foliate blade and petiole (FIGS. 4A and 4B) at three days after sowing/germination. pAt.Erecta::GUS expression was also broadly detected in the trifoliate primordia, shoot apical meristem (SAM) and axillary meristem sites at this early vegetative stage (FIG. 4C). GUS activity was not detected in the fully expanded uni-foliate and trifoliate leaves at ten days after germination or planting (FIGS. 4D and 4E). However, GUS activity was detected at the proximal part of the immature, unexpanded, but fully developed trifoliate blade, and at the adaxial side of the petiole (FIG. 4F). Detailed observation of the developing apical tissue showed that broad expression was retained in the developing immature leaf primordia, axillary meristems and shoot apical meristems (FIGS. 4G-I).

At the early reproductive stage, pAt.Erecta promoter activity was not detected in the mature blade and was reduced in the developing leaf primordia. The GUS signal was not detected in the indeterminate vegetative apex at the shoot apical meristem (SAM) or in the axillary meristem (AM) once these tissues started to form inflorescences (FIGS. 4J-4L). In all later stages, any additional meristematic activity could not be detected in the apex or in the axillaries or flower primordia. However, GUS expression continued in the adaxial side of the petiole and proximal part of the immature leaf blade (FIGS. 4M and 4N), but not in the fully expanded leaf blade (FIG. 40). GUS expression patterns with the pAt.Erecta promoter were also analyzed at the later R1 stages of development (35-40 days after germination). Similar to earlier stages of development, no expression was detected in the mature leaves or stems. However, strong promoter activity was detected in the inflorescence stems (FIGS. 5A and 6A; see arrow) and floral pedicels (FIGS. 5B and 6B; see arrow). In both tissues, expression was detected in vasculature and parenchyma cells (FIGS. 5C and 6C). At this stage, expression was also detected in the stamen filaments (FIGS. 5D and 6D; see arrows) and in the un-pollinated ovules (FIGS. 5E, 5F, 6E and 6F; see arrows in 6F).

Previously, the pAt.Erecta promoter was characterized in Arabidopsis. Interestingly, pAt.Erecta expression patterns in Arabidopsis were comparable to the patterns observed in soy during the vegetative stage, but not during late reproductive stages. In contrast, the pAt.Erecta expression pattern in soybean is diminished in early reproductive tissues but remerges in some later reproductive organs and tissues, including the inflorescence stems and floral pedicels. See, e.g., Chen, M-K et al., FEBS Letters 588: 3912-17 (2014); Yokoyama, R et al.; Shpak, E D et al., Science 309: 290-293 (2005); and Yokoyama, R et al., Plant J 15(3): 301-310 (1998), the entire contents and disclosures of which are incorporated herein by reference. Thus, the pAt.Erecta promoter provides a novel expression pattern in soybean.

### Example 3. Expression of Flowering Locus T Gene, Gm.FT2a, Under Control of a pAt. Erecta Promoter Alters Flowering Time and Pods Per Node in Soybean

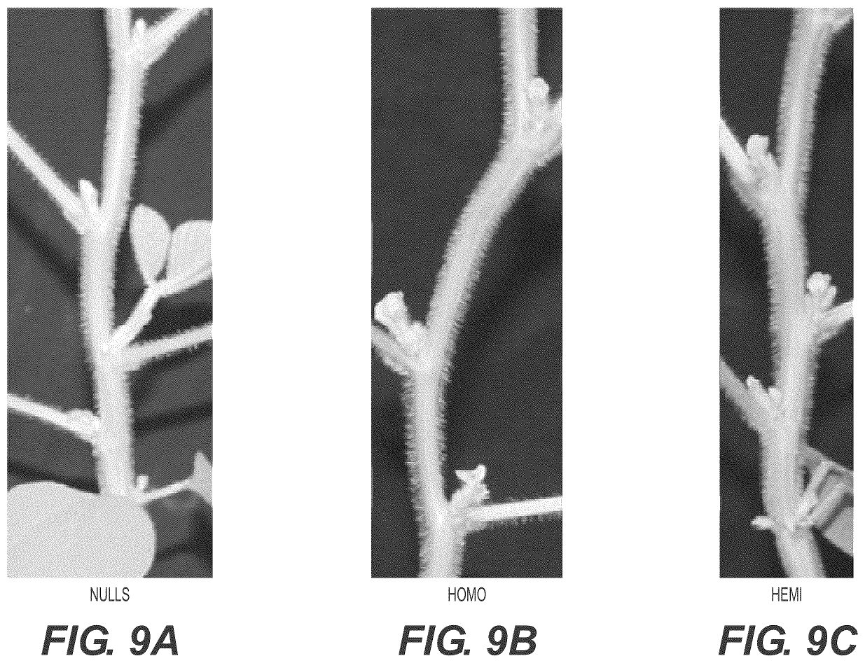

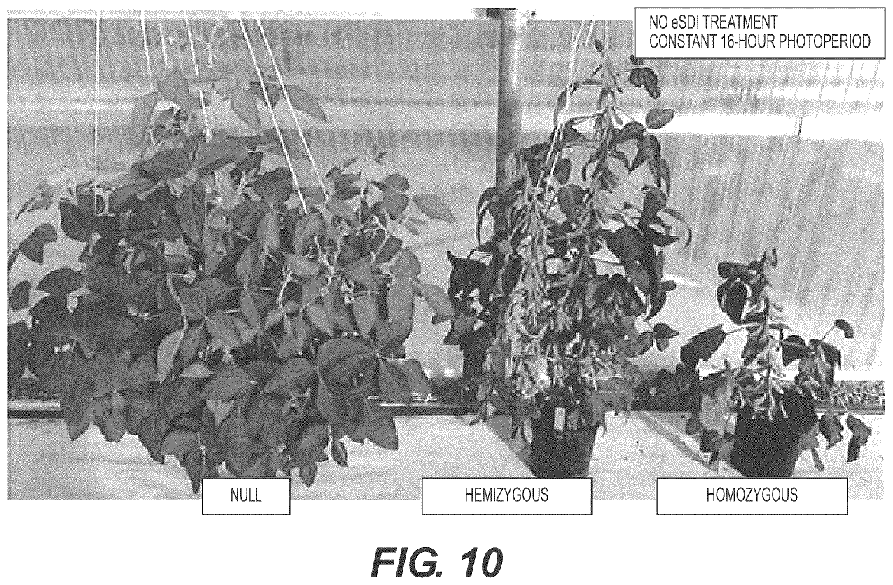

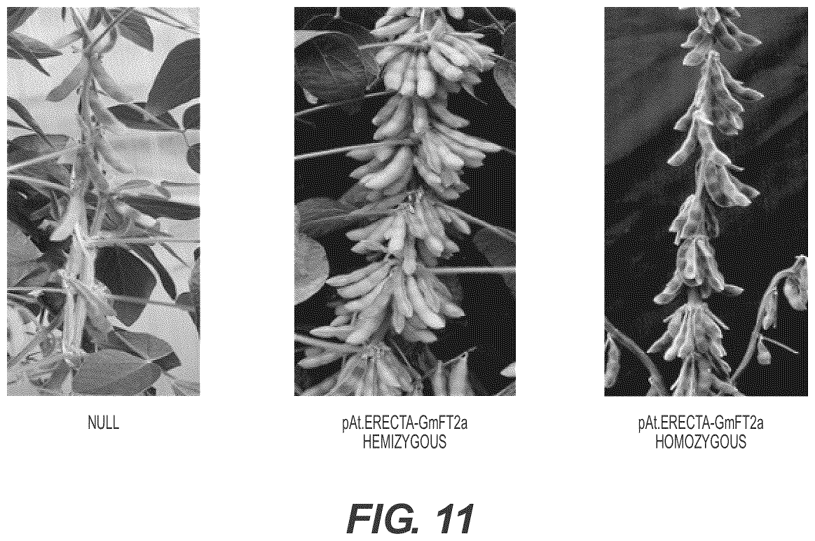

Transgenic soybean plants were produced by transforming soybean explants with a recombinant DNA molecule (i.e., a T-DNA transformation vector) comprising the pAt.Erecta promoter operably linked to the Gm.FT2a gene via Agrobacterium-mediated transformation to generate four pAt.Erecta::Gm.FT2a events that were carried forward for further testing. The effect of FT2a overexpression was immediately seen in R₀ plants, which had very early flowering and termination with reduced seed yield (e.g., only about 8 seeds/plant). These transgenic Gm.FT2a plants also had a short plant height and very few, if any, branches. Segregating R₁ plants and their progeny were subsequently grown in the greenhouse under long day conditions for initial study and characterization. By growing these plants under long day conditions, the severe dwarf phenotypes observed with Gm.FT2a transgenic R₀ plants were improved. In these experiments, both homozygous and hemizygous plants grown in the greenhouse under 16-hour long day conditions (i.e., 16/8 hours of day/night photoperiods) flowered much earlier than wild type null segregants. Gm.FT2a transgenic plants flowered at about 19-22 days after planting or seeding). (see, e.g., FIGS. 9A to 9C). Under these growth conditions, transgenic soybean plants expressing Gm.FT2a further had an increased number of pods per node on the main stem in comparison to wild type controls (see, e.g., FIGS. 10 and 11, discussed further below).

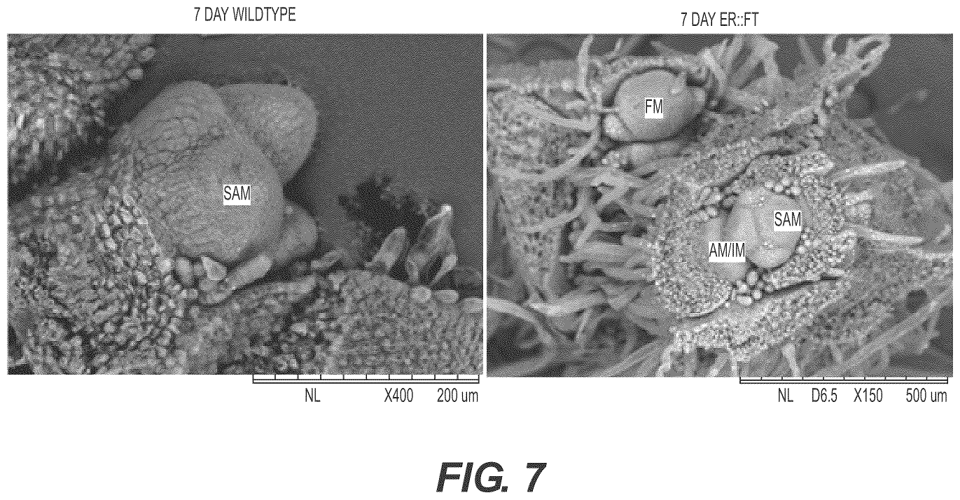

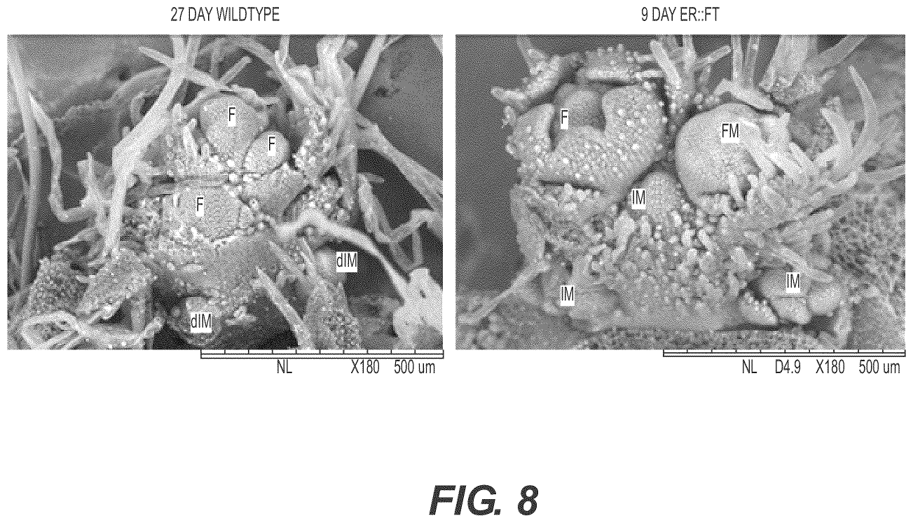

Plants containing one of the pAt.Erecta::Gm.FT2a transgenic events (Event 1) grown in controlled environment conditions were further analyzed via scanning electron microscopy analysis (eSEM). Analysis of the shoot apical meristem (SAM) of these transgenic plants (collected at 7 days after planting) revealed an early transition of the SAM into an inflorescence meristem (IM) and floral meristem (FM) (FIG. 7). In contrast, the SAMs of wild type soybean plants were not differentiated into IM at this growth stage. Similarly, imaging of the axillary meristem of the FT2a transformants (collected at 9 days after planting) indicated the development of dormant inflorescence meristems (dIMs) (or lateral primordial racemes) into IM and FM (FIG. 8), leading to more earlier-developing floral branches (racemes) per node in these transgenic plants. Additional phenotypic characterization revealed early flowering at the V1 stage in Gm.FT2a expressing soybean plants, which was well before the floral transition occurred in null segregating plants (FIGS. 9A to 9C). These data in combination with the pAt.Erecta::GUS expression pattern described above indicate that early flowering, and more particularly the formation of inflorescence and floral meristems, were induced by ectopic expression of Gm.FT2a during the vegetative stage in leaf primordia and the shoot apical and axillary meristems of seedlings. The formation of a higher number of inflorescence and floral meristems is believed to further cause earlier release and elongation of the secondary and tertiary racemes, leading to a greater number of productive flowers and pods being formed per node.

Not only did Gm.FT2a transgenic soybean plants experience earlier flowering and produce more pods per node on the main stem (relative to segregating null plants), the effects of ectopic Gm.FT2a expression in transgenic plants were also found to be dosage dependent. Although both homozygous and hemizygous plants had a reduced height and less branching, plants homozygous for the Gm.FT2a transgene were more severely affected than hemizygous plants, presumably because homozygous plants contain two copies of the transgene (i.e., a higher dosage), as opposed to only one copy (i.e., a lower dosage) in hemizygous plants. Under long day growth conditions, homozygous plants terminated earlier and had a shorter overall height with fewer nodes and branches on the main stem in comparison to plants hemizygous for the transgene (FIG. 10). Unlike homozygous plants, which exhibited a number of sub-optimal dwarf phenotypes including very few (if any) branches on the main stem, hemizygous plants had an intermediate phenotype in terms of their vegetative growth, plant height, and the number of nodes present on the main stem relative to wild type and homozygous plants. Under 16-hour long day conditions, hemizygous plants had a more normal plant height with some degree of branching and a more extended duration of flowering, relative to homozygous plants (FIG. 10). Hemizygous plants also flowered for 40-64 days after initiation of R1, whereas homozygous plants flowered for only 16-24 days due to their earlier termination.

Additional experiments were conducted with plants transformed with the Gm.FT2a construct (3 events) in long day (16 hour) controlled environment conditions to further characterize the dosage response between hemizygous and homozygous plants. Differences in the number of nodes and pods on the main stem and branches, as well as the average number of pods per node and the average height per plant are shown in Table 1 for three homozygous events (Homo-Event 2, Homo-Event 3, Homo-Event 4) and three hemizygous events (Hemi-Event 2, Hemi-Event 3, Hemi-Event 4). These events are distinguished from Event 1 above.

TABLE 1
Event level data for homozygous and hemizygous Gm.FT2a transgenic plants.

| | Avg. # MS | Avg. # BR | Avg. # MS | Avg. # BR | | Avg. |
|---|---|---|---|---|---|---|
| Zygosity- | nodes per | nodes per | pods per | pods per | Avg. Pods | Height (in) |
| Event # | plant | plant | plant | plant | per Node | per plant |
| Homo- | 11.8 | 6.9 | 46 | 9 | 2.9 | 17.5 |
| Event 2 | | | | | | |
| Homo- | 12.3 | 6.5 | 66.4 | 9 | 4 | 21 |
| Event 3 | | | | | | |
| Homo- | 12.5 | 6.8 | 49.6 | 9.1 | 3 | 19.5 |
| Event 4 | | | | | | |
| Hemi- | 25.3 | 12.4 | 183.5 | 47.3 | 6.1 | 37.5 |
| Event 2 | | | | | | |
| Hemi- | 23.9 | 13.2 | 200.3 | 28.8 | 6.1 | 40 |
| Event 3 | | | | | | |
| Hemi- | 25.4 | 15.3 | 186.8 | 58 | 6 | 41.5 |
| Event 4 | | | | | | |

As shown in Table 2, hemizygous plants consistently had a higher number of nodes on the main stem (MS) and branches (BR) and a greater plant height than homozygous plants. Thus, hemizygous plants were generally less affected than homozygous plants and more like wild type plants. Hemizygous plants also had an increased number of pods per node and a higher number of pods on the main stem and branches, relative to homozygous plants. Therefore, hemizygous plants generally had a closer-to-normal plant architecture with a greater number of pods per node (and per plant), presumably due to their lower Gm.FT2a transgene dosage. The relative dosage level of Gm.FT2a based on transgene zygosity was further confirmed by additional experiments showing that Gm.FT2a transcript levels were higher in tissues from homozygous plants, than in tissues from hemizygous plants (data not shown).

The early induction of flowering in these Gm.FT2a transgenic plants was associated with more pods (and seeds) per node on the main stem in both hemizygous and homozygous plants. Homozygous and hemizygous plants containing the Gm.FT2a transgene each had an increased number of pods/seeds per node on the main stem of the plant in comparison to wild type segregants (FIG. 11). The distribution of pods on the main stem was also found to be different between Gm.FT2a transgenic and wild type null plants. Both homozygous and hemizygous plants grown under long day conditions were found to have more pods on at least the lower nodes of the main stem and more pods per node on average, in comparison to wild type null plants (data not shown). Plants hemizygous for the Gm.FT2a transgene contained the highest number of pods per node over the length of the main stem. However, these effects were dependent on the particular growing conditions including day length, etc. In general, experiments performed with soybean under longer day conditions tended to produce greater differences between transgenic and null plants.

The dosage-dependent effects of transgenic Gm.FT2a expression were also observed in field trial experiments. In a field trial experiment, soybean plants hemizygous for two Gm.FT2a events (Events 1 and 2 above) showed an average of about 2.68 pods per node on the main stem, and plants homozygous for these events had about 1.40 pods per node on average, whereas null segregating plants had about 1.63 pods per node. In an earlier field trial, however, plants hemizygous for transgenic Gm.FT2a (Event 2) were found to have an average number of about 3.21 pods per node, as compared to an average of about 3.05 pods per node in homozygous plants and about 2.19 pods per node in null segregating plants. In another micro plot experiment conducted at a different field location, plants hemizygous for the Gm.FT2a transgene (Event 1) were found to have about 2.17 pods per node on average, as compared to an average of about 2.05 pods per node in plants homozygous for the Gm.FT2a transgene (Event 2) and about 1.30 pods per node in null segregating plants. Thus, the number of pods per node on plants containing the Gm.FT2a transgene may depend on a variety of factors including dosage of the FT transgene, environmental and field conditions, and perhaps differences in agronomic practices. However, much like transgenic Gm.FT2a plants grown in the greenhouse, homozygous and hemizygous Gm.FT2a transgenic plants grown under field conditions often had fewer nodes on the main stem, shorter overall plant height, and/or reduced branching in transgenic plants. Indeed, wild type plants typically had more branching and a greater number of total nodes per plant than hemizygous and homozygous Gm.FT2a plants.

Additional physiological data was collected from homozygous Gm.FT2a transgenic plants and wild type (WT) control plants grown in the greenhouse under 14-hour long day conditions (see Table 2). These data provide an average of measurements taken from six Gm.FT2a transgenic plants for each event, or from eight wild type plants.

The following matrices were collected for phenotypic characterization of these plants: Days to flowering at R1 (DOFR1); Days to R7 (DOR7); reproductive duration in days from R1 to R7 (PDR1R7); number of branches per plant (BRPP); total fertile nodes on branches (FNBR); total fertile nodes per plant (FNLP); total fertile nodes on main stem (FNST); number of nodes on branches (NDBR); number of nodes on main stem (NDMS); number of nodes/plant (NDPL); percent fertile nodes on branches (PFNB); percent total fertile nodes (PFNN); percent fertile nodes on main stem (PFNS); number of pods per plant (PDPP); number of pods on main stem (PODMS); number of pods on branches (PODBR); number of pods/node on average; seeds per plant at R8 (SDPPR8); and weight of 1000 seeds (SW1000). Each of these measurements was taken at harvest unless another time point is specified.

TABLE 2
Construct level phenotypic data for transgenic
homozygous Gm.FT2a and WT plants.

| | WT | pAt.Erecta::Gm.FT2a |
|---|---|---|
| DOFR1 | 33.5 | 21.3 |
| DOR7 | 106.9 | 92.9 |
| PDR1R7 | 76.5 | 71.6 |
| BRPP | 20.1 | 1 |
| FNBR | 190.6 | 2 |
| FNLP | 214.6 | 15 |
| FNST | 24.0 | 14.3 |
| NDBR | 211.4 | 3 |
| NDMS | 33.4 | 15.3 |
| NDPL | 244.9 | 16.3 |
| PDPP | 575.8 | 61.2 |
| PFNB | 90.4 | 75 |
| PFNN | 87.8 | 92.0 |
| PFNS | 71.4 | 92.9 |
| PODBR | 487.3 | 3 |
| PODMS | 88.4 | 60.2 |
| Pods/Node | 2.4 | 3.8 |
| SDPPR8 | 1319.6 | 122.1 |
| SW1000 (grams) | 146 | 122.5 |

Consistent with the observations noted above, homozygous Gm.FT2a transgenic plants experienced earlier floral induction than WT plants (DOFR1 about 21 days after planting, instead of about 33-34 days in wild type plants). These measurements further showed that the number of branches (and other measurements related to branching, such as the number of nodes or pods on branches) was greatly reduced. Due to the transgenic plants having a shorter stature with very little branching, the total numbers of nodes or pods per plant were also greatly reduced. However, the number of pods per node on the main stem was increased in transgenic plants (e.g., about 3.8 average pods/node) relative to wild type null plants (e.g., about 2.4 pods/node).

Without being bound by any theory, the larger number of pods per node observed with transgenic soybean plants expressing FT2a in the meristem during vegetative stages of development may be caused at least in part by synchronization of early flowering with early secondary and/or tertiary raceme release and/or better resource utilization to produce more pod-producing flowers per node. Early FT expression in the meristem (see, e.g., FIGS. 3 and 4) may cause early release of the dormant inflorescence meristems to produce a greater number of racemes per node of the plant, such that a greater number of racemes produce mature flowers and fully developed pods at each node. However, subsequent FT expression in reproductive tissues (see, e.g., FIGS. 5 and 6) may terminate floral development of later developing flowers at each node leading to more efficient resource allocation to the earlier developing racemes, flowers and pods. In wild-type soybean plants, a much lower percentage of secondary and tertiary racemes produce flowers and fully developed pods relative to primary racemes, and later developing flowers of the primary raceme typically do not produce mature flowers and/or full-sized pods prior to abscission. Thus, it is theorized that more pods per node may be generated in plants expressing FT proteins in the vegetative meristem by synchronizing early flower development with early release of the lateral racemes at one or more node(s) of the plant. With at least the pAt.Erecta promoter driving FT expression, later developing flowers (that may not otherwise produce fully developed or full-sized pods) may also become terminated by later reproductive-stage expression of FT to direct resources to the earlier developing flowers.

### Example 4. Expression of Flowering Locus T Gene, Gm.FT2a, Under Control of Alternative Vegetative Stage Promoters in Soybean

Based on the phenotypes observed in the preceding Example 3, two promoters were also proposed to drive Gm.FT2a transgene expression that were considered vegetative-stage, leaf-preferred promoters: pAt.BLS (SEQ ID NO: 46) and pAt.ALMT6 (SEQ ID NO: 47). As used herein, a “leaf-preferred” promoter refers to a promoter that preferentially initiates transcription of its associated gene in leaf tissues relative to other plant tissues. Since FT is believed to function as a mobile florigen, early FT expression during vegetative stages in peripheral tissues, such as in the leaf with a leaf-preferred or leaf-specific promoter, may lead to phenotypes similar to the meristem-preferred pAt.Erecta::Gm.FT2a expression. It was further theorized that FT expression with a vegetative leaf promoter might also attenuate the floral induction signal, and thus mitigate the early termination phenotypes observed with homozygous FT expression in the meristem, and increase plant height and branching.

In these experiments, transformation vectors for pAt.ALMT6::Gm.FT2a and pAt.BLS::Gm.FT2a were constructed and used to transform a soybean line by Agrobacterium-mediated transformation. Expression with the pAt.BLS promoter has been shown to start in leaf primordia number 5 (p5) and is expressed in the source leaf veins only until transition to flowering, and the pAt.ALMT6 promoter is also a vegetative leaf promoter with expression at later developmental stages relative to pAt.BLS. See, e.g., Efroni et al., “A Protracted and Dynamic Maturation Schedule Underlies Arabidopsis Leaf Development,” The Plant Cell 20(9): 2293-2306 (2008); and Shani et al., “Stage-Specific Regulation of Solanum lycopersicum Leaf Maturation by Class 1 KNOTTEDI-LIKE HOMEOBOX Proteins,” The Plant Cell 21(10): 3078-3092 (2009). Transgenic soybean plants were produced for each of these vector constructs and characterized for phenotypes in growth chambers under 14-hour photoperiod conditions in comparison to wild type plants. For each of the pAt.BLS promoter constructs, six transgenic events were tested (5 plants per event), and for the pAt.ALMT6 promoter, seven transgenic events were tested (5 plants per event). For each of these constructs, control data was collected from five wild type plants.

The following matrices were collected for phenotypic characterization of these transgenic plants (Tables 3 and 4). The individual measurements are as defined above, and phenotypic characterization was conducted on plants homozygous for the transgene.

TABLE 3
Construct level phenotypic data for
pAt.ALMT6::Gm.FT2a and WT plants.

| | WT | pAt.ALMT6::Gm.FT2a |
|---|---|---|
| DOFR1 | 35.2 | 38.8 |
| DOR7 | 84.7 | 88.8 |
| PDR1R7 | 49.5 | 50.0 |
| BRPP | 7.7 | 8.9 |
| FNBR | 57.8 | 73.3 |
| FNLP | 69.7 | 85.0 |
| FNST | 12.0 | 11.7 |
| NDBR | 78.9 | 96.0 |
| NDMS | 21.3 | 22.5 |
| NDPL | 100.2 | 118.5 |
| PDPP | 120.2 | 141.1 |
| PFNB | 73.2 | 76.8 |
| PFNN | 71.7 | 72.1 |
| PFNS | 57.9 | 51.7 |
| PODBR | 91.8 | 118.1 |
| PODMS | 28.3 | 22.9 |
| Pods/Node | 1.4 | 1.2 |

TABLE 4
Construct level phenotypic data for
pAt.BLS::Gm.FT2a and WT plants.

| | WT | pAt.BLS::Gm.FT2a |
|---|---|---|
| DOFR1 | 31.3 | 35.2 |
| DOR7 | 78.1 | 82.6 |
| PDR1R7 | 46.9 | 47.5 |
| BRPP | 7.5 | 8.8 |
| FNBR | 65.7 | 81.2 |
| FNLP | 80.5 | 94.0 |
| FNST | 14.9 | 12.7 |
| NDBR | 72.2 | 95.6 |
| NDMS | 21.9 | 22.3 |
| NDPL | 94.0 | 117.9 |
| PDPP | 137.0 | 148.1 |
| PFNB | 92.3 | 85.3 |
| PFNN | 87.4 | 80.1 |
| PFNS | 68.1 | 57.3 |
| PODBR | 100.9 | 123.4 |
| PODMS | 36.1 | 24.8 |
| Pods/Node | 1.7 | 1.3 |

Transgenic plants expressing Gm.FT2a under the control of the alternative pAt.ALMT6 and pAt.BLS promoters were phenotypically more similar to wild type (WT) plants than pAT.Erecta::Gm.FT2a transgenic plants. Plants transformed with the pAt.ALMT6::Gm.FT2a and pAt.BLS::Gm.FT2a constructs had flowering times and vegetative growth traits similar to wild type control plants, perhaps with a slightly increased number of nodes on branches as compared to wild type plants (Tables 3 and 4). These data may be interpreted to indicate that both the timing and location of transgenic FT expression are important for producing reproductive and yield-related traits or phenotypes that differ from wild-type plants. Merely expressing a FT transgene during earlier vegetative stages of development (e.g., in leaf tissues) may not be sufficient to alter the reproductive or yield-related phenotypes of a plant (e.g., pods per node). Thus, according to embodiments of the present invention, a promoter operably linked to a florigenic FT transgene may preferably be a meristem-specific or meristem-preferred promoter in addition to driving expression during the vegetative stages of plant development. However, when the expression profiles for the above two leaf-preferred promoters were tested in soybean plants, no GUS staining was observed in the developing leaf with the pAt.BLS promoter, and the pAt.ALMT6 promoter did not produce detectable GUS expression in the leaf until late vegetative stages with much higher expression during early reproductive stages. Thus, it remains possible that expression of FT transgenes in peripheral (leaf) tissues during early vegetative stages using different tissue-specific promoters may be sufficient in some cases to induce early flowering and/or cause other reproductive or yield-related traits or phenotypes, which may also depend on the particular plant species tested.

### Example 5. Identification of Protein Domains of FT Homologs by Pfam Analysis

Gm.FT2a orthologs were identified by sequence analysis and literature review, and a few examples of these FT homologs are listed in Table 5 along with Gm.FT2a. These included other soybean FT genes as well as a few FT genes from other plant species. The amino acid sequences of these FT proteins were analyzed to identify any Pfam protein domains using the HMMER software and Pfam databases (version 27.0). These FT protein sequences (SEQ ID NOs: 2, 4, 6, 8, 10 and 12) were found to have the same Pfam domain identified as a phosphatidyl ethanolamine binding domain protein (PEBP) having a Pfam domain name of “PBP_N”, and a Pfam accession number of PF01161. The location of the PBP_N domains in each of these FT protein sequences are also listed in Table 5. The location of the PBP_N domain in other FT proteins can be determined by sequence alignment. It is thus contemplated that any DNA sequence encoding at least an FT protein comprising the PBP_N domain may be used in a recombinant DNA molecule of the present invention, as long as the corresponding FT protein has florigenic activity when ectopically expressed in the meristem of a plant.

TABLE 5
Location of PBP_N (Pfam) domain in FT protein sequences.

| PROTEIN | | Domain |
|---|---|---|
| SEQ ID NO. | Gene Name | location |
| 2 | Gm.FT2a | 28-162 |
| 4 | Gm.FT5a | 26-157 |
| 6 | Gm.FT2b | 28-162 |
| 8 | Zm.ZCN8 | 26-154 |
| 10 | Nt.FT-like | 25-159 |
| 12 | Le.SFT | 29-161 |

### Example 6. Expression of FT Homologs Under Control of pAt.Erecta Promoter in Soybean

Additional transformation vectors containing other FT homologs (Table 6) under control of the pAt.Erecta promoter were constructed and used to transform soybeans via Agrobacterium-mediated transformation. Transgenic plants generated from these events were characterized for their phenotypes in the greenhouse with a 14 to 14.5 hour natural daylight photoperiod. For each construct, six events were tested (6 plants per event). Six plants were also tested and averaged for wild type (WT) control plants. Different groups of experiments (A-E) were conducted as shown in Table 6 with separate wild type controls.

TABLE 6
List of constructs for some Gm.FT2a and its
homologs with their protein sequences.

| | | PROTEIN | Testing |
|---|---|---|---|
| Construct Description | Gene Name | SEQ ID NO. | Group |
| pAt.Erecta::Gm.FT2a | Gm.FT2a | 2 | A |
| pAt.Erecta::Gm.FT2b | Gm.FT2b | 6 | C |
| pAt.Erecta::Gm.FT5a | Gm.FT5a | 4 | E |
| pAt.Erecta::Zm.ZCN8 | Zm.ZCN8 | 8 | B |
| pAt.Erecta::Nt.FT-like | Nt.FT-like | 10 | B |
| pAt.Erecta::Le.SFT | Le.SFT | 12 | D |

The following matrices were collected for phenotypic characterization of plants transformed with each of the constructs listed in Table 6 for expressing other FT homologs with the pAt.Erecta promoter, in addition to data collected for the Gm.FT2a construct as described above. The individual measurements are as defined above, and phenotypic characterization of transformants was conducted on plants homozygous for the transgene.

Phenotypic data was collected for plants expressing the Zm.ZCN8 and Nt.FT-like transgenes under the control of the pAt.Erecta promoter (see Tables 7 and 8). Trait values for each Event in Tables 7 and 8 are an average of all plants tested containing the Event. A column is also provided with an average of the Event values for each trait.

TABLE 7
Construct and event level phenotypic data for Zm.ZCN8 and WT plants.

| | WT | Average | Event 1 | Event 2 | Event 3 | Event 4 | Event 5 | Event 6 |
|---|---|---|---|---|---|---|---|---|
| DOFR1 | 33.5 | 28.6 | 29 | 29.2 | 27.5 | 27 | 30.7 | 28 |
| DOR7 | 106.9 | 93.5 | 97.3 | 89.2 | 88.2 | 93.5 | 100.3 | 92.8 |
| PDR1R7 | 76.5 | 64.1 | 69.8 | 60 | 59 | 59.5 | 71.7 | 64.8 |
| BRPP | 20.1 | 3.2 | 2.8 | 1.3 | 1.5 | 1.3 | 9.5 | 3 |
| FNBR | 190.6 | 26.9 | 32 | 8 | 7 | 2.3 | 95.3 | 17 |
| FNLP | 214.6 | 54.9 | 67.5 | 28 | 40.5 | 20.3 | 132.5 | 40.5 |
| FNST | 24.0 | 28.3 | 35.5 | 22 | 33.5 | 18 | 37.3 | 23.5 |
| NDBR | 211.4 | 30.2 | 32.5 | 9 | 7.5 | 3.5 | 110.8 | 17.8 |
| NDMS | 33.4 | 30.5 | 36.3 | 24 | 34.3 | 20 | 44.8 | 24 |
| NDPL | 244.9 | 60.3 | 68.8 | 30.8 | 41.8 | 23.5 | 155.5 | 41.8 |
| PDPP | 575.8 | 317.5 | 498.3 | 144.8 | 319 | 76.3 | 658 | 208.8 |
| PFNB | 90.4 | 87.2 | 98.6 | 90.3 | 85.4 | 64.6 | 91.1 | 93.2 |
| PFNN | 87.8 | 93.1 | 98.1 | 92.3 | 97.0 | 86.5 | 88.5 | 96.4 |
| PFNS | 71.4 | 93.2 | 97.9 | 92.5 | 97.9 | 90.6 | 82.1 | 98.1 |
| PODBR | 487.3 | 105.4 | 162 | 19 | 18.5 | 3.3 | 384.5 | 45.3 |
| PODMS | 88.4 | 212.9 | 336.3 | 130.5 | 300.5 | 73 | 273.5 | 163.5 |
| Pods/Node | 2.4 | 5.5 | 7.2 | 4.6 | 7.7 | 3.2 | 4.9 | 5.2 |
| SDPPR8 | 1319.6 | 564.7 | 961 | 200.5 | 562 | 136.8 | 1166.3 | 361.5 |
| SW1000 | 146 | 108.9 | 102.9 | 127.4 | 105.3 | 82.9 | 116.6 | 117.9 |
| (grams) | | | | | | | | |

TABLE 8
Construct and event level phenotypic data for Nt.FT-like and WT plants.

| | WT | Average | Event 1 | Event 2 | Event 3 | Event 4 | Event 5 | Event 6 |
|---|---|---|---|---|---|---|---|---|
| DOFR1 | 33.5 | 31.5 | 39.3 | 27.7 | 25.3 | 29 | 37.2 | 30.7 |
| DOR7 | 106.9 | 93.9 | 115.8 | 90.7 | 80.7 | 83.7 | 102.2 | 90.2 |
| PDR1R7 | 76.5 | 62.3 | 76.4 | 63 | 55.3 | 54.7 | 65 | 59.5 |
| BRPP | 20.1 | 9.8 | 20 | 8.3 | 2.3 | 5.3 | 17 | 6 |
| FNBR | 190.6 | 108.7 | 190.5 | 95.3 | 11 | 54.3 | 223 | 78.3 |
| FNLP | 214.6 | 131.4 | 212.3 | 118 | 29.5 | 77.8 | 248 | 103 |
| FNST | 24.0 | 23.2 | 21.8 | 22.8 | 21.3 | 23.5 | 25 | 24.8 |
| NDBR | 211.4 | 128.7 | 281.8 | 97 | 11 | 54.5 | 247.7 | 80.5 |
| NDMS | 33.4 | 28.9 | 33.8 | 27 | 23.3 | 24.8 | 35.7 | 28.8 |
| NDPL | 244.9 | 157.1 | 315.5 | 124 | 31.5 | 79.3 | 283.3 | 109.3 |
| PDPP | 575.8 | 462.1 | 638 | 511.3 | 150.8 | 296 | 745 | 431.5 |
| PFNB | 90.4 | 92.5 | 68.0 | 98.6 | 100 | 99.7 | 91.3 | 97.2 |
| PFNN | 87.8 | 89.6 | 67.6 | 95.4 | 93.3 | 98.3 | 88.4 | 94.2 |
| PFNS | 71.4 | 81.9 | 64.7 | 83.3 | 91.3 | 95.2 | 70.6 | 86.2 |
| PODBR | 487.3 | 326.3 | 529 | 342.3 | 22.7 | 147 | 633.3 | 283.5 |
| PODMS | 88.4 | 136.7 | 109 | 169 | 133.8 | 149 | 111.7 | 148 |
| Pods/Node | 2.4 | 3.6 | 2.0 | 4.3 | 4.9 | 3.8 | 2.7 | 4.0 |
| SDPPR8 | 1319.6 | 928.7 | 1359.8 | 965.5 | 382.7 | 591.5 | 1714.3 | 558.7 |
| SW1000 | 146 | 149.0 | 143.7 | 121.2 | 133.8 | 179.3 | 142.2 | 174.0 |
| (grams) | | | | | | | | |

Transgenic soybean plants expressing the Zm.ZCN8 and Nt.FT-like proteins flowered earlier than wild type control plants and had an increased number of pods per node (similar to plants expressing the Gm.FT2a transgene). Indeed, soybean plants expressing the Zm.ZCN8 and Nt.FT-like transgenes had several phenotypes similar to the Gm.FT2a transgenic plants, including reduced number of days to flowering (DOFR1), reduced number of branches (BRPP), fewer nodes per plant (NDPL), fewer nodes on branches (NDBR), reduced number of pods per plant (PDPP), and fewer pods on branches (PODBR), along with an increase in the number of pods per node and a decrease in the number of seeds per plant (Tables 7 and 8), relative to wild type controls. However, several of the negative phenotypes observed in homozygous Gm.FT2a plants were less pronounced in the Zm.ZCN8 and Nt.FT-like expressing transgenic plants. Overall, plants expressing the Zm.ZCN8 transgene had shorter plant height and less branching but more pods per node on the main stem. Similarly, plants expressing the Nt.FT-like transgene had shorter plant height, reduced branching and increased pods per node on the main stem, relative to wild type control plants.

Two transgenic Zm.ZCN8 events and four Nt.FT-like events from above were also tested in field trials at two different locations. Phenotypic data were collected for plants expressing Zm.ZCN8 and Nt.FT-like transgenes under the control of the pAt.Erecta promoter (Tables 9 and 10). For field measurements, the traits are similar to the ones listed above for the greenhouse data tables, except DOR8 and PDR1R8 are days to R8 and reproductive duration between R1 and R8. In addition, all other traits are measured at harvest (i.e., at R8 stage), instead of at R7 stage. Events 1 and 2 in Table 9 correspond to Events 2 and 3 in Table 7, and Events 1-4 in Table 10 correspond to Events 1-4 in Table 8, respectively. Except for days to flowering at R1 (DOFR1) and reproductive duration in days from R1 to R8 (PDR1R8), all phenotypic measurements were derived based on data collected from two locations. Similar to the observations in the greenhouse, transgenic soybean plants expressing Zm.ZCN8 and Nt.FT-like proteins also flowered earlier than wild-type control plants in the field. The Zm.ZCN8 transgenic plants had an increased number of pods per node, while the Nt.FT-like plants did not clearly show increased pods per node in the field trial.

TABLE 9
Phenotypic data from a field trial for Zm.ZCN8 and WT plants.

| | WT | Average | Event 1 | Event 2 |
|---|---|---|---|---|
| DOFR1* | 42.4 | 27.9 | 28.0 | 27.7 |
| DOR8 | 110.7 | 95.0 | 92.0 | 98.0 |
| PDR1R8* | 65.7 | 67.1 | 63.5 | 70.7 |
| BRPPR8 | 2.6 | 0.1 | 0.2 | 0.0 |
| NDBRR8 | 9.7 | 0.3 | 0.5 | 0.1 |
| NDMSR8 | 18.3 | 13.6 | 12.5 | 14.7 |
| NDPLR8 | 28.0 | 13.9 | 13.0 | 14.8 |
| PDPPR8 | 44.2 | 35.1 | 30.1 | 40.0 |
| TPBRR8 | 9.5 | 0.3 | 0.5 | 0.1 |
| PODMSR8 | 34.7 | 34.7 | 29.5 | 39.9 |
| Pods/Node | 1.6 | 2.5 | 2.3 | 2.6 |
| SDPPR8 | 99.9 | 67.6 | 54.7 | 80.5 |
| SW1000 (ounces) | 5.1 | 4.1 | 3.8 | 4.3 |

*(single location data)

TABLE 10
Phenotypic data from a field trial for Nt.FT-like and WT plants.

| | WT | Average | Event 1 | Event 2 | Event 3 | Event 4 |
|---|---|---|---|---|---|---|
| DOFR1* | 42.4 | 38.0 | 42.5 | 26.8 | 26.8 | 25.8 |
| DOR8 | 110.7 | 93.3 | 111.3 | 88.2 | 86.6 | 87.1 |
| PDR1R8* | 65.7 | 63.1 | 66.8 | 62.2 | 60.3 | 63.0 |
| BRPPR8 | 2.6 | 0.7 | 2.4 | 0.1 | 0.2 | 0.1 |
| NDBRR8 | 9.7 | 2.7 | 9.2 | 0.5 | 0.8 | 0.3 |
| NDMSR8 | 18.3 | 11.5 | 18.3 | 9.9 | 7.6 | 10.1 |
| NDPLR8 | 28.0 | 14.2 | 27.5 | 10.4 | 8.5 | 10.4 |
| PDPPR8 | 44.2 | 23.5 | 43.0 | 18.9 | 11.6 | 20.3 |
| TPBRR8 | 9.5 | 2.6 | 8.4 | 0.5 | 0.8 | 0.5 |
| PODMSR8 | 34.7 | 20.9 | 34.6 | 18.5 | 10.8 | 19.8 |
| Pods/Node | 1.6 | 1.6 | 1.6 | 1.8 | 1.4 | 1.7 |
| SDPPR8 | 99.9 | 49.9 | 98.6 | 36.3 | 25.0 | 39.7 |
| SW1000 | 5.1 | 4.5 | 5.1 | 4.4 | 4.6 | 4.0 |
| (ounces) | | | | | | |

(*single location data)

Additional phenotypic data was collected for plants expressing the Gm.FT2b transgene under the control of the pAt.Erecta promoter (Table 11).

TABLE 11
Construct and event level phenotypic data for Gm.FT2b and WT plants.

| | WT | Average | Event 1 | Event 2 | Event 3 | Event 4 | Event 5 | Event 6 |
|---|---|---|---|---|---|---|---|---|
| DOFR1 | 43.7 | 34.6 | 41.2 | 34.3 | 22.7 | 33.2 | 37.2 | 39.3 |
| DOR7 | 105.9 | 100.4 | 100.5 | 100.3 | 99.8 | 100.3 | 98.7 | 102.8 |
| PDR1R7 | 62.2 | 65.8 | 59.3 | 66 | 77.2 | 67.2 | 61.5 | 63.5 |
| BRPP | 13.4 | 4.7 | 7 | 5 | 1.7 | 3.3 | 3.7 | 7.7 |
| FNBR | 103.8 | 32.4 | 52 | 29.7 | 12 | 30.7 | 21.7 | 48.7 |
| FNLP | 125.0 | 46.6 | 68.7 | 41.3 | 24.3 | 45 | 37.3 | 63 |
| FNST | 21.2 | 14.2 | 16.7 | 11.7 | 12.3 | 14.3 | 15.7 | 14.3 |
| NDBR | 108.4 | 34.2 | 54 | 30.3 | 12.7 | 33.7 | 24.7 | 50 |
| NDMS | 30.2 | 18.0 | 18.3 | 15.3 | 15 | 19 | 19.3 | 21 |
| NDPL | 138.7 | 52.2 | 72.3 | 45.7 | 27.7 | 52.7 | 44 | 71 |
| PDPP | 387.4 | 143.0 | 167 | 140 | 96 | 145.7 | 108.7 | 200.7 |
| PFNB | 95.5 | 94.6 | 96.4 | 97.7 | 96.8 | 91.0 | 87.7 | 97.7 |
| PFNN | 90.1 | 89.1 | 94.9 | 90.3 | 88.0 | 86.0 | 86.1 | 89.4 |
| PFNS | 69.7 | 79.2 | 91.5 | 74.5 | 82.5 | 77.0 | 81.1 | 68.7 |
| PODBR | 284.9 | 90.2 | 109.3 | 96 | 43 | 94.7 | 55.3 | 143 |
| PODMS | 102.5 | 52.7 | 57.7 | 44 | 53 | 51 | 53.3 | 57.7 |
| Pods/Node | 2.8 | 2.7 | 2.3 | 3.1 | 3.5 | 2.8 | 2.5 | 2.8 |
| SDPPR8 | 1159.3 | 322.3 | 411.3 | 292.3 | 195.3 | 346.7 | 245 | 443.3 |
| SW1000 | 174.0 | 154.0 | 170.4 | 156.7 | 154.1 | 155 | 130.2 | 157.8 |
| (grams) | | | | | | | | |

Transgenic soybean plants expressing the Gm.FT2b transgene flowered earlier and had less branching than wild type control plants. Gm.FT2b expressing soybean plants had a reduced number of days to flowering (DOFR1), reduced number of branches (BRPP), fewer nodes per plant (NDPL), fewer nodes on branches (NDBR), reduced number of pods per plant (PDPP), and fewer pods on branches (PODBR) (Table 9). However, transgenic Gm.FT2b plants did not show an increase in the number of pods per node. Overall, plants expressing the Gm.FT2b transgene had shorter plant height and less branching relative to wild type control plants. Transgenic soybean plants expressing four different events of the Gm.FT2b transgene were also tested in field trial experiments. Phenotypic data was collected for plants expressing the Gm.FT2b transgene under the control of the pAt.Erecta promoter (Table 12). Events 1-4 in Table 11 correspond to Events 3, 2, 1, and 4 in Table 12, respectively. Similar to the observations in the greenhouse, Gm.FT2b expressing soybean plants showed a reduced number of days to flowering (DOFR1) in the field. The other phenotypic measurements also exhibited similar traits as observed in the greenhouse relative to wild-type control plants.

TABLE 12
Phenotypic data from a field trial for Gm.FT2b and WT plants.

| | WT | Average | Event 1 | Event 2 | Event 3 | Event 4 |
|---|---|---|---|---|---|---|
| DOFR1 | 41.9 | 37.3 | 38.3 | 38.3 | 36.3 | 36.2 |
| DOR8 | 115.4 | 109.2 | 111.1 | 110.6 | 110.6 | 104.6 |
| PDR1R8 | 73.5 | 71.8 | 75.0 | 72.1 | 74.1 | 66.1 |
| SDPPR8 | 188.5 | 95.5 | 99.4 | 81.1 | 117.7 | 83.6 |
| SW1000 | 153.4 | 137.3 | 144.0 | 129.6 | 134.4 | 141.5 |
| (grams) | | | | | | |

Additional phenotypic data was collected from plants expressing the Le.SFT transgene under the control of the pAt.Erecta promoter (Table 13).

TABLE 13
Construct and event level phenotypic data for Le.SFT and WT plants.

| | WT | Average | Event 1 | Event 2 | Event 3 | Event 4 | Event 5 | Event 6 |
|---|---|---|---|---|---|---|---|---|
| DOFR1 | 42.9 | 41.4 | 30 | 44.4 | 30.7 | 28 | 60.2 | 55 |
| DOR7 | 108.6 | 103.8 | 90.7 | 106.2 | 99 | 91.5 | 116.5 | 119 |
| PDR1R7 | 65.5 | 64.0 | 60.2 | 71 | 67 | 65 | 56.4 | 64.2 |
| BRPP | n/a | n/a | n/a | n/a | n/a | n/a | n/a | n/a |
| FNBR | 131.5 | 37.1 | 2.7 | 125.2 | 4.7 | 1 | 47.7 | 41.3 |
| FNLP | 156.8 | 50.7 | 15.7 | 142.6 | 18.1 | 18.3 | 56.7 | 52.7 |
| FNST | 25.3 | 13.7 | 13 | 17.7 | 13.7 | 17.3 | 9 | 11.3 |
| NDBR | 140.2 | 38.9 | 3 | 129.4 | 5.4 | 1 | 51 | 43.7 |
| NDMS | 32.4 | 17.9 | 16 | 25.8 | 16.3 | 21 | 12.3 | 16.3 |
| NDPL | 172.4 | 56.7 | 19 | 154.7 | 21.2 | 22 | 63.3 | 60 |
| PDPP | 473.3 | 201.3 | 53.7 | 432.3 | 69.8 | 85.7 | 279.3 | 287 |
| PFNB | 94.1 | 94.0 | 100 | 96.9 | 83.3 | 100 | 92.9 | 90.7 |
| PFNN | 90.4 | 86.4 | 82.5 | 92.3 | 83.7 | 83.5 | 88.8 | 87.6 |
| PFNS | 77.1 | 76.5 | 81.2 | 69.5 | 84.0 | 82.8 | 72.7 | 68.6 |
| PODBR | 366.2 | 141.8 | 3.7 | 361.5 | 15.5 | 1.3 | 238.7 | 230.7 |
| PODMS | 114 | 60.3 | 50 | 73.6 | 57.1 | 84.3 | 40.7 | 56.3 |
| Pods/Node | 2.7 | 3.6 | 2.8 | 2.8 | 3.3 | 3.9 | 4.4 | 4.8 |
| SDPPR8 | 1247.4 | 476.0 | 136.7 | 1036 | 148.5 | 183 | 655 | 697 |
| SW1000 | 167.7 | 153.2 | 170.0 | 182.8 | 157.5 | 148.5 | 131.5 | 128.8 |
| (grams) | | | | | | | | |

Overall, soybean plants expressing the Le. SFT transgene had shorter plant height with less branching and an increased number of pods per node on average relative to wild type plants (Table 13). However, these effects were variable and event-specific. For example, Events 1, 3 and 4 displayed early flowering (DOFR1), while other events were neutral or actually had delayed flowering. In addition, some of the Le.SFT transgenic events showed increased pods per node on average to varying extents, while a couple of the events were neutral in terms of the average number of pods per node. Interestingly, two of the events (Events 5 and 6) had the greatest number of pods per node on average despite having a delay in flowering.

Additional phenotypic data was collected from plants expressing the Gm.FT5a transgene under the control of the pAt.Erecta promoter (Table 14).

TABLE 14
Construct and event level phenotypic data for Gm.FT5a and WT plants.

| | WT | Average | Event 1 | Event 2 | Event 3 | Event 4 | Event 5 |
|---|---|---|---|---|---|---|---|
| DOFR1 | 48.2 | 29.9 | 32.2 | 29 | 28.6 | 29.2 | 30.5 |
| DOR7 | 110 | 92.5 | 96.6 | 90.4 | 91 | 92.8 | 91.8 |
| PDR1R7 | 61.8 | 62.7 | 64.4 | 61.4 | 62.4 | 63.6 | 61.3 |
| BRPP | 12.4 | 2.5 | 7 | 1.7 | 1 | 1.3 | 1.7 |
| FNBR | 105.6 | 7.3 | 20.3 | 4.7 | 3 | 4 | 4.3 |
| FNLP | 126.5 | 24.5 | 41.7 | 20 | 18.7 | 19.3 | 22.7 |
| FNST | 20.9 | 17.2 | 21.3 | 15.3 | 15.7 | 15.3 | 18.3 |
| NDBR | 108.6 | 7.5 | 21 | 5 | 3 | 4 | 4.3 |
| NDMS | 29 | 17.7 | 22 | 15.7 | 16.3 | 16 | 18.7 |
| NDPL | 137.6 | 25.2 | 43 | 20.7 | 19.3 | 20 | 23 |
| PDPP | 304.3 | 131.9 | 214.7 | 111 | 100.3 | 104.3 | 129.3 |
| PFNB | 97.2 | 98.0 | 97.3 | 93.3 | 100 | 100 | 100 |
| PFNN | 98.1 | 97.0 | 95.9 | 90 | 99.1 | 100 | 100 |
| PFNS | 72.1 | 97.0 | 97.1 | 98.1 | 96.1 | 95.8 | 97.9 |
| PODBR | 233.4 | 16.5 | 60.5 | 8 | 4 | 6 | 4 |
| PODMS | 75.1 | 108.6 | 159 | 98.5 | 95 | 92.5 | 98 |
| Pods/Node | 2.2 | 5.2 | 5.0 | 5.4 | 5.2 | 5.2 | 5.6 |
| SDPPR8 | 778.8 | 271.7 | 516 | 232.7 | 175.3 | 182.3 | 252 |
| SW1000 | 151.6 | 126.0 | 143.7 | 122.4 | 122.2 | 121.8 | 116.8 |
| (grams) | | | | | | | |

Transgenic soybean plants expressing the Gm.FT5a transgene flowered significantly earlier than wild type control plants and had an increased number of pods per node (similar to plants expressing the Gm.FT2a transgene). Indeed, soybean plants expressing the Gm.FT5a transgene had several phenotypes (similar to the Gm.FT2a transgenic plants), including reduced number of days to flowering (DOFR1), reduced number of branches (BRPP), fewer nodes per plant (NDPL), fewer nodes on branches (NDBR), reduced number of pods per plant (PDPP), and fewer pods on branches (PODBR), along with an increase in the number of pods per node and a decrease in the number of seeds per plant (Table 14). Overall, plants expressing the Gm.FT5a transgene had shorter plant height and less branching, but more pods per node (particularly on the main stem) relative to wild type control plants.

Without being bound by any theory, these data support a model of FT overexpression acting in a dosage-dependent manner with the degree or extent of associated phenotypes (e.g., early flowering, increase in pods per node, and altered plant architecture) depending on (i) the level and timing of FT expression, (ii) tissue specificity of FT expression, and (iii) the relative activity and target specificity of the particular FT protein being expressed. For example, expression of the FT protein orthologs from other plant species in soybean may produce a more attenuated effect relative to overexpression of an endogenous FT protein (Gm.FT2a) in soybean, which may result from the non-native FT protein homologs having a lower activity in soybean. However, expression of some native FT proteins may not produce significant phenotypic effects if they have a different or specialized role in their native state or context. Different FT proteins may also act on different tissue targets and receptors and thus have differential effects on the various plant architecture and flowering traits and phenotypes.

Regardless of the activity level of the particular FT homolog, altered reproductive and plant architecture phenotypes appear to correlate with the timing and location of FT expression. Vegetative-stage expression of FT transgenes may be necessary to induce early flowering and/or cause increased numbers of floral meristems, flowers, pods, etc., per node of the plant. Indeed, FT expression in meristematic tissues during vegetative stages of development is shown with proper dosing of the FT transgene to cause reproductive changes in plants leading to increased numbers of flowers, pods, and/or seeds per node. In contrast, expression of a Gm.FT2a transgene under the control of leaf-preferred promoters produced very little, if any, phenotypic changes, relative to wild type plants. These data indicate that both the timing, and tissue specificity (or tissue preference), of FT expression are important factors that affect reproductive and/or yield-related phenotypic changes in transgenic plants.

The present data suggest that different FT proteins may have different activity levels and/or target specificities despite being expressed using the same pErecta promoter. While several constructs expressing Gm.FT2a, Zm.ZCN8, Nt.FT-like, and Gm.FT5a each caused early flowering and termination in addition to an increased number of pods per node, other constructs expressing Gm.FT2b and Le.SFT had different correlative effects on flowering. Expression of Gm.FT2b did cause early flowering and termination of plants but without a significant increase in the number of pods per node. On the other hand, Le.SFT expression showed increased pods per node and early termination despite a delay in flowering. Interestingly, increased numbers of pods per node in transgenic FT plants did not correlate with an extended reproductive duration (PDR1R7) and was not always aligned with early flowering (DOFR1) as noted above. These data suggest that reproductive changes in response to vegetative-stage expression of FT proteins in the meristem may operate through one or more independent mechanisms or pathways. Increased numbers of pods per node in transgenic FT plants may depend on the number of inflorescent and floral meristems induced from vegetative meristems at each node, which may occur independently of flowering time and/or reproductive duration. As noted above, however, reproductive duration may not necessarily correlate with the duration of flowering.

### Example 7. Identification of Additional Vegetative-Stage Meristem Promoters

Having observed phenotypic effects with expression of Gm.FT2a under the control of a vegetative-stage, meristem-preferred promoter, pAt.Erecta, it is contemplated that other vegetative-stage, meristem-preferred (or meristem-specific) promoters may be used to drive expression of FT proteins to cause reproductive or yield-related traits or phenotypes in plants, such as increased number of pods per node (and/or per plant or main stem). Using the characterized expression pattern of the pAt.Erecta promoter (see Example 2), other vegetative-stage, meristem-preferred (or meristem-specific) promoters were identified from soybean, potato and Arabidopsis. Two bioinformatic approaches were utilized to identify candidate genes from other dicotyledonous species including, for example, Arabidopsis, soybean, Medicago, potato and tomato, having similar expression profiles to pAt.Erecta: BAR Espressolog and Expression Angler. See, e.g., BAR expressolog identification: expression profile similarity ranking of homologous genes in plant species,” Plant J 71(6): 1038-50 (2012); and Toufighi, K et al., “The Botany Array Resource: e-Northerns, Expression Angling, and promoter analyses,” Plant J 43(1): 153-163 (2005). The promoter sequences from these genes are thus proposed for use in expressing FT transgenes according to embodiments of the present invention.

Examples of gene promoters identified by this analysis include the following: four receptor like kinase (RLK) genes from soybean, including Glyma10g38730 (SEQ ID NO: 33), Glyma09g27950 (SEQ ID NO: 34), Glyma06g05900 (SEQ ID NO: 35), and Glyma17g34380 (SEQ ID NO: 36). Additional examples include receptor like kinase (RLK) gene promoters from potato, PGSC0003DMP400032802 (SEQ ID NO: 37) and PGSC0003DMP400054040 (SEQ ID NO: 38). It is possible that these RLK genes may be related structurally and/or functionally to Erecta and Erecta-like genes from Arabidopsis and other species since they are also RLK genes. Other vegetative stage, meristem-preferred promoters from Arabidopsis genes include the following: At.MYB17 (At.LMI2; At3g61250) (SEQ ID NO: 41), Kinesin-like gene (At5g55520) (SEQ ID NO: 42), AP2/B3-like genes including At.REM17 (SEQ ID NO: 43) or At.REM19, and Erecta-like 1 and 2 genes, At.Erl1 (SEQ ID NO: 44) andAt.Erl2 (SEQ ID NO: 45). Each of these promoters and similar functional sequences may be operably linked to a FT gene to cause ectopic expression of FT genes in one or more meristem(s) of plants at least during vegetative stage(s) of development.

With regard to the At.MYB17 (At.LMI2) gene, see Pastore, J L et al., “LATE MERISTEM IDENTITY 2 acts together with LEAFY to activate APETALA1,” Development 138: 3189-3198 (2011), the entire contents and disclosure of which are incorporated herein by reference. With regard to the Kinesin-like gene, see Fleury, D et al., “The Arabidopsis thaliana Homolog of Yeast BRE1 Has a Function in Cell Cycle Regulation during Early Leaf and Root Growth,” Plant Cell, 19(2): 417-432 (2007), the entire contents and disclosure of which are incorporated herein by reference. With regard to the REM17 and REM19 Arabidopsis genes, see Mantegazza, O et al., “Analysis of the Arabidopsis REM gene family predicts functions during flower development,” Ann Bot 114(7): 1507-1515 (2014), the entire contents and disclosure of which are incorporated herein by reference. Further, with regard to the At.Erl2 gene, see “Special Issue: Receptor-like Kinases,” JIPB 55(12): 1181-1286 (2013), and particularly Shpak, E., “Diverse Roles of ERECTA Family Genes in Plant Development,” JIPB 55(12): 1251-1263 (2013), the entire contents and disclosures of which are incorporated herein by reference.

### Example 8. Expression of Flowering Locus T Gene, Gm.FT2a, Under Control of a pAt.Erl1 Promoter Alters Flowering Time and Pods Per Node in Soybean

A transformation vector containing Gm.FT2a under control of the vegetative stage, meristem-preferred pAt.Erl1 promoter (SEQ ID NO: 44) was constructed and used to transform soybeans via Agrobacterium-mediated transformation. Transgenic plants generated from these events were characterized for their phenotypes in the greenhouse with a 14 to 14.5 hour natural daylight photoperiod. For each pAt.Erl1::Gm.FT2a construct, six events were tested (6 plants per event) in the greenhouse. Six plants were also tested and averaged for wild type (WI) control plants. Four events were also tested in the field with 12 replicate field plots per event, and compared to WI controls in the same field. The following matrices were collected for phenotypic characterization of these plants and expressed as an average for each Event (as well as the wild type plants) grown in the greenhouse (see Table 15) and in the field (Table 16). A column providing an average for all the Events per trait is further provided.

TABLE 15
Phenotypic data for pAt.Erl1:Gm.FT2a and WT plants grown in the greenhouse.

| | WT | Average | Event 1 | Event 2 | Event 3 | Event 4 | Event 5 | Event 6 |
|---|---|---|---|---|---|---|---|---|
| DOFR1 | 46.1 | 32.6 | 40.0 | 32.3 | 34.0 | 29.3 | 28.2 | 31.8 |
| DOR7 | 115.1 | 99.0 | 109.7 | 99.0 | 99.0 | 93.0 | 91.7 | 101.7 |
| PDR1R7 | 69.0 | 66.4 | 69.7 | 66.7 | 65.0 | 63.7 | 63.5 | 69.8 |
| BRPP | 23.5 | 7.4 | 16.0 | 6.0 | 9.7 | 1.3 | 4.3 | 7.3 |
| NDBR | 277.6 | 80.8 | 215.7 | 51.7 | 139.3 | 3.3 | 18.0 | 56.7 |
| NDMS | 29.8 | 32.3 | 30.7 | 33.7 | 32.7 | 30.7 | 32.7 | 33.3 |
| NDPL | 307.4 | 113.0 | 246.3 | 85.3 | 172.0 | 34.0 | 50.7 | 90.0 |
| PDPP | 605.8 | 346.4 | 447.3 | 349.7 | 493.7 | 240.7 | 194.7 | 352.3 |
| PODBR | 503.1 | 173.7 | 332.3 | 164.7 | 323.3 | 8.3 | 42.7 | 171.0 |
| PODMS | 103.0 | 172.7 | 115.0 | 185.0 | 170.3 | 232.3 | 152.0 | 181.3 |
| Pods/Node | 1.9 | 4.0 | 2.1 | 4.1 | 2.9 | 7.1 | 3.8 | 4.0 |
| SDPPR8 | 1290.0 | 747.5 | 1129.0 | 603.5 | 881.0 | 577.0 | 432.3 | 862.3 |
| SW1000 | 157.6 | 157.3 | 187.7 | 142.6 | 173.8 | 144.3 | 144.9 | 150.3 |
| (grams) | | | | | | | | |

TABLE 16
Phenotypic data for pAt.Erl1:Gm.FT2a
and WT plants grown in the field.

| | WT | Average | Event 1 | Event 2 | Event 4 | Event 6 |
|---|---|---|---|---|---|---|
| DOFR1 | 37.0 | 27.4 | 32.3 | 26.7 | 25.3 | 25.5 |
| DOR8 | 114.9 | 110.6 | 113.8 | 110.4 | 107.9 | 110.5 |
| PDR1R8 | 77.9 | 83.2 | 81.4 | 83.8 | 82.7 | 85.0 |
| BRPPR8 | 2.1 | 0.8 | 1.5 | 1.2 | 0.3 | 0.3 |
| NDBRR8 | 8.0 | 3.2 | 5.6 | 5.2 | 1.0 | 0.9 |
| NDMSR8 | 19.8 | 19.8 | 19.5 | 19.1 | 19.5 | 21.1 |
| NDPLR8 | 27.8 | 23.0 | 25.1 | 24.4 | 20.5 | 22.0 |
| PDPPR8 | 56.1 | 57.8 | 57.4 | 60.6 | 53.1 | 60.2 |
| PDMSR8 | 47.9 | 53.8 | 50.9 | 53.0 | 52.2 | 59.2 |
| TPBR8 | 8.3 | 4.0 | 6.6 | 7.4 | 0.9 | 1.0 |
| Pods/Node | 2.0 | 2.5 | 2.3 | 2.6 | 2.6 | 2.7 |
| SDARR8 | 344.3 | 349.9 | 374.1 | 338.4 | 317.8 | 369.2 |
| SW1000 | 5.4 | 5.4 | 5.4 | 5.3 | 5.3 | 5.5 |
| (ounces) | | | | | | |

Transgenic soybean plants expressing a pAt.Erl1::Gm.FT2a construct flowered earlier than wild type control plants and had an increased number of pods per node (similar to plants expressing the Gm.FT2a transgene under control of the pAt.Erecta promoter). Indeed, soybean plants expressing pAt.Erl1::Gm.FT2a had several phenotypes similar to the pAt.Erecta::Gm.FT2a transgenic plants, including reduced number of days to flowering (DOFR1), reduced number of days to R7 (DOR7), reduced number of branches per plant (BRPP), fewer nodes per plant (NDPL), a reduced number of pods per plant (PDPP), along with an increase in the number of pods per node (Table 15), relative to wild type control plants. However, several phenotypes observed in pAt.Erecta::Gm.FT2a plants, such as number of pods on main stem (PODMS), number of pods on branches (PODBR), and weight of 1000 seeds (SW1000), were less pronounced in the pAt.Erl1::Gm.FT2a expressing transgenic plants. Early results in a second year of field testing also show early flowering indicating similar reproductive traits. As may be inferred, these trait measurements may be abbreviated differently, such as PDPP or PODPP for pods per plant, PDMS or PODMS for pods on the main stem, PDBR or PODBR for pods on braches, TPBR for total pods on branches, etc.

The expression pattern for the Arabidopsis erecta-like 1 promoter (pAt.Erl1) in soybean as measured by GUS staining is more restricted than the expression pattern of pAt.Erecta in soybean as described above. pAt.Erl1 drives GUS expression in vegetative axillary meristems and in early floral meristems derived from axillary tissue. However, GUS staining is not observed in the shoot apical meristem at any stage where it can be distinguished from other meristematic tissues of the developing plant. Expression of the GUS reporter under the control of the pAt.Erl1 promoter is not observed in leaf tissue, stem or root at any stage (data not shown). Given that FT expression under the control of either the pAt.Erecta or pAt.Erl1 promoter induced early flowering and increased pods per node, vegetative expression of an FT transgene at or near the meristem(s) of a plant may generally be sufficient to induce these reproductive and yield-related phenotypes or traits.

### Example 9. Expression of an miRNA Targeting Gm.FT2a for Suppression

It is hypothesized that reducing the expression level of a Gm.FT2a transgene under the control of a vegetative stage promoter in reproductive or floral tissues via RNA suppression may extend reproductive duration in soybean plants. To test whether suppression of an endogenous Gm.FT2a impacts reproductive or morphological phenotypes of soybean plants, a transformation vector comprising a transcribable DNA sequence encoding a miRNA molecule (miRNA-FT2a; SEQ ID NO: 67) that targets an endogenous Gm.FT2a expression under control of a late vegetative and/or reproductive stage promoter, either pAt.AP1 promoter (SEQ ID NO: 49) or pSl.MADS-RIN promoter (SEQ ID NO: 72), was constructed and used to transform soybean plants via Agrobacterium-mediated transformation. Transgenic plants generated from these events were characterized in the greenhouse under 14 to 14.5 hour daylight photoperiod conditions. Six plants per event were tested and compared on average to wild type (WT) control plants. Average phenotypic data collected from the pAP1::miRNA-FT and wild-type plants in the greenhouse is provided in Table 17. Table 18 provides phenoptypic observations collected from transgenic pMADS-RIN::miRNA-FT and wild-type plants in the greenhouse and expressed as an average for each Event and as an average across Events and wild-type plants.

TABLE 17
Phenotypic data for pAP1::miRNA-FT2a and
WT plants grown in the greenhouse.

| | WT | pAP1::miRNA-FT |
|---|---|---|
| DOFR1 | 40 | 37 |
| DOR7 | 89 | 89 |
| PDR1R7 | 49 | 52 |

Under greenhouse conditions, the timing and onset of flowering in plants expressing the pAP1::miRNA-FT2a construct was about the same as WT control plants. Transgenic plants expressing the pAP1::miRNA-FT2a or the pMADS-RIN::miRNA-FT2a construct were also grown under field conditions. Six plants comprising four different events of the pAP1::miRNA-FT2a construct were tested and compared on average to wild type (WT) control plants. For field experiments, measurements were made at harvest (R8 stage), except as otherwise indicated. SDARR8 is seeds per area (i.e., per square foot) at R8 stage. Average phenotypic data collected from field grown plants is provided in Table 18 for the pAP1::miRNA-FT2a construct and Table 20 for the pMADS-RIN::miRNA-FT2a construct.

TABLE 18
Phenotypic data for field-grown
pAP1::miRNA-FT2a and WT plants.

| | WT | Average | Event 1 | Event 2 | Event 3 | Event 4 |
|---|---|---|---|---|---|---|
| DOFR1 | 42.4 | 44.7 | 46.7 | 41.0 | 43.7 | 47.2 |
| DOR8 | 110.7 | 114.5 | 115.0 | 111.6 | 114.4 | 116.8 |
| PDR1R8 | 65.7 | 67.0 | 64.5 | 67.7 | 68.8 | 66.8 |
| BRPPR8 | 2.6 | 2.7 | 2.7 | 2.7 | 2.7 | 2.7 |
| NDBRR8 | 9.7 | 11.0 | 10.9 | 10.7 | 10.5 | 11.9 |
| NDMSR8 | 18.3 | 19.3 | 19.5 | 18.4 | 19.3 | 19.9 |
| NDPLR8 | 28.0 | 30.3 | 30.4 | 29.2 | 29.7 | 31.8 |
| PDPPR8 | 44.2 | 39.7 | 37.2 | 43.2 | 40.5 | 37.8 |
| PODMSR8 | 34.7 | 31.7 | 30.4 | 33.5 | 32.5 | 30.5 |
| TPBR8 | 9.5 | 8.0 | 6.8 | 9.8 | 8.0 | 7.3 |
| Pods/node | 1.6 | 1.4 | 1.3 | 1.5 | 1.4 | 1.2 |
| SDARR8 | 282.7 | 251.6 | 247.1 | 259.0 | 264.8 | 235.5 |
| SW1000 | 5.1 | 5.2 | 5.2 | 5.1 | 5.2 | 5.2 |
| (ounces) | | | | | | |

Under field conditions, soybean plants transformed with the pAP1::miRNA-FT2a construct again had about the same flowering and reproductive traits on average as WT control plants, although transgenic pAP1::miRNA-FT2a plants may have had slightly fewer pods (PDPPR8, PDMSR8, TPBR8, Pods/node) compared to WT control plants at the R8 stage.

### Example 10. Modification of Gm.FT2a Expression by an Artificial miRNA Driven by a pAP1 Promoter Alters Reproductive Phenotypes Under Greenhouse Conditions

To show that suppression of Gm.FT2a in late vegetative and/or reproductive tissues following initial ectopic expression of transgenic Gm.FT2a may extend reproductive duration in soybean plants and/or counteract early termination, a transformation vector comprising Gm.FT2a under control of the vegetative stage, meristem-preferred pAt.Erecta promoter (SEQ ID NO: 31) with either an Apx or Lhcb2 terminator region (T-Apx and T-Lhcb2), and a transcribable DNA sequence encoding an artificial miRNA (SEQ ID NO: 67) targeting Gm.FT2a for suppression and under the control of a pAt.AP1 promoter (SEQ ID NO: 49) with a GAPDH terminator region (each construct designated pAt.Erecta::Gm.FT2a|pAP1::miRNA-FT2a) was constructed and used to transform soybean via Agrobacterium-mediated transformation. Transgenic plants generated from these events were characterized in the greenhouse with a 14 to 14.5 hour daylight photoperiod. For each pAt.Erecta::Gm.FT2a|pAP1::miRNA-FT2a construct, six events were tested (6 plants per event) and the data was averaged. The average data for each construct was also averaged across all events. Six wild type (WI) control plants were also tested and averaged as a control. Tables 19 and 20 provide the phenotypic data collected for the six events as well as the average for WT and pAt.Erecta::Gm.FT2a|pAP1::miRNA-FT2a plants with the two different terminator regions (T-Apx and T-Lhcb2).

TABLE 19
Phenotypic data for pAt.Erecta::Gm.FT2a::T-Apx | pAP1::miRNA-
FT2a::T-GAPDH and WT plants under greenhouse conditions.

| | WT | Average | Event 1 | Event 2 | Event 3 | Event 4 | Event 5 | Event 6 |
|---|---|---|---|---|---|---|---|---|
| DOFR1 | 33.5 | 28.5 | — | 30.0 | 28.0 | 28.0 | 28.0 | 28.3 |
| DOR7 | 106.9 | 99.7 | 106.8 | 98.8 | 98.3 | 91.3 | 102.7 | 100.3 |
| PDR1R7 | 76.5 | 70.1 | — | 68.8 | 70.3 | 65.0 | 74.2 | 72.0 |
| BRPP | 20.1 | 13.0 | 20.3 | 16.7 | 13.0 | 6.7 | 9.7 | 12.0 |
| FNBR | 190.6 | 160.2 | 231.5 | 192.7 | 155.3 | 89.7 | 147.0 | 145.0 |
| FNPL | 214.6 | 191.5 | 263.8 | 226.7 | 184.3 | 122.0 | 176.0 | 176.3 |
| FNST | 24.0 | 31.3 | 32.3 | 34.0 | 29.0 | 32.3 | 29.0 | 31.3 |
| NDBR | 211.4 | 177.1 | 268.8 | 210.0 | 170.0 | 90.0 | 168.3 | 155.3 |
| NDMS | 33.4 | 36.3 | 38.0 | 37.3 | 36.3 | 34.0 | 35.7 | 36.7 |
| NDPL | 244.9 | 213.4 | 306.8 | 247.3 | 206.3 | 124.0 | 204.0 | 192.0 |
| PDPP | 575.8 | 647.5 | 642.8 | 732.0 | 645.8 | 569.7 | 653.7 | 641.0 |
| PFNB | 90.4 | 92.7 | 87.4 | 92.1 | 91.6 | 99.7 | 91.3 | 93.9 |
| PFNN | 87.8 | 91.3 | 86.9 | 92.0 | 89.7 | 98.4 | 89.0 | 91.9 |
| PFNS | 71.4 | 86.0 | 84.9 | 91.7 | 80.4 | 95.3 | 78.8 | 84.7 |
| PODBR | 487.3 | 486.2 | 519.8 | 567.3 | 497.3 | 368.3 | 493.3 | 471.3 |
| PODMS | 88.4 | 161.3 | 123.0 | 164.7 | 148.5 | 201.3 | 160.3 | 169.7 |
| Pods/Node | 2.4 | 3.3 | 2.2 | 3.0 | 3.2 | 4.7 | 3.6 | 3.4 |
| SDPP8 | 1319.6 | 1197.2 | 1508.0 | 1163.0 | 1184.8 | 750.8 | 1170.0 | 1406.8 |
| SW1000 | 146.0 | 158.8 | 149.2 | 169.9 | 144.3 | 172.6 | 163.5 | 153.2 |
| (grams) | | | | | | | | |

TABLE 20
Phenotypic data for pAt.Erecta::Gm.FT2a::T-Lhcb2 | pAP1::miRNA-
FT2a::T-GAPDH and WT plants under greenhouse conditions.

| | WT | Average | Event 1 | Event 2 | Event 3 | Event 4 | Event 5 | Event 6 |
|---|---|---|---|---|---|---|---|---|
| DOFR1 | 33.5 | 28.2 | 28 | — | 28 | 28 | 29.2 | 28 |
| DOR7 | 106.9 | 99.5 | 98.3 | 95.8 | 101 | 99.8 | 99.5 | 102.7 |
| PDR1R7 | 76.5 | 71.7 | 68.6 | — | 73 | 71.8 | 70.3 | 74.7 |
| BRPP | 20.1 | 9 | 5.8 | 8.5 | 8.5 | 6.3 | 15 | 10.3 |
| FNBR | 190.6 | 97.8 | 79.8 | 45.5 | 92 | 84.8 | 149.8 | 135.3 |
| FNPL | 214.6 | 130.5 | 112.3 | 80 | 126.5 | 117 | 184.3 | 162.8 |
| FNST | 24 | 32.6 | 32.5 | 34.5 | 34.5 | 32.3 | 34.5 | 27.5 |
| NDBR | 211.4 | 106.2 | 84.5 | 45.5 | 97.5 | 91 | 164 | 154.8 |
| NDMS | 33.4 | 36.9 | 36.5 | 37 | 37.8 | 35 | 39.3 | 35.8 |
| NDPL | 244.9 | 143.1 | 121 | 82.5 | 135.3 | 126 | 203.3 | 190.5 |
| PDPP | 575.8 | 558.5 | 543.8 | 454 | 566.8 | 534.8 | 633.5 | 618 |
| PFNB | 90.4 | 93.7 | 94.6 | 100 | 94.7 | 94.5 | 91.4 | 86.8 |
| PFNN | 87.8 | 92.3 | 94.1 | 96.7 | 93.6 | 93.5 | 90.7 | 85 |
| PFNS | 71.4 | 88.5 | 88.2 | 94.1 | 91.3 | 92.1 | 87.7 | 77.4 |
| PODBR | 487.3 | 329.7 | 309.5 | 157 | 344 | 304.8 | 446 | 417 |
| PODMS | 88.4 | 228.8 | 234.3 | 297 | 222.8 | 230 | 187.5 | 201 |
| Pods/node | 2.4 | 4.3 | 4.9 | 5.9 | 4.2 | 4.7 | 3.1 | 3.2 |
| SDPP8 | 1319.6 | 778.9 | 728.5 | 644.3 | 832 | 964.8 | 699.5 | 804.5 |
| SW1000 | 146 | 155.5 | 158.1 | 182 | 158.7 | 128.4 | 169.8 | 135.8 |
| (grams) | | | | | | | | |

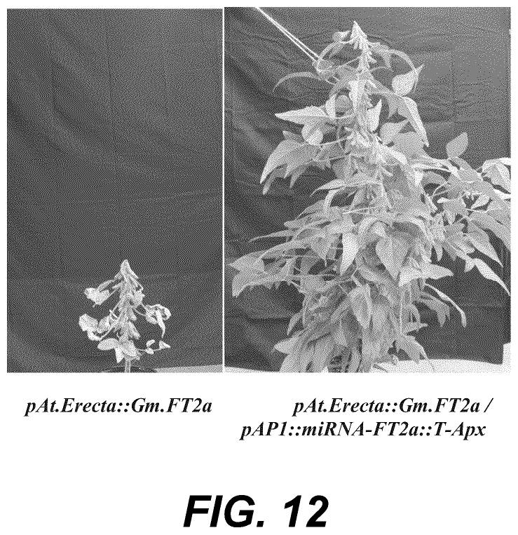

In this greenhouse experiment, transgenic soybean plants comprising either of the pAt.Erecta::Gm.FT2a|pAP1::miRNA-FT2a constructs had an earlier onset of flowering (DOFR1) and an increased number of pods per node (and per main stem) relative to WT control plants (Pods/node, PODMS). Transgenic plants with the combined pAt.Erecta::Gm.FT2a pAP1::miRNA-FT2a construct had increased plant height and branching as well as an increased number of nodes per plant (and per main stem) and increased pods per plant (and per main stem), relative to soybean plants with only the pAt.Erecta::Gm.FT2a transgene without the miRNA suppression cassette (see, e.g., Table 2). FIG. 12 provides whole images of plants homozygous for either pAt.Erecta::Gm.FT2a or pAt.Erecta::Gm.FT2a/pAP1::miRNA-FT2a showing that additional suppression of the Gm.FT2a transgene is effective at mitigating the early termination phenotypes observed with the pAt.Erecta::Gm.FT2a transgene alone, including short plant height and reduced branching.

This data indicates that suppression of Gm.FT2a in later vegetative and/or reproductive tissues following an earlier ectopic dosage of the Gm.FT2a transgene is effective at triggering early flowering and maintaining an increased number of pods per node, while mitigating the early termination phenotypes observed in plants having the Gm.FT2a transgene alone. Interestingly, the increased number of pods per node in these plants was observed without an apparent increase in reproductive duration.

### Example 11. Modification of Gm.FT2a Expression by an Artificial miRNA Driven by a pAP1 Promoter Alters Reproductive Phenotypes Under Field Conditions

Three or four transformation events (depending on the year) of the pAt.Erecta::Gm.FT2a::T-Apx|pAP1::miRNA-FT2a::T-GAPDH construct described in Example 10 were grown and tested under field conditions for two consecutive years. For each of the events and WT control, phenotypic data was collected and averaged (events are numbered for consistency). Tables 21 and 22 provide the average phenotypic data collected from these plants, as well as an average across three events.

Four transformation events of the pAt.Erecta::Gm.FT2a::T-Lhbc2|pAP1::miRNA-FT.::T-GAPDH construct described in Example 10 were grown and tested under field conditions for two consecutive years. For each event and the WT control, phenotypic data was collected and averaged from multiple plants. Tables 23 and 24 provide the average phenotypic data collected from these plants, as well as an average across the four events.

TABLE 21
Phenotypic data for pAt.Erecta::Gm.FT2a::T-Apx | pAP1::miRNA-
FT::T-GAPDH and WT plants grown in the field (Year 1).

| | WT | Average | Event 2 | Event 3 | Event 4 |
|---|---|---|---|---|---|
| DOFR1 | 42.4 | 32.5 | 35.7 | 31.2 | 30.7 |
| DOR8 | 110.7 | 112.5 | 115.1 | 113.3 | 109.2 |
| PDR1R8 | 65.7 | 78.2 | 76.6 | 80.2 | 77.7 |
| BRPPR8 | 2.6 | 0.9 | 1.1 | 1.0 | 0.5 |
| NDBRR8 | 9.7 | 2.6 | 3.7 | 2.9 | 1.1 |
| NDMSR8 | 18.3 | 19.7 | 20.1 | 19.8 | 19.2 |
| NDPLR8 | 28.0 | 22.3 | 23.8 | 22.8 | 20.2 |
| PDPPR8 | 44.2 | 45.8 | 43.6 | 45.6 | 48.1 |
| PDMSR8 | 34.7 | 43.7 | 40.8 | 43.4 | 47.0 |
| TPBR8 | 9.5 | 2.0 | 2.8 | 2.2 | 1.1 |
| Pods/node | 1.6 | 2.1 | 1.8 | 2.0 | 2.4 |
| SDARR8 | 282.7 | 268.6 | 259.1 | 288.7 | 257.9 |
| SW1000 (ounces) | 5.1 | 4.8 | 4.9 | 4.7 | 4.7 |

TABLE 22
Phenotypic data for pAt.Erecta::Gm.FT2a::T-Apx | pAP1::miRNA-
FT::T-GAPDH and WT plants grown in the field (Year 2).

| | WT | Average | Event 2 | Event 3 | Event 4 | Event 6 |
|---|---|---|---|---|---|---|
| DOFR1 | 37.0 | 30.6 | 32.6 | 32.0 | 28.3 | 29.3 |
| DOR8 | 114.9 | 117.6 | 120.7 | 118.9 | 116.0 | 114.8 |
| PDR1R8 | 77.9 | 87.0 | 88.1 | 86.9 | 87.7 | 85.5 |
| BRPPR8 | 2.1 | 0.9 | 1.2 | 2.0 | 0.2 | 0.3 |
| NDBRR8 | 8.0 | 4.7 | 7.5 | 8.1 | 1.5 | 1.5 |
| NDMSR8 | 19.8 | 20.8 | 21.4 | 20.4 | 20.1 | 21.1 |
| NDPLR8 | 27.8 | 25.4 | 28.9 | 28.6 | 21.6 | 22.6 |
| PDPPR8 | 56.1 | 64.2 | 65.5 | 66.0 | 63.2 | 62.2 |
| PDMSR8 | 47.9 | 58.3 | 55.3 | 56.3 | 61.1 | 60.5 |
| TPBR8 | 8.3 | 5.9 | 10.2 | 9.7 | 2.0 | 1.6 |
| Pods/Node | 2.0 | 2.6 | 2.3 | 2.2 | 3.0 | 2.8 |
| SDARR8 | 344.3 | 365.1 | 359.5 | 380.9 | 340.6 | 379.5 |
| SW1000 | 5.4 | 5.4 | 5.4 | 5.6 | 5.4 | 5.4 |
| (ounces) | | | | | | |

TABLE 23
Phenotypic data for pAt.Erecta::Gm.FT2a::T- Lhbc2 | pAP1::miRNA-
FT::T-GAPDH and WT plants grown in the field (Year 1).

| | WT | Average | Event 1 | Event 2 | Event 3 | Event 4 |
|---|---|---|---|---|---|---|
| DOFR1 | 42.4 | 31.8 | 31.2 | 30.8 | 33.0 | 32.3 |
| DOR8 | 110.7 | 110.2 | 110.6 | 109.5 | 111.3 | 109.2 |
| PDR1R8 | 65.7 | 76.2 | 77.3 | 77.7 | 76.3 | 73.5 |
| BRPPR8 | 2.6 | 0.6 | 0.4 | 0.4 | 0.6 | 1.0 |
| NDBRR8 | 9.7 | 1.0 | 0.4 | 0.3 | 1.8 | 1.5 |
| NDMSR8 | 18.3 | 19.6 | 19.9 | 19.6 | 19.4 | 19.6 |
| NDPLR8 | 28.0 | 20.7 | 20.2 | 20.0 | 21.2 | 21.2 |
| PDPPR8 | 44.2 | 49.5 | 49.0 | 49.8 | 48.8 | 50.5 |
| PODMSR8 | 34.7 | 48.4 | 48.5 | 49.2 | 46.9 | 48.8 |
| TPBR8 | 9.5 | 1.2 | 0.5 | 0.6 | 1.9 | 1.7 |
| Pods/node | 1.6 | 2.4 | 2.4 | 2.5 | 2.4 | 2.3 |
| SDARR8 | 282.7 | 287.0 | 285.6 | 288.6 | 272.7 | 300.9 |
| SW1000 | 5.1 | 4.9 | 5.0 | 4.9 | 4.9 | 4.6 |
| (ounces) | | | | | | |

TABLE 24
Phenotypic data for pAt.Erecta::Gm.FT2a::T- Lhbc2 | pAP1::miRNA-
FT::T-GAPDH and WT plants grown in the field (Year 2).

| | WT | Average | Event 1 | Event 2 | Event 3 | Event 4 |
|---|---|---|---|---|---|---|
| DOFR1 | 37.0 | 28.7 | 28.4 | 28.5 | 28.6 | 29.3 |
| DOR8 | 114.9 | 114.6 | 114.7 | 113.7 | 115.0 | 114.9 |
| PDR1R8 | 77.9 | 85.9 | 86.3 | 85.2 | 86.4 | 85.6 |
| BRPPR8 | 2.1 | 0.7 | 0.3 | 0.3 | 0.6 | 1.3 |
| NDBRR8 | 8.0 | 1.8 | 0.8 | 1.2 | 1.4 | 3.8 |
| NDMSR8 | 19.8 | 21.7 | 21.9 | 21.2 | 22.0 | 21.5 |
| NDPLR8 | 27.8 | 23.5 | 22.9 | 22.5 | 23.4 | 25.2 |
| PDPPR8 | 56.1 | 63.6 | 60.6 | 68.3 | 59.4 | 66.0 |
| PDMSR8 | 47.9 | 60.9 | 58.8 | 65.5 | 58.0 | 61.2 |
| TPBR8 | 8.3 | 2.1 | 1.5 | 1.3 | 1.4 | 4.3 |
| Pods/Node | 2.0 | 2.7 | 2.6 | 3.1 | 2.5 | 2.7 |
| SDARR8 | 344.3 | 358.9 | 316.1 | 355.2 | 365.5 | 398.6 |
| SW1000 | 5.4 | 5.5 | 5.6 | 5.6 | 5.5 | 5.2 |
| (ounces) | | | | | | |

Soybean plants having either of the pAt.Erecta::Gm.FT2a|pAP1::miRNA-FT2a constructs (with either terminator) flowered earlier (DOFR1) and had a slightly more extended reproductive duration (in days) between R1 and R8 stages (PDR1R8) and an increased number of pods per node (and per plant) at the R8 stage (PDPPR8, Pods/node) when grown under field conditions relative to WT control plants. However, soybean plants having the pAt.Erecta::Gm.FT2a|pAP1::miRNA-FT2a::T-GAPDH construct also had reduced branching (BRPPR8) relative to WT control plants. Early results in a third year of field testing of plants with the pAt.Erecta::Gm.FT2a pAP1::miRNA-FT2a::T-GAPDH construct also show early flowering indicating similar reproductive traits.

These field results indicate that both of the pAt.Erecta::Gm.FT2a|pAP1::miRNA-FT2a::T-GAPDH constructs with the different terminators are able to enhance yield traits and mitigate early termination phenotypes under field conditions (see, e.g., Example 3 above), which is consistent with the greenhouse data provided in Example 10 above. This data further supports the model that suppression of the Gm.FT2a transgene helps to lessen or mitigate the early termination and plant architecture phenotypes, including reduced plant height, branching and nodes per plant (or per main stem), as compared to expression of the Gm.FT2a transgene alone.

### Example 12. Modification of Gm.FT2a Expression by an Artificial miRNA Driven by a pSl.MADS5 Promoter Alters Reproductive Phenotypes Under Greenhouse Conditions

In addition to the pAP1 promoter, other reproductive stage promoters were used to express a miRNA molecule that targets the Gm.FT2a transgene for suppression. In one set of experiments, a transformation vector comprising Gm.FT2a under control of the pAt.Erecta promoter and a transcribable DNA sequence encoding an artificial miRNA that targets Gm. FT2a for suppression and under the control of a Solanum lycopersicum MADS5 promoter (pSl.MADS5; SEQ ID NO: 71) was constructed and used to transform soybeans via Agrobacterium-mediated transformation. Transgenic plants generated from these events were characterized in the greenhouse for their phenotypes in the greenhouse with a 14 to 14.5 hour daylight photoperiod. For the pAt.Erecta::Gm.FT2a::T-Lhcb2 pSl.MADS5::miRNA-FT2a::T-GAPDH construct, six events were tested (6 plants per event). Six plants were also tested and averaged for wild type (WT) control plants.

Table 25 provides the average phenotypic data for each event and WT plant, and an average for each trait across all events tested. (“--” means data not collected.)

TABLE 25
Phenotypic data for pAt.Erecta::Gm.FT2a::T-Lhcb2 | pSl.MADS5::miRNA-
FT::T-GAPDH and WT plants under greenhouse conditions.

| | WT | Average | Event 1 | Event 2 | Event 3 | Event 4 | Event 5 | Event 6 |
|---|---|---|---|---|---|---|---|---|
| DOFR1 | 33.5 | 28.0 | 28.0 | — | — | — | — | 28.0 |
| DOR7 | 106.9 | 87.1 | 83.3 | 86.7 | 89.7 | 87.5 | 88.3 | 87.2 |
| PDR1R7 | 76.5 | 58.5 | 56.0 | — | — | — | — | 61.0 |
| BRPP | 20.1 | 2.1 | 1.0 | 1.5 | — | 4.0 | 2.0 | 2.0 |
| FNBR | 190.6 | 7.9 | 3.0 | 5.0 | — | 16.5 | 7.7 | 7.3 |
| FNPL | 214.6 | 29.6 | 11.0 | 33.5 | 31.5 | 41.8 | 24.3 | 35.5 |
| FNST | 24.0 | 25.8 | 10.3 | 31.0 | 31.5 | 33.5 | 18.5 | 30.0 |
| NDBR | 211.4 | 8.4 | 3.0 | 6.0 | — | 17.0 | 8.0 | 8.0 |
| NDMS | 33.4 | 26.6 | 10.8 | 31.5 | 32.3 | 33.8 | 19.8 | 31.5 |
| NDPL | 244.9 | 30.6 | 11.5 | 34.5 | 32.3 | 42.3 | 25.8 | 37.5 |
| PDPP | 575.8 | 252.4 | 34.8 | 244.0 | 265.3 | 253.3 | 157.8 | 559.5 |
| PFNB | 90.4 | 94.6 | 100.0 | 87.5 | — | 98.1 | 95.2 | 92.1 |
| PFNN | 87.8 | 96.6 | 95.8 | 97.5 | 97.8 | 99.0 | 94.9 | 94.9 |
| PFNS | 71.4 | 96.8 | 95.8 | 98.5 | 97.8 | 99.2 | 94.5 | 95.1 |
| PODBR | 487.3 | 12.0 | 3.0 | 5.5 | — | 33.0 | 9.7 | 8.7 |
| PODMS | 88.4 | 246.8 | 34.0 | 241.3 | 265.3 | 236.8 | 150.5 | 553.0 |
| Pods/Node | 2.4 | 7.6 | 3.1 | 7.1 | 8.2 | 6.3 | 6.4 | 14.6 |
| SDPP8 | 1319.6 | 335.3 | 38.0 | 321.3 | 359.3 | 527.8 | 305.8 | 460.0 |
| SW1000 | 146.0 | 178.6 | 180.8 | 167.2 | 173.0 | 183.3 | 182.3 | 185.0 |
| (grams) | | | | | | | | |

Similar to the previous examples, limiting the ectopic expression of Gm.FT2a using an artificial miRNA expressed in reproductive tissues resulted in soybean plants that flowered earlier (DOFR1) than WT control plants. However, transgenic soybean plants comprising the pAt.Erecta::Gm.FT2a::T-Lhcb2|pSl.MADS5::miRNA-FT2a::T-GAPDH construct have fewer pods per plant (PDPP), fewer nodes per plant (NDPL), reduced number of branches (BRPP), fewer seeds per plant at R8 stage (SDPP8), and a reduced reproductive duration between R1 and R7 stages (PDR1R7), as compared to WT control plants, although these transgenic plants did have a greater number of pods per node (and per main stem) relative to WT control plants (Pods/Node, PODMS), and improved plant architecture phenotypes relative to pAt.Erecta::Gm.FT2a expression alone (see, e.g., Table 2).

This data further supports the model that suppression of the Gm.FT2a transgene helps to lessen or mitigate the early termination phenotypes with the Gm.FT2a transgene alone, but expression of a miRNA-FT2a with the pMADS5 promoter may not be as effective as the pAP1 promoter at mitigating the early termination phenotypes.

### Example 13. Modification of Gm.FT2a Expression by an Artificial miRNA Driven by a pSl.MADS5 Promoter Alters Reproductive Phenotypes in the Field

Transgenic plants comprising three or four events of the pAt.Erecta::Gm.FT2a pSl.MADS5::miRNA-FT2a construct described in Example 12 were grown and tested under field conditions for two consecutive years. Data was collected for six plants for each event and WT control (events are numbered for consistency). Tables 26 and 27 provide the average phenotypic data collected from each event and WT control, and an average for each trait across all events.

TABLE 26
Phenotypic data for pAt.Erecta::Gm.FT2a::T-
Lhcb2 | pSl.MADS5::miRNA-FT2a::T-GAPDH
and WT plants grown in the field (Year 1).

| | WT | Average | Event 2 | Event 3 | Event 4 |
|---|---|---|---|---|---|
| DOFR1 | 42.4 | 27.6 | 27.7 | 27.5 | 27.7 |
| DOR8 | 110.7 | 100.8 | 100.3 | 101.8 | 100.2 |
| PDR1R8 | 65.7 | 73.1 | 73.2 | 74.0 | 72.0 |
| BRPPR8 | 2.6 | 0.1 | 0.1 | 0.0 | 0.1 |
| NDBRR8 | 9.7 | 0.2 | 0.2 | 0.1 | 0.3 |
| NDMSR8 | 18.3 | 15.2 | 14.5 | 15.7 | 15.3 |
| NDPLR8 | 28.0 | 15.4 | 14.7 | 15.8 | 15.6 |
| PDPPR8 | 44.2 | 43.2 | 41.7 | 45.4 | 42.5 |
| PDMSR8 | 34.7 | 43.0 | 41.4 | 45.3 | 42.2 |
| TPBR8 | 9.5 | 0.2 | 0.3 | 0.0 | 0.3 |
| Pods/Node | 1.6 | 2.9 | 2.9 | 2.9 | 2.8 |
| SDARR8 | 282.7 | 243.7 | 233.8 | 264.4 | 233.0 |
| SW1000 (ounces) | 5.1 | 4.6 | 4.4 | 4.7 | 4.7 |

TABLE 27
Phenotypic data for pAt.Erecta::Gm.FT2a::T-
Lhcb2 | pSlLe.MADS5::miRNA-FT2a::T-GAPDH
and WT plants grown in the field (Year 2).

| | WT | Average | Event 2 | Event 3 | Event 4 | Event 5 |
|---|---|---|---|---|---|---|
| DOFR1 | 37.0 | 24.5 | 24.3 | 24.7 | 24.3 | 24.8 |
| DOR8 | 114.9 | 106.7 | 106.3 | 106.8 | 106.2 | 107.7 |
| PDR1R8 | 77.9 | 82.2 | 82.1 | 82.1 | 81.8 | 82.9 |
| BRPPR8 | 2.1 | 0.3 | 0.3 | 0.2 | 0.5 | 0.2 |
| NDBRR8 | 8.0 | 1.1 | 0.8 | 0.8 | 2.0 | 0.7 |
| NDMSR8 | 19.8 | 15.2 | 15.4 | 15.3 | 15.4 | 14.7 |
| NDPLR8 | 27.8 | 16.2 | 16.2 | 16.1 | 17.3 | 15.1 |
| PDPPR8 | 56.1 | 45.7 | 46.4 | 48.9 | 50.1 | 37.4 |
| PDMSR8 | 47.9 | 44.0 | 45.2 | 47.6 | 46.3 | 37.0 |
| TPBR8 | 8.3 | 1.8 | 1.2 | 1.4 | 3.8 | 1.0 |
| Pods/Node | 2.0 | 2.8 | 2.9 | 3.0 | 2.9 | 2.5 |
| SDARR8 | 344.3 | 254.9 | 280.4 | 236.5 | 267.9 | 235.0 |
| SW1000 | 5.4 | 5.3 | 5.1 | 5.5 | 5.4 | 5.0 |
| (ounces) | | | | | | |

Soybean plants having the pAt.Erecta::Gm.FT2a pSl.MADS5::miRNA-FT2a construct flowered earlier (DOFR1) and had a slightly more extended reproductive duration (in days) between R1 and R8 stages (PDR1R8) and an increased number of pods per node at the R8 stage (Pods/node) under field conditions, relative to WT control plants. However, soybean plants having the pAt.Erecta::Gm.FT2a| pSl.MADS5::miRNA-FT2a construct also had reduced branching (BRPPR8) relative to WT control plants.

### Example 14. Modification of Gm.FT2a Expression by an Artificial miRNA Driven by a pSl.NOD Promoter Alters Reproductive Phenotypes Under Greenhouse Conditions

A third promoter (pSl.NOD) controlling expression of a miRNA-FT2a was also tested under greenhouse conditions. A transformation vector comprising Gm.FT2a under control of the pAt.Erecta promoter (SEQ ID NO: 31) and a transcribable DNA sequence encoding a miRNA-FT2a (SEQ ID NO: 67) under the control of a Solanum lycopersicum NOD promoter (pSl.NOD; SEQ ID NO: 70) was constructed and used to transform soybeans via Agrobacterium-mediated transformation. Transgenic plants comprising six events of this pAt.Erecta::Gm.FT2a pSl.NOD::miRNA-FT2a construct (six plants per event) were grown in the greenhouse with a 14 to 14.5 hour daylight photoperiod. Data was collected from six plants for each event and wild type (WT) control. Table 28 provides the average phenotypic data collected from each event and WI control, and an average for each trait across all events.

TABLE 28
Phenotypic data for greenhouse-grown pAt.Erecta::Gm.FT2a
| pSl.NOD::miRNA-FT2a and WT plants.

| | WT | Average | Event 1 | Event 2 | Event 3 | Event 4 | Event 5 | Event 6 |
|---|---|---|---|---|---|---|---|---|
| DOFR1 | 40.0 | 20.4 | 20.3 | 20.4 | 20.8 | 20.7 | 20.0 | 20.2 |
| DOR7 | 107.3 | 90.9 | 87.2 | 86.0 | 96.8 | 86.0 | 86.0 | 99.6 |
| PDR1R7 | 67.3 | 70.5 | 66.8 | 65.6 | 76.0 | 65.4 | 66.0 | 79.4 |
| BRPP | 17.9 | 3.3 | — | 2.0 | 7.7 | 1.5 | 2.0 | — |
| FNBR | 182.3 | 34.3 | — | 19.0 | 106.0 | 4.5 | 7.5 | — |
| FNPL | 205.9 | 44.2 | 20.0 | 27.7 | 132.3 | 20.8 | 21.0 | 19.0 |
| FNST | 23.6 | 20.2 | 20.0 | 21.3 | 26.3 | 18.5 | 16.0 | 19.0 |
| NDBR | 231.0 | 37.3 | — | 20.0 | 114.0 | 5.5 | 9.5 | — |
| NDMS | 34.4 | 22.6 | 21.3 | 22.3 | 31.0 | 20.3 | 18.7 | 20.7 |
| NDPL | 265.4 | 48.5 | 21.3 | 29.0 | 145.0 | 23.0 | 25.0 | 20.7 |
| PDPP | 703.0 | 228.3 | 133.3 | 188.0 | 563.7 | 108.0 | 139.7 | 142.3 |
| PFNB | 78.9 | 85.6 | — | 95.0 | 93.3 | 75.0 | 78.9 | — |
| PFNN | 77.5 | 90.8 | 94.3 | 96.1 | 91.3 | 90.3 | 84.5 | 91.8 |
| PFNS | 69.8 | 89.7 | 94.3 | 95.2 | 84.8 | 91.1 | 85.8 | 91.8 |
| PODBR | 584.8 | 126.5 | — | 100.0 | 390.3 | 5.0 | 10.5 | — |
| PODMS | 115.1 | 141.7 | 133.3 | 154.7 | 173.3 | 105.5 | 132.7 | 142.3 |
| Pods/Node | 2.7 | 5.6 | 6.3 | 6.5 | 3.9 | 4.8 | 5.9 | 6.8 |
| SDPP8 | 1413.0 | 437.0 | 307.5 | 409.7 | 973.0 | 243.5 | 282.7 | 276.3 |
| SW1000 | 126.2 | 113.4 | 120.5 | 117.5 | 83.6 | 137.5 | 118.4 | 109.8 |
| (grams) | | | | | | | | |

Soybean plants comprising the pAt.Erecta::Gm.FT2a|pSl.NOD::miRNA-FT2a construct flowered earlier (DOFR1) than WI control plants and had a shorter reproductive duration (PDR1R7), fewer branches per plant (BRPP), fewer nodes per plant (NDPL), and fewer pods per plant (PDPP), in the greenhouse as compared to WT control plants. However, these transgenic plants did have a greater number of pods per node (and per main stem) relative to WI plants (Pods/Node, PODMS), and improved plant architecture phenotypes relative to pAt.Erecta::Gm.FT2a expression alone (see, e.g., Table 2).

This data further supports the model that suppression of the Gm.FT2a transgene helps to lessen or mitigate the early termination phenotypes, but expression of a miRNA-FT2a under the pSl.NOD promoter may not be as effective as the pAP1 promoter at mitigating the early termination phenotypes, although these transgenic plants did have a higher number of pods per node (and per main stem) than WT control plants (Pods/Node, PODMS).

### Example 15. Modification of Ectopic Gm.FT2a Expression with a Heterologous miR172 Target Site Alters Reproductive Phenotypes Under Greenhouse Conditions

As an alternative approach to suppressing Gm.FT2a, constructs were made comprising a Gm.FT2a transgene and an engineered miRNA target or binding site (or sensor) that is recognized by an endogenous miRNA in soybean, which serves to attenuate and/or refine the ectopic expression of the Gm.FT2a transgene. Transformation vectors comprising a Gm.FT2a transgene under control of the pAt.Erecta promoter (SEQ ID NO: 31) with either an Apx or Lhcb2 terminator region (T-Apx or T-Lhcb2), and further comprising a sequence (SEQ ID NO: 98) encoding a miR172 miRNA target site (SEQ ID NO: 99) in the transgenic Gm.FT2a mRNA were constructed and used to transform soybean via Agrobacterium-mediated transformation. Transgenic plants generated from these events were characterized for their phenotypes in the greenhouse with a 14 to 14.5 hour daylight photoperiod. For each of the two pAt.Erecta::Gm.FT2a with miR172 binding site constructs, six events were tested (6 plants per event). Six plants were also tested and averaged for wild type (WT) control plants. Tables 29 and 30 provide the average phenotypic data collected from each event and WT control for each of the two constructs, and an average for each trait across all events tested.

TABLE 29
Phenotypic data for pAt.Erecta::Gm.FT2a::T-
Apx/miR172 target site and WT plants.

| | WT | Average | Event 1 | Event 2 | Event 3 | Event 4 | Event 5 | Event 6 |
|---|---|---|---|---|---|---|---|---|
| DOFR1 | 33.5 | 28.0 | — | 28.0 | — | — | — | — |
| DOR7 | 106.9 | 88.3 | 91.3 | 89.7 | 86.3 | 89.0 | 88.2 | 85.3 |
| PDR1R7 | 76.5 | 64.5 | — | 64.5 | — | — | — | — |
| BRPP | 20.1 | 3.9 | 1.5 | 4.0 | — | 11.5 | 1.5 | 1.0 |
| FNBR | 190.6 | 21.6 | 4.0 | 21.3 | — | 75.0 | 3.3 | 4.5 |
| FNPL | 214.6 | 37.0 | 39.0 | 50.3 | 26.5 | 64.3 | 16.5 | 25.5 |
| FNST | 24.0 | 26.8 | 37.0 | 34.3 | 26.5 | 26.8 | 13.3 | 23.3 |
| NDBR | 211.4 | 26.8 | 4.0 | 25.3 | — | 96.5 | 3.5 | 4.5 |
| NDMS | 33.4 | 28.8 | 38.0 | 38.0 | 26.8 | 32.0 | 14.3 | 23.8 |
| NDPL | 244.9 | 41.3 | 40.0 | 57.0 | 26.8 | 80.3 | 17.8 | 26.0 |
| PDPP | 575.8 | 227.9 | 294.0 | 331.5 | 201.8 | 297.0 | 68.0 | 175.3 |
| PFNB | 90.4 | 93.1 | 100.0 | 83.7 | — | 88.3 | 93.8 | 100.0 |
| PFNN | 87.8 | 95.0 | 97.6 | 91.5 | 99.1 | 90.7 | 93.5 | 97.9 |
| PFNS | 71.4 | 93.4 | 97.4 | 90.9 | 99.1 | 82.2 | 93.4 | 97.7 |
| PODBR | 487.3 | 60.8 | 4.5 | 82.0 | — | 206.0 | 5.0 | 6.5 |
| PODMS | 88.4 | 198.8 | 291.8 | 270.0 | 201.8 | 194.0 | 63.0 | 172.0 |
| Pods/Node | 2.4 | 6.4 | 7.4 | 6.7 | 7.5 | 6.1 | 3.9 | 6.8 |
| SDPP8 | 1319.6 | 330.0 | 443.8 | 460.0 | 368.5 | 409.3 | 47.8 | 250.8 |
| SW1000 | 146.0 | 169.5 | 166.8 | 176.3 | 177.7 | 171.8 | 152.7 | 172.1 |
| (grams) | | | | | | | | |

TABLE 30
Phenotypic data for pAt.Erecta::Gm.FT2a::T-
Lhcb2/miR172 target site and WT plants.

| | WT | Average | Event 1 | Event 2 | Event 3 | Event 4 | Event 5 | Event 6 |
|---|---|---|---|---|---|---|---|---|
| DOFR1 | 40.3 | 27.2 | 28.0 | 25.0 | 25.0 | 33.3 | 26.3 | 25.3 |
| DOR7 | 116.5 | 99.9 | 96.6 | 88.0 | 97.0 | 123.0 | 102.4 | 92.7 |
| PDR1R7 | 76.3 | 73.0 | 70.8 | 63.0 | 70.8 | 89.3 | 76.6 | 67.3 |
| BRPP | 17.5 | 5.3 | 3.0 | 1.3 | 1.5 | 13.5 | 6.3 | 6.3 |
| FNBR | 162.5 | 48.6 | 33.0 | 3.8 | 4.5 | 153.5 | 46.3 | 50.3 |
| FNPL | 185.8 | 65.7 | 69.0 | 24.3 | 32.0 | 128.3 | 70.3 | 70.3 |
| FNST | 23.3 | 27.8 | 36.0 | 20.5 | 27.5 | 26.0 | 24.0 | 32.5 |
| NDBR | 249.5 | 55.1 | 37.0 | 5.5 | 7.0 | 173.5 | 50.8 | 56.7 |
| NDMS | 33.5 | 31.9 | 38.3 | 21.8 | 28.5 | 36.7 | 31.8 | 34.3 |
| NDPL | 283.0 | 74.9 | 75.3 | 27.3 | 35.5 | 152.3 | 82.5 | 76.8 |
| PDPP | 628.5 | 317.3 | 407.0 | 132.0 | 198.5 | 433.3 | 338.3 | 394.8 |
| PFNB | 65.2 | 81.9 | 85.3 | 87.5 | 67.8 | 87.7 | 77.5 | 85.5 |
| PFNN | 65.7 | 85.3 | 93.8 | 91.6 | 90.2 | 69.2 | 73.9 | 93.2 |
| PFNS | 69.0 | 86.4 | 94.5 | 94.7 | 96.4 | 66.4 | 71.3 | 95.1 |
| PODBR | 504.8 | 158.8 | 131.7 | 8.0 | 8.5 | 478.5 | 150.5 | 175.3 |
| PODMS | 123.8 | 192.4 | 275.3 | 124.0 | 190.0 | 114.3 | 187.8 | 263.3 |
| Pods/Node | 2.2 | 4.8 | 6.3 | 5.1 | 5.6 | 2.2 | 3.6 | 6.2 |
| SDPP8 | 1261.3 | 559.5 | 712.0 | 118.8 | 655.7 | 593.3 | 603.5 | 673.5 |
| SW1000 | 135.0 | 128.8 | 105.2 | 144.5 | 122.6 | 120.2 | 175.9 | 104.6 |
| (grams) | | | | | | | | |

Soybean plants comprising either of the two pAt.Erecta::Gm.FT2a/miR172 target site constructs flowered earlier (DOFR1), but had reduced branching (BRPP) and fewer pods and nodes per plant (PDPP, NDPL), as compared to WT control plants. However, these transgenic plants did have a greater number of pods per node (and per main stem) relative to WI control plants (Pods/Node, PODMS), and improved plant architecture phenotypes relative to pAt.Erecta::Gm.FT2a expression alone (see Table 2).

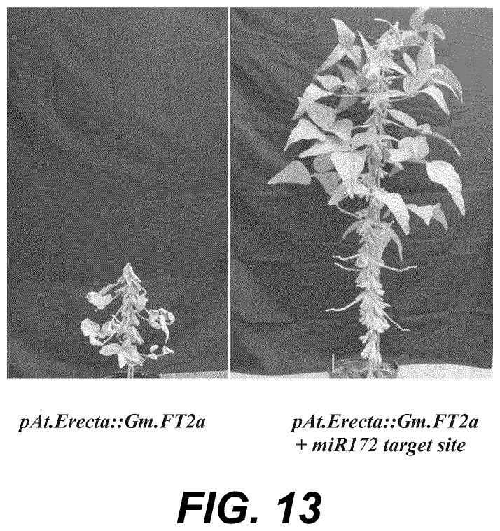

This data supports the hypothesis that suppression of the Gm.FT2a transgene via a target site for an endogenous miR172 is effective at lessening or mitigating the early termination observed with expression of the Gm.FT2a transgene, but suppression of the Gm.FT2a transgene via an engineered miR172 target site may not be as effective (at least in some cases) as expression of an artificial miRNA to suppress Gm.FT2a using a late vegetative and/or reproductive stage promoter, such as the pAP1 promoter, at mitigating the early termination phenotypes, although Gm.FT2a transgenic plants containing the miR172 target site did have a higher number of pods per node (and per main stem) than WT control plants (Pods/Node, PODMS) with improved plant architecture phenotypes relative to Gm.FT2a expression alone. FIG. 13 provides whole images of plants homozygous for either pAt.Erecta::Gm.FT2a or pAt.Erecta::Gm.FT2a/miR172 target site showing that additional suppression via the endogenous miR172 target site is effective at mitigating the early termination phenotypes observed with the pAt.Erecta::Gm.FT2a transgene alone, including short plant height and reduced branching.

### Example 16. Modification of Ectopic Gm.FT2a Expression with a miR172 Target Site Alters Reproductive Phenotypes Under Field Conditions

Transgenic plants comprising four events of the pAt.Erecta::Gm.FT2a::T-Apx/miR172 target site construct described in Example 15 were grown and tested under field conditions. Events 1-4 in Table 29 correspond to Events 1-4 in Table 31, respectively. Data was collected for six plants for each event and WT control. Table 31 provides the average phenotypic data collected from each event and WT control, and an average for each trait across all events.

TABLE 31
Phenotypic data for field-grown pAt.Erecta::Gm.FT2a::T-
Apx/miR172 target site and WT plants.

| | WT | Average | Event 1 | Event 2 | Event 3 | Event 4 |
|---|---|---|---|---|---|---|
| DOFR1 | 42.4 | 26.8 | 26.5 | 27 | 27.3 | 26.3 |
| DOR8 | 110.7 | 91.9 | 93.7 | 92.5 | 91.3 | 90.1 |
| PDR1R8 | 65.7 | 66.1 | 68.5 | 66.4 | 65.2 | 64.2 |
| BRPPR8 | 2.6 | 0.1 | 0.2 | 0.1 | 0.1 | 0.1 |
| NDBRR8 | 9.7 | 0.3 | 0.5 | 0.2 | 0.3 | 0.3 |
| NDMSR8 | 18.3 | 10.8 | 12.5 | 11.2 | 10.7 | 8.9 |
| NDPLR8 | 28.0 | 11.1 | 13 | 11.3 | 11.0 | 9.1 |
| PDPPR8 | 44.2 | 23.2 | 30.1 | 25.0 | 21.7 | 15.8 |
| PDMSR8 | 34.7 | 22.7 | 29.4 | 24.7 | 21.3 | 15.3 |
| TPBR8 | 9.5 | 0.5 | 0.7 | 0.2 | 0.4 | 0.5 |
| Pods/Node | 1.6 | 2.1 | 2.4 | 2.1 | 2.0 | 1.7 |
| SDARR8 | 282.7 | 130.3 | 148.7 | 155.3 | 129.3 | 88.1 |
| SW1000 | 5.1 | 4.6 | 4.7 | 4.5 | 4.6 | 4.6 |
| (ounces) | | | | | | |

Similar to the greenhouse data, soybean plants with the pAt.Erecta::Gm.FT2a::T-Apx/miR172 target site construct flowered earlier (DOFR1) and had an increased number of pods per node (and per main stem) at the R8 stage (PDPPR8, Pods/node) under field conditions, relative to WT control plants. However, these soybean plants with the pAt.Erecta::Gm.FT2a::T-Apx/miR172 target site construct also had reduced branching (BRPPR8) and reduced pods and nodes per plant (PDPPR8, NDPLR8) relative to WT control plants.

### Example 17. Modification of Ectopic Gm.FT2a Expression with a Truncated pAt.Erecta Promoter and a Heterologous miR172 Target Site Alters Reproductive Phenotypes Under Greenhouse Conditions

A truncated pAt.Erecta promoter was used to drive an attenuated level of expression of a Gm.FT2a transgene, and coupled with a miR172 miRNA target site to possibly further attenuate and/or refine the ectopic expression of the Gm.FT2a transgene. Transformation vectors comprising a Gm.FT2a transgene under control of a truncated pAt.Erecta promoter (SEQ ID NO: 32) with a Lhcb2 terminator region (T-Lhcb2) and further comprising a sequence (SEQ ID NO: 98) encoding a miR172 target site (SEQ ID NO: 99) were constructed and used to transform soybean plants via Agrobacterium-mediated transformation. Transgenic plants generated from these events were characterized for their phenotypes in the greenhouse with a 14 to 14.5 hour daylight photoperiod. For the pAt.Erecta_truncated::Gm.FT2a with miR172 target site construct, six events were tested (6 plants per event). Six plants were also tested and averaged for wild type (WT) control plants. Table 32 provides the average phenotypic data collected from each event and WT control, and an average for each trait across the events tested.

TABLE 32
Phenotypic data for pErecta_truncated::Gm.FT2a::T-Lhbc2/miR172
target site and WT plants under greenhouse conditions.

| | WT | Average | Event 1 | Event 2 | Event 3 | Event 4 | Event 5 | Event 6 |
|---|---|---|---|---|---|---|---|---|
| DOFR1 | 40.3 | 33.4 | 32.5 | 25.0 | 34.7 | 35.7 | 38.7 | 33.8 |
| DOR7 | 116.5 | 107.1 | 105.5 | 92.2 | 109.4 | 107.7 | 116.8 | 111.3 |
| PDR1R7 | 76.3 | 74.1 | 73.0 | 67.0 | 75.0 | 72.0 | 80.0 | 77.6 |
| BRPP | 17.5 | 14.2 | 15.3 | 2.0 | 14.8 | 15.8 | 20.8 | 16.8 |
| FNBR | 162.5 | 148.3 | 171.3 | 5.7 | 180.5 | 156.8 | 177.3 | 198.5 |
| FNPL | 185.8 | 175.0 | 201.3 | 36.5 | 207.0 | 180.0 | 203.0 | 222.3 |
| FNST | 23.3 | 26.9 | 30.0 | 32.3 | 26.5 | 23.3 | 25.8 | 23.8 |
| NDBR | 249.5 | 211.9 | 220.8 | 5.7 | 256.0 | 258.0 | 281.8 | 249.5 |
| NDMS | 33.5 | 38.2 | 39.0 | 33.3 | 36.0 | 35.0 | 48.3 | 37.8 |
| NDPL | 283.0 | 249.9 | 259.8 | 37.5 | 292.0 | 293.0 | 330.0 | 287.3 |
| PDPP | 628.5 | 635.0 | 752.5 | 324.8 | 727.8 | 615.0 | 676.3 | 713.8 |
| PFNB | 65.2 | 75.7 | 77.8 | 100.0 | 70.1 | 62.3 | 63.0 | 81.0 |
| PFNN | 65.7 | 74.8 | 77.8 | 97.5 | 70.5 | 62.8 | 61.5 | 78.5 |
| PFNS | 69.0 | 72.1 | 78.0 | 97.0 | 74.3 | 67.2 | 53.0 | 63.2 |
| PODBR | 504.8 | 469.6 | 577.3 | 12.7 | 581.5 | 508.0 | 531.5 | 606.8 |
| PODMS | 123.8 | 165.9 | 175.3 | 315.3 | 146.3 | 107.0 | 144.8 | 107.0 |
| Pods/Node | 2.2 | 3.5 | 2.9 | 8.7 | 2.5 | 2.1 | 2.1 | 2.6 |
| SDPP8 | 1261.3 | 1144.0 | 1550.3 | 691.5 | 1053.8 | 984.0 | 1107.3 | 1477.3 |
| SW1000 | 135.0 | 114.1 | 126.5 | 116.6 | 96.0 | 98.0 | 115.3 | 131.9 |
| (grams) | | | | | | | | |

Soybean plants comprising the pAt.Erecta_truncated::Gm.FT2a/miR172 target site construct flowered earlier (DOFR1), but had slightly reduced branching (BRPP) and fewer nodes per plant (NDPL), as compared to WT control plants. However, these transgenic plants did have a greater number of pods per node (and per main stem) relative to WT control plants (Pods/Node, PODMS), and improved plant architecture phenotypes relative to the pAt.Erecta::Gm.FT2a expression alone (see, e.g., Table 2).

This data further supports the hypothesis that suppression of the Gm.FT2a transgene with a target site for an endogenous miR172 is effective at lessening or mitigating the early termination observed with expression of the Gm.FT2a transgene, and/or that expression of the Gm.FT2a transgene with a truncated pAt.Erecta promoter may further mitigate early termination phenotypes with Gm.FT2a expression alone.

### Example 18. Modification of Ectopic Gm.FT2a Expression with a Truncated pAt.Erecta Promoter and a miR172 Target Site Alters Reproductive Phenotypes in the Field

Transgenic plants for three events of the pAt.Erecta_truncated::Gm.FT2a::T-Lhbc2/miR172 target site construct described in Example 18 were grown and tested under field conditions. Events 1, 2, and 5 in Table 32 correspond to Events 1-3 in Table 33, respectively. Data was collected for six plants for each event and WT control. Table 33 provides average phenotypic data for each event and WT control, and averages for each trait across all events.

TABLE 33
Phenotypic data for pErecta_truncated::Gm.FT2a::T-
Lhbc2/miR172 target site and WT plants in the field.

| | WT | Average | Event 1 | Event 2 | Event 3 |
|---|---|---|---|---|---|
| DOFR1 | 42.4 | 30.4 | 29.8 | 27.7 | 33.8 |
| DOR8 | 110.7 | 105.3 | 108.1 | 99.8 | 108.0 |
| PDR1R8 | 65.7 | 74.3 | 77.0 | 73.8 | 72.2 |
| BRPPR8 | 2.6 | 0.7 | 1.0 | 0.0 | 1.1 |
| NDBRR8 | 9.7 | 1.6 | 1.5 | 0.2 | 3.0 |
| NDMSR8 | 18.3 | 17.3 | 17.8 | 14.8 | 19.2 |
| NDPLR8 | 28.0 | 18.8 | 19.3 | 15.0 | 22.2 |
| PDPPR8 | 44.2 | 43.9 | 45.5 | 41.4 | 44.7 |
| PDMSR8 | 34.7 | 42.4 | 43.9 | 41.1 | 42.1 |
| TPBR8 | 9.5 | 1.6 | 1.6 | 0.4 | 2.7 |
| Pods/Node | 1.6 | 2.4 | 2.4 | 2.7 | 2.1 |
| SDARR8 | 282.7 | 263.4 | 278.2 | 242.7 | 269.2 |
| SW1000 (ounces) | 5.1 | 4.9 | 5.0 | 4.5 | 5.1 |

Similar to the greenhouse data, soybean plants with the pAt.Erecta_truncated::Gm.FT2a/miR172 target site construct flowered earlier (DOFR1) and had an increased number of pods per node (and per main stem) at the R8 stage (PDMSR8, Pods/node) under field conditions, relative to WT control plants. However, soybean plants with the pAt.Erecta_truncated::Gm.FT2a/miR172 target site construct also had reduced branching (BRPPR8) and reduced nodes per plant (NDPLR8) relative to WT control plants.

### Example 19. Modification of Ectopic Gm.FT2a and Native Gm.FT2a, Gm.FT5a and Gm.FT5b Expression in Soybeans by an Artificial miRNA Alters Reproductive Phenotypes

A transformation vector was constructed and used to transform soybean plants via Agrobacterium-mediated transformation, wherein the vector comprised the Gm.FT2a transgene as described above under control of the vegetative stage, meristem-preferred pAt.Erecta promoter (SEQ ID NO: 31) with the Lhcb2 terminator region (T-Lhcb2), and a transcribable DNA sequence encoding an artificial miRNA (SEQ ID NO: 102) that targets not only Gm.FT2a (ectopic and endogenous), but also the endogenous Gm.FT5a and Gm.FT5b genes, for suppression, wherein the transcribable DNA sequence encoding the miRNA is under the control of a pAt.AP1 promoter (SEQ ID NO: 49) with a GAPDH terminator region. Transgenic plants generated from these events were characterized in the greenhouse under 14 to 14.5 hour daylight photoperiod conditions. Data was collected for six plants for each event and WT control. Table 34 provides the average phenotypic data collected from each event and WT control, and an average for each trait across all events.

TABLE 34
Phenotypic data for pAt.Erecta::Gm.FT2a::T-Lhbc2 | pAP1::miRNA-
FT2a/FT5a/FT5b::T-GAPDH and WT plants grown in the greenhouse.

| | WT | Average | Event 1 | Event 2 | Event 3 | Event 4 | Event 5 | Event 6 |
|---|---|---|---|---|---|---|---|---|
| DOFR1 | 39.3 | 32.2 | 34.0 | 32.0 | 33.0 | 30.3 | 31.7 | 32.3 |
| DOR7 | 107.0 | 98.0 | 104.2 | 99.3 | 97.8 | 93.7 | 94.5 | 98.5 |
| PDR1R7 | 67.7 | 65.8 | 70.2 | 67.3 | 64.8 | 63.3 | 62.8 | 66.2 |
| BRPPR8 | 16.8 | 9.9 | 13.8 | 12.7 | 12.2 | 1.8 | 9.0 | 9.7 |
| NDBRR8 | 225.8 | 60.9 | 64.3 | 72.3 | 93.8 | 9.2 | 55.3 | 70.7 |
| NDMSR8 | 29.7 | 29.7 | 34.8 | 33.8 | 33.5 | 15.8 | 33.3 | 27.2 |
| NDPLR8 | 255.5 | 90.7 | 99.2 | 106.2 | 127.3 | 25.0 | 88.7 | 97.8 |
| PDMSR8 | 74.8 | 177.2 | 212.8 | 222.2 | 161.8 | 92.3 | 218.8 | 155.2 |
| PDNDBRR8 | 1.8 | 2.6 | 3.2 | 3.4 | 2.6 | 1.8 | 2.6 | 2.2 |
| PDNDMSR8 | 2.3 | 6.0 | 6.2 | 6.6 | 5.2 | 5.4 | 6.6 | 5.7 |
| Pods/Node | 1.9 | 4.2 | 4.3 | 4.5 | 3.6 | 4.5 | 4.5 | 4.0 |
| PDPPR8 | 482.7 | 349.0 | 411.3 | 470.8 | 418.2 | 111.8 | 346.3 | 335.5 |
| PHTR8 | 1016.3 | 955.5 | 1133.5 | 1156.3 | 1032.5 | 475.5 | 1049.3 | 885.8 |
| SDPDR8 | 2.1 | 1.9 | 1.9 | 1.8 | 1.9 | 1.6 | 2.3 | 1.9 |
| SNUM | 1012.5 | 694.9 | 798.3 | 825.3 | 814.2 | 220.3 | 789.0 | 722.3 |
| TPBR8 | 407.8 | 171.8 | 198.5 | 248.7 | 256.3 | 19.5 | 127.5 | 180.3 |
| SW1000 | 141.2 | 135.4 | 144.3 | 150.8 | 141.5 | 107.1 | 149.5 | 119.4 |
| (grains) | | | | | | | | |

Some additional trait measurements are provided in Table 34 including PDNDBRR8 (pods per node on branches) and PDNDMSR8 (pods per node on main stem) at R8 stage, PHTR8 (plant height at R8 stage in millimeters), SDPDR8 (seeds per pod at R8), and SNUM (number of seeds per plant). Soybean plants expressing the pAt.Erecta::Gm.FT2a cassette with combined suppression of Gm.FT2a, Gm.FT5a, and Gm.FT5b flowered earlier (DOFR1) and had a significantly increased number of pods per node (and increased pods on the mainstem), but decreased branching, relative to WT control plants.

### Example 20. Expression of Ectopic Gm.FT2a Under Control of a pAP1 Promoter Alters Flowering Time and Pods Per Node in Soybean

A transformation vector comprising Gm.FT2a under the control of a pAt.AP1 promoter with a Lhcb2 terminator region was constructed and used to transform soybean via Agrobacterium-mediated transformation. Transgenic plants generated from these events were characterized in the greenhouse with a 14 to 14.5 hour daylight photoperiod. Data was collected for six plants for each event and WT control. Table 35 provides the average phenotypic data collected from each event and WT control, and an average for each trait across all events.

TABLE 35
Phenotypic data for pAt.AP1::Gm.FT2a::T-Lhbc2 and WT plants in the greenhouse.

| | WT | Average | Event 1 | Event 2 | Event 3 | Event 4 | Event 5 | Event 6 |
|---|---|---|---|---|---|---|---|---|
| DOFR1 | 38.3 | 27.4 | 26.7 | 26.7 | 29.3 | 28.2 | 27.0 | 26.5 |
| DOR7 | 106.7 | 94.0 | 91.2 | 92.8 | 96.7 | 93.7 | 94.2 | 95.7 |
| PDR1R7 | 68.3 | 66.6 | 64.5 | 66.2 | 67.3 | 65.5 | 67.2 | 69.2 |
| BRPPR8 | 26.2 | 8.5 | 3.3 | 6.3 | 17.2 | 10.0 | 6.7 | 7.7 |
| NDBRR8 | 352.5 | 90.5 | 14.7 | 75.2 | 175.3 | 106.8 | 91.2 | 79.7 |
| NDMSR8 | 31.5 | 31.8 | 28.8 | 34.5 | 34.3 | 31.7 | 27.5 | 33.8 |
| NDPLR8 | 384.0 | 122.2 | 43.5 | 109.7 | 209.7 | 138.5 | 118.4 | 113.5 |
| PDMSR8 | 69.3 | 165.8 | 122.5 | 212.3 | 120.7 | 182.0 | 158.7 | 198.3 |
| PDNDBRR8 | 1.7 | 3.2 | 1.7 | 4.2 | 2.5 | 4.3 | 2.7 | 4.1 |
| PDNDMSR8 | 2.1 | 5.2 | 4.2 | 6.1 | 3.5 | 6.3 | 5.2 | 5.8 |
| Pods/Node | 1.7 | 3.9 | 3.4 | 4.9 | 2.6 | 4.8 | 3.1 | 4.6 |
| PDPPR8 | 645.2 | 433.9 | 148.8 | 513.5 | 544.5 | 485.7 | 394.2 | 517.0 |
| PHTR8 | 1197.0 | 1153.4 | 1065.3 | 1280.5 | 1240.8 | 1177.5 | 961.1 | 1195.0 |
| SDPDR8 | 2.4 | 2.4 | 2.3 | 2.4 | 2.5 | 2.4 | 2.5 | 2.4 |
| SNUM | 1522.7 | 1059.9 | 349.7 | 1221.7 | 1363.7 | 1172.8 | 985.3 | 1266.3 |
| TPBR8 | 575.8 | 268.4 | 26.3 | 301.2 | 423.8 | 303.7 | 236.8 | 318.7 |
| SW1000 | 196.7 | 180.3 | 213.4 | 170.0 | 181.6 | 171.9 | 174.0 | 171.0 |
| (grains) | | | | | | | | |

Soybean plants expressing the pAt.AP1::Gm.FT2a::T-Lhbc2 cassette flowered earlier (DOFR1) and had a significantly increased number of pods per node (and increased pods on the mainstem), but decreased branching, relative to WT control plants. Early field results with this construct also show early flowering, thus indicating that similar reproductive traits will be observed in plants grown under field conditions.

### Example 21. Expression of Ectopic Gm.FT2a Under Control of a pSl.Nod Promoter in Soybean

A transformation vector comprising Gm.FT2a under the control of a pSl.Nod promoter with a Lhcb2 terminator region was constructed and used to transform soybean via Agrobacterium-mediated transformation. Transgenic plants generated from these events were characterized in the greenhouse with a 14 to 14.5 hour daylight photoperiod. Data was collected for six plants for each event and WT control. Table 36 provides the average phenotypic data collected from each event and WT control, and an average for each trait across all events.

TABLE 36
Phenotypic data for pSl.Nod::Gm.FT2a::T-Fhbc2 and WT plants in the greenhouse.

| | WT | Average | Event 1 | Event 2 | Event 3 | Event 4 | Event 5 | Event 6 |
|---|---|---|---|---|---|---|---|---|
| DOFR1 | 38.3 | 38.1 | 37.5 | 37.0 | 38.3 | 38.8 | 39.5 | 37.7 |
| DOR7 | 107.8 | 106.4 | 105.2 | 105.0 | 105.5 | 109.2 | 104.7 | 108.8 |
| BRPPR8 | 25.3 | 25.3 | 29.3 | 23.8 | 25.2 | 23.8 | 24.3 | 25.3 |
| NDBRR8 | 312.7 | 337.9 | 373.5 | 315.2 | 336.5 | 333.0 | 317.0 | 352.3 |
| NDMSR8 | 38.2 | 36.4 | 34.7 | 37.2 | 37.2 | 39.3 | 36.3 | 33.8 |
| NDPLR8 | 350.8 | 373.3 | 401.8 | 352.3 | 373.7 | 372.3 | 353.3 | 386.2 |
| PDMSR8 | 89.5 | 91.3 | 89.3 | 96.7 | 100.0 | 97.8 | 93.2 | 70.5 |
| PDNDBRR8 | 2.0 | 1.8 | 1.8 | 1.8 | 1.8 | 1.9 | 1.8 | 1.7 |
| PDNDMSR8 | 2.3 | 2.3 | 1.7 | 2.6 | 2.7 | 2.5 | 2.6 | 2.0 |
| Pods/Node | 2.0 | 1.9 | 2.0 | 1.9 | 1.8 | 1.9 | 1.8 | 1.7 |
| PDPPR8 | 700.8 | 677.5 | 750.8 | 664.3 | 681.5 | 676.7 | 640.8 | 650.7 |
| PDR1R7 | 69.5 | 68.3 | 67.7 | 68.0 | 67.2 | 70.3 | 65.2 | 71.2 |
| PHTR8 | 1324.2 | 1256.6 | 1136.6 | 1220.8 | 1315.2 | 1321.7 | 1295.3 | 1249.8 |
| SDPDR8 | 2.5 | 2.4 | 2.1 | 2.4 | 2.4 | 2.4 | 2.5 | 2.5 |
| SNUM | 1713.3 | 1591.3 | 1517.7 | 1584.5 | 1642.7 | 1600.8 | 1588.7 | 1613.7 |
| TPBR8 | 611.3 | 586.2 | 661.5 | 567.7 | 581.5 | 578.8 | 547.7 | 580.2 |
| SW1000 | 187.6 | 188.0 | 197.5 | 187.2 | 189.4 | 185.2 | 187.6 | 181.3 |
| (grains) | | | | | | | | |

In these experiments, no significant change was observed between pSl.Nod::Gm.FT2a::T-Lhbc2 and WT plants. Early field results with this construct further indicate no change in the onset of flowering.

### Example 22. Modification of Gm.FT2a Under Control of a pAt.Erl1 Promoter with an Artificial miRNA Driven by a pSl.MADS-RIN Promoter in Soybean

To test the hypothesis that suppression of Gm.FT2a in late vegetative and/or reproductive tissues following initial ectopic expression of transgenic Gm.FT2a may extend reproductive duration in soybean plants, a transformation vector comprising Gm.FT2a under control of the vegetative stage, meristem-preferred pAt.Erl1 promoter (SEQ ID NO: 44) with GAPDH terminator region (T-GAPDH), and a transcribable DNA sequence encoding an artificial miRNA (SEQ ID NO: 67) targeting Gm.FT2a for suppression and under the control of a pSl.MADS-RIN promoter (SEQ ID NO: 72) with a T-Apx terminator region was constructed and used to transform soybean via Agrobacterium-mediated transformation. Transgenic plants generated from these events were characterized in the greenhouse with a 14 to 14.5 hour daylight photoperiod. Data was collected for six plants for each event and WT control. Table 37 provides the average phenotypic data collected from each event and WT control, and an average for each trait across all events.

TABLE 37
Phenotypic data for pAt.Erl1::Gm.FT2a::T-GAPDH | pSl.MADS-
RIN::miRNA-FT2a::T-Apx and WT plants grown in the greenhouse.

| | WT | Average | Event 1 | Event 2 | Event 3 | Event 4 | Event 5 | Event 6 |
|---|---|---|---|---|---|---|---|---|
| DOFR1 | 39.3 | 33.8 | 33.0 | 34.0 | 35.7 | 35.0 | 33.3 | 31.5 |
| DOR7 | 107.5 | 99.9 | 98.7 | 98.5 | 99.0 | 105.8 | 101.0 | 96.5 |
| PDR1R7 | 68.2 | 66.2 | 65.7 | 64.6 | 63.3 | 70.8 | 67.7 | 65.0 |
| BRPPR8 | 16.5 | 14.4 | 13.3 | 14.2 | 13.2 | 17.8 | 16.0 | 12.0 |
| NDBRR8 | 180.2 | 131.8 | 100.3 | 128.8 | 124.5 | 187.0 | 141.2 | 108.8 |
| NDMSR8 | 31.7 | 32.2 | 31.0 | 32.2 | 28.8 | 36.3 | 31.7 | 33.0 |
| NDPLR8 | 211.8 | 163.9 | 131.3 | 161.0 | 153.3 | 223.3 | 172.8 | 141.8 |
| PDMSR8 | 100.7 | 127.3 | 135.7 | 136.0 | 106.0 | 112.3 | 116.7 | 157.0 |
| PDNDBRR8 | 2.2 | 2.3 | 2.6 | 2.3 | 2.2 | 1.9 | 2.3 | 2.6 |
| PDNDMSR8 | 3.2 | 4.0 | 4.4 | 4.3 | 3.7 | 3.1 | 3.8 | 4.8 |
| Pods/Node | 2.3 | 2.7 | 3.4 | 2.7 | 2.5 | 2.1 | 2.6 | 3.1 |
| PDPPR8 | 480.0 | 423.9 | 420.2 | 419.2 | 388.2 | 449.5 | 429.0 | 437.5 |
| PHTR8 | 1122.5 | 1118.6 | 1081.3 | 1152.5 | 1114.2 | 1185.5 | 1063.3 | 1114.9 |
| SDPDR8 | 2.2 | 2.3 | 2.3 | 2.3 | 2.3 | 2.3 | 2.4 | 2.4 |
| SNUM | 1060.8 | 983.4 | 980.5 | 954.7 | 894.7 | 1023.3 | 1003.7 | 1043.4 |
| TPBR8 | 379.3 | 296.6 | 284.5 | 283.2 | 282.2 | 337.2 | 312.3 | 280.5 |
| SW1000 | 152.2 | 154.1 | 163.6 | 151.6 | 159.8 | 148.4 | 153.7 | 147.3 |
| (grains) | | | | | | | | |

Soybean plants expressing the for pAt.Erl1::Gm.FT2a::T-GAPDH|pSl.MADS-RIN::miRNA-FT2a cassettes flowered slightly earlier (DOFR1) and had a slightly increased number of pods per node (and slightly increased pods on the mainstem), and a moderate decrease in branching, relative to WT control plants. Early field results with this construct also show moderately early flowering, indicating that similar moderate changes in reproductive traits will be observed with this construct in plants grown under field conditions.

### Example 23. Modification of Ectopic Gm.FT2a Expression with a pAt.Erl1 Promoter and a miR156 Sensor Alters Flowering Time

A transformation vector comprising Gm.FT2a under control of the vegetative stage, meristem-preferred pAt.Erl1 promoter (SEQ ID NO: 44) with a miR156 target site (SEQ ID NO: 106) and GAPDH terminator region (T-GAPDH) was constructed and used to transform soybean via Agrobacterium-mediated transformation. Transgenic plants generated from these events were tested in the field. Data was collected for six plants for each event and WT control. Early field results with this construct show early flowering, indicating that moderate changes in reproductive traits including increased pods per node will likely be observed with this construct in plants grown in the field or greenhouse.

### Example 24. Modification of Ectopic Gm.FT2a Expression with a Truncated pAt.Erecta Promoter Alters Flowering Time

A transformation vector comprising Gm.FT2a under control of a shorter truncated pAt.Erecta promoter (SEQ ID NO: 48) with a Lhcb2 terminator region (T-Lhcb2) was constructed and used to transform soybean via Agrobacterium-mediated transformation. Transgenic plants generated from these events were tested in the field. Data was collected for six plants for each event and WT control. Early field results with this construct show slightly earlier onset of flowering, indicating that slight or moderate changes in reproductive traits including slightly increased pods per node will likely be observed with this construct in plants grown in the field or greenhouse. Thus, more attenuated reproductive phenotypes and mitigated early termination effects may be achieved with this more minimal, truncated pAt.Erecta promoter (compare Examples 3 and 17).

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

What is claimed is:

1. A recombinant DNA construct comprising a first expression cassette and a second expression cassette, wherein the first expression cassette comprises a polynucleotide sequence encoding a florigenic FT protein operably linked to a first plant expressible promoter, and the second expression cassette comprises a transcribable DNA sequence encoding a RNA molecule comprising a targeting sequence that is at least 80% complementary to at least 15 consecutive nucleotides of the polynucleotide sequence of the first expression cassette, and wherein the transcribable DNA sequence is operably linked to a second plant expressible promoter.

2. The recombinant DNA construct of claim 1, wherein the targeting sequence of the RNA molecule is at least 85% complementary to at least 15 consecutive nucleotides of the polynucleotide sequence of the first expression cassette or an mRNA encoded by the polynucleotide sequence of the first expression cassette.

3. The recombinant DNA construct of claim 1, wherein the transcribable DNA sequence comprises a sequence that is at least 80% complementary to SEQ ID NO: 65, 68, or 69.

4. The recombinant DNA construct of claim 1, wherein the florigenic FT protein comprises an amino acid sequence having at least 60% identity to a sequence selected from the group consisting of SEQ ID NOs: 2, 4, 6, 8, 10, 12, 14, 16, 18, 20, 22, 24, 26, 28, and 30, or a functional fragment thereof.

5. The recombinant DNA construct of claim 4, wherein the florigenic FT protein further comprises one or more of the following amino acids: a tyrosine or other uncharged polar or nonpolar residue at the amino acid position of the florigenic FT protein corresponding to amino acid position 85 of SEQ ID NO: 14; a leucine or other nonpolar residue at the amino acid position of the florigenic FT protein corresponding to amino acid position 128 of SEQ ID NO: 14; and a tryptophan or other large nonpolar residue at the amino acid position of the florigenic FT protein corresponding to amino acid position 138 of SEQ ID NO: 14.

6. The recombinant DNA construct of claim 4, wherein the florigenic FT protein does not have one or more of the following amino acids: a histidine at the amino acid position corresponding to a lysine or arginine at the amino acid position corresponding to position 85 of SEQ ID NO: 14; a lysine or arginine at the amino acid position corresponding to position 128 of SEQ ID NO: 14; and a serine, aspartic acid, glutamic acid, lysine or arginine at the amino acid position corresponding to position 138 of SEQ ID NO: 14.

7. The recombinant DNA construct of claim 1, wherein the polynucleotide sequence is at least 60% identical to a sequence selected from the group consisting of SEQ ID NOs: 1, 3, 5, 7, 9, 11, 13, 15, 17, 19, 21, 23, 25, 27, and 29.

8. The recombinant DNA construct of claim 1, wherein the first plant expressible promoter comprises a polynucleotide sequence that is at least 70% identical to a polynucleotide sequence selected from the group consisting of SEQ ID NOs: 31, 32, 33, 34, 35, 36, 37, 38, 39, 40, 41, 42, 43, 44, 45, 48, 49, 50, 51, 52, 53, 54, 55, 56, 57, 58, 59, 60, 61, 62, 63 and 64, or a functional portion thereof.

9. The recombinant DNA construct of claim 1, wherein the second plant expressible promoter comprises a polynucleotide sequence that is at least 70% identical to a polynucleotide sequence selected from the group consisting of SEQ ID NOs: 49, 50, 51, 52, 53, 54, 55, 56, 57, 58, 59, 60, 61, 62, 63, 64, or a functional portion thereof.

10. The recombinant DNA construct of claim 1, wherein the second plant expressible promoter comprises a polynucleotide sequence that is at least 70% identical to a polynucleotide sequence selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, and 94, or a functional portion thereof.

11. The recombinant DNA construct of claim 1, wherein the first plant expressible promoter is a vegetative stage promoter and the second plant expressible promoter is a late vegetative stage promoter and/or reproductive stage promoter.

12. The recombinant DNA construct of claim 11, wherein the first plant expressible promoter is an early vegetative stage promoter.

13. The recombinant DNA construct of claim 11, wherein the second plant expressible promoter is a reproductive stage preferred promoter.

14. The recombinant DNA construct of claim 1, wherein the first plant expressible promoter initiates detectable expression of the polynucleotide sequence encoding the florigenic FT protein at an earlier developmental stage than the second plant expressible promoter initiates detectable expression of the transcribable DNA sequence.

15. A transgenic plant comprising an insertion of the recombinant DNA construct of claim 1 into the genome of at least one cell of the transgenic plant.

16. The transgenic plant of claim 15, wherein the transgenic plant is soybean.

17. The transgenic plant of claim 16, wherein the transgenic soybean plant produces more pods per node than a control plant not having the recombinant DNA construct.

18. The transgenic plant of claim 15, wherein the transgenic plant produces more flowers per node than a control plant not having the recombinant DNA construct.

19. The transgenic plant or part thereof of claim 15, wherein the transgenic plant produces more seeds, bolls, siliques, fruits, nuts or pods per node of the transgenic plant than a control plant not having the recombinant DNA construct.

20. The transgenic plant or part thereof of claim 15, wherein the transgenic plant flowers earlier than a control plant not having the recombinant DNA construct.

21. The transgenic plant or part thereof of claim 15, wherein the transgenic plant has more floral racemes per node than a control plant not having the recombinant DNA construct.

22. A recombinant DNA construct comprising a polynucleotide sequence encoding a florigenic FT protein operably linked to a plant expressible promoter, wherein the polynucleotide sequence comprises a sequence that encodes a target site in a mRNA transcript encoded by the polynucleotide sequence, and wherein the target site of the mRNA transcript is at least 80% complementary to an endogenous RNA molecule.

23. The recombinant DNA construct of claim 22, wherein the target site of the mRNA transcript is at least 17 nucleotides in length.

24. The recombinant DNA construct of claim 22, wherein the target site of the mRNA transcript is at least 80% complementary to the endogenous RNA molecule.

25. The recombinant DNA construct of claim 22, wherein the target site of the mRNA transcript is at least 80% complementary to SEQ ID NO: 95, 96, 97, 103, 104, or 105.

26. The recombinant DNA construct of claim 22, wherein the polynucleotide sequence encoding the florigenic FT protein comprises a sequence that is at least at least 80% identical to SEQ ID NO: 98, 99, 100, 101, 106, 107, 108, 109 or 110.

27. The recombinant DNA construct of claim 22, wherein the target site of the mRNA transcript is at least 80% identical to SEQ ID NO: 99 or 107.

28. The recombinant DNA construct of claim 22, wherein the florigenic FT protein comprises an amino acid sequence having at least 60% identity to a sequence selected from the group consisting of SEQ ID NOs: 2, 4, 6, 8, 10, 12, 14, 16, 18, 20, 22, 24, 26, 28, and 30, or a functional fragment thereof.

29. The recombinant DNA construct of claim 22, wherein the plant expressible promoter is a vegetative stage promoter.

30. The recombinant DNA construct of claim 22, wherein the plant expressible promoter is a meristem-preferred or meristem-specific promoter.

31. The recombinant DNA construct of claim 22, wherein the plant expressible promoter comprises a polynucleotide sequence that is at least 70% identical to a polynucleotide sequence selected from the group consisting of SEQ ID NOs: 31, 32, 33, 34, 35, 36, 37, 38, 39, 40, 41, 42, 43, 44, 45, 48, 49, 50, 51, 52, 53, 54, 55, 56, 57, 58, 59, 60, 61, 62, 63 or 64, or a functional portion thereof.

32. A transgenic plant comprising an insertion of the recombinant DNA construct of claim 22 into the genome of at least one cell of the transgenic plant.

33. The transgenic plant of claim 32, wherein the transgenic plant is soybean.

34. The transgenic plant of claim 33, wherein the transgenic soybean plant produces more pods per node than a control plant not having the recombinant DNA construct.

35. The transgenic plant of claim 32, wherein the transgenic plant produces more flowers per node than a control plant not having the recombinant DNA construct.

36. The transgenic plant or part thereof of claim 32, wherein the transgenic plant produces more bolls, siliques, fruits, nuts or pods per node of the transgenic plant than a control plant not having the recombinant DNA construct.

37. The transgenic plant or part thereof of claim 32, wherein the transgenic plant flowers earlier than a control plant not having the recombinant DNA construct.

38. The transgenic plant or part thereof of claim 32, wherein the transgenic plant has more floral racemes per node than a control plant not having the recombinant DNA construct.

39. A transgenic plant comprising a polynucleotide sequence encoding a florigenic FT protein operably linked to a vegetative stage promoter, wherein expression of the florigenic FT protein is suppressed in a late vegetative and/or reproductive tissue.

40. The transgenic plant of claim 39, wherein expression of the florigenic FT protein is suppressed by a small RNA molecule.

41. A recombinant DNA construct comprising a polynucleotide sequence encoding a florigenic FT protein and operably linked to a vegetative stage promoter, and at least one transcribable DNA sequence encoding an RNA molecule comprising a targeting sequence that is complementary to at least a portion of the polynucleotide sequence.

42. A transgenic plant comprising an insertion of the recombinant DNA construct of claim 41 into the genome of at least one cell of the transgenic plant.

43. A transgenic plant comprising a recombinant polynucleotide sequence encoding a florigenic FT protein operably linked to a vegetative stage promoter, wherein expression of the polynucleotide sequence is spatially and temporally restricted by a small RNA molecule.


SEQUENCE LISTING (110 sequences)

SEQ ID NO: 1 - DNA, 531 bp, Glycine max
  source 1..531: mol_type = genomic DNA
atgcctagtggaagtagggatcctctcgttgttgggggagtaattggggatgtattggatccttttgaatattctattcctatgagggttacctacaataacagagatgtcagcaatggatgtgaattcaaaccctcacaagttgtcaaccaaccaagggtaaatatcggtggtgatgacctcaggaacttctatactttgattgcggttgatcccgatgcacctagcccaagtgaccccaatttgagagaatacctccattggttggtgactgatatcccagcaacaacaggggctagtttcggccatgaggttgtaacatatgaaagtccaagaccaatgatggggattcatcgtttggtgtttgtgttatttcgtcaactgggtagggagaccgtgtatgcaccaggatggcgccagaatttcaacactaaagaatttgctgaactttacaaccttggattgccagttgctgctgtctatttcaacattcagagggaatctggttctggtggaaggaggttatactaa

SEQ ID NO: 2 - protein, 176 aa, Glycine max
MPSGSRDPLVVGGVIGDVLDPFEYSIPMRVTYNNRDVSNGCEFKPSQVVNQPRVNIGGDDLRNFYTLIAVDPDAPSPSDPNLREYLHWLVTDIPATTGASFGHEVVTYESPRPMMGIHRLVFVLFRQLGRETVYAPGWRQNFNTKEFAELYNLGLPVAAVYFNIQRESGSGGRRLY

SEQ ID NO: 3 - DNA, 519 bp, Glycine max
  source 1..519: mol_type = genomic DNA
atggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggttatggtggatgcagatgcacctagccctagcaaccctgtcttgagggaataccttcactggatggtgacagatattccagctaccacaaatgcaagctttgggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 4 - protein, 172 aa, Glycine max
MARENPLVIGGVIGDVLNPFTSSVSLTVSINNRAISNGLELRPSQVVNRPRVTVGGEDLRTFYTLVMVDADAPSPSNPVLREYLHWMVTDIPATTNASFGREVVFYESPNPSVGIHRIVFVLFQQLGRDTVITPEWRHNFNSRNFAEINNLAPVAAAYANCQRERGCGGRRY

SEQ ID NO: 5 - DNA, 531 bp, Glycine max
  source 1..531: mol_type = genomic DNA
atgcctcgtggaagtagggaccctctagttgttgggcgtgtgattggggatgtattggacccttttgaatgttctattcctatgagggtcacctacaataacaaagatgtcagcaatggatgtgaattcaaaccctcacaagttgtcaaccaaccaagaataaatatcggtggtgatgatttcaggaacttctacactttgatcgcggttgatcctgatgcacctagcccaagtgatcccaatttcagagaatacctccattggttagtaactgacattccagcaacaacggggcctactttcggtcatgaggttgtaacatatgaaaatccacgacccatgatggggatccatcgtatagtctttgtgttatttcgtcaacagggtagagagacagtgtatgcaccaggatggcgccaaaatttcattactagagaatttgctgaactttacaatcttggattgccagttgctgctgtctattttaacatccagagagaatctggttgtggtggaagaaggctatgttaa

SEQ ID NO: 6 - protein, 176 aa, Glycine max
MPRGSRDPLVVGRVIGDVLDPFECSIPMRVTYNNKDVSNGCEFKPSQVVNQPRINIGGDDFRNFYTLIAVDPDAPSPSDPNFREYLHWLVTDIPATTGPTFGHEVVTYENPRPMMGIHRIVFVLFRQQGRETVYAPGWRQNFITREFAELYNLGLPVAAVYFNIQRESGCGGRRLC

SEQ ID NO: 7 - DNA, 528 bp, Zea mays
  source 1..528: mol_type = genomic DNA
atgtcagcaaccgatcatttggttatggctcgtgtcatacaggatgtattggatccctttacaccaaccattccactaagaataacgtacaacaataggctacttctgccaagtgctgagctaaagccatccgcggttgtaagtaaaccacgagtcgatatcggtggcagtgacatgagggctttctacaccctggtactgattgacccggatgccccaagtccaagccatccatcactaagggagtacttgcactggatggtgacagatattccagaaacaactagtgtcaactttggccaagagctaatattttatgagaggccggacccaagatctggcatccacaggctggtatttgtgctgttccgtcaacttggcagggggacagtttttgcaccagaaatgcgccacaacttcaactgcagaagctttgcacggcaatatcacctcagcattgccaccgctacacatttcaactgtcaaagggaaggtggatccggcggaagaaggtttagggaagagtag

SEQ ID NO: 8 - protein, 175 aa, Zea mays
MSATDHLVMARVIQDVLDPFTPTIPLRITYNNRLLLPSAELKPSAVVSKPRVDIGGSDMRAFYTLVLIDPDAPSPSHPSLREYLHWMVTDIPETTSVNFGQELIFYERPDPRSGIHRLVFVLFRQLGRGTVFAPEMRHNFNCRSFARQYHLSIATATHFNCQREGGSGGRRFREE

SEQ ID NO: 9 - DNA, 525 bp, Nicotiana tabacum
  source 1..525: mol_type = genomic DNA
atgccaagaatagatcctttgatagttggtcgtgtggtaggagatgttttagatccattcacaaggtctgttgatcttagagtggtttacaataatagggaagtcaacaatgcatgtggcttgaaaccttctcaaattgttacgcaacctagggttcaaattggaggggatgatcttcgcaacttttacactctggttatggtggatcctgatgctccaagcccaagcaaccctaacctgagggagtatctacactggctggtcacagatatcccagcaactacagatacaagctttggaaatgaagttatatgctacgagaatccacaaccatcattgggaattcatcgctttgttttcgtgttgtttcgacaattgggtcgcgaaactgtgtatgcaccaggttggcgtcagaatttcagcacaagagactttgcagaagtttacaatcttggtttgcccgtttctgctgtttacttcaattgccatagggagagtggtactggtggccgccgcgcatattaa

SEQ ID NO: 10 - protein, 174 aa, Nicotiana tabacum
MPRIDPLIVGRVVGDVLDPFTRSVDLRVVYNNREVNNACGLKPSQIVTQPRVQIGGDDLRNFYTLVMVDPDAPSPSNPNLREYLHWLVTDIPATTDTSFGNEVICYENPQPSLGIHRFVFVLFRQLGRETVYAPGWRQNFSTRDFAEVYNLGLPVSAVYFNCHRESGTGGRRAY

SEQ ID NO: 11 - DNA, 534 bp, Solanum lycopersicum
  source 1..534: mol_type = genomic DNA
atgcctagagaacgtgatcctcttgttgttggtcgtgtggtaggggatgtattggaccctttcacaagaactattggcctaagagttatatatagagatagagaagttaataatggatgcgagcttaggccttcccaagttattaaccagccaagggttgaagttggaggagatgacctacgtacctttttcactttggttatggtggaccctgatgctccaagtccgagtgatccaaatctgagagaataccttcactggttggtcaccgatattccagctaccacaggttcaagttttgggcaagaaatagtgagctatgaaagtccaagaccatcaatgggaatacatcgatttgtatttgtattattcagacaattaggtcggcaaacagtgtatgctccaggatggcgtcagaatttcaacacaagagattttgcagaactttataatcttggtttacctgttgctgctgtctattttaattgtcaaagagagagtggcagtggtggacgtagaagatctgctgattga

SEQ ID NO: 12 - protein, 177 aa, Solanum lycopersicum
MPRERDPLVVGRVVGDVLDPFTRTIGLRVIYRDREVNNGCELRPSQVINQPRVEVGGDDLRTFFTLVMVDPDAPSPSDPNLREYLHWLVTDIPATTGSSFGQEIVSYESPRPSMGIHRFVFVLFRQLGRQTVYAPGWRQNFNTRDFAELYNLGLPVAAVYFNCQRESGSGGRRRSAD

SEQ ID NO: 13 - DNA, 528 bp, Arabidopsis thaliana
  source 1..528: mol_type = genomic DNA
atgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttag

SEQ ID NO: 14 - protein, 175 aa, Arabidopsis thaliana
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQLGRQTVYAPGWRQNFNTREFAEIYNLGLPVAAVFYNCQRESGCGGRRL

SEQ ID NO: 15 - DNA, 528 bp, Arabidopsis thaliana
  source 1..528: mol_type = genomic DNA
atgtctttaagtcgtagagatcctcttgtggtcggcagtgttgttggagatgttcttgatcctttcacgaggttggtctctcttaaggtcacttatggccatagagaggttactaatggcttggatctaaggccttctcaagttctgaacaaaccaatagtggagattggaggagacgacttcagaaatttctacaccttggttatggtggatccagatgtgccgagtccaagcaaccctcaccaacgagaatatctccactggttggtgactgatatacctgccaccactggaaatgcctttggcaatgaggtggtgtgctacgagagtccacgtcccccctcgggaattcatcgtattgtgttggtattgttccggcaactcggaagacaaacggtttatgcaccggggtggcgccaacagttcaacactcgtgagtttgctgagatctacaatcttggtcttcctgtggctgcctcttacttcaactgccagagggagaatggctgtgggggaagaagaacgtag

SEQ ID NO: 16 - protein, 175 aa, Arabidopsis thaliana
MSLSRRDPLVVGSVVGDVLDPFTRLVSLKVTYGHREVTNGLDLRPSQVLNKPIVEIGGDDFRNFYTLVMVDPDVPSPSNPHQREYLHWLVTDIPATTGNAFGNEVVCYESPRPPSGIHRIVLVLFRQLGRQTVYAPGWRQQFNTREFAEIYNLGLPVAASYFNCQRENGCGGRRT

SEQ ID NO: 17 - DNA, 540 bp, Oryza sativa
  source 1..540: mol_type = genomic DNA
atggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctag

SEQ ID NO: 18 - protein, 179 aa, Oryza sativa
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYP

SEQ ID NO: 19 - DNA, 525 bp, Populus trichocarpa
  source 1..525: mol_type = genomic DNA
atgtcaagggacagagatcctctgagcgttggccgtgttataggggacgtgctggaccccttcacaaagtctatctccctcagggtcacttacagctccagagaggtcaacaatggttgcgagctcaagccctctcaggttgccaaccagcctagggttgatattggcggggaagatctaaggaccttctacactctggttatggtggaccctgatgcacccagcccaagtgaccccagcctaagagaatatttgcattggttggtgactgatattccagcaacaactggggcaagctttggccatgaaactgtgtgctatgagagcccgaggccgacaatgggaattcatcggtttgttttcgtcttgtttcggcaactgggcaggcaaactgtgtatgcccctgggtggcgccagaacttcaacaccagagactttgctgaggtctacaatcttggatcgccagtggctgctgtttatttcaactgccagagggagagtggctctggtggtaggaggcgataa

SEQ ID NO: 20 - protein, 174 aa, Populus trichocarpa
MSRDRDPLSVGRVIGDVLDPFTKSISLRVTYSSREVNNGCELKPSQVANQPRVDIGGEDLRTFYTLVMVDPDAPSPSDPSLREYLHWLVTDIPATTGASFGHETVCYESPRPTMGIHRFVFVLFRQLGRQTVYAPGWRQNFNTRDFAEVYNLGSPVAAVYFNCQRESGSGGRRR

SEQ ID NO: 21 - DNA, 519 bp, Glycine max
  source 1..519: mol_type = genomic DNA
atggcacgggagaaccctcttgttattgggggtgtgattggggatgttctcaatccttttacaatctccgtttcttttactatttcaatcaataatagggcgattagcaatggcttggaactgaggccctctcaagttgttaatcgccctagagtcactgttggtggtgaagacctaaggaccttctacacactggttatggtggatgcagatgcacctagccctagcaaccctgtcttgagggaataccttcactggatggtgacagatattccagctaccacaaatgcaagctttgggagagaggttgtgttttatgagagcccgaacccttcagcagggattcatcgacttgtgttcatattattccagcaactgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 22 - protein, 172 aa, Glycine max
MARENPLVIGGVIGDVLNPFTISVSFTISINNRAISNGLELRPSQVVNRPRVTVGGEDLRTFYTLVMVDADAPSPSNPVLREYLHWMVTDIPATTNASFGREVVFYESPNPSAGIHRLVFILFQQLGRDTVITPEWRHNFNSRNFAEINNLAPVAAAYANCQRERGCGGRRY

SEQ ID NO: 23 - DNA, 525 bp, Gossypium hirsutum
  source 1..525: mol_type = genomic DNA
atgcctagagacagagatcctttggttgttggtagggttataggagatgtgttggacccttttacaaggtctatttcacttagggtcacttatgctactagggatgttagcaatggtgttgagcttaagccatctcaagttgttaaccaaccaagggttgatattggtggggatgatctgaggaccttctacaccttggttatggtggatcctgatgctccaagtccaagtgacccaaacctcagggaatacttgcactggttggttactgatattccagccacaactggtgcaagctttggtcaagaggtggtctgctatgagagcccacgaccaacggtcggtatccatcgttttgtgttcgtgctgttccggcaacttggaaggcaaacggtgtacgctccagggtggcgccaaaacttcaacactagggactttgctgagctttacaacctcgggttgccggtggctgctgtttactttaactgccagagggagagtggatccggtggccgtaggacatga

SEQ ID NO: 24 - protein, 174 aa, Gossypium hirsutum
MPRDRDPLVVGRVIGDVLDPFTRSISLRVTYATRDVSNGVELKPSQVVNQPRVDIGGDDLRTFYTLVMVDPDAPSPSDPNLREYLHWLVTDIPATTGASFGQEVVCYESPRPTVGIHRFVFVLFRQLGRQTVYAPGWRQNFNTRDFAELYNLGLPVAAVYFNCQRESGSGGRRT

SEQ ID NO: 25 - DNA, 528 bp, Brassica napus
  source 1..528: mol_type = genomic DNA
atgtctttaagtaatagagatcctcttgtggtagggagagttgtaggagacgttcttgaatgtttcacaagatcaatcgatctaagggttacttatggccaaagagaggtgacaaatgggttggatctaaggccttctcaagttctcaacaagccaagagttgagattggtggagaagacctaaggaacttctatactttggttatggtggatccagatgttccaagtcctagcaatcctcacctccgagaatatcttcactggttggtgactgatatcccagcgacaactggaacaaactttggcaatgagattgtgtcttacgagagtccaaggcccaactcgggtattcatcgtatcgtgctcgtattgttccgacagctcggtaggcaaacagtgtatgaaccaggatggcgccaacaattcaacactcgtgagtttgcttccctatacaatctcggccttcccgtggctgcggttttctacaattgtcagagggagagtggctgcggaggacgaagaagttag

SEQ ID NO: 26 - protein, 175 aa, Brassica napus
MSLSNRDPLVVGRVVGDVLECFTRSIDLRVTYGQREVTNGLDLRPSQVLNKPRVEIGGEDLRNFYTLVMVDPDVPSPSNPHLREYLHWLVTDIPATTGTNFGNEIVSYESPRPNSGIHRIVLVLFRQLGRQTVYEPGWRQQFNTREFASLYNLGLPVAAVFYNCQRESGCGGRRS

SEQ ID NO: 27 - DNA, 543 bp, Triticum aestivum
  source 1..543: mol_type = genomic DNA
atgtcggcagcggatccattggttgtggctcatgttttacaagatgtgcttgatccatttacatcaactgttccgctcaggatagcctacaacaataggctagttctggcaggtgctgagctaagaccatctgcaattgtaagcaagccacgagttgatatcggtggcagtgacatgagagtcctctataccctgatattggtggatccagacgccccaagcccaagtcacccatcactaagggagtacttgcactggatggtgtccgacatccctggaacaactagtggcagcttcggccaagagcttgtagtttatgaaagaccagaacccagatctggtattcaccggatggtatttgtgctgttccagcaactaggcaggggaacagtttttgcaccagatgtgcgacacaatttcagctgcagaaactttgcacggcagtaccacctcaacattgtggctgcctcatatttcaactgtcaaagggaaggtggatctggcggaagaaggtttaggccagaaagttctcaaggggagtag

SEQ ID NO: 28 - protein, 180 aa, Triticum aestivum
MSAADPLVVAHVLQDVLDPFTSTVPLRIAYNNRLVLAGAELRPSAIVSKPRVDIGGSDMRVLYTLILVDPDAPSPSHPSLREYLHWMVSDIPGTTSGSFGQELVVYERPEPRSGIHRMVFVLFQQLGRGTVFAPDVRHNFSCRNFARQYHLNIVAASYFNCQREGGSGGRRFRPESSQGE

SEQ ID NO: 29 - DNA, 531 bp, Pisum sativum
  source 1..531: mol_type = genomic DNA
atggcaggtagtagcaggaatcctctcgctgttggtcgtgtaattggtgatgtgatagacccctttgaaaattcggttcctctccgagtcacctatggtagtagagatgtgaataatggttgtgagcttaaaccctctcacgttggaaatcaacccagagtgaatgttggtggaaacgatctcaggaacatttatactctagttcttgtggacccagattcacctagcccaagcaaccctacttttagggagtaccttcattggttggtgactgatattccagctactactgaggtcagtttcggtaacgaaattgtgagctatgaaaggccacgacccacctcagggatccatcgtttcgtgtttatactattccgtcagcagtgtagacaaagggtttacgctccaggatggcgacagaatttcaatacaagagaatttgctgaactttacaatcttggatcaccagttgctgctgttttcttcaactgtcaaagggagagtggctctggtggaagaacatttagataa

SEQ ID NO: 30 - protein, 176 aa, Pisum sativum
MAGSSRNPLAVGRVIGDVIDPFENSVPLRVTYGSRDVNNGCELKPSHVGNQPRVNVGGNDLRNIYTLVLVDPDSPSPSNPTFREYLHWLVTDIPATTEVSFGNEIVSYERPRPTSGIHRFVFILFRQQCRQRVYAPGWRQNFNTREFAELYNLGSPVAAVFFNCQRESGSGGRTFR

SEQ ID NO: 31 - DNA, 901 bp, Arabidopsis thaliana
  source 1..901: mol_type = genomic DNA
aaaccgaccggagccaaccaaaccggttaacatcctaaaaccaatcatattttattaagttttgtgttgatgctaaaccaaaaatcattggcatgcatatttctaaatttagtaataaacaaaaacacttagaaatcacacgttcactatactaaaaaacgttgacaaaaacacaacaactatactaataattaaagaagagaaaactgaaccaaactttttgtaaactcctgaatttaaattagtaattgaagtaagaagatgaagaagaacatgttaagcaaacaaaaaaattacactaaaatcatataaaaatacataattacaaaagtacccataagatggatttattgatatgggtcatctgtgaaacaagccacagagagacaaagactcgtaagtattgggcaacgaaagcgacctcctttattcaccactgccattaacatgttcttcttctccttcttcttctacattttatgaccgttttacccttcaagagagagaaacaaaatcactccctctcactcactctatctctctcttctgcaaagcttcagaactctggcagagagataaaagatgatggggtttttaactttatcctccccaaataattcttcttcccttcatctctctctcttacacaacaggtccctacatttgtacaatctcctctctttaaagactctctctctttctctctccatctctatcttactctgtatttctgtcgtctgagcactcaatgaaaccactgtaaatttccgccagaatttgatgtgatggaacgataaaaatcattttttctcggttaaagtaaaaaaacaaaaacaaatttctgtagaaatcataataaaagaaagaaaaaaaatctaatgtcggtacataatacggttct

SEQ ID NO: 32 - DNA, 507 bp, Arabidopsis thaliana
  source 1..507: mol_type = genomic DNA
aaaccgaccggagccaaccaaaccggttaacatcctaaaaccaatcatattttattaagttttgtgttgatgctaaaccaaaaatcattggcatgcatatttctaaatttagtaataaacaaaaacacttagaaatcacacgttcactatactaaaaaacgttgacaaaaacacaacaactatactaataattaaagaagagaaaactgaaccaaactttttgtaaactcctgaatttaaattagtaattgcacaacaggtccctacatttgtacaatctcctctctttaaagactctctctctttctctctccatctctatcttactctgtatttctgtcgtctgagcactcaatgaaaccactgtaaatttccgccagaatttgatgtgatggaacgataaaaatcattttttctcggttaaagtaaaaaaacaaaaacaaatttctgtagaaatcataataaaagaaagaaaaaaaatctaatgtcggtacataatacggttct

SEQ ID NO: 33 - DNA, 2000 bp, Glycine max
  source 1..2000: mol_type = genomic DNA
catgagcaagtaagtaaacattttatctctgttacactccaaacacatacactaacttaagcagagtcctttttcggctattcactccatcaccaaagaatgagctcatccataaaaacatatacatgtcaacatgtcgaaagatgtagaacctggtaacaataataaagaatgataatttttttatttttaaaacgaagcactaaaatagatttaattatatttatcatataaaattaccaattaatcatatatctagtgaaatgatcacttcaagtataaaaaaagattttatacttttcgtctttctgtatttagaaatattagcaaagtcttggataaaaaagtggtcgtagcctgtccctgtagtttccttgttagtcttgatgaacaagaagttctcagttctccccaccctattctgattcggtttacatggaagtagtaagtaaccatacaccattatagaaataatacgataaccacacgccatgtcttacctcatgcgtttactgaagttgttttcttcttttatttttctttgatggagttatggtattaatattcaatattagattggaacttgcagatcaacttcaagaggcactctttgataaggataccccaggcattgctttttgacataacgccaaaaccctcctaaaaaacccttcattttctatctcttagttccattttatgcaatgaaataaaacttctcaaatagtgtcaaagcccgaaaattgcctaccatatatttatcacgattcatgaagggtatcttaattccttttttttttttttcctgaaatgttttttttgaaggaattttcttgaaatgttttaatgcctttttttttactcaagaaatgtcttaatgcttgtttacttacataaagtaatatcgtttgtctttttttacgcaatattatattctagtactctgtctctccaatcttattatttttaaaatttttcttccttcctatcctattatgcacaaaaaggtgtaattttaacatttttctactagtaaaaaacctacaaactttttctattgttaaaattaaatataaaagtaattatttttattttatataaaaatacaaagattttatggaaaaatatgtaagatataaaaatatgattaattattttactttcatcttaacttagcaaatactttctgatcagtgccttatctcgcacaatccacaaacattatctcgcacaagccacaaacacgcgcttcatgatccaaaattgtacgagcgacgctgtccatgtctcctagaacgcgcgtagtaagaaataagtgtccctttgatttcatgttgcatagttaatttttagtttaagattaattttagatagttttccatatttttaattttatattaaagataaagtcaaaattaatgtttagaattaattgagtttaagtcattttaggtaccttttggataaagaaatttaaattaaattttaattcaaaattaatataaaccaaaattattaaaacataaatcatattactttaaaattaatttttcgaaaaagcacattcaaagctccactaaaaattgtctttgattaatagccgtggatgagatttgatttattagtgagaaaagacaaagaggtttaagcgcacgcgaagagaggcgcgtaagtaaataggagaaactttagctgtcaaatatgctgggaaacggcgagtacgaatgacggcggctaccacccttatattacagtgacagtctcactctcacctatctagcctaacgtcgcttcaccgccgtttcccattcttattctctctcttcataacactcttcctatttacagttcacgccaaatgcctgcactctttctctactattaccaagcattggccacaccaacaccaacgaataacctttgttattgtaactaataaccactgcatttttcccatactcgttgatctcttccactaagtgctgtggttggtgaccgca

SEQ ID NO: 34 - DNA, 2479 bp, Glycine max
  source 1..2479: mol_type = genomic DNA
ttggattgtgtagaagaaaaaaattgaaaaaaaatatgacatccatgttaatttttgaaattttcttataataattttccgtcttcaaccaaaccagactcctattaagtatagtaaatactattatatttttgtggacttcttaataaatagtagtaatattttgaaaagcttttttatttttacgaaagtcataataaattataaacaaacttataaaaaatattaaatcttatagtactacttttatataataataataataataataataataataataataataataaaacgtgttcaaaattattaatattcctcttgaagttccgtttcatattctgtaaaaaaaagttgcgtttgatattaaaatataacagtactaaaaaaacaacataaaaaagaaaatcatgtttgatgaaagaaaatacaaatatattttcataaagagaacttcacaattactcgcaatgctgtgtgaaatagggatataacctttatccaagacacgttcccatcattgaagtataattaaatcttttacggttaattataatgaaatcattttggatttgcttttgcctattatcacttttcacacgatgatacttaattattcatagacctttttgtcgagtaagaggggaaatgctaaacttttctgcttagattttttggcatagttaatggattttagcctttttctttcttattaatttttttctttcataagcatagtcccggtaaaattctcactttcagttgatactttacctcctccgaaaagtttcccatattagagactcaatggcgtataaaatcatcttaaacatttacttataatgaatggaaataaaatctaaaaagttagctactaattctttcacggccattacgaagactttgcttaaaaatggaaaaaaagcaaaatataaaagagtgtacattgtctatttttataattgacttggctctgtatgtattatgtaattaatttttaatcttatattttgattatttatagagatataaaatgaatttgatagttaaagaaagaaaagaagagatgaaaaattgtgtgttcgatcctctaataaaactaacattttaataaattaatatttatcatttttttaattatttgagttttggaagtgtaatgagtcgagtaattttatttgatggttgtttggttcacttatctatgttgatcaagtaatcgatcaatttatctccatgaataatgatgatttttaagaatatttaacatttgaccatcaattccttaaatcatgtaattatttttgtcaaccatgcaacctctataaatataggtcctacatatgtttgacattcatcatagtgtgtaatgtattttttttattaaaaaaaacagagatgatgaactcgtgataaagaatcacctaacacattactgatactctctataaatatcacatgacaaccttaaacaaatacgcacaattcatatatcaatatccattactttgtcatattctaatttgagtgtaaaagtctttattattacagtcttttaagttggtttagagcaatttgagttaatatctttatacaaaaataacttaattttttaatattatttttaagacatatttctcataaaaaatcacataatttagtttataatttttaatttaatattatcttcatttttatttataaaatttaattacctaattcatcaatattaaaaaaataaattaatttaattaatatcaataaatttatcctaaacttataaacattatcattaatgctcttctctcttaaatgtttatggatataacttcttttatttaattaaaatgtttattttaaattaaattaatgtaagaaataatacaaattgaatattgtataaaggaacaaacataattttgttttgtattagaccataagtaatactccatattagattatatatataacttttattttaaaattatagagtatactttttttagaggaaattatagagcaaactacattcatatgatttctcttttataaatattgaaaacaaaatagggatatgcaacagcaaacgagggaggtttgaggagagagggagagagagagaatgtaggcgcgtgtggcacagttatgagttaagacttaggagaagtacacattggcataggcattgttattggattatgtgtagagtccgatagactagaatgacggctactagttactactctctctcttcataaacacaccatttatgtttttcccttcccttcacgccaaacgcctgcactctacactctactctctcgtgctctgtgactactgtcactctctcataaaccaaacatgcccttaatccattttccatagtagttagtgttgttactcatctcttccatcttcaatctctcttctttccttattgttgctcaccaaggtggggttttttgtacgtgtggtggca

SEQ ID NO: 35 - DNA, 2000 bp, Glycine max
  source 1..2000: mol_type = genomic DNA
cattaaataattctaaaaaagatataaatttttgtatacaaatctatatttaagaaacttttaatctagatgtcgattttaaaaaatattatttaattaaaaaatattagatggtgtaataattaatcaaaattatatcaagataatctgattcctttctatacacacataatattatttcatccttagtccctaatattttcaattctcattttgttaccagacttgtaccgaacaaaaacaaaatatctaatcatagttttcattcaacaaaaatgatttttaactcaatttgaaacacttttcattcatttttaaaactaagaaaaaatttgtgatttatttttataattttgaaaaacattctaccatcattattattgtttctactaccatcattattaatgttactactatcaccttcctagttataaccgtaagcattatatatttttattattattgttattatgttattttgttaatatatttatttttgttctaaaaaattattattttttcatatctttcactatttttgttattattttagcaagtttgattattttttattttaaatatttttatgtgtcactttttatatcacattatttaacagtgtgaatcgataaaaaatataataattatctttaatttgtaagaattttttcaaaattaaaactgattttagttcttgaaaaatgtaaaactaaaaatgaaaaccacctaacggggccttagtaattaggacatggtctccctggttacccacgggattttttcacatcaaagaagacctggtattttcattttcatgagatttttgcatatcgaacaaggcattaagacaggggttgtcattgtcgtgatagtataatttacatggtcgaagtgatagaaactttaaccatcatttaccttgtaccttactataacacaaaatactacgatttccaaacactagatcgcgcgcttatgttttcagacacattattcttcttcattcataaataaatttgcagctagtatatgataattgtaccaatttatgtaagttttttacaaaggacattcttatctcaataaaaaactaaatgtttaaaatattctctagcacattttttaaatacattttgtctaattaattaaaattaaaagaggatataaaaaatatgctgctaacatcttgaacatttccgcaatcaataatttctcaatctatctgaatatttttgcaactgtatacaaaaatctcagaacagaaaattattgattaaactggaagaatttaataacatttgattcacgtttgtttagtgattaaaaaatcataacattacactatctaacaaatgcagcatccataactaccaaacattaaacaagagaaacagacaaagtccaataatcacagagacacgcagtgacaaagaaaagaaagagggaacggtaaagagaaaggtgtctctgtcatctcaaatagattgccataactccctccttctctctcacaagctcttgcagagtgaaagcgaccactttccgatctcaattaaaagtatggcataatttgcaatggcggaactgaacgaataataataagagataccatagttaagagagagaaacacaaacatggaaaaagctgggcctcactccctgggtacacatagatagagactatggtgcagtgttgcaggttgtagcagaagctctgccaaatagtgttaactttattcgagaaaattattattattattattattattattattctctctctctagtctattatcagtggtaattcagtaatgttgttgcattatagagagagcgtggtctatgtgccagggtgatgtgatgtcatttcactaccttcaaagccagaaaaatgcaacagaaaaagctttcatcccatcacatcatttgaaccatgaatcatgaactagttttctaaactaaaactataacaacaccttcggttgttgttgttgttggct

SEQ ID NO: 36 - DNA, 2000 bp, Glycine max
  source 1..2000: mol_type = genomic DNA
aacatttaagatcttaaagatgccaagagcttcatatgaaaatgtacaaaagagattttaaaggcaatatcaatgctgtgacgccatattaaaataaaagggatggtttctcctgtatattgagcaatttgtattacttatatacacaaaatctaaattgattcttaacaaatatgtaaagaaattaataatatgatcaagttacctgaagaagctaaaataaaatagaaaattaagtaaaagaaatgaggagtagaatataagataacatcaaaaaattatttcagcatattttaagaacatcaaatttacctttcatcaaaattaatcttaaaagactaaaacatttaattaagtttataaatactcacacaaaatattaatttattttgtaattattattttttatatttttatttactattgcctcaaaatttgcactaaacaagagaccctagagatttcgttagaacaataatagacacggtattaaataattaaattaatacgaggatgcataactaccaaacaaatgcgataaataaatgagacgacgagagagcacaacgcgggaatgagataattaagaaaaaaaatctaataaattaggaaaaaaaagacataatatcataagcttgaatccaatgtacaaagagaggttggcaataaagagaaagagaaaagacgtccctgtcacctcaaatggattgcattactcattgaaaaggacattattacttccgactttttatattaacttactaattataaaatatataaaaaaatacttcaaagatgcatatattttattttattacataattacataacagaataatataaaataatgtaactacacattaaaacattaaaatagtgattggagtagtggtataagaggacgttgaattcacgcggaagagaaggatatatttcatgtttaatttgttgtcatgcctagttcaatgtaatctaataagtaaaaataaaatacaaacaaaataaagattttggtttcttaacaaaagtacttttactttaaatatatatttttatctggtttttaaacatgcacatatttaacataaaagttcatattaaactttttcctacatacttggatcaaatagtcacgtattgcaggtaaaaaataatagtgtagcttatagaaatcgtagaaataagtctataaaccagaagaaaaaaaacattaaaataatagtatagaaatctatatcagtgtccccagttcttacattcatgacccatttccccataaactctttgcagataatgcaatggcaaaaccacacagaaagtgacccctgggaatcaaaagttaaaaccaatggcacagcatagcacagtgtacagtgtttatttactatatagcaaaacactcactggcataacactttagggagagagagagtgaaaacaagtgtaaaaagagagaaagttaggagggggatagagagtgtgtgtgtgtgcagagtttgcaggcttgtagcagaaatggtggcagatggttttaactttatgtgtgaaataattttcttctatctcttttctctttagtgttttctctctctctctctctcttctttttcttcctgcatcttcttgtgtttagggagtgtgatgttttgtggcagaagaacgatgtgattggacacagccaaagctgtggacttgttctgttactactttgtaattgtaatcacataaaaggctagagggtatgaagagtgcacagaaaaatactagtactagtttcaaacaaaactcaccttactactacccttccatctcaagccatagttgagttgagtggtgcacagtgtcactatacataccactaacacccttttttggttcttgttctgtggctccttgtgctttgagcaagagctttttgagaaagagcttggtggtggtggttgttgttgagtggtttcatggttaggctgttgttaagttgaagttcatcagttgcagct

SEQ ID NO: 37 - DNA, 1399 bp, Solanum tuberosum
  source 1..1399: mol_type = genomic DNA
aacgaaaaatttagaaactattagtgatccaaatgttcgtgattacctgcaacgagaacaacaacgaatacttgaaaaaagaaatcgacaatcacaaccacaaccataatcgcaacaattctcagaatcatatcctaattttttttcgaatagtgctaaatttgaaaacgacctaccgaatttctaaattattgttgtgatcaattaattattatgttatgtattgtattttatcttgtatttaaattattatgttatgtattgtattgttatcttgtatttaaattatcatatcatgtattgtatttttaaattaattttttttgcatattctttataatgaaaattaataataaaacaattttattattcacgaaaattagaaaaaaagttaaaatactattaatttgaaattaaaatagtatatattaaataatatttttaaaaatattatattatatttaaaaagaattatgaatattagatatttaattaatggaattatatgtaaaataatatgttaattagaaagtaatagaaaaataataaaataatgaaaaagtagaaatagagagtgtgaatagtagaatttggagaactattcaactctctaaatttgaagaatatagggtgatttggaggtgggttggagtgtccattctctattttactctcaaaatatagagaatggagagaaaaatagaggtggattggagatggtcttagtgacatttttgattccgccaatgctcagttggcgtagtcgctgtcaaacttgagaaaggattacccctttaggcttgcacagacagtgacttatgatgaaatgaagccagagaaggcactctgttataacacttaaatgaaaatacatgtgtatggactagcaataaaaggggcactagtaattttagtaattgaaaagcaagtgtatagagagagataatgagagagaaagagtaagtacactactactgctactatcccatatagctgtaatgttgcaggtctgatttttgcagttgcagacccccttcttggcacaagctcttttaacttttatcttctcaaataattctctctctctctctctctcttttttctctttttacattgtgaggaaactgaatacccattgtatgtattagtgtgaggcctatctgccacaaggatgtgatggaacactatgcttcctctgctaaaaccccacaaccccaaaactctttttcacttcacatttaatcacaattcctcagtgaaattattctgttgctctctctaatttcaatttcaatgtcggtaagtccaagaactggtttttcaattcaaaggagctgagttagtgcaaacacttgaggttttgagttttgacagagacttgagtctcagagaaactacc

SEQ ID NO: 38 - DNA, 2000 bp, Solanum tuberosum
  source 1..2000: mol_type = genomic DNA
accttatataagttacaatttagttatgtatataagttaaaattaaattaaaagacatttcgaaataatatgattataccatttcgaaattaattagagagagaaataagatctcgcaaaattaagtgtcttcttgaaattaagaaccatttttaggagataattatgtattttttcatttttaatttgacacgtatgcatatccactattttgttttattccaaagtgacccctacttcttttggtaatttctttgagtattttaaactctagtccccctttctcaagcaaaaaggctcactcgcgcacgcgcgaagagacattgtgacgcgctggatggaaaatccagaagcgtaactgtcaaaaaatagaacaactttgggaaacggggtgacggccgctgccaccacttttttcatttccaaacactcattaactaacgtcgtttcaccgccgtttactgcttaatgagtatgaattacactctaatagtctatttttacttatttttaatgtgtttatcaaattatatttttaaatataatactttaaaaatattatcatcaataataagagtaaattaaaaaataaatgacaaattgtttcttaaattgttaaattaaacaattaaaactgaatatttacaaaatacctcttaacttgctaaattaaacaattgaaactatatttatattaataaattgaactgacaaaaataaataaaggaactatatattttctcaattatatctttttactaaaatattatttttctaatactagttaaacttttaaaaaacatctaataaagaaaaagaatttgttcaattatactttagaagcttttattattattattattattagtagtagtagtagtagtaataaattagattaaattaaagagagaagtattcaaaactcccaaaactattgtattagttttatttcagaactattgacaatcttaatttttttttttttaatttgactaggtgaacttaaatatacttcattttttgcaaaacaagtgaagtacactcttaaattttcatcaagtttagaaatgttttcaacaatttactagactctttattaagaacttcatgttctttcaagagtttatgagcacttgctatgtcatgttacagatcaagaatatctacagagtgtatctaaatttagtactagtaaagtagaaaatgtattacttatctctcaaacaataggtattcattatactattttgagatgtccaacaattttttttcactttatgaaatcaatgaataatttaacacttagttcctaattcccagtaagcattaattatagttatttacttattatatttttcaacacattatattgaaaaagtgatatagtaaatctatctttttattttattatttcttaaaatttgtacaaacttaataatagacaaatattgttgaataggaataataatttacattaaatccaatatatttttcaatagttgtcactaaatgaaaatacttcatctgtttcaatttatgtgatagttttcatttttcaaaagtcagacaattatatatttataaattaagtaaaaaatattataagtcacactaattaacaattcgaaatattcggtacggaggaactaacacttatgtttttagaccatattagtcttttctctctatttattatataatattgagaggagagtgcaaccaccatggcaactttctctgtcttcataaaacgcagctgacattaaaaacacagacacacacttcgcatttcatatccctctcactacacgccaaatgcctgctcttcctatttctcttcttcttctttttcttcttctctctcattcacataacacacattcttgtactaactctgcatcataaactctaccccactttcttcttcttctccggtcatattgctctgaaactccacttattgctctctcccggcatttatttttagtttctcagaaata

SEQ ID NO: 39 - DNA, 471 bp, Arabidopsis thaliana
  source 1..471: mol_type = genomic DNA
taataagagacgaaaaaaaaataactaactgatcattaccatccataaataaatagttgctgccataaaccaaacacattgtgcttatcaaaaagaagaaatttgtacttaatgaaacattcattattagcaaagtgtaaaaccaaagaaaaacaaactttatttctcattttattagtaaaagtgaagaagagtaaagaaaaagagagactgagatgaggctgagagcctgagtctgcgggtggagagagagagaaagaaagcctctttacacgtgatttttaaaagagaccaaaaccccaaaagcaaacctcttttgcatgcgtccttaaaagacataaatttctctcaaaattttctacatcacaaaatcaatctttttctcttcttcttcgtcttcatcatcatcatcatcatcttcctctttctcttcctactgagatattttctccacattgagaggaaagctat

SEQ ID NO: 40 - DNA, 1231 bp, Arabidopsis thaliana
  source 1..1231: mol_type = genomic DNA
aggataaatttcatctattaagatatcagtcaattataatgtgttacgtgattcgataaaaaaaaaagaccaaaaaaaaaaagaagataactattggtaagcgtaagaaatgtgtttacattttggcattttgccaaaacacataaagatggttagtgatgagacgagacgagtcatgcgctacttttaaaacaaaatgaaaaacatcattaagctaacaaaccaaacacacttgttttgataacatgttctagggaactagttatgccaaatctaatccgcataagaagactaagtcacaacataattcagtaatttggttgagattaaatcctataaatatgattttaaggtataagagagaagagactcttttgatcaacacaatcaaacatctacaaagaaaattatctcacatagctacttcttaatctaattttttcattaatccattttattttaaatgtgaagaatcgcatctagatgtgacctctcatgataaaaaattaaaccattgtaaaaaaaatgttgtgtaaaactaaatataataaattattaaaaaaatacaaattcaatccactaggttaaaaactcctatgtagaacatttttttatattaaaatgtaaatacatgaatcttatttttcgaaaaactaaagacatcttttttttatatattaattaccaaaacaaaataagacgacaaaaatattctttgatatagtaaaagaaaactagaaaactagaaaacaataaattaccaaaacaatctagaaaacaataaatcctactttgcattactttattataaaatcccgaaatgaatctataaatgtagaaaatattatacaaaagttgtaagagattttaatatacataattacatatatatacaagtaaatacaccgtatatacatacgaaggagtaaacagtattatttggtatatagttacgtctctatatacgaagggttcaaacttcaaagtaataatttaatcaacaatgtgtacatattgataagtagtagtatatatgtaaaggtctcacgtctctataataaagtatgactcgtcacgtgacctcctcttcttcgcagagacagagataggatgagacagaaagaaaccaacaaaaccaaaccccaaaacccaagaaaaagagaaaaacactctcttctcttctctctctctctttctatttaagagacttcactgtctctctcagtctttt

SEQ ID NO: 41 - DNA, 1548 bp, Arabidopsis thaliana
  source 1..1548: mol_type = genomic DNA
ttcacgtgtttatttatttatttgggttattaaacataaatcatgtaaatctgaatcctgtggagatctctccctagttgatgaatagatatgatgaatttaattctttcatgaaataaaaatatatgaaacatatgtagcagaaaaagaagcatatctatgaaacaacaaacattcaaaaaaaaaaggaaaacggaaaattattaatatgaaaactacggctttgacttgtagctgactacatttacgacatatatatataaatggaccccactgagtgtctgcaaggtctttacacaacagtatcttcttctgtttctttgactctttgtgatccctaagcctacccataatacgtgtctacattttattggattgtttcgtgactctgtaatcttttttataagaaaacaagtaatagtgaaattgaagtaaatagctcagcacagaaacttcgacaaaaataactcacagattagaaaagaaaatatatgcataaatagccatggttcatttatgaacaatttattcgtttttttagtttataatttcattaaaacatgtttgtcacatcacatttcatgtccttcggctcctactacaacaacaagtcactgtcatctccattacttccacttctgctcctttctttattaacttgttcaaaaacaattctaagataaataacaataaatgttggtctctctttattatttcccggctaaagaaggaggatgtctcgtattatccgccatcaatgctcttttgtttcctgtttcttgcaatttgaatccctgagaatcctagcccacttatttactactttgccttagctgttttcgacatcaaaattttggtcatatgactcatatcaatcttcaaatttgataaaatatgttcccaattcacaaaaacaaaaaagttttcgaaagctcaaaaacctttaccatttcaatagtagataggattcttttagatttgcatttcacgaaaagagaagaaaaaaaatcgaaaaatatttgcaatcatgattttttgtttctgaaggagacctgtagttgctgtcatgaacattaaatacaaatctaataaatgttgtacgaattttgcgtgtaataaatggtcagggccggctcgaagctcgctgatcgtccttttttcgtgtctctatagcaacacacaatcgtatttatttcaaactttttttactttgtttcccatccatcaaatataagtataaaaatgtaaagaatcatcatatatagatcgtaaattcattgcttcctttggctttttatttcatctagacgacgttaaaaccagaccagaccaaatacatttatcatttttcccttttttctaaaattctctctttgattcctatcttcttctctttattttcactttgtgctttctctgtctctcctattatgagtctaaaagtctactagctgttcaatagttttgtctttctgtgtttcttcttcttcaaaaccgaaagaaattcaaaaagagtctttcgctgcttgttagtggggtgaggaacaa

SEQ ID NO: 42 - DNA, 440 bp, Arabidopsis thaliana
  source 1..440: mol_type = genomic DNA
aaacccgaacccgaaccaaacccgaaccaaaatcttaaattacccgaatgggtcttaaatttctaaatccgaaaaacccgaacccaaaatacccaacccgaatctgacccgaatatccgaacgcctaatttttctatgttaatgaaatcaattatatgacatgtttataaagagaaataaattacggtgagaattaagcccatttacgttacggaaataaaacacccatttaaaaaagcccaacacgtgaagcccatttccgagtgcgtcccacatttactccaacggtcgaatcgactcaaacattcaaaatacaaaaacgctatctttatcgtcttcctctgtctctctctcacaacacataacgttcaaatcctctctctctctatctcgtctcttatctctagatctaaaaatctcttctttcctcaatctctgtt

SEQ ID NO: 43 - DNA, 317 bp, Arabidopsis thaliana
  source 1..317: mol_type = genomic DNA
acagacatttacttatacggttattgaggttgaactggaccggagtagcaataaattatcggttcagtttgggagatcaaaccgtttaaaagaaaataatttgaaatggccacgcagaatacgagggtctgaggattgtacctcctttctctgcaaaaacttaaacgttgatttgactcaagcgtcaaggtaaggtactctctcttcatacaacattttagctttactttttctctttactcttctctctctctttctctttctctttctctttcactcgttctctctcactcactctcttcacacacagatccaag

SEQ ID NO: 44 - DNA, 3912 bp, Arabidopsis thaliana
  source 1..3912: mol_type = genomic DNA
acaccaataaaaatacacagcaataaaatcgctacgtatatatatatataatatgtattatctattacaagatagtaatagagtatagcaagttgtatcatctaacaaactatgcgaataaaatttgaacattgtgacatgtagatgtagtgtaatttagctaagtgcttatcatcagtaacatagaccgacttaactttttacgaaaaaaaaaaagtaacatagaccgaaaaaatgcatatcgtaaatttaatggaaaacacaatttacgataagtaaaaaacaaaaagaaattacgataagtcgagaaaaatgcaacaaattgagataaagtattgataaaaccatgaaagtgtcggcgtatgtaaatgcggtgattaatgtgatcattagagcgtgtgtgttaaacgcggcggttttagtggagattgatcagctgataacactcttaccgggacgaatctaattccatattcatggcttgttaaaacctaagacatacgcaatctctaatttgctagtatagttagttctatattatttttcgactaataatgtaaacatatgattattaagtcgcaaaaagagtgcttaacaaccaaaaagtggattaattaacttggtgggaaaagttacaaaacctttaatgattactctttgtaccaagaatagtggcgaagcactataagagcagagaaaagaagctcaataatgtactaaaagttgtagatttttacagcttaaatacaccaaaattaatagaaaagttggtaattttttaattcatggctactgatttagattttagaaaacaatagtagtatcattgtcacatcttaaacacacaataggtatgttttaaatcaaaggccgtagttaatttgtcaaaaatgtatgcatttggtatttggatgtctccgaaaggatggatatatggacttgttagataatttcatacctcagtatcaatagtcatggagcccaaattgctcaaaaacatatttttaattccaagactttgatgaagacgtaataatgagtccaatgggccatcagatacaatgttcggaatttaacgggtttgttagttataagtattgggcttgacctatctggttcaatgatatgtaggaacaacccaatttgcaaagctttattaaaagactctttagttgtcgtcaaggtttaacttgtagtagttggtaagaaattctacgtgaaataggcaacattacaaaaacaaaaatcaattcgaaatcatacaaaacgaaaccaagtagtaaccaactacactattatgacattaatgattagacattcccaaatcatacaagttcctgtcatgaaggaaacaatggtccgtatttgcaaacgattacaaaaattcaaaccaaaaatgaaaaaacgagttaaattatttggtttataaaaatagtaatgtcaacagaagactagattgggaaacctgaagcgaacagagcttttaaaaacgagtttgaacggctgggatcatttggtacaatacccaccgtaagtttgtttaccctagggatgcaagccaaaggcccaaatcagttactacttactgctacaaccatcgtctcagctttttgtctcagctttttactaatgaagcatacaatttcttgggcatgtcacatctcgacacgtgtccactattctcttctcttattggctactcgttcgtaggcttctgttaatagatgatctctctataactctaacagtcttttctttctctttatttcgttttggtattttaagtttcaaattgaaaataataggaggaaaagtctagttttaaatattgtttttttacaagtgaacgtgaaccaatttacctctttttttttatatatcctatcggctaatctggttagtatcggtagaaatgcaccgaggtgctacagagattaatgctagggatagtcagaccgcttgtatttctgactatcaagtaaatctacgcccaactcacatatttcccaaacaaatgtgattttttttttttttttttttttttttttttttttgtaacaaatgtgattttgttttcaaggaaaatagaacttacgtttgggaatttcacccttcactaaagcttccttctgccattagaccacaaaggcttgggcaatttaccatttttgtaaaagtagaaaacaaaatgcctaaaatgttcatacttcattacatcaacaaggttatgcccacgatatagaggcatgtaacatttatatatatagtggaagaagcctacgagctttattaataagtataaactctgattattaggtaaataaattacttaaaacgattactcaactgacaaaaccgtagttgaataataaggttactatgaataccgattgaatattgcaaagccggaattgaaaaatatataacagatcaaatgttcaagtgtggtcataattctcacataggtcatatagctgaacccatgcatctatttactagtctatagaaagtactagagacgcatacagctgaacctactctattcttttattaattttggttctcgtggatacaaaattcctccaacatttattagaacgaataaaaccaatatgatgatgattagttattggtaaacatataaacgttgagtaaacttcaaaatagattgaagtactattaagacttgcattttttccccttgggttatattcttgaatcgtttcgaagtattttaactttcaagaatagaaggttcctcaactataaacaattacattaatcaaaaccatttctatgtaaacaacataatttttgtatattttagtcttccccaaaagtttgaccgatagggcggtttagaccgtatagtacgactgtacaacaaaaaggactctggagacctaaagatccaaaactatgcaaaataaagatacggtcggaccaatttaatctaacaaaaccaaatccttatactaaactatttaccgatacatttccatataacacagtacacacaattaaatcaaacattattggaagaacaagatagaatattggcttaatctcgaacgattagagttatcctagagcctcggagcttttgtcacatataatataaactatggtatatataaacatgactctcatttgtatttatcgcaaggtacaattccaccaatttttttcgtcccactcatacagctttaattgtgaaatcaatccataaaaaaccaacatgtgacatggtctctataactataactataagatagtaaaaaattcacatcaacataaaagaaaaccaatcatattggctaaaaaaaactaacggtcgaaaaacgtataaccacaaaaccaaaccggtccaaccggtgtccccaatcactatcaaagcattaactaactttcacaaggaaaagcatagttcagtttctctacatcgcttcccatcctcttaaccctgtttactcgaatcatccaccgttggatcaaacacgcgctacaaatctagcgcgtgaccgaggtttttacacagtggaatattaccatgcattggaaagcggcgtctacaacaaacggcgggtcatgtcaccgtcaaaatcaacctttcttaattcctaacgccgttacttatctccgtttactaaaaatgttaatgcgtgtgagagtgaagatcatatactaattagaagtggctaatgttttaacgtgacattattatcatagttaatggttcgatcagagttttaagtagtaaatgatataagtgtgtgtatataattgcatacatatatactctcacactctgacagatttgtcgtggtcttagtattctctttcatggctagttatatagggctctagtacattatctctctctccccatttctctgtctctctcttctttaa

SEQ ID NO: 45 - DNA, 2000 bp, Arabidopsis thaliana
  source 1..2000: mol_type = genomic DNA
atctttatggtcaccgagtctactgatataattttactgtcgcagtttgtttccactacttaagtttctataatttcacagtttgaaagaaaaattactggttattcagctaaattacaaagattagtttaattagtttagccagtataatgttttagtaaagtattaaacggcatttttcgttgggagaattatgttatggtataatctactaatacatacttttacacatatatcaaaaagtttgaccatagtaggtagtacaacatagaagaatcaagatcggaaccagcaaggaagaagatacggtcggtccatattaagctaagaggaccaacgtaactcgatatatatttttctgttctctacgttaccaccatataaattttaatattgaaaaaatcatcttttggcattgtgtttgatgtcggattcggaatatggaaagaggagagatatgagattttggcacaaaggaagctgccaaagcattagggcaaccgagtagtaacgagatcaaacatcgtttcaatcggacggtcggggtttgaccaatatttctcggatatcttttggaccctacgttctgacttgaacttgatcagtcacttcagtaccttagttttcattttcaatgtgatcatgagtttttttttacatgttagcttcaaaacaaatactaatattcattaactatggatcggcatagttttcatgtaatcagctgagcgtttatcatattgattgaagctaacatgtaaaattctcatgatcacattgacttttgcctacaaattttaaaagagtatacaaataattgcttaatgaagatagcttccatagagaaagagtaacagctttatacggaggcatagctttagacacgatctctgctcttgtgttttttgtttaacactgaatccacagtgaaattactgctcattttttttcattttttattacatttttttttttacttttttatttattacaatctacagttctaccaacttattcaacctagtggtaccatatcgaccccaaaattaatcaatctaattacaaggtagaaatagaaagattttatcaaaggagacaactctgatcgataatatgttgcaataaaaccatgaaaaactgtaaaaaatattgaaagctgaagaaaaattttcaaatcgataaaaggatagtactaaaccaatccggtttgtggcatctttttcacccaatcactatcaaagcattaactaaaaatcacaaggaagagcataatttgattctctacatcgcagtccacgataggatttctctatccaccgttggatcaaatttaataatgatgcacgcgcgcgtcaacgggattttacccagcaaaggaatgtcttttcaccggactcttaaaagacgttcttcttttttcacctttgcattggaaaacggcgtttcttcttagaaccgtcgccgtcaaatcatacggcctaataaatctccgtttaacgccgttactttaccgttaagtactaaaaaaacaaaaaaaaatcatttcgatcactgtctcattaagatgatcggagatgttttagcagggtttaacaagtgatgatagtaatgtatgtatatatgttactgacattattttgtcgttgtctaataggagggtactaaagtttctctctctcatggcgtcggagctcagcctctagtaatgtagactgtcctctctttctctctctcttctttaaacatctctgctctgttttccttccagttcacgctaatctcctgtgtcggtcccctctctcttttcctttggtctctcccaacaatggcagaacgactttgtacccttcttttgctctttgtttgaatttcgtttcttgctacaaagcttcaaaggatctgacttttccctaaacagaaaaagaggtctttaaccaaaaaaggttgttacttgttttctgggtttcgtggtgttactcttgaggaagaagaagaagaag

SEQ ID NO: 46 - DNA, 1470 bp, Arabidopsis thaliana
  source 1..1470: mol_type = genomic DNA
ctacctaatataactagctagggatttctactcttgttttcataatcgatctacggacatttctcggaacgtggtcaagattcatgagtcttctgttttttatgtctctgttcaatttggtttagagattagtatgcttatttgtttatttcatatatggttatgagaggagaggctaatggcatatactctgatgtttgtgatggctgctaatatcgttgaggagttattcacgttgtttcatgcgcaaaaatcaacagaaaaaattctgattatgagccaactctgtgaacccttatagtgcgcccagaggtttgcgaggcaaaatcccgatgaaccagaaggaattttagatctctatcaacaataactatgatggagctcgtttaaattcatcacagcgacaacatcattaggctgcccaacgtctatgtctcctggaggtgatggtacttgatctctcaaccaattttcttgaaaatatcatgccttgtgagcgctttcatattgcgcctaaaatacccaatacgcaatgaacctacttccaaaggcatagaaaaaaaactgataatgataatgagatttgtcactatacttatcctatccctacataggagccgtttgattgtttagtccatgttttcattttgtttagtctaatgctatataacttttctttatcagtctattgttatatgacttatatatatctcaagagataaggccaataaatcttcttcttaattatatctgaagactcaaaacatattttgagtttaataaaataaataacgtccaaatgctacatacaaacggaccaaattcatggaggtataaatttaaattattttttgttccaaagtgtatgcagtgatttattgatgaatgcgatagagcggcgaaagagaataatcgtcacctagaagacaaattgatcggccgtacatatatacataaatacaaacctgccacttcacatgtcacccacctttaagcacccccttcacatacatactttctataacaaaaatatcagcttctagttcatatttatgttacaataactcgagtgaatcatactaaaaaaatgtaatgctttctctaaataggagataaaatgcaccctccgacctaactaaagattccttattttagctatttaagacatattgcacatgtatagagatacataaacacatatgcaatatgcacatcttctatacattgaaaaaagctgatcttgcaaatatttgtcttacacaacacaagcgaccaaagcgatgcgtttcccaatgataaggttacgacatacttacacgactctctctattgtctcgtctctttctttcctcatccctctcctttgtctcctttcactctatttttcacttttcagaatacttttacgtaaaaatcatggacatgtcattgtctccaccctactatactctttttttgttctttttgttt

SEQ ID NO: 47 - DNA, 1793 bp, Arabidopsis thaliana
  source 1..1793: mol_type = genomic DNA
ttggttgtctggcatcatcattttgtaccgtttctcccaaagtaagaaacggtacaatcttctcttatatagatttcataccccaaaaccctaaattcattagggttttcaaaaaaaaaaatcacgtttacctctaaaccaatcttctcttatatagataaatcataacgtttgtttgatttttcagttttctacttaaccaatattaaactaaagtcgaattgagatgagtggtagcaaaccacagtttaattaagaagttaattataggccacatgattgagcaagcctttttgttttgtaacacatcttatcagctgcttaaaattttggctgcctcccattggccaactggtctaaacatcattgcattggcattctcataatcaatcaatctaatgagaaactttgaatatttatgaaaaactgaataacaacataacataaacgaacaatgtaaaaaagaaaaacacaaaaaaaaaaacactttaaaaaacaaaaaccaaaaactcttaaactataaactcatgaacacttagtgatgaggtctgaaagggtgtaaccaccacctgttgtcaataggtgacaacttcttcttgggaacattcgggaaagtgaaggcttaggtgacggttgtcttaaaagtctcttttagttaattcatcgtattttcgatgggcattacgtttgatgcataaaggcccatatgggctatacatgtactgcgtttgagtggctttctaaggattgatgtattgtctctatgagagtattcgtttaactcatggagatctactctccacgatattttctgtaaacttttctttttgttgattagataaatagaaaattgtgtagagcgaaacttttaatgaattaaaatgcggaagcgattaaagcatgaatagataaattggacaagagattaaacgagggatcatctagtttttacactgatcactagtcatctgcttgcagaagaagtatatcattaatcaagcaaaacgagggcataaatttcttacaaataacttttacagtaggttaatgattttttaataacttgtccatttcacatgcatgtgtatctttgtactatacatgctaagtgtttcattaatcaagataaacgtgtctacgaataacttaaaacagtacaacttccctaaaaaattcattaaatgaaaggtttttatagattatacattgcacggtacggttcggttaccattcgaagtctaaaaagagaatgacggttctgataatgcttttaatcgcttttgtattgtaaatcattaaaacagtaagccggataccgaattactaatcagacccaaaaaagaatctataggaaaaatatcaactgaagagcgggtaggcttgaccttgaaaggaagaatggtgagcgagcggtggatagatatgtaataaattgtaacgctttcaaaatgtcaaagtcacaagtcacattactcacgagccaacactaaccatgcaacttttgttttgacattttcctaaactttaggtataaaatacccgcgtaataaataacctcttcataattgggtccacccactcacaggtccacacataaggaaccgaaaaaggtaaaattcaaaaacttacaaagttttttagagatgatgtggtgaagtattgcattaatggaataatgggaaaagaaagtaattgcaacgtacgtatagattaatccattgacacaaatgaaaagtttctttctatttaatgtacacaacaaaggttctcttcagagtaatttaggggaaaaa

SEQ ID NO: 48 - DNA, 257 bp, Arabidopsis thaliana
  source 1..257: mol_type = genomic DNA
acacaacaggtccctacatttgtacaatctcctctctttaaagactctctctctttctctctccatctctatcttactctgtatttctgtcgtctgagcactcaatgaaaccactgtaaatttccgccagaatttgatgtgatggaacgataaaaatcattttttctcggttaaagtaaaaaaacaaaaacaaatttctgtagaaatcataataaaagaaagaaaaaaaatctaatgtcggtacataatacggttct

SEQ ID NO: 49 - DNA, 1787 bp, Arabidopsis thaliana
  source 1..1787: mol_type = genomic DNA
aagggatatttaatgggaagaaaagaagggtggagatgtaacaaaggcgaagataatggatattcttgggatgttgtcttcaaggccacgagcttagattcttttagttttgctcaatttgttaagtttctacttttccttttgttgcttactacttttgctcatgatctccatatacatatcatacatatatatagtatactatctttagactgatttctctatacactatcttttaacttatgtatcgtttcaaaactcaggacgtacatgtttaaatttggttatataaccacgaccatttcaagtatatatgtcataccataccagatttaatataacttctatgaagaaaatacataaagttggattaaaatgcaagtgacatctttttagcataggttcatttggcatagaagaaatatataactaaaaatgaactttaacttaaatagattttactatattacaattttttctttttacatggtctaatttatttttctaaaattagtataattgttgttttgatgaaacaataataccgtaagcaatagttgctaaaagatgtccaaatatttataaattacaaagtaaatcaaataaggaagaagacacgtggaaaacaccaaataagagaagaaatggaaaaaacagaaagaaattttttaacaagaaaaatcaattagtcctcaaacctgagatatttaaagtaatcaactaaaacaggaacacttgactaacaaagaaatttgaaacgtggtccaactttcacttaattatattgttttctctaaggcttatgcaatatatgccttaagcaaatgccgaatctgttttttttttttttgttattggatattgactgaaaataaggggttttttcacacttgaagatctcaaaagagaaaactattacaacggaaattcattgtaaaagaagtgattaagcaaattgagcaaaggtttttatgtggtttatttcattatatgattgacatcaaattgtatatatatggttgttttatttaacaatatatatggatataacgtacaaactaaatatgtttgattgacgaaaaaaaatatatgtatgtttgattaacaacatagcacatattcaactgatttttgtcctgatcatctacaacttaataagaacacacaacattgaacaaatctttgacaaaatactatttttgggtttgaaattttgaatacttacaattattcttctcgatcttcctctctttccttaaatcctgcgtacaaatccgtcgacgcaatacattacacagttgtcaattggttctcagctctaccaaaaacatctattgccaaaagaaaggtctatttgtacttcactgttacagctgagaacattaaatataataagcaaatttgataaaacaaagggttctcaccttattccaaaagaatagtgtaaaatagggtaatagagaaatgttaataaaaggaaattaaaaatagatattttggttggttcagattttgtttcgtagatctacagggaaatctccgccgtcaatgcaaagcgaaggtgacacttggggaaggaccagtggtccgtacaatgttacttacccatttctcttcacgagacgtcgataatcaaattgtttattttcatatttttaagtccgcagttttattaaaaaatcatggacccgacattagtacgagatataccaatgagaagtcgacacgcaaatcctaaagaaaccactgtggtttttgcaaacaagagaaaccagctttagcttttc

SEQ ID NO: 50 - DNA, 1731 bp, Arabidopsis thaliana
  source 1..1731: mol_type = genomic DNA
atttctcttaaaataactaaattatatgaagaaaaaacaagagagataacgacgatcttgatctaattaggaaagttttctaatgatggtaatgtgaaagagtgcttctttcactctttatttaaagaagagatattttggaaatagcggtttgaaagaaagctataacttggtattggggaaggaaataaaccagagttggtttctaggatgtgttgtcgacttctcattggtctgattagacttccattgcctaatgtcgagtctatgttttctcgtatataattttcaataagtggaacattgataaaaaaaataacaataattgattatcgacgtctagtgacgtgaaaatgggttagctagacaagtgaaatatgcacagaccacagacttcagagcttccccaagtgtcgcattcgatttgcattgacggcggatatttcttaaataattttctttcattttctaactatattattggttgttgatatatattttttgtaaataaaaatgtaataatgtaaccaagtagttatagaggttttaattgctcggcggtaagaacagtgaagtacatatagatgtttcttggtagagttgaacttcaattgacaactgtgtattttgttgagtcaatagatttcacgttggatataaagatacagcagaagaaaattaattttgaagtttgggttaattaaggaaatatccaaaatacaaattaccataagttttttttgtactactaagcaaattatatactcaacttttctttttgctaaaatttgtctttgatggaattctagggaataaattaagagaactacgtattatagattaatacaccatgatcactttaagcagaccaaatgataataacttattgaacacgatttattcatgtagtacgtacatagatccaaagatcggaataagatacgagagaaaatggttaccccatttatttatgtggagataagaagcaagcaactaaagcaattaaaagtaccaaagaagaacatttagagcaaactaaaagaatctaaagctcgtgcccttggcgacactatccgctgatctccattatcaacacttttcgtacatatgcagtacttcctcctttgtccattattaaaatttagcatatatgtttgaatcgttgccgctttcaattttttttttttttgagtatattgttttgagtgttgtatatccgtcaacatattacacatctatatttttcaactttttggatgaatttactaagttggcataaattatcaaaaaacaaattggtaaatgatctgttattaaattgtgattaaaataacattttgaccaaaacaaacctcgcatcacacataattatgatattttattatatggtgcatgcaatatctgttaactaagatgctatttgaatgttaatttcaatgtgtcatccaatccaatcgagtttttttttctttttaaaaaaacaacaaaattcatggtcactataaatactcttttattcttaagaatttttttctcctgtattttccaaaaataatagtccatttttaccttatttatctcccgaagaaattggatcgatcaatgaaggaaagaaatcaaatggtcttatgatcaaagcaacattgagaaattcaatctacaaaaagcagacacatctcgacgtaagaaagaagaaattatcatagtgatcaaaagtacgtaaaatcggaa

SEQ ID NO: 51 - DNA, 2001 bp, Arabidopsis thaliana
  source 1..2001: mol_type = genomic DNA
atagaaaaaagtggttatccaagctacacaaactttttagacttatttctctcattcttctagagatttttaatgaaatttgtatatacttgaaatgcatataaaataaataagaaaacaatatctacttaatgtcctgttcaagaattattaagaaatttcactacgttgttaagtccattttactccaccttctccactggagttagactctactaagtcgcgttatcctgcagagtaaagattgactatgcattggaccttgtcgatgtgaaaatgttaaaaagataattattatgaaatctctactataacatatttggtttcctcaattttaaaatacctatttttttctcagtttaccattaaccaatctattccgtgatatttaatgtttaaacccttcttcaattctatttaacctacattctaaacaaggtgtcctcattttcaaaatcctaaactcatcttcatctatatttctattttctgccaaaatcataccacaaaaaacatatacgaaaaatgaccacggaaattgtcttttattgtttatttccaaagtcaaccaagttgactgttctttatataatgatttttttttaaaaaattatatattgagctaagaaaatattaagtgtaattttcaaaaagagaaaatatatttattataaaaatctatagtggtggatcaaaattctggtttaaattattgtatcaattttgaggttcaaattttaggatggaagagaaacgtagaatgcacttaaagttctgtcgatttttgttagtacataatccctcatataaccatacatatacttattgcaataaccatacgttaagaatagggaccaacttcatcggtgcttctgttagacatttcaagaatgatatatcacattcatgtatctccgtatatgtattaagaccaatgactaaactcagccattgtagatatagttcaggtaaaactgtcaattgtggatcagtatgttgaaaattgttaactatggttcgcttgtaacagaatataaatataataataatatataatactaatagtaatatatataataataataaaaagcaagcactaattattttatagaaacactttaaaaagtattaaaatctcatttaaaacaactctccaagttggtagtccattgatctacaagacagctccacctgaaccacatccacaatgtaaaatttaaccgtacacataaaccatgatcataagatgcaacatggtaaaatctctggtgaagttttcgtaatcgatgatgaatatatatatatatatatatatatatatatatatatatatatatatatatatatatatatatatacaatatatttttattttcaatctatttagtaagtacatatttgttatgaaagtacaaaaataacattgtaataggtcaagctctttgtggtggtttacattgtaataggtcaagctctttattattattttactatatgtatcttcttatccattcttatattttatatcatatatggtcaaaagaaccaatcgtatcacagcagcatttgttaatagaactttgatcttggtaattacaggttctatattgaaatcgcaggagccaaaccagcaacttgggttaattattaacttcattcaccaataataataattgatcgtcaagactatttcggacacaaagacataaatacttgttttctgctctccctttccctaaatgtatcatttgatactttctctttttccctccatatgccttttgtgtgtataaatatatgtatatgaataagttcacaaaaatagaaaattaaatcaaatcttcgcgtaatttctcatcaaactccacttttcttctttttaagagaatctagggtttcttctactacttcttctttcttctttaggtggtcaaatttccaagttcttcttttagctctctcttttgtttccaagaaacctgagaagaatttttgtagata

SEQ ID NO: 52 - DNA, 1814 bp, Arabidopsis thaliana
  source 1..1814: mol_type = genomic DNA
tctacagtgacgtaagtgatgtggtataaatacggtatgtgacgtgaatacatagacgatgatatttgtggaaaaaaaagaaagaaaaaaaaaacacaaaagctacatacgagtggaagaagagaccagagtggagataaagttttaagggctgtttgggaaataaaccatttgatgtgttgtcgagtcctcattggctactttttactgtgcttgtaactcgttcgttagttttgtgcctccctttagcttacattgtttccgtaccgtacggacttttgttattgtttttaaggttaagaaatcagaccaaagaactcaatagatattaaaggagaataatctttaactccttaatactttctacagtaacactataacgactatatatagggtttaaatattctttcaaaatgcaaattgtattttggttttttaatttttctttttgcatggagacaagtttttttctggttaaatgcacggagaaattttaattttatttaaggtgactttctgtttttcctttaaacagggagaaacattgtgcttacaatttcaaagatatatatattagataaaattaaatatagtgtgttttaataagtacacgaaagaatttgacccaattgtaataaaaccttaggaaaaatagtaattttataattgcaaacacaaaaataacacggaacattttacaatattttatcctgctaaattggaaggaaaaaaactaaaatgatgtccttttcttcaatttcaagttaagtatcgatcttctctttgctctggaaattgatgaacctacaaagtggaagctaggctgaaatttagctgctgaaccaacaaaactcttaacatcaaatgaagcatgttctatagatcgtggcgttgatctttttggaccaaccaatatgcaaaatgcatatattgttgtaaaaaaaatgcctatattccataacttattcacttgatatactataatttttcttttgaccgggaaaaagtggcctatttatgggaagagaatctcaaacgtatgatactctcaatgtaggaaaagtctctagactaccattttcttttactcctttagacccattggatttgggtagatggatttattaaatacaatgtaaggagtttttaaatgaaatataaagatactttaatgattaactggtaactggtaaattggtaatgtactactattgtagtcaatgtaagaaaatagttacgaaaataaaaccttttctgtcaattaataactcactactataatttttgtaggattaacacatcacactaagctattttatgactaaataagtaaatataatttcttccgaaggttcaactttttgagtggttattgaatagggaacgtgttccgataagaagttaacaaaacaacattcatgtcggtcccatgactattatatgtctgactctgtgtgatatcgatatatacataacatatattggtctgaaaagtggaaaatctattttaagatttggtaactgatcaatgaattgataactgatagtctgataccaaacataaactagttttgtacactaacaaaataagaatgaaatatcaaattttgtaagaattggtaactgatataatgaatcatcgtaactgattattttcttttaccttccgacgtaaatttgtttatgttgctcttaattatgttcgtaccttaattgatcataaacacataaattttagtgattccttatgtcgagttgtcaatggtttggaagattggtagctcctgacatttcatcggaatttcttctctcca

SEQ ID NO: 53 - DNA, 2001 bp, Glycine max
  source 1..2001: mol_type = genomic DNA
tttcgtgtgtgtgaacaaaataaatgatctgcacatgtatcatggttatagtaacaagtcccagctcccaagggtagtagctgcttaaagacacacggaaaccctcgaggataatggtggagagagagtgttgaaatttcaaccatctcacttttttcttctaccttattctttagccgaaaattaagttctaactttaaaatacagatttgcttcagaagttgattttttaggtttttttttttctggaagaccaatattaatttgtgatagttagttaaaaaaattatattatgttttgtgatagttagttgaaaaaattatattgtgttttgctactaatttactttcaaaatataaatatccaaatatatatataaatcttatcacttgaaaatagatggtgggtatatggactcaaatctatggatcggtctgtttaacccacgaattttgcagataacaagcctttttttaaaagaaaattcgcataactatatccaagtttttcttagttcggtccattaagctagcacatctagtagtctttatccatggacatattattaatttgttatgttaaaattttgatttttaatctagtggtatattttagtatgatagattttaacttaaaaaatgaagtcaattttttattactaattttatacatttttcacttttaaaaaatatttatttcataagaaatatttttaaattactatttgggtccatggatcaatttatttatctgagtttttatgggctagatacaaatcttggaaaaaagccaatttatttcatggattggcttatttgatccgtctaaaatgcaagcttcacaagacaaaccttaaacataccataccatccttttatctatctctacttcaaaatcaatttttgcaacattcactcaatggtgcacttaattaacaaaccctcatccatatacacatatttagagatcaacaatatgattatctatcaaaaatacacaaaatcagtgtgtgtttgggtaggcgttgacaaaaattagttttgaatgaaattgattttataattttttttttgttaaaattgattttgaagtaatattatttatgtttggatgttttattaaaaaattaagttatgaataaaatgaagtacataattttggaccaaaaattattcaaaattatttcaacccaaaattaattctgtatccaaagtcaattttaaatttttctttgatgtgaaactaaacatgtaaaaatgtatttaaattaaaattaattttagacttataattaattctctgtggtcaatccaaatacacactcacacttagaatgtatgaaatttcaatttttaactttctcatctatgagctgttcctattcctccttcccctcctatgccctcactagggagccagccagccatattccaaaagcccttattatcacacatgggtccctccatagtcaaaataaaaataatatcatgatcactgtttggccataaagagctatacgacacacatggacacagtagtacactgcccaaccaatcacacgtcgacagcacaccgttcccaaactacttcacctttcccaaaccagaaaccaaaaccacttgtcatcaaacccctgcccagatagtttttctccatttcaatattttacttcaccttttgaggctttgtgggtactacaaaacataacacaattgaactcactgtgctttcccatgacacacatctatacttgtccagaagaagaaagatccatgaactaacattcccacacgctcgcttcaccatatttgccacccttttaaccctcacttcttgggtttattttgctccttttttttttccttgtttggggtttgcatttttcctggttgaaaaagggaagaacttgaactaattggttaagttacctatctatctatctatctagggtataatattttatatcagttattaaaggaaagaaagaaagaaacgaagat

SEQ ID NO: 54 - DNA, 2001 bp, Glycine max
  source 1..2001: mol_type = genomic DNA
tcactagggtagtagttgcttaaagacacacggaaacatttaaggataattgtggagagagagtgttgaaatttcaatcatctctctctctaccacggtatacttcaataaaaaaaattaaggactaattttaaaatacagagatttattctagaggttgattcttttagtgtgtttgattttatgttaatacctcaaacatacatttagaatcccacaaaattagtttggtatcatgtttggattttcaaaagttaattttgtaacgtacgtttgggataaacctaggtttgggtttggatcagaccaacaaatttgtagcttttaaagtgaaattactttttatttgtatatttaggatagaaacatgcatgaagtaaaaatacttttgattagtatatatccaaacaaatatcaacacttttgaacaaaaagggtaatcatgttctattaatattttttttttttgtgataaagaagtagctgaagcttcttatttgacaattaaataattcttctcctatcggattggtccatttttaaagtaaaatgatacctcaaattgtgactatatacacaagccgtgaatcaaatcttagaccttggttaaaagactcaagtgctaagtcatttgtaccaataacttttggtataagtaaactcattttttacaacatacttttgattcaaacttatgttcgcaaactttatacaaatatgcacttagtgtgtttggatatttcctagaatgaattttgagtgccaaatcaaggtggaaatattgcaaaggttaaagtaaatatttgttactttgaccttttcaagttaccatgttattgataaagagtatttatcgatttttattaatgattaatctttgtcaaaaaatttagttagttagttttagcgggattttttcctcctaattatatttttttcatccacaatacttgaatttgacaccttcattaaaaggattaagtctgtattactcagattgttactttgcctgctttaagttgattttgataagattgaaagctaatccaaatatatattttcacattaaaaattttattgcatttaaaattaattggcttaaaaataaattttgaattatatatatatattgaatttgttataacttttagcaagaaaaaatatttaaacacagaccacattatctcaaaataaattttaattaaaattattttattcaaaaatcaatttttatcaatattcacccaaacacacaaacttaatttaaaaaaaaatcctcatttatattagagatccacaatatagttatctatatatcaaccatacacaaaatcataacgtaagaaatttcaattttcaaccatctcatgtgatctatgcatgctattattcctcctacccctcctattccctcactagggagccagccatattccaaaagctcttaacacacatgggtccctccatagtcataataaaaataatatcatcatcactgtgtggccataaagagctacaagacacacatggacacagtagtacactgcccaaccaatcacacgtcgacagcacaacgatcccaaactacttcacctttcccaaaccagaaaccaaaaccacttgtcaccaaaccccagtcccagatagttttctcaccatttccgtattttacttcacctttagtgggtagtggttacatgatatcagcacactttcactcactgtgctttcccgtgacacgcatctatacttgtccagaagaagtaagatccatgaactaacattcccacacgctcgcttcaccatatttgccacccttttaacctcacttcttagggttattttgctcccttttccttgtttgggaattgggtttggtttggtttttcttggttgtaaaaggtaaagaagaactaattggttaagttactatatatctagctagggtatataatattatataattattgttattaaaggaaacaaagaaagaactaagat

SEQ ID NO: 55 - DNA, 2001 bp, Glycine max
  source 1..2001: mol_type = genomic DNA
acaaaagatcacaaaattgaacaataatgacaatacctcgcattgaagtagttcaatacatattctgagtatgactcgcgagtacatgtatctcatgtttcagtttttaaccacagctggatgccatggacacttttaattctattcttttaaatgagatacctggcctatcaattttaagggtaaattaaaactcagaaatcaacatactaaaaaattgcatgcatgtccgcaaaagaaaatgtttaaggcagtatatattcactagtgcacaaagttttgaccttaaatattgaaatagtgaccatacgagtgtaatgtttcagatatatataatgctaattatggactaattaattaaaatagaggattgctttcaataggaaaatagtagtgtaatggaaaccggaaagtgaactagaaagaaatgttaaaaatggtctagggaaaaagttaaggagaaaatggattatacataatgtcaaaaattttataccgctgattataatttatatattatgtaggaaaaatttattaaattttaaaataattaaaattataatttatgattgaatgatattataaaacaattttacacaatcaatttataaatattaaacttaaaagataaacctaaaaaatactatttttctttatcataatattagtataaaataattctactaaaaaaaataattaatatttataaaaatacatataaaataaatattttttttataacataatttattttatatataaaaattaatcaaatattttaactttggattacttaattaaaaaatgattcctaaaaaagaccatttgaatattatttggtggacgaagggatggaccagagttcaatactcgtgggttttggcggcgtcaatgagaggatgcacgtaatgttcgggtccgtacggcattgccactgtttaaaccatcggacacttgaaagctcaaagcagtgactgtgatgagcagctcgcaatagagaatccaaaattcccaaaagcaaaaacaaaacccaaaaagcaaaagagatatcattgtgtggctgttagataccacagacagcaacatggtaactgaccacccaatccgggctcgacagcgcaacgttagcaaaaccgccctggttttttcccaaaccagaaaccactttatcgccaaaccccaatgtgattagagtgttgctaggtgcacctactgttattgctggtgcacccaaccttctttgataatgataaaattatccgtcttctttttccctttacggatcaagttgatccgcaagttgatttttaagacttacagatcaagttaatcggtaagtcttttatggatcaacttgatccgtaagtcttaaaaattaatttaccaatcaacttgatccgtaagaagcttgcagatcaagttgatccataaaagacttaaaaatcaatttgcggatcaacttgatccgtaagaagcttacgaatcaagttgatccgtaaaagacttacggatcaacttgatccgtaagtcttaacaatctacttgcggatcaacttgatccgtaagaaacttgcagatcatctatatcaactgtagatcaactaaaacatatgcggatcgactcattacatatgtggaccaacctgaatgagctaggtgcacaaaaatatttttaaaaatacatagggatatttttgtcttttcatgttaagtgttaggtgcccaacaacaataatgctgggtgcacttagtaacacccatgtgattagggatatataacaacatagcatactcacactactactcactgcggtttttttctcctccagaagaagaaagatccttaaatcaacattctctctccctcccttggttataatagcaagcttttcaagcatatttccggggtttgttcccttttccttgtttagggttttctttggaaaaaaaaggtaattaggtataataatataattaaagcaaaaaaaaaaaaaataacat

SEQ ID NO: 56 - DNA, 1162 bp, Glycine max
  source 1..1162: mol_type = genomic DNA
agcgcaccgggaaccaaaacgcgtgtacgcgtaccacgtatcccaattccataagaaattctaaaaacccacacgaaccattttgggcaggaacaaaatcccccagacgacgacacctcagaaagttaggggcattttggtactttctgcatgtgttagagccttgtttgtataaaggatatagatatttgtctaaggtgtatattgtaaacaaaatatgactttcctattcttacatggtcttttgtcttataaaagatgatagtgaccgagttgataattgttcacaataataatatgtgtaaagtgatagtcagttaacaagtgtcgactacccgtcgattccgtacaagatagtaatcatacgagtgtaaagtttcctatagagagagagacatgaatactaagggggattatttaattaaaatagaggattgttttaaataggtaaataataataataataataataataataataataataataataataataataataagcaaaggagaaagagatgttaaaaatggtggaaggaaaaagataaccctaaaaaggaacatttgaatatttggtggacgaagggatggaccagagttcaatactcgtgggtcgtggcgccgtcaacgtaaggatgcacgtaatgttctggttcatatacgacattgacactgtttaaaccatcagacacttgaaagcaaagcagtgagtgtgactgtgagagagcagcaatagagaatccaaaatcccgaaaatcaaaaacaaaacccaaaaagcaaaagagatatgattgtgtggctgttagttaccacgacaacaacatggtaactgacaagccaatcagggatcgacagcgcaacgttaagaaaactaccccggtttttcgtaaaccagaaaccacttgtcgccaaaccccaatgtgattagtgatatataataacatagcatactcacactaccactcactgcggtttttctcctccagaagaagaaagatccgtaaattaacattctctccctcccttcgctataatagcaagcttttcaagcatatttctggggtttggtcccttttccttgtttggggttttcttgggaaaaaagaagtaattaggtataataatagtataactaaagcaaaaaaaaagacagtaatat

SEQ ID NO: 57 - DNA, 2001 bp, Solanum lycopersicum
  source 1..2001: mol_type = genomic DNA
atctttcaaagagacggatttagagcgagttctaatctaataaaaaattattcaataacaccatacttgaaattaataacgtttacaattacttttttttgaaacgtaggggcatgtgaaattaaaattaaagcgtaaatgaaaattcacaatgtctaaattcggaactaacagatgctatgcaggagagcaacaaaagaagaaaccctaattgctaaatggaaaaaagtgcaaggaaaaatcaagtttctatatgtgtcttttaagtcaattattcatggtgtcaactgtcaatatatacggggataatgcacaaatacctctcaacttatgcccgaaatctcaaagacacacttatactatactaaggtcctattattatttttgttaataattttttatcccttttctgcttacgtggcactaaattgtgggaccaacgttgattggcttttttttccaagctagtgtcacgtaggccgaaaaggggcagaaaattattaataaaaataagtttaggggtaataggactttagtataatataaatatgtctctgggatttcgggcataagttgagggatacttgtgcactatatgcggtatttttatttaggggtggtttggtttgaagaaagttatgatgctatgattagttattttttagtgaatgtttgatttgttgcattaaaaataatatgtatggcataatttttaagaataatttatttcttcgattttcttcgtaaatgtgaaagtgataatataaaaatagcttaagaggaaaattttgtttaactatttttccagttatctcatcattaattcattattactcgactcaagacaataataactcaaaattatagggtcctttggtgcgagggatcaaaaataacgataatgtgataaaaaaaaaaatcttgtttagttgtcatgtttagaataacttatccaccatttacattatagtgatgagatatgttattcgtaaataactaccttcaaaataacctatcctgtaataacttattcccaacgatagtaataaaaatttatgttacgattatttttctttatccatcactggactaaacgtgttggaagaactttaatttagagttggtgggctaactgtcgttattgggaaacctgtatgcatattgaaaaagtaaaaaaaagacaaagagtgttgtttgtaggtacaattggtgtcgaatagtaattggagagtactgctgtgaagtgactctagttgtttaactgtttcttcactatgaaccacacgatgacaaacgtagtacaatcgaagccaaaaatagattcaaggttattacaaaacttaaataacaaaaccacaaacactgcaattatactacctagtatattagtactagcagtgagactgtttcgtttttgatcaaaaatttcgcgctcgagtttaatataggttgtatcaatggtgaagctatctcacccttgatcagaaatttcgagttttgaatataaaaaaaaattatattaagagcaccactcagaatgaattttgcagtgtacgattcaaatttaattgggggctctaatacgaaattccgcattgggtggaaaaggaaaaagaaaaagctatgatcaatctactctccactgttgcagtttaccctgctatgttgtactaaattgtattcttgcatgaatctcaaaggataatatttgattttcttttaattataagtatttaaaattatatccaaaaaaaaatatagcttatttgtgattccgagatctgtgagtagaaaatcctaaaaactatttcgagccaatttttgagaactatgaaaatatttaaagtaaaaaattataccatttgatgaatataactaaaatagatatttaaaagattaattatcaacgtttaattcaaaaattgatgaccaagcactagattggtcatatagttttgagattataacttcttaattattgggtattgttaaactttcaac

SEQ ID NO: 58 - DNA, 2001 bp, Solanum lycopersicum
  source 1..2001: mol_type = genomic DNA
cccaccctttggttcttcgaaccccctacccgcacccctttcttcttctttttcttcggtgatctcgtcccccgtccccccccctcacccccgcctccttctttcttctttttcggaacaccctcacctatttcttcatttcaaattattcttacttattcttaaaagtgatgaataagaaaaaaatgaaaaacccaacaacttgttacatatcgaagggtttgttgattatgatgaacttgaaacattttttaataaaaaaatttgtaatttcttcatctcaaattagttttttaatttatattatttatttattttgataatgtgttttaaaaattgatgtttctagttttgaatacgttagagtttgattttatttttataatcttggattaattcataattttcttttctgtttttatctttctaaaaacgtataaataaaaataaataaaaaagaattaaaaaaagtcaaaatttttaattttcaatgtggcactgacgtgacactaatgtgacaataaaatgacgcgtgtgtaatacacttgctactgtgagactggtattgtagtattagggttgaaaaaaatcactttaaagatgttaagggataatttaaacgctagtgtagtttaagtgctaaagtaagttttgcagccaagttcgagagaacttttatgtattttctctttaaatatatactcttctcactgacgattatcgatgtaatgatctgactgttctttaattttcttcaaagttttatttggattagtgattaaatggctaagacttaattgtcaaagtaactccctaaccatgatatttcttttattttctttttaaaattgatctctttattggcttatttctcctattttatttcacctctttcttccctatttcctctaagacgcaataaaaagagtgctttgaaagaaataaaagaaattgtatgcttaaaaaagtattaggaagagaactattcccaatatttatggacaagatagtatattgctaagatggcggaatttcaataatttttaatgaaatatataatgtatttatgaactaaattagaattgaagatttatttttatacaattaaattttaattattaatttaatttatatcatgtatcactagttcaagtaaaaatttagagaaaatggaaagaaaataaacgaagaagagccaaatctaattaggcatatgctacaatggaaccgcattaacaaacaaaaatatttaaaactactcctaaaataaaggaggaaaacatatattataaatcacaaaaaatattagtatcacaaaaatgaacctgagggacatattatgaaacttttatataaagtaataataatcaaattttagaatttaaattagccaagtgaaatatatacctttaattataattttatttttattaaaaaaacgatgtcatatatgaaatgtagagagtactacatagtgcagaagtactgaatgtcctaggaccttagcatataaggagtatatactaaaaataagaatgacgtaaaacaccatcaatgcaactattatacgaataaaaactcaataagtatggcccaaccattacctgccaacccgttgaaatctcgattaatttaaatttacgtacaaaataaataaaaatttcaaactaaaactatctaatcacattttgtctggtggtatgtagcataattttgtggctcttaaccaccagatactagcagtgtgtacgttttatccaatcacaaatcgacaacgcaacaccccttaatgcttaaaccactcttgtatcaggttattattattccctttcactcaatcaatcaaaaatcaacaaataaaacctccccccccaaccctcaatcatgcttcttcttccatatagtctctcaattagtccaaacaaacctctctatctatgctaggtagcttatatgatttaatagttcgattaatttaaatacgcattaaagatagaggat

SEQ ID NO: 59 - DNA, 1812 bp, Solanum lycopersicum
  source 1..1812: mol_type = genomic DNA
tttcgtcaaaagtgttgtcaaatttctattggttcacaatactcttcaactcatttaacttttactcaccgtttcaaaaacaattaactctagtctgacttgattgactagacacggagtttaagagtataaagaagatttttaaatatataattgtaaactaaaacatatttagaaaaattgagatagtaataattaacaaaaacggactcactctctcccttttctcattattactcggccgcaaactcttaaaaatacacgccttaattaatactcttgcctaatagagtattcatttagcacatttttaagaaaattgtaaattactaaggatatgattttttatataatggacaattgttattaaaaagacgaagatattttttaaaacttttattcgaaacaaatatgagaaaattggaaaaaaaattcagaaaatgtttttttcataccaaacacactcctaagtttaagtagggaaaatcgtctgatatacctctcaactttgtcatttagggttgatataccccttgttatgaaagtggctcatatatacccctacttgtaaacaaatggctcacatataccctttttctctaacggaaatgaaaacaaaataattttagtctaaatttttattatttttttctaaaaaatataatcccatatgaataaatttaatcctcgtcaaacatatttttttttactttattttgtttcaataactaatttataattattattttgatgataaaatttatttatgtttcactaatattcttgtaaaacttattgtagatgatcaaattttttcttcgaatccgaaattaaattacaatacacatacagaaaaatagtttaattttttattctttaaactaagaaatgaaagaaaaaaacaaaataagataagaaattcaaataattataataaaagaagtcaaaaaataatttatgtatgaaaaaaaattaaaatataccttgaactttgatagaagaatcatatttacccctaaataatttttttttaaaaattagaagtaataaatataaatttaaaattaattttttaaaatttgttaaatgaagggtatatgcgagccattttgtaacggcaggggtatatgttagccgtttgtataacggtaagggcatagatgagccacttttataacgaggggtatataagctccaaatgacaaagttgagaggtatatcaaacccttctcctatttaagtatttagcatatcatttaatagaataaattaaataaatactttatatgagaaaattgaaaaaaaaaattcaaaaaatgttttccttcatactaaacacacttctaatctaagtatttagcatatcatttaatagaataaattaaataaatactttagatgaatattaattggtatcttgatttagatagataattgggacgggacctcatgttcaaattttaagagagataaaaatattaaatgattcttttcattgttttagtgttgttgaatagaattatttaatacattttattaaaggatgatgataattattttatcaaaattaatctaactgtacgaaagatcatcaaaatattaattagtatcatccaaaaaacaatttcttaaaaatcaggcaacaagatatggaaataagatcttttgttttttcttatatttgaataaaagaaaaagactacgtatttaatatctcatatgtaatatataatgaataatcttttttttttaataaaagtatcataaaattatagagcactagaaaaatcatttctttttgactaatatggc

SEQ ID NO: 60 - DNA, 1880 bp, Solanum lycopersicum
  source 1..1880: mol_type = genomic DNA
atctgaaataacttttgttgggcaaaatatcccagatgtggaagagagagaatatagtggtttttaaaaaaccagagtggtttcgtcaaatgtgttgtcgatttttcattggacagagaagatggattatttgttatgtgtctcaataaaacccacaagtttttttttgtacagtagacaatagccgtgcgcatcttgtagccaggtgatgcgaaacaatggaataacgcccaccatctttttgaaatttcggtgaaaatagtagcttcgtcacgaattaatatttgataaatattattatacattcataatcactgcttcttttaaataaaatatttttttcgttttatatcaattgattttttttatatgtttattgaaaaaaaattataagtaagaagatgatattgctaattccacacatattacttttagtatgatgataaactaatataaaactgagggagtagattatttgttataataagatagagatactgttaaaaaatatgatatagtaaatgaagttggtataataaaaaatattaaaaggaaatgcttcactaatgaggccttatatagcacgatttttttttatgatagattatatatattatagttagggaattacatggctttgagacatattggcctctacatagtttcgaagcctattggcttccacgtattttttatatattagttattacggctaccttatcaaaacatgctaatttaatacaaattattcttaaaacgagcctagtagatataactctctgttggtcatcttttacaagcgccatagtatgctcggaggggtagatagtatagtaatttgggaggacgatgttagcgtggtacctagtcttgacaaaaactctgcttcatggtatatataacttaagactgatgtttttatataattggtatagtttaatttatgattccattacgtttaattgtgtgattgtatatttaaaacttaaatttctataacagtttataaaattttgtattgctattaaatatattttttaaaataaaacaaaagaaattaataaggaagtatgtcttttttattcggacatgatgattcataaactgggataaagatttataatagattgtagggtaaatcttatatttaggtatttcattttcttataatttttatcagctgtatataagtttcaatggtttaatttattgtagtaataggttattgttggtgcttctgttgaataaataacaaaatcaagcattatgggtatatagttgtttataatttctttgatacgatggtacaaaattatcttatgacttgaggggggtgtttggtaggcaatatttcggaggaatatttcaaaattaaattttaaataaatttatagttactctcttcttaattatttgttgatattttaatttaaaattgtctcaattatttataaaaattttaaattcttaattaatttaattcataattaataaagataaaataataaattactatatcaactatcattttttaataatatattatttaaaaattgaataaatatttaaaaataaaaaaatttatatttaattgataatatatcaatcaaacgcaatataaatttaattcgcaaaaatcaaagcgtaaagttaaccaattcattggctgggtggatagcagtcgtttgtatggacaagaattaatgtacggctaatactattggttaatttattgagcaaattgtgtggtccaactggatattcactctcttatccctttcgttttcctcttttagtattttatttttcaccccatagtaattgttattttttaaatgtgcattggataaatttatatatatatatatatgtaataatta

SEQ ID NO: 61 - DNA, 2001 bp, Zea mays
  source 1..2001: mol_type = genomic DNA
cccgccgagcaagtcaatcgccccatcatgcggacttgctcggcaaatgggctagagagaggtttatgggcctcgccttgggtaccctgttcccggtacccgacaatgaccttcctcggatgctgataggtcaattaaagaaacaacaatggatatatatggataggtatagaggtgtaaggctatctctagaacgttgcctattcttatacccatattcaaactttattgataaaatgttggacaggtctggtgcccttggaacaagtgttgtttccattctccagagtggactacttcttgcgctgatttgtttggtgagttaccgaaggagatttaggggaagaaaaacagggcacttataagtgatatattgtttatctcagatgtattgatcacttctctggtattggtgcaatgtattggggtaccgatcactgagtaatcacgcaatgtattggggtactaaatcctctctggtatcgattattcatgcaatgtattgacgattttaataagtgaatcgccaatgtatatgatatttccactggcggtgtacttaatacagccgccagtgtatatacatcatttccactgacggttcagttaagtgaaccgcaagtgtatatgacatttccactggcggtgtactttatagaaccgccagtgtatatacatcctttacactgacggttcagttaagtgaaccaccagtgtatatgatatttccactggcggtgtacttaatacagctgccagtgtatatacatcctttatactgacggttcagttaagtgaaccgacagtgtatatgatatttccactgccggtgtacttaatacagctgccagtgtatatacatcctttatactgacggttcagttaagtgaaccgccagtatatattatatttccactggcggtttacttaataaaaccgcaagtgtaaatacatcatttacactgacagttttgttaagtgaaccaccagcgtatatatatttacactgccggttcgttaagacgggcccgtctgtttttttcactggcgtgctgtaactgaaaccgccattataaatttctacgtgccgccaccttagagctcttttctactagtgttaacttcttttcttgtagaccatttggaaaacaggaaacaacgcggtactgtattcaacaacagatggttgtccacacctatgacaatcatggcgtcaatgcagtagtaagtttgtcgtttttgtgtgtgtgtgtttattagccgtttctttgttttttttcttctgttgagctccaactttatgaaacgtcgtaagctggtaattatgaaatgtaaggatttggagagagaaaaaaaacgggagggaaaaccatgcatgctgctgacgcgacggccggacgcagacgcaacaatgcccccggtgcggcgttgtcgagcagccactgcaccaccccacgcatcacctgcagtaatctagcgacgggtttttcttatttatttatttatttatttatttttcttctctccctccctccctcagatttgttttcgttttcattaatcgttattaccagcaattaattaactttatctattgatttaccaaaccgcaataaagaatatatatattcttttattaaggtccagtaataagcagcacagaagcgcaggtgcagcagcagcagcgtcagcgcccgaggcgcgcacgagagaaacagaggctgacgaggtggggcccgtgcgggccttgaccaatcggagttcgacaacagcctggccacccacaaacacacactccttcgcctcgcgccggccgtcgtcgcctccctccaccgaacgatccctcctcctcctcctcctcctcctcctcgcatcccaccccaccccaccttctccttaaagctacctgcctacccggcggttgccgccgccgcaatcgatcgaccggaagagaaagagcagctagctagctagcagatcggagcacggcaacaaggcgat

SEQ ID NO: 62 - DNA, 1610 bp, Zea mays
  source 1..1610: mol_type = genomic DNA
tagtgttatccatggccacattattttgtttgtttgagttagtttcattattcaagcaacagtccattgatgcaataattgacatataacagttgtacacattatcttgctaaaaccggaactattgtttccagtatcaaaaaaatataacctgataatgaattttccattggattttatgcaatatccagattctcaagatctcttgggaaccaaacgccgccaaaaatcggttccacaaacgaattcgataaagcaagtgacaacaacgatttcagtgagattttttttaaaatacactactctactccaggaatgactaggacaacatagatagatagtgaagggaaaaatagtggctgcccttgccaacatcttttggttatgatagcaagtgtaataaggtgatgtagatggactacatgagacgccacgtcagatttaaaaatgtggaacaaagagaagtggagaaaaaaattgggttgctcgctagtagccaactgagagtgggttggacacggactccaaagactccctgttagagagagagaggggttgaccaaatattaaatatacaaccggtatcctcgaagagttattatacaaatgattgtctatacgtgacatgacaacatcacaaaccaactatagactacatattactctatctatactaaaatataattcattttaacgctaattacatatttattaattaacttatgaatgtattttgtatgtatatctacattcattatcttctattcgaatgtagatagaaaaagagcaatagaactatattttgggacagaagaagtattaattagagagtatttggtttctaaagacttagtccctcaattttattcttattcagtccctaaattgacaaatacaaaaactgaaattaagttttagattttatatttgacaattttatactataataaaatagagagattaaaaattaatacctagaaatcaaatatactcttaaacttcacttcgtgccagaggctagctgcgactagctgcgagattggaatggatccggtttcgaccgtacgaggaaggacgctggcccaggcagtggggccttgtgatgggagatgcggtggggcccgacgggtccggccgcggggcatcggctaatgggagttcgacaacggcagcctcggaacccatcccggtttcgcgataccccctcactgctccagtgctccctcctcccctcccctccgatcaagtcggggcaacgcgcatcactcgctttaaatccgcacctcccggccggtccccttatcacctcaccttctcctttgagtcctctctctccgccgccgcagctagctgtgacgttatgctctcgccggcgccatagcgccagcgcctaccgtctacaactatccagccttaggcttacctatcccgtcaatcaagcctctcgtaaggaacaaggaaggtagctagctagttctatagctgctgtcgtcgtcgtcatcggcggcggcggcgcctgttcttagaggataaggttgtcctagcggagagggagctagccaggatttcggttgagatcaagagggggagcaggcggcggcggcggcgat

SEQ ID NO: 63 - DNA, 2001 bp, Zea mays
  source 1..2001: mol_type = genomic DNA
cccccctctaggcgactatcaaggatccctatccaaacggccctaagtgcgagacgggtatggactagaggtcggtgccgaggaggtagcagggaggtgcgagattctgttacagagatcgcggggtgctcgggccacacaacggcagcgcgaggcgtttggcatggtccgccaaactcgcgaagacgacgaccgggaggcgtggggcccactgcgcagcgtgatagtttggtcgcgcagcgggtgcggtgagcggctgacgagagggtcccgcccgccagcgagccggggtgtgcgatgggccagcgagcgcgagaacgtagttgggccgcgcgaagcgaggagttggctcgaaattgggccgaatagagtggaattggcccatgaagcctttttattcttttctttttatattttctgttttctgttttcttttctttttatttttttcaattcccaatttgaacttcaaaatatgagtatgaaactagtacccaatttgaagttcaagtatgactttgaatttgtactcaaattagatttttaatcataccagtatggttaagtttgtgtatttataaactttgttttatattttattttatccctttctttctttttttcctcaaatactcttctcattatcatttttatttaattcatattattattgattttaatgcacaaacaaaaaatccaatatgatgcaatggtttatttgtgtcttattaaggatctacttttttttacatgagtggtcacatgtaatgataactagaggtacacatacatatataaaggaaacaatttctccttttattcttccttacaaagtgggtattacaatcctttcatttgcactttcttcgatgcattgattgttgattatcccatgtttctatacctcttagaggttggacattgctcctttatataaaaacacacctagaacgagtgaccttagagttgttctccatctttggatgctgaataagttgcatgttagaagctcattagccgatgaagtgtaaacatctctagtgtgaatactttatggtggaaagtgtttctgttgctttgttgattgtcacagtttgtgtcccatgtaattctaactctagccatggacttttttcttcggtctattagcttgagtgctatgttaggttgctaccaagacaaaatacactcacttgtgagtgttgaagttggattttgttcctttagggctagcttgaaacttaaatcccctccagagtttttggggattgagtgaaaaatgagctattttcctactcaatcttcgagaattatagagggaatttaagtttttaaactagccattgtctaaaaaatgtcatcttcttccccgtgtctaactcggccatgtcgctcgtgctaggggaggttcgactgttgtaacacccaaaatttctattttgagaatcaattaaatttctttaaaaatatttgaattaattcttttatgtgtattatactgctaaaaatatcttcttcttctcggccgtacaagtaggtcgctttccaatatatattctcgttttacaatattttcaacgcattttttccttttattaaggtccaagtaataagtagtagtaccggtggggaaagagaaagagagagaagcgcggccgcagcgagcagcgtcgtcagcggttggggcgcggacgagagtgagaggctgacgaggtgggcccgtgcgagccttgaccaatcggagttcgacagcagcctgcccccaaacccacgcacgctcatcacacagacacacacactctctgtgtctctcgcgtcgcgcccggccgtcgcctcccctccctccctgcaccgaccgatccctcccctgaacccctcctcacatcctaccacctctttaaagctacccagctacctgcctgcctcgcctcaacctcgccggcggccgcagccgcatagaccggaagagaaaggaagcagagatcggaggcaggcgaagat

SEQ ID NO: 64 - DNA, 1589 bp, Zea mays
  source 1..1589: mol_type = genomic DNA
ggtttcgctcgtcccgctgtcgtgttttgattggacgcgggggttttggagcggcgtggaggcagctagaccgtcggatcatgacggggatcgttattgcgggccagaaggatgggcctagcgaggccggcgttcgagtgatgcgctcgtcagggcgcgcggggcagggattggtcgagcctggcgtgggccggaagggtgcggttcagtggtgagatatggttttttccattttgcttgttccagttccagtccagccttccagggtcggtcgggcacaagggagcgagatggcaagtcaaacgtcgcgtttgctgcagggcgtctggctgatgggtccacacgggtgtaggaccggatgtctgtgaggctattatcaataaaagttttatgggagtttcatgcacatttaatggtatgtcacgtcagcattttggacgttatgccatatcatttaaaatgaatgggtttcagagataaaattagtttgatgtggatgaaatcatgtcaacccgttttcaaagtcttggaaactatgtgaaatgtccattgatagtggtttgtttaatcttaccatattttactaacatctattggttatttagttgcatcatttaattagtatattaatctatgaaaatgaaagtttgcattgagaagtattatttcatttttagtttcataacacttttgatgatgtggcattgttggaaagagcgatatgaaatccctattgtgaacagcctgagtgtcctcccctgtttctttttccctcggagaaactgtgattgagggttttgaataacaaggaattttagcccactcaataccgtttaattctctttgtccaaacaaatcattattgtagattattcattattattaggaaagattcaggttaccctccatacctattaggctttatttggatactttgtatagtgttttatatattataatttgtggcttctgtacagtgttatatcatcactagatgtgtggtttctgtattgtcaccaaataacatgggggtcatgtgtggtttttgtagaataattttatagtacgatgttgtcatgactctacaagggaactttacatgaattaaacctgcacaaaatttacaagcttttgagcctctgtcatgaatgtacaatgttatagaaatacaagaagtgtagccttgcatatttgtataaattgtttgggggaaagaatctatgcttcaaaaaatgtggccttgtagcttgcacaaagtacatgttgtacaatgctacagaagaaacatgtcttgccgctcagccaaaaggaccgaccgtgagaatcagagcactgcccctgagtctaagtaaacagaaaaagaggacaaaatgagttattacaatacagagaggcaaagattcttggaactatagaccacaaactttagggtcgtcaaccccaccacccacccgtgcccctcgcacgcgggcccgcctccttgcgcactaccaccggaccatggtcccttttaaatacttcaccgccacccactccccctttttgcgatgacctcattgtcatctttccggtaaactaggcacctcttt

SEQ ID NO: 65 - DNA, 21 bp, Glycine max
  source 1..21: mol_type = genomic DNA
tgtaggtaaccctcataggac

SEQ ID NO: 66 - RNA, 21 nt, Glycine max
  source 1..21: mol_type = mRNA
tgtaggtaaccctcataggac

SEQ ID NO: 67 - DNA, 328 bp, synthetic construct
  misc_feature 1..328: note = Description of Artificial Sequence: Synthetic polynucleotide
  source 1..328: mol_type = other DNA
ggcagagccgtgcccgtctcatcccctgcccgtgcaagcagctaggtaggacgatttgagcgtggtgttaggccgaaccgctgaaggaagattgctccactgttgactgcattgtaggtaaccctcataggacaaatgtattgcttatattcagcaatataatgttcgtcctatgatttttacctacaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 68 - RNA, 21 nt, Glycine max
  source 1..21: mol_type = mRNA
gtcctatgagggttacctaca

SEQ ID NO: 69 - DNA, 21 bp, Glycine max
  source 1..21: mol_type = genomic DNA
gtcctatgagggttacctaca

SEQ ID NO: 70 - DNA, 3058 bp, Solanum lycopersicum
  source 1..3058: mol_type = genomic DNA
aatcataaacacaaatataagaaaaaattttgtgatagattaataattaacgtaaaactcatcaatttacaaaaatctcttagaataatcagtgaacgattcatataatcgaccctaactgctcctaattggtgtgttatcattgttataagctaaaagtagttatgatgaactatacattatgtggtaaacaaattacttcaaaccataatatattgtggggcttgatgggattttgtcaatttcatcaaagaattttgttttgggccactttgacattatgcatccaaaaaggtccaatagataactagcccacttaataagttcatttactcctcttgctgattttggactaaacagaagagccagaaatcacagacttatccattttgtggtccagactgtgtcaccacctttgagaaaaggtaacaacgacgacatattcagtgaagttgcacgagtagagttttaaaaggagagcgtgtacattgtcttacgaaaaaaatttgtgttacatagcgcgtcatttgaatgagacctaatgaggtacaaattcaaaataatcagactctaatatagaaaacatgataaatacttgatttatcccatactatgtgatacattttaatatatagtttattttaaaagatatgatacatatatcacataaactgaaacgtcggaatgaataaaaaatgttgttattactaggtttcttgtgactaataatctaaagctcctttgaagaagtaattcctagaaattaggtttacctaccactacttttttctttttttttgacacaagtgaaggtataagttgaaacaatgaccaattactcatgggaagttgtttattagcccacaggtctcattggccacttatattttagcatagttacttcgagagatcaatcaaaaaacttacaaaaatatttattcagtagagtcgatagtaaatatttatcattcaccatcgtatgatatacatttgatatatattttaggtgttgattttctcatacatacaagttgtataatataacatacacttcagatacatatagtttgaagtcaaaatttatatgcaaaatatgaggtatgttaacacgtgatctctccataaagctaaataatttatgtatatattttttaaatatacatataatattatattctaatttgttttgcccttttttagtggtacaattatatttttaaaattttaaattcgtctctagctaacttgcacatatagtttgaagtcacatacacttggtactctcttcacctacattcaacttctgctttgaaaacatacatacatagacataatatattcgtataattttaataaaaaaatatcgtatacacgcaaatatattattttatttttaaagaaaatgaaaatccaaaatatgacaatcccaagtttgtggccaggtgttaaacacttgtggactaaacatatatatttattttttcaatttatccatagggcacacacttagaaaaaatgggcatttccccatagttttggcctaaagttcctaaaaaccactggcttcttctgcattctccaaaactgtggaaccaaaagacttataagtactgtttcttggtttaattatttttacgtattttatttttaaagaatataatatttaaattttaatattttattaaaattatgtatcgtcttttaaatcatataattatattgaaagttgaaattaaaaaacttactaaatataaataaaaatgcttaaattaaaacagaaggagtaatatctaaaatgagttatatatatatatataaaagctttatttaatacgaatattttattcatatagtgtgagctcataaaatttattcataatttgagaatagttaatgacttatttttcgatataatattgttattctcttctcaaaagatttactagtaatgcgaggactgttaatgacgtattctttaagctcgaatgatttagatttaacctttatataaatatttaatgacttttatgaaatatatcaagatattcgaaatggtaagtatcaaatatatattttgttgcataaagtttttttctaaactaaagtgcagagaactttatacactataggagtaatatttactacttatataagtagtacaatattgagtaatatttactgtttattaattattatattatattaatatactccctatccaagaataactcgactttaccaataaaaagtcactgtaatttctctttgctcctgagttaaatatttttattcggttattctcgtagccgattaaagttatatagatattaagaaaatatatttaggctttttatattatatttaaaagttaattatgtttattttagttatttatcaaatattatcttataccctgcgtataaactaaagtaaatatcacattaaatttacgtatagtaaaagtaatgtgattattaattatataaattttattaaatatgtgaataatttagctcctgatctactatcaaacgaattattatagtattagtgacggagccatcatatttttaagggtttattcgaaccttcttcagcgaaaaattatactatttctatatgattaaaataactttttagatatatacaataaatgtcaaaccccattcaatcattttaagtatttatttcttcaaattatcgaactcttttatcgaaaattttgaatcgaccgctatatactaatattttatttaaaaccacagatttatcattcatgaaccgacaaatgtatttaaatttactgaccatataacagaacctgataacacataaactacttattattattattattattattgttattataatcatagtaccaaaaaaaatataaaaataatattactcacgtactgagaattaaataataaataaataaataaagtgataaacaaaaacaagtgactggaacattggaaattatccaaaaccatcaaaacaaaatatctataaatagatagaaattcattcttatttcttacttcacttacaaaaa

SEQ ID NO: 71 - DNA, 2245 bp, Solanum lycopersicum
  source 1..2245: mol_type = genomic DNA
tcttgtcaatatgatctcaaataacaaataattctatgacaatatgtattaaccattttaaaacatcgatcttatgttctttcaattataattaatttactaaatattaattatattttacaacatcgtataatatatatgtgatacacatgtcaaagcacgtgcggtgaagctaaatatttaaataactttaaagtaactttcaattaatattagtttgaattaaaaaaaataatgaattagtcatgagatttgcgtactcttacttttgaagagtgtgttatcactcttataataggtaaaagggaaaagggtctgatatacccctcaactttgtcatttggagctgatatacccctcgttataaaagtggctcatatatgcccttaccgctatataaacggctcacatatacccctgccgttacaaaatggctcacatatacccttcatttaacggaagttaaaacattagttttaaatttatatttattacttctaattttttaaaaaaaattatttaggggtatatatgattcttctatcaaagttcaaggtttattgtaatttttttcatacataaattattttttgacttcttttattatagttatttgaatttcttattcttattttgttttttttctttcattccttagtttaaagaataaaaaattaaactatttttttgtgtgtattgtaatttaatttcgtattcgaagaaaaaatttggtcatctataataagttttacaagaatattagtgaaacataaataaatttgattatcaaaataataattataaattaatcattgaaacaaaaaaagtcaaaaaaatatgtttgacgaggattaaattactcatatgggattatattttttagaaaaattattatttttcatttccgttagaggaaaatggtatatgtgagtcatttgtttacaagtaggggtatatatgagtcactttcataacaaggggtatatcagctctaaatgacaaagttgaggggtatatcagacccttttccctatgtaaaatctaaaattaagtttgaactcacataaaattatataggccgtaacctacttaacccattgtcatcactaattatatagagatcttgatttgatatattaaaagtaaatgcatcacatattttttttaaaaattatttatttgtaaaaataaggtcgacaaatactgtgaaatcaatgtgaaaaaggttttgatcgttgagtgtatctttaactatggtgatgcacaacgtaacgaaatcattttcattaccaatattatgaatttttacattatatataaaataagatatataactaatatccatatattatattacatatataaattaaaaatatataacgaacaaaaatactacaaaattaatggaatacactctatgaatccagcatacacaaatagagcctctacgtaagccagttgttatctaatctgtttagtcactatgacatcagataagatatctgtttagggtacccgtttacacatcagcttttaaggtttcctacttaccaaacacttctccttcaccaaattgtttctccaatctcattcaaacaaacacacaactaggtggttcgaacaaactcaataatttttttttaaaaaaaatttatatttatactaaaaattaaataaatatacatgtacatttgtttcttaactcataacaaaattaattaacgactcagtgggatcgtgaaaatgttttttacggctaaagttatgaatttaaaccatagtatccacaaaaagttaagtttccattttttttcccaaagaaaatttaaaataacaaactcttaatttcaaaatatggtgcacaaaatataaggggaaagggttaaattgagataatccggtgtcattttaaagcgaacaaaattgattttttctaaaactaaaaatcaagaaacaaaatagaaacatatcaaataagacaacaaaagtgtgtaacgtgacaatgtgatataatacgaaataccctaggaataattgaacacataaaattcactataaaaggacaatgtcataattaatttttgtaaataaaatattcaccactttacatgatacgtaattggtatttggtacaaaatttgagtaatagtaagataaatgtagtagtagtgtggtacttactatggttatgtctatatatacatattgcatatgtatatataaaaagattgaaacaaagaaaaaa

SEQ ID NO: 72 - DNA, 2234 bp, Solanum lycopersicum
  source 1..2234: mol_type = genomic DNA
tatgatctttttcaatgaagtgattcagttcaatggaactcaaattttatatgtggtaggaattttatatcaagtcactaatttattttgataaccatcgagttagtagttaaaattatgatattgatatgttttaagatagaattgaatacataaaaggtagatctggtacataaactattgtgcttatgtagaatttggggaagaaacgtcaaggaatataatgtaaagtatagtagacaatttattttatcgtatacatattaataattattttcacgattcgaatatatataaccgatagatcacacaataataaatattagtgttgctcatcgaaaactccgatgcactaatgtttgccactaattcttaagatagataacaaacacatctaaacattattaattaagtgtatatatacaacatattttaaacttattctataactggatttcaatttaaaaaaaataatgatgtgtcatgtcccaaagttagttgcactctaaaaaaagttaaaaggtttttaaccaaaaataacttcttgactataacaaattagagttggaattaataatcaaaacatataaaaattgatatttttaaacaagttttacaccataatgtagcaatccatcctgttagtgatattgtctgctttaaatctaggaatgtacgtctttaaaatgcgtcattagtgggtaagacatgcttacttaaaacacgtcattaatgaataagatttgtttacttatatactcaacatctctcatatattttactgatgtgaaattagttatcttaaaccggaatgtcagtacacttcatttgtatctttttttatatgagccattatcatttacatgtaaaagtgcaccttaaagctggttaagcttataaactataaattgttcattttttctcgtttaataatcaatatctacttaacaaggcctgtttaatagatgataatagtttaagtagaaaaatgaaattgtaacttttttacgacttttaacatttcaactatcagttagtaatatgctcatccattacatattttaaagagaacaaagaaccattaaaaggttaaaaacttattataaagttaaatattttttcagtatatatgaaaggaccttacaagttacaactaaatcttttgaaagaaaagtatcggtcactactaagttttccaagaaaaacaacaacaaaggaacaatctttttctaccacaaggggatgtgactattgatagaatccattcattttaatgggagggcaatttttttttaagcggattcaaaatataaaaaagtaaatatacggacaaaaaaaaataagaaaatttatcaacgtatacataagaaaagttgcatacttccaaatagacatgatacataaacatgatctttaacttgacgtcagttggcaactatatgtgcacaagtaggcacttaaacttgtataagattgaacaattgacacattcatcctacaggcaccctacatgaaaattttgtgtcctgcgtggcgtcctacgtgtatcatgtcatgcatgacatgtgtggctacttgttcaattttatacaagagtaagtgcctacttgtgcgcatccaaagttgagggtcatagttaccgactgacgtcaagttaagagtcatgtttatgtattatgccctccaggtaacatagatttgaagaagcatggaatgcatgtagatcttacttctcgtgaaaatggttttcaaatacgaatagattagtctcggctcaagtaaaacattttaaaagtaagtacttaagacaaaataatacaataataaaaaagttatgataatattaaataataaaaactatagcaaaatataatatattatcgaagcaaacataaatgtctaattcaagcctcgataaatgaaaaaaataatctaatttgaacaccgcaactttcttttaaatgggccctccacgacacttcatgatatatagtaaaacaaaaaagtctttattctcttttcttcttgactagggaaccattagattttaaagacattaaatctattacccttaccctaagaataagaagatgtaaagtagaagagaaaacaaccaaaaccatatatatacatatatataattacattatattgtcttataacatatagtcttttaaggaaaaacaaatttagaaaaaaataatattattttacattttttttcttcatacaat

SEQ ID NO: 73 - DNA, 1302 bp, Arabidopsis thaliana
  source 1..1302: mol_type = genomic DNA
actatataccctccaattattgtgtaccaaagaccctcataggatttattcaaatattgcaaaattgaaagaaaaaatgttaattaccctatccatgcttagatattttcaaacctaaaaatttggtgcatttttgtgtttatacataattagtaaaggagagaaattagagttccactctcaccaacactttattctagctagtggtttaatttttcaatttcagtccatatttagtttagagaaaaatgtgattctgccaagaaagtttgatgcttttttacttagttcatcgaattatagagattaataattgaaaatcctacaaaaaaccctaacatttaaaaccatttttatttaatacgttgagagagacagagatattaaaatcgtatgattcgtaagagtgtgatgttgaaaatgagagattaataatggaaggtggagatggcgcgtgtgcgatgagtgattgcaaccctaaacagaaaatagtagggcaatgaatgagtttggacacgtgtcgacatctcgatggtagggtctgataaatccacctgatttttttatataaaaaattttgattttataaatttttcttttttacttataaaatcgagttcagggaatcatgtgacctaaccttaactaggtgaagcctcgtacgattttcccaacttacggaactacccttgtgattggttaaaaatgtaaagtaaaacactactcgtttgtgatgttttcatgatatctaaaactcttactgctagttttgataaatcttactgctagatttgcttttccttttccaatctatagtactgctaagattatttttgttagtgaaattaacagaaatgtcttttgtattagtctactttataaaccaccattttgatgtggccaaaaaaaaaaccatgttgataaattcagtttggtaagagagtctgatgagacacaaaaaaaattatgggaaagaaagaaaacataaatggataaaataaacagagatttgaatataagatatagtttattttggttatcttagggttcgtccgatgttagagatcatgtctgaccgaaagtcttagtgctttttggttgcagacgtaattgctttattctgaccggtaagtaggtgatggacacaaaggtatgattcaattagaaaaactaaacatagcattattattctgtttggttatatttaaacccattagcttcttttctcttttgatttgcacattgaatcgcttattaatctcacttggtcatggtctcatagaagcattttaagcaaatttg

SEQ ID NO: 74 - DNA, 2001 bp, Arabidopsis thaliana
  source 1..2001: mol_type = genomic DNA
aataaaaaactactagattacagctcttgattggttgataacttgatatcatatatcgtgtttgtagttgaagatgtaaagatttatcatcttgtatttgaattttgcacaatatataatgtgatgattctatgtttgtttgtaaacttatgtgcaaatgggcgataactaaacgttcgtaaaatagttacatgtactaattaatcgaagcacataaatattatatatatatatatgtatatatatgaaacgattcattgattttaagtatttaatttattttttaagattttagttgtcttaacctcgtacgaagaagaaggatatatactatttaactcgtggagatgatgaggttcggatatttttccttaagaccacaagacactgacacgccgactgatcgataaaaaaagttaaggcctacaatacaaaagagtagcagattcccttacacgtgtttcatttatgatctgttaattaactccattgagcttcaatatctcattagccgaaaattaacttgacgttttccattgaactgtcgaaaaaatttgtagaaaaagtgaaatgtcccctatttaacttccaagtttaaaacctaaaattaattagttaccgttttgatgaataacgttgaaagaatcgtcaacatatcaaatgatgcagatacaacaagggacaaaaacatcaaaaaattgattaaaaataatcattcagatattagcaattaaactgtaagtaaatgattgcatatatatatatgaatgaatacatatatatgtatcaaactgtaagtaaataatacatacatatatatatatatatataggaaaatatctcatttttcaatcattttcagaggtaaacatttttaaagacaattttacctaacccaacatcttactatatttaaccagctctgcgagagcttcaaaacaatgatgaattttctgccccttttcattgaccaaataaaagttcacaaatttgtaaaaccaatcagcattaatttcgcacgtagtttacttcgtccgattttagatcaagtagacattttaaccaaacatattgaatcacggttcaatcaagatacatggttgcgagtataatatatcaatccttcaccgcattagaaaaccaaattttctatttaatttagcgtgttcttttaatgaacgcagcgtcagcgacaacaaagaagaaaaacagatctgcggcgagaaaaaatacattgattagagaagaactgccgcaaaagatgtacgatgatgaagattgacgtgacgctgcttaatttcagcgattcatttgatgcaattgcaattcatcggctatcatcggcgactcatccgaaattgttattttcaagctttaatgtcgttttacttacctactacttctcatgacacgtcgtttcagtccgctattgccgctggcgcagcatcaaataaaagaacacgctaggatagcttgaaaagggtacaataactagttatagaagaacatgagccataacatttggaaacaaaatgagttagtttgaatttgaaacatcgcgctctatgtaaaaaaagcaaaaaagaaaagaaaagagtcctaataattaaaaagagaaaataaaaggaattttcaatggtccgactgttacaagtagatttccatttttaggaatctagctataacgatgaagacagagttatcattttgttccatcaaatatatgacgatatttatatttaatttcttcaacaacaaatatctcatttgttggtgccactttcacatacttaggacgactaataattaattaaaaaggatttatgtaataacgcaccatattaacctgtaacaagaagaattgaaagtaatacaagtttatatatatagcaacacatttatcgtgtacgatactttattccttttatctattcttgaaaaaaagttaccaattcttgagaagaagaagaaatcagaatcaagagaaggagagagaaagat

SEQ ID NO: 75 - DNA, 1597 bp, Arabidopsis thaliana
  source 1..1597: mol_type = genomic DNA
tacaatatctctctctctacttctttactctttctctctcttccatcttttacacagaaagtaaaaaacattctcacaccttctctcccaagtgcgtgccacgcagacaccattatttgccatgagtagttagttgctagttgctactaattaagaaaaatgaattaagtttttaaaacaaacgttgtctagttatataatccatgtccatatacatctagatatatgacttagataccaatgtttcgactcttttaggtctacacgtacgttgcccaaacttcatatttgagacatctttatttatttgtcaacagaaatattgaaacatgcatggatcttgatattctaaaggtgcctgaattaaaatagaacgataacgttttaaaacctaattcccaatttgtagtttatatatttcttgtttgatccatattattacaagcctatatatagtgtatataaaactcaattaagcttcaaaaagtcagaaaactcatcaatagctcaagttccactaattaatgcccgaatttaaagttcatctctatagtttaaaatttgttcgtagctagattttgcatcttggagtggttagttatgtttattagacacttggaatgcttttgaatgtacaatgtccggaaaaaaaaactaatactgaaaaggggatttgtccaagtggtggtagataggaggaggcgcgtgagattggggaaacattggtttcttctgcaatgaatgaaacgaaagcagctatcatcattttctttttcttacttcacaagccgcagaaaagcctctcctaccttaaaatggacatgtacgtgtcgcttttctattgcttcctatctattcgacaacattgaagttttgttctattactttcgtattcaatttttaatttttgtttgagaataccaattttgttttggatctaaactcgttctaatgtaaaaaataataatcagccgcagtatatcaatatgttacattgtatctggacgtgggatatctgaccatgtctccatgaatccatgatgattgacagatttatagggataagtatcctggtttaggtaatccataagtatttatgaatattatgaaaataaattttgatcagaaagattaattagttaagactatgatgtagggtattttaaaatattgctaaaaattttgcgcgctttcagtatgtatatttattttactagttttcggttgatatatgatttcttgaagtaattaaggttattagctaaatggaagagaacgtgaggaatataacacatatgtgatcacatacgaatatatgtaggtttaacttaattggttcgttgattattctttataaaagtgaaagtagatttaaatttgttttccaatcttctatgtttggccattgtttgcaagagttttgggaattaaagtctttttaggtgagaccaagaagggtaaaatatgagtttttgggtctagaatttataagttaaatagtttattctcatttctaattattgttgaccaaaaaaaaaaaaaactgtttggttttgaggtttaattacttattgtatatgtacactctcaatcataa

SEQ ID NO: 76 - DNA, 1552 bp, Arabidopsis thaliana
  source 1..1552: mol_type = genomic DNA
tttactttcattcccacgctctctttaagcacgtgtgatacagtcaccaaataattggctcttgtgatacaattaccaattaaacttgtatatatatgtttatatgttatttaaaggataaatttttatttcgtattacttactttagagttttcattatagggttattatttaatttgaacatctatgtgtttctcctcaggtagaattcatgtttgtgtatatatgggtgtctactatttatggaatatttatacaaaacatctaaacatatcattgcttgaaaattctatgtttatatcataatagcaaattgtttatgtttagttatatataacacaacaatagtactctaagaaaaacagcatcatgcacgtttgatggttgactagtaactagacacacgaggcacaaaggttatcagttgattaacatgtgtatgtatatacggtctttcacttatcacattacaaacttgaaagtaaaatttgtttagcgatattaagaaagtttatatatagagacctcgtactagctggaaatgtatatctaaaatgaaagtacactccctcttacatacagtttaatatattttcaacataaaacatatgtacatagtactagcttattttttaaccaaaacttttgggtttgatagtttaacaactcggtcaagaatctcatattttcttacttcaaataattgccaatatattctagttctttttgtttttaaattatacctggtaattttctaacgagagtctcataattgcaagttgtagtagtcttgcagctggatatgaagcgcgtgaattaaaactaaataatacttacaataaatgaaagcaacgtatcatatcattttctagttgttttcagaagccgcagaaaagcatcttctaccttaaaatgggtttccatgtgtcgatgccttattggatcatactttcttactttttaattcagtttaaaataaattgaaaaatgagataaacatttaagtggttcaattatcataaaaaacactaaaactgataaaatagccacataatataggtcatatcaaataaatgagaaataatatttgtatgcctaaatcaagttatccatgtttgaaggtgtttgcagtgaggtatatatgtttttgtctaataaagattacagttatgattatgtaacataaattagataatatttacagctcgcgatacactacaattgaaacgtggttaattagtgaaactgaaatttgaagaagaggatgaaatatgaatgatattcgttaaaatgatctcttagattgtatagtcattgtcccccattgccaaaagatcaaccacttacataaagtaaaacgcatctttagttaaaagctaaccaaaagagtggaacatatttatttttggattgcatttgcgaatttcatcaatatatggttttgttcaattgggaaatttaaataactccactaaaatgtacaaagtaattttttttcgacaaagaatctatgaagctaaaaaatctacccataagtcacttttagcagaaa

SEQ ID NO: 77 - DNA, 2001 bp, Arabidopsis thaliana
  source 1..2001: mol_type = genomic DNA
catcaatctatatgttgcatgttgtaaggtcacaacttggaaccataaaagaatggtggaggatagcaagtgcgtagaagataaggatcattttacatgataatctggataagacatgcgcatatacatcaagtttccatcaacaagatttatagtacccaaaagaaagaaagaaaaaaactaatattccatcgatctatagaagttgaacaaaataactaaggtaccaagtgaaatatgagtcagcaagcaaagttaaaaaaaaagttgtgtgagacatatcacatgagtaaattaacaacaaccatcatcatcatgatgtagatagtataagatgccatgagaagaagtgaggaaaaacaaattaacctgacaagaagcatcctaaaaaattaacctgtcaaacaattaccaacctttaaagtgaaaaaaaaagttgaaaacactaaggctttgagagcttaaatgttcataaacaattttcctttttcttgccaagatcaatctgcaatttttgtgtcaagcatgtacgagttattgttataaataaatctagagtcgttatgtgagttctactgtattttatgaaaatcgattaactagtaaaatacatcagtcttcttaagaagagaacctaaacacgaaaccctctaataaatgctagattgcatattcttacacatctatattagtgtcaaaaacatgattggactgattttatagtattgaacaagaacgtgactaaattttcagaaattggaaagaaaacaaaaacatgatatatttcaatatctaaatttcaccatatatatataaaaagacatagtgcatgtgagacgtaaaaacctttgtaaataaattttctctttaaatagtaaatagtaccatatcagattaaccatttcagagtgacataatttgtttgtaaccgtatagatccgttttatgcattaaaacgatcgtgaagtagctgtcatacgaaagaacttgtcggcaagagaagcaaagcataagcagaaaaaaacatatcttttagaagcctatcattttcttacttttattcacaaacacacacaatatacattataagtgttatctacataatctaaacgtgtcaatacctgaaaaaacaacaaaactttatttgcacttctttcatttcatgaacaatacataaccagatattagataggacttggacttgtgaatatgctctatttaaggcatctttataggttatatttagaatctttttcttatttcttttttgagcatattattaaactatggactagatctgatgaaagaaagcaatctatttatatattatggtgtaaatattatctttaaaaaatccaatggaggagagtgaaagagaaaagttgaagcattttgtttggactatagagagagagaaaaaaagatcttaactctccttagaaaagtactaggaatcaatttttattttctgcaaaaacaaaagtgtaaaagaaaattaataataatatatattccctgatgaagtaagactaaaataatgtaactacacaaaaatactttttagaatttataatattttatattaatttttaaaaaaggaaagttgataagattggatataagtttggaagcatttatttggccaagaatggcttagcatctctgcaaaaccaacacaaagctatattctcttaaaaatcagttcaacaaaagaagaacctgaacacagttggcccaccaccaccactaccacaatctcctcctccaccaccaccacctagtcaccggaaaccaattcatgccctagattaagaaccagaatccttttgagacaagatctatatatacactctacacatttacttatacactttatctagatatagatgtgtgtgtgtataaaggttctttttcattacttgttggtttagtgataaaaaggaggagagttttcagttgggttttggtttcttggtgagagaagagagaaaaagagaggat

SEQ ID NO: 78 - DNA, 1558 bp, Glycine max
  source 1..1558: mol_type = genomic DNA
acacgtaaagtggacctaaggatatacttataataaatttttataatttatatttttttccatcacatcactacttatatctattttttttctttttattctaatctttctcgtaattaaaatgtaaaatttaatgtatgagtaacattgtatattaaattttaaaagtaaattaaaattatttttatcagcaaaataaattttattaaacttgggtaaaggggtgccacaacttatgcccatagtatcatgtcattaccttcagtcctagtaccagattttcatatattctctaaacatattttcattgtggattcaatcatggatcaaccgttagttgatgttttcgtacttttcgtgggagacattcttcgtatcataaaacaatttcctttattatcttcttaaactctgttctttttttttaaaaaaaaaaatctgttaaataactatttttaggagatgaatagtaattgaattacatataattataagaaaatgattttacaaataattaccttgagaacaatgattaagaaatcgataaataaaaaattaggtaagatgcagtagaaacattaattaaatgtcaatatatatatatactaatttcttaacaattgatcaatctcaataatactgtcaaatgtgcatatcaattggcattagatggttttagtttagatagtgatattgagtttttttttttatgcaagtgatattgagtctgtacatatggcctctaaaaataacactcaatgattaacaaaattgtattttactggtgaaatggaagtattattattaaatataatttcagggtattaaattaagaaaattgttgaattaagtgactccgttttatgtcataaagcactaataatacatacattaatttttaatgaataaattttacgtctctagtttaatattaattatgtactaatagtgttaggattgctatcaaggagagtctaaatatatcatttaaggaaataaaagtacaaagtttttctggtaaaagatagcatttattatgtcaaaaattaatgaaactcgtctaattaatgcataatttgttaagtgatttaaaatctttaatatggtgtatttaatatttattaactactatccttaattagcaggacactacttaacaagaccgtaatagttcaaaattacgtcatagaaagtttaaagaataatgggagaaatcataaattatgttaggattgatgaaaatatgattgattagaacgaagcgaaataaaatttcaagttaatcatcatttggatttttttaaaatgcaatagaatgataaataatgaaatgaatttcatctcatttcatccaaccctactcataaaattatagtatatattactttttacttgtattcaatcgcaattctattctttttaaattaaaaactcagacataattatcatttctacgttgcattttttacaaacacgtctacctacttaattactcttgcagctagcttgttacttttaacttataagagtatcaacggaacatgattgcttaaatgga

SEQ ID NO: 79 - DNA, 1545 bp, Glycine max
  source 1..1545: mol_type = genomic DNA
acacgtaaagtggacctaattttaattaagactaaggatatacttataataatttttttataatttatatttgttttctatcacatcactggctttatctatttatttttcttttttattctcatctttatctaaattaaaatgtaaaattttatgtatgagtaccattatcaaattttgaaagcaaactaaaattatttttttatcggcaaaataaaattatttttttatcggcaaaataaaatttattaaacttgggtataggggtatcgcaacctatacacatagtataatgtcagtgccttcagtcctactaccaaattttattctattctataaacatattttcattgtcgattcaatctctttaattttatcaaattttcaattgagttttatcttcctctcatccacatttttttgctggggttaaagagactaaattaatgttattaatttgaagtataaaaattaactaattcgttaaattaattcataagaggaaagtgggattgcattggagggtagcaagaaagccattgaaagaaaacgcagcagcgatcatttgtataaagtataaactacttcctacgtactatatgtatgaagtgccattctaacatttttttttcttctaaattttatattgtgttatagtatgaaaataaaaattactcactagatttggcctagtgatgggatttgaatagtttgtacaagatgaaggtttaaactttgttgttgacattatacatagaaaaatacaagaaaaaaattattatcatcttaaactctgttctttaaaaaaaaaacagtagtgttaaataactgtttttaagagatgaatagtaattacaaataattataagaaaattattttactaatcattactttgagaacaatgattaagaaatcaataaataaaaaatataaagaaataactatttaacttgcgtttcaattttttttaagattacaatattatagtccgtgaatacacattttccttttgtgaataaaggaagtaaacattggtagggaaccaagaagtctaaagcagtttgcttgtttgccttaaatttttctttagctcgaaggcatgtgaaatgaatatttcattgtgagtaattatgagattttcttccaattaaggcaccaaagatgtaaaaaaatgaaatagtttatttttgtttaatcagtattagagttgcttttaatttgatttttcctgactgaatgctaacaacttttgctaataatagttttcatatgaattggcgttgaaagttaaatcttcagatttaattcacgcattattattattattattataagaattggattagataaaagaatggttaaatttattattattttgttaggcttatacctttacatataatattattattaatttgcagtaggaaacaatattctataataagaatgattattaaaatatgaaataagaacttttcaaaatctgaaattatgcagccacgctgggcgagtagtatctcgctgagcaagcagtgcgcattaca

SEQ ID NO: 80 - DNA, 2001 bp, Glycine max
  source 1..2001: mol_type = genomic DNA
ttaaatttaaattgacgtgctgaactgacatttcgatttcaaattgaccacaagaaaaagagaacgcgtaaacattattcatttattttatttgttctttatttatttcttctaaagtacttagactaactaataacgagttaaatattcctaaaaaaaactaacgagttaaatagctatatggttgaagtgatgttaagattttagttgtgggatgatttgttcttgaataaaaaatacttaatgttcattaagtatcttccataaaaatatctttttctcctcttgatttagttatttattaatattattgtgtaagtgtttataagtttgtgatttatcgtaactttagaatgaatcaaagttctatgatcctaaaaatgtttacacctagacctagttgttaaaaaataaactttcattaagattccttgtttacttttaaaaatcgacttttaaattttttacacatatttttatgtcatttattttaatatcatgtaatacacattattaatcccaaattatactctgtaattaattgtaaaaaatatacaatattatacatctacaattttgttcgtagggtattatatgtttatttataagaatttaagttttatataatataaaaatgacaaaattacattttttttgtctcccagcttatttccattttttctaatttttgtctctcggtaatttaattcagtcctcttattttataaaatcctataatgttagttcccaagccacaatacgttgatcgttcatgtgtcatatctttattggatgatgaccgacacatattatgtttttattggtcaataaaaataacaatgtatttcatcttttatgcagttgacattcaatcacaacataacatatgacagaatcatcatctaataagaatgtgacatctaacagtcaacatcatttgttaacggtcaatatctaaatgtgacattagaagactaatattgcacgattttacaagataggaggattaaattcgtgaattaaattaatgggggaccaaatttataattttccctataaaaatatttataatatcaatcaatttttaaaaaatgtttcaaatatatctcctaaattacttattattataaaaattaaaattctcaccatataataatctataactacatgagaatataaaattttatattaaatccaaaaatatcggaaagaataggggtagttgggtaaatgcatcatgtaacatacgagctacctacgactatgagggttcacctcgtttaaggttagttcctgaatccgaaacagtgagctgaaaatcaagcccgctccccacttcacctttcgacacgtggcactgttgtgacatattctctccaatcatcgttagtcatcagggaccctatttagggcttctcccatttttcttggaaatttccattaccaacccctccctcctcaatctccttatgttccttaatttggtccttctttctccaccgtctgattcttcctacttttcaatcaaccgcgttcccaccacagtagtatccacattaccctgaattttaatttgagcagtgtttacttggcccggtaatcaaagcacaggacacaggtgatttatgaagcaaattaagctaagagaaaaagacagctagagagagagaaagagatatttgattttggagttagcatccattaccattcccatttgacctgataaggatgcttttaatttgctgggattcttaataacaaggtagaatacaattaataatactcgtacatacctcttagtatcacactttagactttacaccatataaaatacatacatgttacagtatatgttatatacaaggcctacatatatattatagagattatatatatatatatatatatatatatattctaaattttgtgagtggatattattactttgagcgtgagaaggggaagagtagagagagagagagagagatgggaaggggaaga

SEQ ID NO: 81 - DNA, 2001 bp, Glycine max
  source 1..2001: mol_type = genomic DNA
gagataaatgtggttttgtacctagtcaacttccgcgtatgatatgagacacgattacattaagcgtgacatgtacgtattggaaatgtatgtgtccaagaaaataaggaaatataacctcaaaaatttgtaagagaatggatttcccatatttgttcaaaagctaagcttacagctttgccgtgctattctgctattgcacagctatgtatatctatatccaaatgcgatatatagcaaaatgaaataaaatttagcaagattaacttaagtataataacatgtttttttacttttatataattagaaattattttgtacattgattaataattacttataagtacagataaaatatacaaaaatatagatttgaaaagtattttaagtaagtactagtttatttaataaaaatatgtgagaagatccataattacatgagaagagtatttttcactctcaagaaaatgaagagacggacttattaaatagataaataatttttaatatatctatgttgaatgaacgaatttagaaacacttcttgaatttatttttttttaaaaaagatgcgaattttccaaattatttttccctattagaagctattttctaaacaatcttttttacaaacaattttttctaataatctcttgtagttaagaaaatagttccacatcctagcgtagggaaaaggtagctaagtaggtaggtgctagaaattgtaaccttcaagatcacatcttatttttcattgtactgatgagtgatgacaacgtgagtttcaattccaaagataacaactaatattagggatgttgacaattatatataaatatatatagattcaattcatatctacgaaaattacttatgatatatagcaaggtaattattattattaataatatcaagagtatgaagccggataaatatgctttttttaaaaccggaggatactatagtaaccttcatatatagatctaccccattgccaccctaattaataatgtgctggaaaatatcttactagtcactttcaaatttcaaccaaaaatgtaaatatacattatttctcctattgattgttttcacatcaagtactaccagctttccatttaattaccacttactggcaatgaaaaaaatctcatctgttacttttccaataattctgctattttaacaagtgaagaacaatgcttcaaggtgtggcttcactaatgaagttatttatactatagtgaatataagccagtgataaagtgacacgtgtggaatccattttaaggtatgtgacgacgttatgatactcttgctgtgcctctctgtagacagtagaaaatgacacagattttcactgcaaacagaaaattataggtaacggagagataggttgaaaaaacaaggtcacaagaataaaaaagtttaacgtaggaaaaataatatcatttttgtaagtaatagtatctatctatatgacagctgtcactacgcgccagttttctcccactggagcaacgagtgttgagtgtctcacacgcgcctatgttgcgtgtgggggaggtgattgtcttccaaaaatacgttaacatggaaaatcatgggaaagagaaagttacatgaaactagaaagaactccaaccctaagctgcactccatttttttctggtggagatggaaaatcctgaggataaaaaggaaacttagaaaagagagtacatatacaatacaagacaacttcacacacacaacactttcctctctctctctctctctctctagggttccgagtttgaagttgaaagtgttgaactatctctagctagcttttcacaaatttggttgcacttgcaactgaggcaggaaagaggggcacgagaggaggaagaggaaagagaagaaaaaccaagaaaagtggagaaagggataggtgttgtacagaaatagaaattaaaattgagtgaaaattaaagagaagaaggagtgatagggagatat

SEQ ID NO: 82 - DNA, 1643 bp, Glycine max
  source 1..1643: mol_type = genomic DNA
taaaaatatatataacatggttttaaataataaaacattaaaataaaataataacgtatcaaataaaatcataaaaatatactaataaataagtcaacttaatattatttattatatatttaaatatatatatatatatatatatatatatatatatataatttttttatttaaaataaaaataaaaccgtaaacaatttaaattaaaaaaacacaggttaacaaaacaaaaacaagttttccaaatgaaaaaaagcaaaaaaatattttacgactttaaagtcataaaataaattttttttttaatgaaatcgtaaataattttataactttgatttaaagtaaaaaaaaattacgactttaaaatcttaaaaaaaataaaatttaaactgaagttgtaaataattttacgacttcaattaaaaaaaaataaaaagtcatatcagatgttacgacttttgagttagaagtcataacatctgttataacttatcccattttggataatttttcaaaatatacaccttattggaaaattgggtaaaattttacccctaattgataaaaaagtcaacgatataatttcccatttagattccaaccattaacagatttttaattgatgaatagtataaatttttatagttgctccttataaattaataacttgaaaaaataatgatatttaatggaatctgattaaaattttgaaaaacaaaatataagaactctgtcaacagcttttaaattagagatttatcttaaaactcaataaaattataatatatggatcgatctaaattagtaaattctgaatagtactaatgtaaccttaatactattattatccatgcctatctgaaagactaaattcccgttttgcatgctagaaaatgaaagccagtgataaagtgacacgtgggggtccagtttaaggtatctgacgacgttcttgcagtgctctactaacgtgaacacagtggaaaaaaatgacagaattttcttaatatcatccacagttaaagacagctgtctccacgcgcccgtgtattagaggcgtcccacaagccaataggttataatgagcgtgtttcatacgcgctataagtgagaacgggaaaatattttttgccaaaaataccttaacccagtaaagcatgataaaaagaaacttgtatgaaacaggaaactcggaccaaccctagcaagatgcactccatttaatggtggagattggaacaaagagaaaaatagaaaaagaaaaaaagtgaacatctgaggataaaaaagccaagtttgatagacacaactgtgttgttgttgttgttgatgatgtttgctttgttctctttctctctcctcaaattttttcctctctctctagggttccaagagtgtcttgaactactccagaaccctctcttgtttttcacaaatttgggtttagtagagtactgacccagaaaagaaggttcaacaggtacctaagagaggggatagaaagaaaaaaatagaaactgtacgtataaaagaagaaagaaacaaagatataaggaaaaagggaaaatattttgaataaggaagatataaggcaatatataaaaatattagtatttgataataattaggtgtagaaatattat

SEQ ID NO: 83 - DNA, 2001 bp, Glycine max
  source 1..2001: mol_type = genomic DNA
agctgtttcacacaaactattaattactggccaggttaattactcaagaagtagtaatactatcagtattgatattaggttatattttcctattacaagtttatattgtcctgtacaactttgtagttttactaagtttctaaactaagtaaataattacaatataaaaaaatctttacattactccgtctatcctataacaattattgtcttagaccattttttatacaaataaaaaaaataaaaaagtttaattacttatttacttcttatactttttaaatttatcttttttagtttctataattagtaagtatattttttaatccacaaaatatatattttaatttctaaaatgttcctgcagttaacttttttttaactaacaaagttaaaaaaattaacgagataaataccttttaggaattaaaatataaatttgaaggattaaaaaatttatttattaaattttagagactaaaaaattcatttattaattataagaactaaaaggaataagtttaaaaattataagaattaaatgggtaattaaaccaaaaaataataaatagataaaataaaataatatttttataaaattaatattatataattattaatttatttttagattttgtagttgatcgttaatatagccaacgttcctgctgcaagaagctaccccggatattccgtccccatctaccaaaacaatcggaaatgtttcagattttatagttgatctcttaatatataagagatataggtggaaaattcattaatgttatattaaaaactaaaattacaattatattaggataaaaatattttcttataacggatgaagtattgattaattagaaattttttatgtatgatgattttgttaatttttatgataattattttaaaatcatttataacagatttttaattaatccgtagtataaattttttatgctatttgtgtatcgaaattaaactatgttaaattactccttataatttaataacttgatagaaaaataatgatatttaatgaacagggatctgattaattttttttaaaaaaatataagaactctgtcaactgcttttaaattagatatttatcttaaaactcaataaaattatatgtaatggatagatctaaattctaaatattaatgtaaccttaatactattattatccatgcctatctgaaagactaaatttccgttttgcatgctagaaaatggaagccagtgataatctgacacgtggggtccagtttaaggtatctgacgacgttcttgcagtgctctacttacgtgaacacagtaaaaaaaatgacagattttttcttaaaagcatccacagttaaagacagctgtctccacgcgcccgtgtaaaacacgcgaccccgcatcaaagagggttacactccgagtgtgtttctcacgctctataagtgcgattgggtaaatatttgtttgccaaaaataccttaaccgagtaaagcatgataaaaaaaaacttgtatgaaacaggaaactcagaccaaccctagtaagatgcacaacattttatggtggagattggaacaaagagataaaaagaaagagaaaaaaagtgaacatctgaggataaaaaagccaagttagataacaggagagatgaacacaactgtgttgtagttgttgttgttgatgtttgctttgttctctttctctctctctctagggtttcaagagtgttttgaactactccagaaccctctcttgtttttcacaaatttgggttcagtagagtactgacccagaaaagaagattcaacaggtacctaagagaaggaaagagggggatagaaagaaagaaaaagaaatagaaactgcacgtataaaagaagaaagaaagaatcaaagaaaataggaaaaaagggcaaatattttgtttgaataagatataaggcaatagtaatattagtattgttgataataattaggtagaaaaaatattat

SEQ ID NO: 84 - DNA, 2001 bp, Solanum lycopersicum
  source 1..2001: mol_type = genomic DNA
aataagatgaaagttatttttacgctaaataagatgaaagaatcttatttttatgttaaataagatgaatattttattttaaagctaaataagatgaaagtttaagttaaatgagatgaacatttaaattttaatatatacgacataatcatggcaatacacacaaaaaaaatatttaagctaaataagaggtaatttttatttgtaagaatgaataactaaatcttgtacaaaaaaaaaaaactaaataaggtgagggtatttttgtaaacaaccaacaacttattctcaaaaaaaataaataataaatattaatttaaatatgacaaaccaaacaaacaactaatccctatacaacatattccagcataactagtattcaaatcaaacaacccctctatgaaatccataagacaagaaataaaacaaaattgcaaaacaatcaatagattaaggtctaataagacataagtagataagtgaatcgttgaagaagacattgaattttgatatagtacaactccatacctttttatggttattcatgttaatcttagggtattcgatataactttacaagtaaaatctgtaaaagatatacattattcttatctttatttagaaagattatttttgattgactctcaaatcaaaaatagtgtaatcgagtatgatttttcaaaaaaacaaaaaaacatcaatataacaatatgagcgaaataaactaagtaataggcattgtaaaaaaggaagaaaaagatgcttatgacttatctccgttactgaattggttttcctcttctccctaataagcaaagagccaaagactaatattgaaatgagttatttcctcatatgatattcgacattaatcctttcatcatcaattagttttttcgaatgaagttagactcaaaatcccttgtgaaactttcaaaaaattaggtttatgagttgagttggtttcagaatgctaaaaataaaatgcataatacatatattgtatcctaaatttggcttcaaattttaactttgacctctaactttcataatgcacaaacaaacactttgactatctaactttcaaataaataaacacatgagtcttacatgacaaaaatatacatacgacaccacgtaggacgaaaaatgacatgtaagacatgtgtgtctatttgttcaattttatacaagtttaactgtttaagtgtctacttgtgcacacccaaagtggaagggcataaatgttatttgaagtcaagttaaagccatacaaaataaaaattgaattatgtcttatttaagatggataagtagtttagctaaataaatttttgttagtaagttttccatttctataaatttacagtgaagaagtgagttatttgcttacgcatgatatgtacgtaatatccactaatacaccgacgcgtgtccagccaatttagtacacgcctttgttctttcactgatggaccgcacaaatgagaagcaaaagctccaacaaactaaccaagcgcgtgtatcccagacaacgaatagttgttgtgcgtgtactccacgcgcatcaacagaagcgtaagactagtccctataatgcattggctaaatacatgaaacttgaaaaagtagtggaaataagaaccaaccctaaagctctaaggtatctaaatttggagtacctaagcaaatcctaaagttaaaacaatgaatccctaaagaagataggaagaaatgccatagataaaaacaacccatgttcactttttctctctctaaacattgaaattcaaccaaaacaaaaaacaaaagttgataagaatcctttctttctttctttgtgtgtgtgtgtgtctagctagggtttgcatttctttcacaattttggttgtttcagtaggagagaaaagaggatctaagagttagccaagagaagaaattagtgagaaaataaagtagaaaaagatcatcagaggaaggagggatgggtagaggaagagt

SEQ ID NO: 85 - DNA, 1817 bp, Solanum lycopersicum
  source 1..1817: mol_type = genomic DNA
ttacacccccactaaaaaacccaaaagttaaaacaaagagcatctaactattaatatcacgttagaaacgtgcgatcatgtcatttaaaagtttatatcgatagagatagtacatttttgttatttaattatagggggaaaaaaacaaatatatctttgaattatcattaataatatgcagatatcttccgtcatacttttgggacgatattggtgtccccaccgtccaaaaactagagcatatatatcctttactctaataagactgaacagagacacgtgacacaatcatattcattgatcatatattcaatatgaaaatgacaagtcggtggataagattatgacaattgtatgtctgttagtttaaagggtatatatgttctatttctgggtggcagggcaccaatatgtcaaaagtatgacgaaaagtatttgcttacgacaaaaacgatacttgggggttatatttgtcttttttcccttaatggtatcctcaacaattgtcaagctaataaagcgctacttctaattattcaaatgatcgagttattttatgtatgttaatattgcaagcgacttaatatctccaaacattgttcgaactcagcacgtacaattatcaaaccaaagagcaaagtaggtctcgacaaaacgaagagtatcataatacctcgttcgttattctttcctaaaatataatttttatgttgaaagtaaaaaaaattagcataaaaattaatctattacgactgacgcgttgttcaaagtcgagagaataatttaaagtggtaggaaatcaaaattgatttgggtatttatttaactcatattataataggctttttttgttttaataattcactaggaatatattctctttttcaagataaattaggttttgcaatgctattatatataaatagaaacattactagcaaattttttgagtgttagaagtagtagaaaattatttatttaagtaaatgtatagaattttgaatgatggagaaatgttgaaagattattaaactgtgaaataaaaggattgtgttggcttattggacggtttttaattaatgaaggaatattggacggttgctgattgatgaagacagaaaataatttgaacgaaaatgatgaatattgtgaaaatatctaattatgtcattttaagggaaaaattaatttcacttcaaatataattagagtattattagaattttctttttctttctaaataacacaacaatttccttttgtcaaatcaaccaagttattagcatcctatggatcacacctaacaaatgtacataacttcttctattctgatttttatgtctcatttttctttttagttattaaaaaaaatatattcagttttattttatttttaaaattctcatttcacactttgtaattattaattatttattttaattcattatcaaattatatcctataaagtagaactagtgtaaaaacaaattatctttatttttaatcgagtcaactcgtccttcatacccaaaatgtatcccacactcactctattaacatacagtgagtagtacatgataagtttgttaaataaattttataccatcagtacatgcaattttatgtcaataaattacaaatatagatatttgtgtagtgcacccttcttttatagtaatagatttttagagttagctagtaagtgtataatatcccttgatttaataacaattattcactatattatattgagatgtttatcaatatttttttactttataaaattaataaataattttatattaagtttttaattt

SEQ ID NO: 86 - DNA, 2001 bp, Solanum lycopersicum
  source 1..2001: mol_type = genomic DNA
ttcaagaatttatgttcttacatatttatctatctatctatctatctatctatctatctatctatctatctatatatatatatatatatatatatgaagtctcccacttatcggtaagtgtaatttcttttattttttttaatctattaacaaatttttaaatttttccctgagccataacaattgtataatttaaatatgttctttttcttacgaaatgtcataacaattattctcttctttatttttattttaatattgattaaagaaataataatttattattttctcaataattcatttattagaaattcgtgagttatgtgataatgatgaataatgaaatttatgtcaaaatattattaatattttgtataattattttttctataatttttagtgtatgaatattaaaatattaataaaggattatttgcaagttaataagaaaaagacagctcgtaagggaagcatcgacacgttggtaattaaacactggcgattgtactattacgttgatgttcgtgtccaaaaggtaatacgatgtatcaatgtgaaacattaatttacccaaaaccacaaaaaggacctacacttattacatatagggtaactccatgtacacctatatatatacatataataataaaataaataatatacattcataaataattgcatatatatgtagagacacactttttaaggtgtataaaaatataactttttagagttcagttctcaggtgaaatttattagtatctgctctatcattattaataggtctatgtttgagaactatcaccaagttgattgggctattttagaagggtagaggtttgacacaaaagttaatttagataatatttctaattgatgaatataagaaatatgtttttattaatcgtacagatacttatagttttacttaattttgctattagattgatttgtacaattcgtcttataagtataaaatttaaaatttgtataattttatttttgattatataaatcaagaattttatgtatgtttgtcctaactatttgtatataatttataaaaaaattgataataaattgtcttgtttgtatactgagaaacgaaatatacaaacaaatttctgaaaaaattctgaccgtataaatacagaatttatatatatttaacgtatttatatattcgcaagtgaaacttataagcagacataaatatgcataccaacataaatatacaaacctcaatcttaatagcaaataaatataaatataaaacacaattatcgaaactataagtctaaaatcttaatatatctattttatttgctattagtaagatcatgagggggaggcagatgtgagttatgaggattcactagctttgactaaattgttaaatagttaataagtatacgtaacaaatttgtgtacttagtaggcgtttaattatagaacatttttttaagaaaataaaaatttctttatttaagtttttcaaaaaaaaaatcatctcttaaaatttgtaaaagaattcatatttgacgtgaaaagattattacgtcatatcagggaattaaagaataaccagtttaaattatattattgtttatttattgactagcggatatttatttaattatattctcagtattattttatatgacatcatttttttagttgatctcaaaaaaatgttaccttacttcacacaaatattcaagatttatattaaactacaagtttccaaaaaataaaaataattcttaaaaataaaaaatgtgtcacataaaatgaaacatatatatttaaacatttatatgtgacttatcaatttgagttgggttatatgaaagtgacaaacgattagatagggcataacagtgcttctctctctttccattaaatttggtagtccacatacagagagagtagaaaaagtgtgttaacctagagtggaataatagtgtttttcttgttagagacaagaaaagagagaaaaat

SEQ ID NO: 87 - DNA, 1882 bp, Solanum lycopersicum
  source 1..1882: mol_type = genomic DNA
gttatggttttggttcttttttctgtccttttgttcttagggtaataaataaatagctttgttgggccatggggtttcctaatattacaacattgtatctatttttcttatatctgacgtataaggtataataatttcaggataaattttatcgtgtgtttagttgaaagcttaggtagttattcttagattatttctcctaccccatttatattttaataacggaatatgttatctcatatgtatagcaaataaaataataacttattttgaagtaattaattaattctgaaataatttattcacgatcaaatgatccttattaatattactttagacccttttaaatattgcatttttggtaattctgcaaactgcttttgttatttcttctgcctgtgtcccttgtctctctataattaatatataagtaaaatgtgttttgatgtttagggccaaaaaaagaagacaagtcaatactattaaccattattatttttggtttctcaccaactaaccttttaatatcaagtttttgcccctattttggggtggtatttaatttgtatcaaattacaaaataaataaaaggagacataagtttagaattttttttaggtataagttgtgtagtacctaatttggaagacataagttaaaagaattttgaagttagattgatttggaatcaaaattaaaattttatttaaataggtaattagattataaaaaataatatttttattggaaaacaacataatttgttgaaaactattaaatattatgaaaattttaattatatatttttatacaattttttaaatgagtaaatcacgattcgtaaataaagtatcagaaacgtttgtaattacattttattaaaaagtttccaattctcattattttataaactactaaaaataaaagtttttcccacatttccattttaatggaaattgtaagacataatattaaaggttgtaggtatttgtatcccttactgggattggggagtgtttgaaaagacctttatatggagatacattattagccccttatattggaaatatcatatctataaaattcaaaagttaattcatgacgaaaaaaaatatctaaattatattaaagagatcacatattttttctagctatgactggacatatagtgcgtggataattttagtatgaagatctaacatcaaaaagcaagaatgggatgaatcgtgtattaatatcatgtaaggagcgaactcataagaaaaaaattgttcagatcatattaaaatatataccaattcttaaaccatcgagacggagacttttcacaaattacatacatactagacattataaatatctttttacatcatcaatatatagtataggtaagtatatcttactacactatcaatttaaattcatcaataattactgaacaaataattcgatcgtgaatatcttttacactgtcaacacatataattgcaagtcgaaaagtatttagcaatcaagggttttacaagaatttttggtgctaataaaatgtataacaaccttaaatgttgtttgtttttccttaaacaagacatagcacactcccccttgatgtatctttttgggacataccacatgtttctgttacataagttggatattttgatattagatacctaagaatgtgtcatcatgttactccatacatatcaaacaatatttcacttcaatttgaaatatttaaaatcaagatttgtaatcacataaactaaaatacgttaacgtaattaatcaatcttgattcaacacaaatattaatgagagaaaatattgattcaaaagataaaagcacatagtgtatgggaattgatattcaaatacctaa

SEQ ID NO: 88 - DNA, 1574 bp, Solanum lycopersicum
  source 1..1574: mol_type = genomic DNA
aatgaaaaagaaaaaataaagcaatagatcccatcttatttaaaaagggaaatagtacaacccactttttttttttgtatttatatttttgattttgtttctctctatttcattcgttttaatttatgtggggttagtgcgatgtatttatttcaaaaataataatgtgggtgaaatagtgatctttctattaaattagatattttatgtatacattttttgtcgaattaatataatattttctcattaaacttacatgttccaaaaggatcgaatgatgtacttggttgaattgcgaagtaattttttttagagaaacaacgatcaattctcacttaattttttttttatattcacgttctaaaaatcctaaatctaagaaaaaaatgggagtaatttggaacttaaatctaagacaagtcttaattatgtcttgtttagctaattaaactatcaaaataaaaatattttttatttaaaatagtatccaaaaaagaattacttttggagagtcaatttttgaataccttgttaatttgataaatatttgtatttatattataacaataatttatacttgaacaaagttttgaaaatactttccattttgccctactatacgtactactcaaaatattaaaacccatttttttcttaaaagtttggccaaaacacttcaatttcctttaaaagttggccatactaactacatttaatttgtacaaatcaaaattattttataactcatatcattggcggaacttgacgtgagttgtctaaaataaacaaagtttaataaaagtgtataaattaaaattactgttaactttagaaaaataccaatgagtttgaccttttttacaagttgaacaatacaaactttaatcaatattattaaaatatattttcattttattgatataagaaattactacaatttataatacttttcctataatttcaaatttttaaattttctgaaatattaaaccaatctaatctaattcaatttaatcaaattaattcatataaagcaaaattaaataggaaaatattccatatcaaagtgagtagtttctctattctatttttatatttcaaataggaaaaattcacttttctataattaagaataattcaattttaattctttcccttttaactcttatgaaataatttataatcacacaactattcaagtattattttacaccataaatttcaaatcttttcattttttcttcttaaatattatactaaatcaaataatatcatataaaataaaacataaaaaaatatatattttgagaaattaaaaagaaaataatgtcaataatttgtttctttttttttctcttaaatagagtacatgaataggatagaataggtgtggaaaaaggtgtaaataataaataaacaatttaacaagtactatgaaaaatggtattataattatttaaaatgaaaaccagatggtatttaagaatgtgttgtcgagttttgattggttgaaaggacaacatagtatggtactcatttttgtatggtggatatataatgtctatcacagattt

SEQ ID NO: 89 - DNA, 2001 bp, Zea mays
  source 1..2001: mol_type = genomic DNA
agtctcttgttgggctggacgcctccatcaaggcgcgaggtcaagggcacagttaaccgttgtgctaactggtggagatcaattccaacacactggactagtgctgggactaacttaaaaataggagcctaaagcacggtccaacaagaaataaaataggtcgggctaccacgactcgaaggtgggtctagacctgacctcaagttacggcctgttgggcccagcacggcccacacatatgggtcggtttgggcctacacggccccaatgaagcctatattatttaatttctttagtttcgtaaatttataaactttatattgttgtgatatttggactttatgcggtcaaatgatgctagcattgtttaatattgtggttgcaatatttggattttacgaggtttgaatatataggacgagcttggaccggcacgattcaacaaaagcacggcttgctttagagtagaactgaccattgtttctacttttcaggccctaaaagtttttttttatcttcttagcccgatcccagcactagactggactggactgagaccttgtactttgaagtaagatgcatgagttcgttatgcggattcgcaaggcgcgcgtacagtacagctcggcacccaacagctagtagtacgcacgttccgttaatccgctggatggatggatcgatcggagacggacaggggcggacgcgcggcaaacgtacggtgcagttattgtcgtccgccggatcgatcaatcgaccggcgcggcggacggatcgaacagtgcccgaacatgcacgtccgtcccctacgcctgcggcgtgcagcatgcgcgcggatcgtagtccccggtcgatcggatgcgcgggccggaccccggcgctgccgaccgggaggcgggagacttggttcgtttcgcacctgctgccttgtctcgcgctcgcgcggcgcgtcaggggtggttggtcgcgtacttgcgttggctgctgcctgggtggctctccgcctctcctggccacggcgagactgatgcgcgcgctggcccagctttggtcgctgttgcgagctggtctggacagcgacccggcccggccggccggccggccgccgagaccgaaaggaagcaacgtacaaccagcaggaagcaaggggtgagagagagcgagagaggaggggcgtgcagccgtccggtccagcaggcgacggaatggaggacacgccgggcaggtcgctgtgcgcctgtgcctgcgtgcgcgatcgcgagtggccagtcaccagcaggccggccattaaaggagagcacgtgacggcgcgccagtcgcttccttcgcttcgcttgctcgggcgccggcggggaccaccagggtaaaagccgagcgcgcaggacgcgacggcgacggcgacggcgacggacgggacgggtcccatgagcccatcaccacgagcggcgtggacgtggaggtggatggaatgaccgatcgaccgatcgatcgcgagtgatgactgatgagtgtggcgtgactccgatccctgatccctccccatccctagctttccggcaacgcgctaccgggccgggggcctagggtttccccccctacggatgctttgccggaaacggcaacctgacgccgaggcgcgcgcaccacccctgcgcccaccggctccttccctgcgccgcgctgatgataactcagtccctgcacaggccccggccccggccccagccccaccaccgctactccactaggccctggttgctagccagctcgcttgcttgcttcgattcctatcctagcccccgtgccatcgctttcctctcgttatttagccctccgttcccgaccctcatcctccgctccagacttccagcatctccgctccggctgcgctctgccttgctttcctgctacctgctctagcgcgagcgagagaggtacggcggccgatctggcggcgcaggcggagggctcggccggggccggcaagtcggcgccgaacat

SEQ ID NO: 90 - DNA, 1032 bp, Zea mays
  source 1..1032: mol_type = genomic DNA
acatgaggagtgggttgattccactaaaattgatggaatgaacttattatgcatcaactcataaagcatagagtgattccacaaaccaaacacaccattagtttattgaccaacctcagttctttgatatataaaagttgtttgatccgtgtgcaacaaatagtttttcaggaatttgggacgtaccccatctcttaaatgtctagactgattggacatattaatctaattttagtagctaagtattagctctcaaacacctcctgggaatgctgccctaaaaatggaatatcggtacgtcggatcggagaaaggttggggtatcgtggtccaaagcatgcgaaagcaacggcgcagggctggaaacctgagaccaggtgaggtgcacgacgacatgcatatggtttgggttaggcctaggagctggttctctctccatgcatggtcagctcgccgcttttgccgccttcgtgtatggcttgccccatgccatgcatcgcgccgtgtacacttgtggcgtaggggcggggccgccgcatcggagcgcccccgtttcggcacggtcctcccagttttagggtaaacccagctagggtagtgggggtaactggccagcgccactccaaatctaccctccttcaatttaaagctgagaaatactgtagtatatactagagtagagcgagagaagggagatgtggatagatgggtggatggggacgcgtgaaaaagatgcgagagagaagagacgaccggacaggcagccacacacagtaacagtagtgaaccctgccccttttcccggtctctccactgatattccgctcctgtccctgtcctccccggacggagctaatagagccggctctgctcatcattatatcgtcgcgcacaaatgcgaagcctagcagcacttgtgtccggcggccttgtgttcgtgggggatgtgtggcatattagctagctgataagcggccggccgaagagcaagcgcagtgagaagaagaagctcggatcggaggaggtcggcat

SEQ ID NO: 91 - DNA, 2000 bp, Zea mays
  source 1..2000: mol_type = genomic DNA
tgtctcgatctctcctctcctctcctctcctcccgttggtgtgactgtagtagatcctttgcccgtgtcagaacaagctgctcctcggaccgggtaatgttaaacatcggaggagcctttgcctaggatccgtaacggggaggaaagagaaaaaaaactaaggatgattatggataccgtgtaataactgctaactacagttagcccatctcagcggactctctgccctatattgtatgtcactttctattataaactacactatacaacctatgatgtaaaataatgttttgcacgttcatatataaatcagtcgaagaaagggtgcctcactacagggaatggtttctattggacaccttagcattcaatcagtcatgtcccccccccccccaaaaaaaaaatgcacccatccagtcgatttttgtcatatttgaattcggtggtgctccatgcacgcgtacctgctttgaccaatttatacgatcaatatataacttacgttcttacggttcttagactttatgagactttgcaagtatgtttggatacaaatcacactaatgtgcatctttgtaaactaaattcttttgattaaatttgtaattttaaggtttaacctgtttttgttgtgtagacgacgttaggcaccgatcgtcgcttcgctatatatctttgttgtagacgacgttagactcctagattaaataagcgaaaaccgatcgtcgcttcgctatctttgtttatttgtttgtggctgctctacgctgaagagcccacaggccacagccccacacgacacgttaggcacccccacccaccatccgcgcataatataagctactgcaaaatatatgccggcggagcccgagcgagctttgtacttgctccgccgtggcctggctccaggatgctttggatttcgtgcggcgccgtacgtccaggcaaacagacaagtggagctgcatgtcctaaaagcccggcaatcaaacacgctctagcagcagcatggatcacagatatcagtcatggggtggcgctggcgcgggtgggtggccaggtggaggtgggtgcatgtcgtcgtcgtcgtcccatacagaaattggctcacgtatgtatacgctgcgtacaggcagtagtacacaattactagcaccaatgcaatccaacggatggatcttcgcacacccgccacccggttaaattaagctactcctacctctcccagtctcccttggcctgcctctatatttttgggcagcctccaccagccgggcggatggggttggatcgtcgtatctgaggcggcgtggtcgtccaaggcgaaagcaacggcgcagggctgggaccctagtaggtgcatgaggtcgtgcatggcgcgcgagatgcatggtttgggttaggcctaggaggttctctctccatggcatgggtagctcgcgccgcttggctgccgttctcgtgtatgcgcatgcaccaggcatttgcaccgcgccgtgtatatttctggcgtgggggccggcgccgcattggagctgcagccccgtttcggcacggacacgggacacctcccgttagggtaagcccggggcagtgggtaactgcccagcgccactactccgaatttaccctccttttatttttaaagcttgggagaggggagaatggatggatggatggatgtagacgcgtgaaaaagatgcgcgagaccggcagcgtgtgctacaggggcagccaggcacacacacacacagtgaccctgccccttttcccggccgtctcgctctccaccgatattccgctcctcctgtccagtcctcctcccccgagccggctcattatatcgtccgtcgcgcagcacaacgcaagtttgctagcggccggatcagcagccacaaaacgaggagagcaaccacgctgcacacagagacgcccgtgtgtgagatatagagcaagctcgatcgaaggaaggagggaagctagagatcgtacgtcgccat

SEQ ID NO: 92 - DNA, 2001 bp, Zea mays
  source 1..2001: mol_type = genomic DNA
gtcgaaacgccgacaatttgttacacggttaccactgcacagcgagtacgacaacaagcctaagatagtccacttcttcgcattcctatacttctcactgtgatctacggcactacgacactatttcgaggcaaaaacagagggaagatgcctaaccaaaattctaatgaagaattacaccgtgaatgatggcggcggtggacgacggtggcaatggcaaaaccctaaccctgcatcattttcccttcacctttctattttttccacgtgcatagcatggcagtccctcgacaatagaaacacatgtaaggtttcagtggatatatcccaatcgttgtacttaagcagcattcataaggtgatttagtttttttccctaaatcttgcaacgtgcataacttcgcgattgaaactttaattaatacatgtaatcagtcgaatggaatgcactggtttttaggcatgtatatgacctcaaatcatccacgcaaggaatggataaaatgacaaatcttcattgtcttcttaaattcaggcgatcttcgtaaaaacagttgattcatataaaaatagaaaatatatttggagaaattttctgcatgtccttgtaattttgcttagtaccatatttatcatcgtaaatgttataatcccttctatataccactctgatacaatttcgtacctttttaattatgccaacgcaagtgggcgagtgatatagtagggactagaatatctgtgatgataaataatgtactagacaaaattacagtagcatacatgaaattgtcccttatatttgccatgcattttttgctattacatcttagcataataatcttggaagcacatcgcatgagttatcgattcaagttagatatatagatgcatagtagacaacatttagttacgcaacggcatctggttccaagaggagctttgcaagagatcaattgaatttagggcctggaatcgttaccaaatcagtgatgattgccggttgggttcacatgagcagataaaaaaatggagatgatcatcgtcctttcagaatcgctaacagatatggaagtgatgaagtccaggactccacatgatcttactacacacatgccagaagtctggaacaagaacatgccggatccctaagcagaaaacggatcgtatagcggtctcctttctggaaaaagcgacgcaaaacggatcgatgacctctctcaccatgtcaacccgtgctgaggagctccgtcctgtctgcacgcactatggcaatggcgaccggcctcttccgagctgcgaatcataacggcatgtctgcaagctcgatcgctgcaagcatgccttcccgttggaaggatcgagctgctcgatggaggaaagaggaaagcagcgcgccgcgcatggctatagcagttggcaggtgagggcctctagcggcaacgggggcctttgcccaaaatcacggcaagccgaggttccacctggaaaaatccactccgttgctctctcccttttctctctctctagcatcgacctgtcgaatcctcactggtctattcagttcggcaacgaggagagagatagacagagagacggacgggaggctgacacatggccggatattggggtgcggaattgaatttggttaatgcaaaaggtggcgtgtgaggacggaccgacggaggcaatcacgagacgggaggggttggccttttgcctctagggttccgggtcgcccaccaccttaccggaaatggcaatgcgggacgcgccccaatcactcaccacaccacccctttcttctcgcccttttaacccaatcgtctctccaccacccaactcctcctgcctgtgctctcctcctttttctccactccgcttttgcttggttccatcgggccggagaggagaagctagctaactagcagcagctctgttggtgtgtgtgtgtgtggagggtagctgcagctggggctagctggaaaggtcgggagccat

SEQ ID NO: 93 - DNA, 668 bp, Zea mays
  source 1..668: mol_type = genomic DNA
atcgaagatagggttgctcgaaacaaaaaaaaaatctgcagccgcagactgtatgccctacgctattttgccgtaggagagttggaacctttgatgtagcgtttgtactactcggggtacgctatccactatttggttgaggtatggtgggaaagaaatgataaaatatatggttgttggtgtagagttgatatatgaaataaatatgactgcagatgattattgtgatatatggtaaatacttttgatgataaaaataaaatatttttttagagtagtacggatagccttatcgacctatgtacctggcagcgctgtgatccatcagctagcgaccaatcactcgcccaccacggcatcgccaggagctgttgctttttgcgagctgcatggtgggggcctccatcgaaggcgaggggacggggcagagcgccctgatcgacgcgtgataaccgattgatgtgcccccagttcgcccgtaccagcgcgcgccccctcggcctcgtcctgcgtccgcctgcgtgctggtgcggcgcgctcgcgctggcggacgtccagaccgcaataatctcgcggcctcgcgcgaggaggaccgaggacaggaggtgcgatgcgatgcaataaaggttgccgtacgcgagtgagtcggacagagagagagagagaga

SEQ ID NO: 94 - DNA, 718 bp, Zea mays
  source 1..718: mol_type = genomic DNA
ttatttggtgcttcaccgccggagccaggctagctatacactttgtagttgtttgtaggggtatataggagcagtatctacagtagcagcactcttcaccggtccagctagtaaatgcggtatctgtcgtctgtcgagctaacctatcatatcagcgccatgcgccccggccggtcgtaacctagctaggagtagctagctaggactagctagctaggagcctaagcgcccagcccagtgggtggtagcctaccaagggtgaggaagaggagctgagctaggaccgcgaggcgagcgagatccaatctgcaccccacgaacgaaaagattcttccccgccggcgtcgctctctcatccgtcgacgtctcgctccttccttcctctctctctctctctctctctctctctctctctctctctttctctctctcagccagcagcaagcatatagcccagcaccaccacatgcccaagggccctccctctccctcgtcgtcgggccaattgagtgtgagagctcgaaaaaacccaggggatcggccggaccagcagcgagcgagcgcgagctctgcgtgcgtgtgtgtgtctgcgcgctagattagatcacctccatcgtcaataattgcaggcagatccatatagtctgctggtggcgagacaaagcaacggatcgtcgtcgatccggagagcggaaagcgcgcagatcgcggcggccat

SEQ ID NO: 95 - RNA, 21 nt, Glycine max
  source 1..21: mol_type = mRNA
agaatcttgatgatgctgcat

SEQ ID NO: 96 - RNA, 21 nt, Glycine max
  source 1..21: mol_type = mRNA
ggaatcttgatgatgctgcag

SEQ ID NO: 97 - RNA, 21 nt, Glycine max
  source 1..21: mol_type = mRNA
tgaatcttgatgatgctgcat

SEQ ID NO: 98 - DNA, 21 bp, synthetic construct
  misc_feature 1..21: note = Description of Artificial Sequence: Synthetic oligonucleotide
  source 1..21: mol_type = other DNA
atgcagcatcatcaagattct

SEQ ID NO: 99 - RNA, 21 nt, Glycine max
  source 1..21: mol_type = mRNA
atgcagcatcatcaagattct

SEQ ID NO: 100 - DNA, 21 bp, synthetic construct
  misc_feature 1..21: note = Description of Artificial Sequence: Synthetic oligonucleotide
  source 1..21: mol_type = other DNA
ctgcagcatcatcaagattcc

SEQ ID NO: 101 - DNA, 21 bp, synthetic construct
  misc_feature 1..21: note = Description of Artificial Sequence: Synthetic oligonucleotide
  source 1..21: mol_type = other DNA
atgcagcatcatcaagattca

SEQ ID NO: 102 - DNA, 328 bp, synthetic construct
  misc_feature 1..328: note = Description of Artificial Sequence: Synthetic polynucleotide
  source 1..328: mol_type = other DNA
ggcagagccgtgcccgtctcatcccctgcccgtgcaagcagctaggtaggacgatttgagcgtggtgttaggccgaaccgctgaaggaagattgctccactgttgactgcatttgggctaggtgcatcgggataaatgtattgcttatattcagcaatataatgttcatcccgatatgcctagcccaaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 103 - RNA, 21 nt, Glycine max
  source 1..21: mol_type = mRNA
tgacagaagagagtgagcaca

SEQ ID NO: 104 - RNA, 21 nt, Glycine max
  source 1..21: mol_type = mRNA
ttgacagaagatagagagcac

SEQ ID NO: 105 - RNA, 21 nt, Glycine max
  source 1..21: mol_type = mRNA
tgacagaagagagtgagcact

SEQ ID NO: 106 - DNA, 21 bp, synthetic construct
  misc_feature 1..21: note = Description of Artificial Sequence: Synthetic oligonucleotide
  source 1..21: mol_type = other DNA
gtgctcactctcttctgtcaa

SEQ ID NO: 107 - RNA, 21 nt, Glycine max
  source 1..21: mol_type = mRNA
gtgctcactctcttctgtcaa

SEQ ID NO: 108 - DNA, 21 bp, synthetic construct
  misc_feature 1..21: note = Description of Artificial Sequence: Synthetic oligonucleotide
  source 1..21: mol_type = other DNA
tgtgctcactctcttctgtca

SEQ ID NO: 109 - DNA, 21 bp, synthetic construct
  misc_feature 1..21: note = Description of Artificial Sequence: Synthetic oligonucleotide
  source 1..21: mol_type = other DNA
gtgctctctctcttctgtcaa

SEQ ID NO: 110 - DNA, 21 bp, synthetic construct
  misc_feature 1..21: note = Description of Artificial Sequence: Synthetic oligonucleotide
  source 1..21: mol_type = other DNA
agtgctcactctcttctgtca